(19) United States
(12) Patent Application Publication
(10) Pub. No.: US 2024/0245691 A1
(43) Pub. Date: Jul. 25, 2024

(54) 2-METHYLENE-4-OXO-BUTANOIC ACID DERIVATIVES FOR THE TREATMENT OF INFLAMMATION

(71) Applicant: Sitryx Therapeutics Limited, Oxford (GB)

(72) Inventors: Matthew Colin Thor FYFE, Oxford (GB); Michael Liam COOKE, Nottingham, Nottinghamshire (GB); David COUSIN, Nottingham, Nottinghamshire (GB); Saleh AHMED, Nottingham, Nottinghamshire (GB); Alessandro MAZZACANI, Oxford (GB)

(73) Assignee: Sitryx Therapeutics Limited, Oxford (GB)

(21) Appl. No.: 18/557,183

(22) PCT Filed: Apr. 26, 2022
(86) PCT No.: PCT/GB22/51044

§ 371 Date: Oct. 25, 2023

(30) Foreign Application Priority Data
Apr. 26, 2021 (EP) 21170511
Dec. 22, 2021 (EP) 21217211

(51) Int. Cl. A61K 31/5375; A61K 31/18; A61K 31/192; A61K 31/337; A61K 31/38; A61K 31/41; A61K 31/4418; A61K 31/444; A61K 31/445; A61K 45/06; A61P 29/00; C07C 233/13; C07C 233/14; C07C 311/04; C07D 211/46; C07D 211/96; C07D 213/26; C07D 257/04; C07D 295/088; C07D 305/08; C07D 401/12; C07D 409/12

(52) U.S. Cl. CPC A61K 31/5375; A61K 31/18; A61K 31/192; A61K 31/337; A61K 31/38; A61K 31/41; A61K 31/4418; A61K 31/444; A61K 31/445; A61K 45/06; A61P 29/00; C07C 233/13; C07C 233/14; C07C 311/04; C07D 211/46; C07D 211/96; C07D 213/26; C07D 257/04; C07D 295/088; C07D 305/08; C07D 401/12; C07D 409/12

(57) ABSTRACT

The invention relates to compounds of formula (I) and to their use in treating or preventing an inflammatory disease or a disease associated with an undesirable immune response:

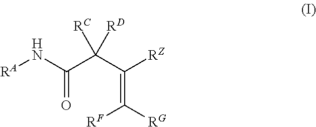

wherein R^{A}, R^{Z}, R^{C}, R^{D}, R^{F} and R^{G} are as defined herein.

### FIELD OF THE INVENTION

The present invention relates to compounds and their use in treating or preventing inflammatory diseases or diseases associated with an undesirable immune response, and to related compositions, methods and intermediate compounds.

### BACKGROUND OF THE INVENTION

Chronic inflammatory diseases such as rheumatoid arthritis, systemic lupus erythematosus (SLE), multiple sclerosis, psoriasis, Crohn's disease, ulcerative colitis, uveitis and chronic obstructive pulmonary disease (COPD) represent a significant burden to society because of life-long debilitating illness, increased mortality and high costs for therapy and care (Straub R. H. and Schradin C., 2016). Non-steroidal anti-inflammatory drugs (NSAIDs) are the most widespread medicines employed for treating inflammatory disorders, but these agents do not prevent the progression of the inflammation and only treat the accompanying symptoms. Glucocorticoids are powerful anti-inflammatory agents, making them emergency treatments for acute inflammatory flares, but given longer term these medicines give rise to a plethora of unwanted side-effects and may also be subject to resistance (Straub R. H. and Cutolo M., 2016). Thus, considerable unmet medical need still exists for the treatment of inflammatory disorders and extensive efforts to discover new medicines to alleviate the burden of these diseases is ongoing (Hanke T. et al., 2016).

Dimethyl fumarate (DMF), a diester of the citric acid cycle (CAC) intermediate fumaric acid, is utilised as an oral therapy for treating psoriasis (Brück J. et al., 2018) and multiple sclerosis (Mills E. A. et al., 2018). Importantly, following oral administration, none of this agent is detected in plasma (Dibbert S. et al., 2013), the only drug-related compounds observed being the hydrolysis product monomethyl fumarate (MMF) and glutathione (GSH) conjugates of both the parent (DMF) and metabolite (MMF). DMF's mechanism of action is complex and controversial. This compound's efficacy has been attributed to a multiplicity of different phenomena involving covalent modification of proteins and the conversion of “prodrug” DMF to MMF. In particular, the following pathways have been highlighted as being of relevance to DMF's anti-inflammatory effects: 1) activation of the anti-oxidant, anti-inflammatory, nuclear factor (erythroid-derived 2)-like 2 (NRF2) pathway as a consequence of reaction of the electrophilic α,β-unsaturated ester moiety with nucleophilic cysteine residues on kelch-like ECH-associated protein 1 (KEAP1) (Brennan M. S. et al., 2015); 2) induction of activating transcription factor 3 (ATF3), leading to suppression of pro-inflammatory cytokines interleukin (IL)-6 and IL-8 (Müller S. et al., 2017); 3) inactivation of the glycolytic enzyme glyceraldehyde 3-phosphate dehydrogenase (GAPDH) through succination of its catalytic cysteine residue with a Michael accepting unsaturated ester (Kornberg M. D. et al., 2018; Angiari S. and O'Neill L. A., 2018); 4) inhibition of nuclear factor-kappaB (NF-κB)-driven cytokine production (Gillard G. O. et al., 2015); 5) preventing the association of PKCθ with the costimulatory receptor CD28 to reduce the production of IL-2 and block T-cell activation (Blewett M. M. et al., 2016); 6) reaction of the electrophilic α,β-unsaturated ester with the nucleophilic thiol group of anti-oxidant GSH, impacting cellular responses to oxidative stress (Lehmann J. C. U. et al., 2007); 7) agonism of the hydroxycarboxylic acid receptor 2 (HCA2) by the MMF generated in vivo through DMF hydrolysis (von Glehn F. et al., 2018); 8) allosteric covalent inhibition of the p90 ribosomal S6 kinases (Andersen J. L. et al., 2018); 9) inhibition of the expression and function of hypoxia-inducible factor-1α (HIF-1α) and its target genes, such as IL-8 (Zhao G. et al., 2014); and 10) inhibition of Toll-like receptor (TLR)-induced M1 and K63 ubiquitin chain formation (McGuire V. A. et al., 2016). In general, with the exception of HCA2 agonism (Tang H. et al., 2008), membrane permeable diester DMF tends to exhibit much more profound biological effects in cells compared to its monoester counterpart MMF. However, the lack of systemic exposure of DMF in vivo has led some researchers to assert that MMF is, in fact, the principal active component following oral DMF administration (Mrowietz U. et al., 2018). As such, it is evident that some of the profound biology exerted by DMF in cells is lost because of hydrolysis in vivo to MMF.

Recently, it has been discovered that, during inflammatory macrophage activation, the CAC becomes anaplerotic and is diverted such that the unsaturated diacid itaconic acid, “itaconate”, is generated (Murphy M. P. and O'Neill L. A. J., 2018; O'Neill L. A. J. and Artyomov M. N., 2019; Yu X.-H. et al., 2019). Instead of being hydrated to isocitrate by aconitate hydratase, the CAC intermediate aconitate is decarboxylated by the protein product of immune-responsive gene 1 (IRG1), one of the most highly upregulated genes in macrophages under proinflammatory conditions, subsequently named aconitate decarboxylase 1, to produce itaconic acid (Michelucci A. et al., 2013). This unsaturated diacid is an inhibitor of the bacterial enzyme isocitrate lyase and, as such, it exerts anti-bacterial activity. In addition, itaconic acid has been shown to inhibit the CAC enzyme succinate dehydrogenase (SDH) (Ackermann et al., 1949), leading accordingly to succinate accumulation (Cordes T. et al., 2016). By inhibiting SDH, an enzyme critical for the inflammatory response (E. L. Mills et al., 2016), itaconate ameliorates inflammation in vitro and in vivo during macrophage activation and ischemia-reperfusion injury (Lampropoulou V. et al., 2016).

Like fumaric acid, itaconic acid is an α,β-unsaturated carboxylic acid. As such, it is a Michael acceptor which induces a global electrophilic stress response. In this regard, the itaconic acid diester dimethyl itaconate (DMI), like DMF, produces an anti-inflammatory response, reducing the expression levels of pro-inflammatory cytokines IL-1β, IL-6, IL-12 and IL-18 in lipopolysaccharide (LPS)-stimulated bone marrow-derived macrophages (WO2017/142855A1, incorporated herein by reference). This response appears to be mediated, in part, by NRF2 activation, via alkylation of KEAP1 cysteine residues by the electrophilic α,β-unsaturated ester moiety (Mills E. L. et al., 2018), which enhances the expression of downstream genes with anti-oxidant and anti-inflammatory capacities. Nevertheless, not all of the pronounced immunoregulatory effects engendered by DMI can be attributed to NRF2 activation. In particular, the modulation of IκBζ by DMI is independent of NRF2 and is mediated via upregulation of ATF3, a global negative regulator of immune activation that downregulates various cytokines, such as IL-6 (Bambouskova M. et al., 2018). Moreover, by inhibiting IκBζ protein production, DMI ameliorates IL-17-mediated pathologies, highlighting the therapeutic potential of this regulatory pathway (WO2019/036509A1, incorporated herein by reference). Further highlighting its pharmacologic potential, DMI has recently been reported to 1) demonstrate a protective effect on cerebral ischemia/reperfusion injury, thereby offering potential for the treatment of ischemic stroke (Zhang D. et al., 2019); 2) provide protection from the cardiotoxic effects of doxorubicin (Shan Q. et al., 2019); and 3) protect against lippolysacchride-induced mastitis in mice by activating MAPKs and NRFrf2 while inhibiting NF-κB signaling pathways (Zhao C. et al., 2019). Furthermore, DMI is said to have utility in preventing and treating ulcerative colitis and canceration thereof (CN110731955, Sun Yat-sen University Cancer Center); and has been reported to protect against fungal keratitis by activating the NRF2/HO-1 signalling pathway (Gu L. et al., 2020). Nevertheless, it should be noted that DMI is not metabolised to itaconic acid intracellularly (ElAzzouny M. et al., 2017). Other α,β-unsaturated esters exhibit IL-1β-lowering effects in macrophages by inhibiting the NLRP3 inflammasome (Cocco M. et al., 2017 and 2014), and have been demonstrated to inhibit the TLR4 pathway, leading ultimately to suppression of LPS-induced stimulation of NF-κB, tumour necrosis factor (TNF)-α, IL-1β and nitric oxide release (Zhang S. et al., 2012).

Other itaconic acid derivatives have been demonstrated to elicit anti-inflammatory effects (Bagavant G. et al., 1994). A notable example is 4-octyl itaconic acid (4OI), an itaconate derivative with improved cellular uptake. Since the α,β-unsaturated carboxylic acid is not esterified in 4OI, this electrophile exhibits low reactivity with biological thiols (Schmidt T. J. et al., 2007), much like the situation encountered with itaconic acid itself. As a result of its low reactivity/electrophilicity, the NRF2-activating effects of 4OI are not attenuated by GSH, in contrast to the findings with the much more reactive DMI. In this latter case, the α,β-unsaturated carboxylic acid is esterified and, as a consequence, the IL-6-lowering and NRF2-activating effects of DMI are reversed by the thiols N-acetylcysteine and GSH, respectively. Through the reaction with KEAP1 and the resulting NRF2 activation, as well as GAPDH inhibition (Liao S.-T. et al., 2019), 4OI has been demonstrated to produce a wide range of interesting biological effects, including: 1) protection of neuronal cells from hydrogen peroxide (Liu H. et al., 2018); 2) inhibition of proinflammatory cytokine production in peripheral blood mononuclear cells of SLE patients (Tang C. et al., 2018); and 3) protection of human umbilical vein endothelial cells from high glucose (Tang C. et al., 2019); 4) inhibition of osteoclastogenesis by suppressing the E3 ubiquitin ligase Hrd1 and activating NRF2 signaling (Sun X. et al., 2019); 5) induction of repression of STING by NRF2 and type I IFN production in cells from patients with STING-dependent interferonopathies (Olagnier D. et al., 2018); 6) protection against renal fibrosis via inhibiting the TGF-beta/Smad pathway, autophagy and reducing generation of reactive oxygen species (Tian F. et al., 2020); 7) reduction of brain viral burden in mice intracranially injected with Zika virus (Daniels B. P. et al. 2019); and 8) protection against liver ischemia-reperfusion injury (Yi F. et al. 2020). Furthermore, itaconate has been reported to modulate tricarboxylic acid and redox metabolism to mitigate reperfusion injury (Cordes T. et al., 2020). In addition, raised plasma itaconate levels demonstrate a clear correlation with reduction in rheumatoid arthritis disease activity scores following commencement of therapy with conventional disease modifying anti-rheumatic drug (cDMARD) therapy (Daly R. et al. 2019).

Artyomov et al. (WO2017/142855; WO2019/036509) disclose the use of itaconate, malonate or a derivative thereof as an immunomodulatory agent.

WO2020/222011 and WO2020/222010 (Sitryx Therapeutics) disclose certain itaconate derivatives.

In spite of the above findings, there remains a need to identify and develop new itaconate derivatives possessing enhanced properties compared to currently marketed anti-inflammatory agents, such as DMF. The present inventors have now discovered, surprisingly, that certain itaconate monoesters are highly effective at reducing cytokine release in cells. These properties, amongst others, make them potentially more effective than DMI and/or dimethyl fumarate. Such compounds therefore appear to possess excellent anti-inflammatory properties.

### SUMMARY OF THE INVENTION

The present invention provides a compound of formula (I):

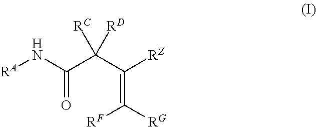

wherein:
R^{A} is selected from the group consisting of C₆₋₁₀ cycloalkyl optionally fused to phenyl, CH₂(phenyl) and CH₂(pyridyl);
wherein when R^{A} is C₆₋₁₀ cycloalkyl optionally fused to phenyl, the cycloalkyl ring is optionally substituted on an available carbon atom by one or more R^{A2}, wherein each R^{A2} is independently selected from the group consisting of methyl, halo and trifluoromethyl, and/or two R^{A2} are attached to the same carbon atom and join to form a C₃₋₆ cycloalkyl or a 4-6 membered heterocyclic ring; and the fused phenyl ring is optionally substituted on an available carbon atom by one or more substituents selected from the group consisting of C₁₋₄ haloalkyl and halo;
wherein when R^{A} is CH₂(phenyl) or CH₂(pyridyl):
   the CH₂ group is optionally substituted by one or two R^{A3} wherein each R^{A3} is independently selected from the group consisting of C₁₋₄ alkyl, C₃₋₅ cycloalkyl, C₁₋₄ haloalkyl and C₁₋₂ hydroxyalkyl, or two R^{A3} groups are attached to the CH₂ carbon atom and join to form a C₃₋₆ cycloalkyl or a 4- or 5-membered heterocyclic ring; and
   the phenyl or pyridyl group is optionally substituted by one or more R^{A4}, wherein each R^{A4} is independently selected from the group consisting of C₁₋₄ haloalkyl, halo, SF₅ or SC₁₋₄ haloalkyl;
R^{Z} is selected from the group consisting of C(O)OR^{B} and tetrazol-5-yl;
   R^{B} is selected from the group consisting of H; C₂₋₃ alkyl substituted with OH or N(R^{B2})(R^{B3}); and

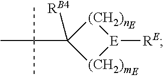

wherein:
   when R^{B} is C₂₋₃ alkyl substituted with N(R^{B2})(R^{B3}), R^{B2} is selected from H and methyl and R^{B3} is selected from methyl and SO₂(C₁₋₂ alkyl) or R^{B2} and R^{B3} together with the nitrogen atom to which they are attached, combine to form a 4- to 6-membered heterocyclic ring optionally containing a further heteroatom selected from O and N;
   wherein, when R^{B} is

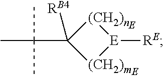

R^{B4} is H or methyl;
E is N, O or SO₂;
wherein when E is N, R^{E} is selected from the group consisting of R^{9B}, C(O)R^{9B} and SO₂R^{9B}; wherein R^{9B} is C₁₋₄ alkyl; and when E is O or SO₂, R^{E} is absent;
nE and mE are independently 1 or 2;
R^{C} and R^{D} are each independently H, C₁₋₂ alkyl, hydroxy, methoxy or fluoro;
each of R^{F} and R^{G} is independently selected from H, phenyl and C₁₋₄ alkyl optionally substituted with phenyl, OR^{K} or N(R^{K})(R^{L}), wherein each of R^{K} and R^{L} is independently selected from H, methyl and ethyl, provided that at least one of R^{F} and R^{G} is H; and
   wherein when the CH₂ of the CH₂(substituted phenyl) or CH₂(substituted pyridyl) of R^{A} is substituted by one R^{A3} the stereochemistry of the carbon to which R^{A3} is attached is as follows:

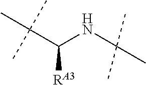

wherein the dashed lines indicate attachment to the remainder of the compound of formula (I); and wherein when R^{A4} is Cl, the CH₂ group is unsubstituted or is substituted by one R^{A3}; or a pharmaceutically acceptable salt and/or solvate thereof.

The present invention provides a pharmaceutical composition comprising a compound of formula (I) or a pharmaceutically acceptable salt and/or solvate thereof.

The present invention provides a compound of formula (I) or a pharmaceutically acceptable salt and/or solvate thereof for use as a medicament.

The present invention provides a compound of formula (I) or a pharmaceutically acceptable salt and/or solvate thereof for use in treating or preventing an inflammatory disease or a disease associated with an undesirable immune response.

The present invention provides the use of a compound of formula (I) or a pharmaceutically acceptable salt and/or solvate thereof in the manufacture of a medicament for treating or preventing an inflammatory disease or a disease associated with an immune response.

The present invention provides a method of treating or preventing an inflammatory disease or a disease associated with an undesirable immune response, which comprises administering a compound of formula (I) or a pharmaceutically acceptable salt and/or solvate thereof.

Also provided are intermediate compounds of use in the preparation of compounds of formula (I).

### DETAILED DESCRIPTION OF THE INVENTION

### Compounds of Formula (I)

Embodiments and preferences set out herein with respect to the compound of formula (I) apply equally to the pharmaceutical composition, compound or salt and/or solvate thereof for use, use and method aspects of the invention.

Embodiments and preferences for one variable in the compound of formula (I) (e.g. R^{A}) may be combined with embodiments and preferences for other variables in the compound of formula (I) (e.g. R^{B}, R^{C}, R^{D}, R^{F} and R^{G}).

In one embodiment, there is provided a compound of formula (I) as described above.

The compound of formula (I) may be a compound of formula (Ia):

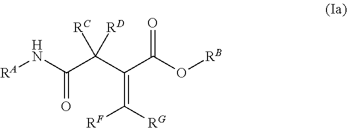

wherein:
R^{A} is selected from the group consisting of C₆₋₁₀ cycloalkyl optionally fused to phenyl, CH₂(phenyl) and CH₂(pyridyl);
wherein when R^{A} is C₆₋₁₀ cycloalkyl optionally fused to phenyl, the cycloalkyl ring is optionally substituted on an available carbon atom by one or more R^{A2}, wherein R^{A2} is selected from the group consisting of methyl, halo and trifluoromethyl, and/or two R^{A2} are attached to the same carbon atom and join to form a C₃₋₆ cycloalkyl or a 4-6 membered heterocyclic ring; and the fused phenyl ring is optionally substituted on an available carbon atom by one or more substituents selected from the group consisting of C₁₋₄ haloalkyl and halo;
wherein when R^{A} is CH₂(phenyl) or CH₂(pyridyl):
the CH₂ group is optionally substituted by one or two R^{A3} wherein R^{A3} is selected from the group consisting of C₁₋₄ alkyl, C₃₋₅ cycloalkyl, C₁₋₄ haloalkyl and C₁₋₂ hydroxyalkyl, or two R^{A3} groups are attached to the CH₂ carbon atom and join to form a C₃₋₆ cycloalkyl or a 4- or 5-membered heterocyclic ring; and
the phenyl or pyridyl group is optionally substituted by one or more R^{A4}, wherein R^{A4} is selected from the group consisting of C₁₋₄ haloalkyl, halo, or SC₁₋₄ haloalkyl;
R^{B} is selected from the group consisting of H; C₂₋₃ alkyl substituted with OH or N(R^{B2})(R^{B3}); and

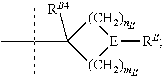

wherein:
when R^{B} is C₂₋₃ alkyl substituted with N(R^{B2})(R^{B3}) R^{B2} is selected from H and methyl and R^{B3} is selected from methyl and SO₂(C₁₋₂ alkyl) or R^{B2} and R^{B3}, together with the nitrogen atom to which they are attached, combine to form a 4- to 6-membered heterocyclic ring optionally containing a further heteroatom selected from O and N;
wherein, when R^{B} is

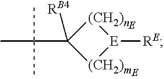

R^{B4} is H or methyl;
E is N, O or SO₂;
wherein when E is N, R^{E} is selected from the group consisting of R^{9B}, C(O)R^{9B} and SO₂R^{9B}; wherein R^{9B} is C₁₋₄ alkyl; and when E is O or SO₂, R^{E} is absent;
nE and mE are independently 1 or 2;
R^{C} and R^{D} are each independently H, C₁₋₂ alkyl, hydroxy, methoxy or fluoro;
each of R^{F} and R^{G} is independently H or methyl, provided that at least one of R^{F} and R^{G} is H; and wherein when the CH₂ of the CH₂(substituted phenyl) or CH₂(substituted pyridyl) of R^{A} is substituted by one R^{A3} the stereochemistry of the carbon to which R^{A3} is attached is as follows:

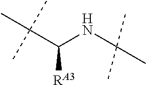

wherein the dashed lines indicate attachment to the remainder of the compound of formula (I); and wherein when R^{A4} is Cl, the CH₂ group is unsubstituted or is substituted by one R^{A3}; and
wherein, when R^{B} is H, R^{A} is not unsubstituted cyclohexyl;
or a pharmaceutically acceptable salt and/or solvate thereof.

Alternatively, the compound of formula (I) may be a compound of formula (Ib):

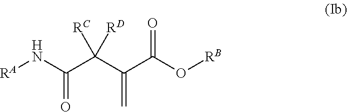

wherein:
R^{A} is selected from the group consisting of C₆₋₁₀ cycloalkyl and CH₂phenyl;
wherein when R^{A} is C₆₋₁₀ cycloalkyl, R^{A} is optionally substituted on an available carbon atom by one or more R^{A2}, wherein R^{A2} is selected from the group consisting of methyl, halo and trifluoromethyl, and/or two R^{A2} are attached to the same carbon atom and join to form a C₃₋₆ cycloalkyl or a 4-6 membered heterocyclic ring;
wherein when R^{A} is CH₂phenyl:
   the CH₂ group is optionally substituted by one or two R^{A3} wherein R^{A3} is selected from the group consisting of C₁₋₄ alkyl, C₃₋₅ cycloalkyl, C₁₋₄ haloalkyl and C₁₋₂ hydroxyalkyl, or two R^{A3} groups are attached to the CH₂ carbon atom and join to form a C₃₋₆ cycloalkyl or a 4-6 membered heterocyclic ring; and
   the phenyl group is optionally substituted by one or more R^{A4}, wherein one R^{A4} is in the 4-position, wherein R^{A4} is selected from the group consisting of C₁₋₄ haloalkyl, halo, or SC₁₋₄ haloalkyl;
R^{B} is selected from the group consisting of H and

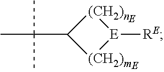

wherein:
   E is N or O;
   wherein when E is N, R^{E} is SO₂R^{9B}; wherein R^{9B} is C₁₋₄ alkyl; and when E is O, R^{E} is absent;
   nE and mE are independently 1 or 2;
R^{C} and R^{D} are each independently H, C₁₋₂ alkyl, hydroxy, methoxy or fluoro; and
   wherein when the CH₂ of the CH₂phenyl is substituted by one R^{A3} the stereochemistry of the carbon to which R^{A3} is attached is as follows:

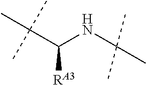

wherein the dashed lines indicate attachment to the remainder of the compound of formula (I); and wherein when R^{A4} is Cl, the CH₂ group is unsubstituted or is substituted by one R^{A3};
or a pharmaceutically acceptable salt and/or solvate thereof.

The term “C₁₋₄ alkyl” refers to a straight or branched fully saturated hydrocarbon group having from 1 to 4 carbon atoms. The term encompasses methyl, ethyl, n-propyl, isopropyl, n-butyl, isobutyl, sec-butyl and tert-butyl. Other alkyl groups, for example C₁₋₄ alkyl, C₁₋₃ alkyl and C₁₋₂ alkyl are as defined above but contain different numbers of carbon atoms. The term “C₁₋₄ alkyl” also encompasses “C₁₋₄ alkylene” which is a bifunctional straight or branched fully saturated hydrocarbon group having from 1 to 4 carbon atoms. Example “C₁₋₄ alkylene” groups include methylene, ethylene, n-propylene and n-butylene.

The term “C₁₋₄ haloalkyl” (e.g. C₁₋₃ haloalkyl group, C₁₋₂ haloalkyl group or C, haloalkyl group) as used herein refers to a straight or a branched fully saturated hydrocarbon chain containing the specified number of carbon atoms and at least one halogen atom, such as fluoro or chloro, especially fluoro. An example of haloalkyl is CF₃. Further examples of haloalkyl are CHF₂ and CH₂CF₃.

The term “C₃₋₁₀ cycloalkyl” (such as C₃₋₅ cycloalkyl, C₃₋₆ cycloalkyl and C₆₋₁₀ cycloalkyl) refers to a fully saturated cyclic hydrocarbon group having from 3 to 10 carbon atoms. The term encompasses cyclopropyl, cyclobutyl, cyclopentyl, cyclohexyl, cycloheptyl and cyclooctyl as well as bridged systems such as bicyclo[1.1.1]pentyl.

The term “4-6 membered heterocyclic ring” refers to a non-aromatic cyclic group having 4 to 6 ring atoms and at least one heteroatom selected from N, O, S and B. The term “heterocyclic ring” is interchangeable with “heterocyclyl”. The term encompasses oxetanyl, thietanyl, azetidinyl, pyrrolidinyl, tetrahydrofuranyl, tetrahydrothienyl, tetrahydropyranyl, piperidinyl, piperazinyl, morpholinyl and thiomorpholinyl. Other heterocyclyl groups, for example, 4-5 membered heterocyclyl are as defined above but contain different numbers of ring atoms. 4-6 membered heterocyclyl groups can typically be substituted by one or more oxo groups. Suitably, thietanyl is substituted by one or two oxo groups. Bicyclic heterocyclic compounds are also encompassed.

The term “hydroxy” (which may also be referred to as “hydroxyl”) refers to an —OH group.

The term “C₁₋₂ hydroxyalkyl” refers to an alkyl or alkylene chain having one or two carbon atoms, wherein one of the carbon atoms is substituted by an —OH group. Examples include —CH₂C(H)OH, —C(H)OHCH₃ and —C(H)OH.

The term “oxo” refers to a ═O substituent, whereby an oxygen atom is doubly bonded to carbon (e.g. C═O) or another element (e.g. S═O, S(═O)₂). The carbon or other element is suitably an atom of an alkyl, cycloalkyl or heterocyclyl group.

The term “halo” refers to fluorine, chlorine, bromine or iodine. Particular examples of halo are fluorine and bromine, especially fluorine.

Where substituents are indicated as being optionally substituted in formula (I) in the embodiments and preferences set out below, the optional substituent may be attached to an available carbon atom, which means a carbon atom which is attached to a hydrogen atom i.e. a C—H group. The optional substituent replaces the hydrogen atom attached to the carbon atom.

Where an entity is substituted by more than one substituent, e.g. two or more R^{A2}, R^{A3} or R^{A4} substituents, the substituents are independent of one another. For example a first R^{A4} substituent may be the same as or different from a second R^{A4} substituent.

In the compounds of formula (I), it is preferred that when R^{A} is CH₂(phenyl) or CH₂(pyridyl), the phenyl or pyridyl group is substituted by one or more R^{A4}, i.e R^{A} is CH₂(substituted phenyl) or CH₂(substituted pyridyl). Suitably, when R^{A} is CH₂(substituted phenyl) or CH₂(substituted pyridyl), one R^{A4} is in the para-position with respect to the linkage to the remainder of the molecule.

In one embodiment, R^{A} is C₆₋₁₀ cycloalkyl optionally substituted on an available carbon atom by one or more R^{A2}.

Suitably, in compounds of formula (I), R^{A} is cyclooctyl optionally substituted on an available carbon atom by one or more R^{A2}.

In one embodiment, when R^{B} is H, R^{A} is not unsubstituted cyclohexyl or unsubstituted benzyl. In one embodiment, when R^{B}, R^{F} and R^{G} are all H, R^{A} is not unsubstituted cyclohexyl. In one embodiment, when R^{B}, R^{C}, R^{D}, R^{F} and R^{G} are all H, R^{A} is not unsubstituted cyclohexyl.

In one embodiment, R^{A} is unsubstituted C₆₋₁₀ cycloalkyl. In another embodiment, R^{A} is unsubstituted C₇₋₁₀ cycloalkyl. In another embodiment, R^{A} is C₆₋₁₀ cycloalkyl substituted on an available carbon atom by one or more (such as one, two or three e.g. one) R^{A2}.

In still another embodiment, R^{A} is C₆₋₁₀ cycloalkyl fused to phenyl, wherein the cycloalkyl ring is optionally substituted on an available carbon atom by one or more R^{A2}; and wherein the phenyl ring is unsubstituted or is substituted on an available carbon atom by one or more substituents independently selected from the group consisting of C₁₋₄ haloalkyl and halo, for example, the phenyl ring is substituted on an available carbon atom by a trifluoromethyl substituent.

In one embodiment, R^{A2} is methyl. In a second embodiment, R^{A2} is halo. In a third embodiment, R^{A2} is trifluoromethyl. In a fourth embodiment, two R^{A2} are attached to the same carbon atom and join to form a C₃₋₆ cycloalkyl or a 4-6 membered heterocyclic ring. Suitably, two R^{A2} are attached to the same carbon atom and join to form a C₃₋₆ cycloalkyl ring. Alternatively, two R^{A2} are attached to the same carbon atom and join to form a 4-6 membered heterocyclic ring.

In one embodiment, R^{A} is CH₂(phenyl), especially CH₂(substituted phenyl). In another embodiment, R^{A} is CH₂(pyridyl), especially CH₂(substituted pyridyl).

Suitably, the CH₂ group is optionally substituted by one or two R^{A3}. In one embodiment, the CH₂ group is not substituted. In a second embodiment, the CH₂ group is substituted by one R^{A3}. In a third embodiment, the CH₂ group is substituted by two R^{A3}.

In one embodiment, R^{A3} is C₁₋₄ alkyl, such as methyl or ethyl. In a second embodiment, R^{A3} is C₃₋₅ cycloalkyl, such as cyclopropyl. In a third embodiment, R^{A3} is C₁₋₄ haloalkyl, such as trifluoromethyl. In a fourth embodiment, R^{A3} is C₁₋₂ hydroxyalkyl, such as CH₂OH. In a fifth embodiment, two R^{A3} groups are attached to the CH₂ carbon atom and join to form a C₃₋₆ cycloalkyl or a 4-6, more suitably a 4- or 5-membered heterocyclic ring. Suitably, two R^{A3} groups are attached to the CH₂ carbon atom and join to form a C₃₋₆ cycloalkyl (such as cyclopropyl, cyclobutyl or cyclopentyl, especially cyclopropyl or cyclobutyl). Alternatively, two R^{A3} groups are attached to the CH₂ carbon atom and join to form a 4-6, more suitably a 4- or 5-membered heterocyclic ring (such as oxetanyl).

Suitably, the CH₂ group is substituted by two R^{A3} and each R^{A3} is C₁₋₄ alkyl, such as methyl.

When the CH₂ is substituted by one R^{A3}, the stereochemistry of the carbon to which R^{A3} is attached is as follows:

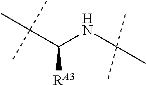

wherein the dashed lines indicate attachment to the remainder of the compound of formula (I).

In some compounds of formula (I), R^{A} is CH₂(phenyl) and the phenyl group is unsubstituted. In more suitable compounds of formula (I), the phenyl group is substituted by one or more (such as one, two or three e.g. one) R^{A4}.

When R^{A} is CH₂(substituted phenyl), the phenyl group is substituted by one or more R^{A4}, wherein one R^{A4} is suitably in the 4-position (i.e. the para-position with respect to the linkage to the remainder of the molecule):

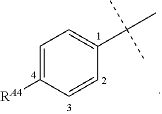

In some compounds of formula (I), R^{A} is CH₂(pyridyl) and the pyridyl group is unsubstituted. In more suitable compounds of formula (I), the pyridyl group is substituted by one or more (such as one, two or three e.g. one) R^{A4}.

When R^{A} is CH₂(substituted pyridyl), the pyridyl group is suitably substituted by one or more R^{A4}, wherein one R^{A4} is suitably in the para-position with respect to the linkage to the remainder of the molecule, that is, at the position on the ring that is furthest away from the attachment to the rest of the molecule. The pyridyl group may be, for example a 2-pyridyl group:

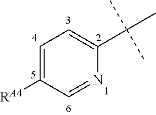

or a 3-pyridyl group:

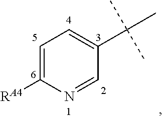

especially 2-pyridyl.

Suitably, the pyridyl group is substituted by one or more (such as one, two or three e.g. one) R^{A4}.

In one embodiment, when R^{A} is CH₂(substituted phenyl) or CH₂(substituted pyridyl), the phenyl or pyridyl group is substituted by one R^{A4}. As noted above, the R^{A4} substituent is suitably in the para-position with respect to the linkage to the remainder of the molecule.

Alternatively, when R^{A} is CH₂(substituted phenyl) or CH₂(substituted pyridyl), the phenyl or pyridyl group may be substituted by two R^{A4}, one of which is in the para-position with respect to the linkage to the remainder of the molecule as shown above. In this case, it is preferred that the second R^{A4} substituent is in the meta position with respect to the linkage to the remainder of the molecule.

In one embodiment, R^{A4} is C₁₋₄ haloalkyl such as CHF₂, CF₃CF₂ or CF₃, especially CF₃. When R^{A} is CH₂(substituted pyridyl) and R^{A4} is C₁₋₄ haloalkyl, R^{A4} is suitably a perhaloalkyl group. In a second embodiment, R^{A4} is halo such as fluoro, chloro or bromo, especially fluoro or bromo. In a third embodiment, R^{A4} is SC₁₋₄ haloalkyl. In a fourth embodiment, R^{A4} is SF₅. In a further embodiment, a first R^{A4} is C₁₋₄ haloalkyl and a second R^{A4} is halo.

Suitably, when R^{A4} is Cl, the CH₂ group is unsubstituted or is substituted by one R^{A3} wherein R^{A3} is defined above.

In some compounds of formula (I), R^{Z} is C(O)OR^{B}.

In one embodiment, R^{B} is H.

In a second embodiment, R^{B} is C₂₋₃ alkyl substituted with OH or N(R^{B2})(R^{B3}),

Suitably in these compounds, R^{B2} is H and R^{B3} is SO₂(C₁₋₂ alkyl) or, alternatively, R^{B2} is H or methyl, especially methyl, and R^{B3} is methyl. In further suitable compounds, R^{B2} and R^{B3} together with the nitrogen atom to which they are attached combine to form a 4- to 6-membered heterocyclic ring, especially a 6-membered heterocyclic ring optionally containing a further heteroatom selected from O and N. For example, R^{B2} and R^{B3} together with the nitrogen atom to which they are attached may combine to form a morpholine ring.

In a further embodiment, R^{B} is

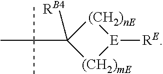

In some compounds of this embodiment, R^{B4} is H and in other compounds of this embodiment, R^{B4} is methyl.

In one embodiment, E is N. In a second embodiment, E is O; and in a third embodiment, E is SO₂.

In one embodiment, R^{E} is absent such as when E is O or SO₂. In a second embodiment R^{E} is R^{9B} C(O)R^{9B} or SO₂R^{9B} such as when E is N.

In a one embodiment, R^{9B} is C₁₋₄ alkyl such as methyl.

In one embodiment, nE is 1 or 2. In one embodiment, mE is 1 or 2.

Suitably, nE is 1 or 2, mE is 1 or 2, E is N and R^{E} is SO₂R^{9B}, wherein R^{9B} is methyl.

In alternative suitable compounds, nE is 2, mE is 2, E is N and R^{E} is R^{9B}, wherein R^{9B} is methyl.

In alternative suitable compounds, nE is 1 or 2, mE is 1 or 2, E is N and R^{E} is C(O)R^{9B}, wherein R^{9B} is methyl.

In further suitable compounds, nE is 1, mE is 1 and E is O or SO₂.

In a further embodiment, R^{B4} is H, such that R^{B} is:

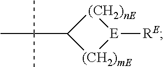

wherein mE and nE are as defined above and E is N or O;
wherein when E is N, R^{E} is SO₂R^{9B}; wherein R^{9B} is C₁₋₄ alkyl; and when E is O, R^{E} is absent.

In an alternative embodiment, R^{Z} is tetrazol-5-yl, i.e the tetrazole is joined to the remainder of the molecule via the ring carbon atom.

In one embodiment, R^{C} is H. In a second embodiment, R^{C} is C₁₋₂ alkyl, in particular methyl. In a third embodiment, R^{C} is hydroxy. In a fourth embodiment, R^{C} is methoxy. In a fifth embodiment, R^{C} is fluoro.

In one embodiment, R^{D} is H. In a second embodiment, R^{D} is C₁₋₂ alkyl, in particular methyl. In a third embodiment, R^{D} is hydroxy. In a fourth embodiment, R^{D} is methoxy. In a fifth embodiment, R^{D} is fluoro.

Suitably, R^{C} is H and R^{D} is H.

In one embodiment, R^{F} is H and R^{G} is H.

In another embodiment, each of R^{F} and R^{G} is independently H or methyl, provided that at least one of R^{F} and R^{G} is H.

In an alternative embodiment, R^{F} is phenyl or C₁₋₄ alkyl optionally substituted with phenyl, OR^{K} or N(R^{K})(R^{L}) and R^{G} is H; and in a further embodiment, R^{F} is H and R^{G} is phenyl or C₁₋₄ alkyl optionally substituted with phenyl, OR^{K} or N(R^{K})(R^{L}), wherein R^{K} and R^{L} are as defined above for Formula (I).

Suitably, in embodiments where one of R^{F} and R^{G} is other than H, there is an E (trans) configuration at the double bond such that R^{F} is phenyl or C₁₋₄ alkyl optionally substituted with phenyl, OR^{K} or N(R^{K})(R^{L}) and R^{G} is H and the compound is a compound of formula (Ic):

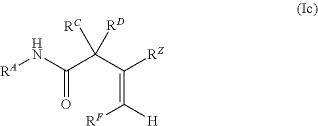

wherein R^{F} is phenyl or C₁₋₄ alkyl optionally substituted with phenyl, OR^{K} or N(R^{K})(R^{L}) and R^{A}, R^{C}, R^{D} and R^{Z} are as defined for formula (I).

Suitably, in embodiments where one of R^{F} and R^{G} is other than H, either R^{F} is phenyl or C₁₋₄ alkyl optionally substituted with phenyl or N(R^{K})(R^{L}) and R^{G} is H; or R^{F} is H and R^{G} is phenyl or C₁₋₄ alkyl optionally substituted with phenyl or N(R^{K})(R^{L}).

R^{K} and R^{L} are more suitably each independently H or methyl, especially methyl.

In some compounds of the invention, R^{Z} is C(O)OR^{B}, R^{F} is phenyl or C₁₋₄ alkyl optionally substituted with phenyl, OR^{K} or N(R^{K})(R^{L}), R^{G} is H and the compound is a compound of formula (Id):

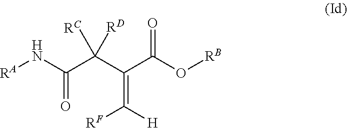

wherein R^{F} is phenyl or C₁₋₄ alkyl optionally substituted with phenyl, OR^{K} or N(R^{K})(R^{L}) and R^{A}, R^{C}, R^{D} and R^{B} are as defined for formula (I).

In one embodiment there is provided a compound of formula (I), which is:
1-(methylsulfonyl)piperidin-4-yl 4-((4-chlorobenzyl)amino)-2-methylene-4-oxobutanoate;
1-(methylsulfonyl)piperidin-4-yl 4-(cyclooctylamino)-2-methylene-4-oxobutanoate;
(S)-2-methylene-4-oxo-4-((1-(4-(trifluoromethyl)phenyl)ethyl)amino)butanoic acid;
2-methylene-4-oxo-4-((2-(4-(trifluoromethyl)phenyl)propan-2-yl)amino)butanoic acid;
2-methylene-4-oxo-4-((1-(4-(trifluoromethyl)phenyl)cyclobutyl)amino)butanoic acid;
2-methylene-4-oxo-4-((4-(trifluoromethyl)benzyl)amino)butanoic acid;
2-methylene-4-oxo-4-((2,2,2-trifluoro-1-(4-(trifluoromethyl)phenyl)ethyl)amino)butanoic acid;
(S)-2-methylene-4-oxo-4-((1-(4-(trifluoromethyl)phenyl)propyl)amino)butanoic acid;
1-(methylsulfonyl)piperidin-4-yl 2-methylene-4-oxo-4-((1-(4-(trifluoromethyl)phenyl)cyclobutyl) amino)butanoate;
4-((cyclopropyl(4-(trifluoromethyl)phenyl)methyl)amino)-2-methylene-4-oxobutanoic acid;
2-methylene-4-oxo-4-((1-(4-(trifluoromethyl)phenyl)cyclopropyl)amino)butanoic acid;
(R)-4-((2-hydroxy-1-(4-(trifluoromethyl)phenyl)ethyl)amino)-2-methylene-4-oxobutanoic acid;
2-methylene-4-oxo-4-((3-(4-(trifluoromethyl)phenyl)oxetan-3-yl)amino)butanoic acid;
2-methylene-4-oxo-4-((1-(4-(trifluoromethyl)phenyl)cyclopentyl)amino)butanoic acid;
(S)-4-((1-(4-chlorophenyl)ethyl)amino)-2-methylene-4-oxobutanoic acid;
(S)-4-((1-(4-bromophenyl)ethyl)amino)-2-methylene-4-oxobutanoic acid;
(S)-4-((1-(4-bromophenyl)ethyl)amino)-2-methylene-4-oxobutanoic acid;
(S)-4-((1-(4-(difluoromethyl)phenyl)ethyl)amino)-2-methylene-4-oxobutanoic acid;
(S)-4-((1-(3-fluoro-4-(trifluoromethyl)phenyl)ethyl)amino)-2-methylene-4-oxobutanoic acid;
(S)-2-methylene-4-oxo-4-((1-(4-(perfluoroethyl)phenyl)ethyl)amino)butanoic acid;
(R)-2-methylene-4-oxo-4-((2,2,2-trifluoro-1-(4-(trifluoromethyl)phenyl)ethyl)amino)butanoic acid;
1-(methylsulfonyl)piperidin-4-yl (S)-2-methylene-4-oxo-4-((1-(4-(trifluoromethyl)phenyl)ethyl)amino)butanoate;
4-((cyclopropyl(4-(trifluoromethyl)phenyl)methyl)amino)-2-methylene-4-oxobutanoic acid;
1-methylpiperidin-4-yl (S)-2-methylene-4-oxo-4-((1-(4-(trifluoromethyl)phenyl)ethyl)amino)butanoate;
2-(methylsulfonamido)ethyl (S)-2-methylene-4-oxo-4-((1-(4-(trifluoromethyl)phenyl)ethyl)amino)butanoate;
2-(dimethylamino)ethyl (S)-2-methylene-4-oxo-4-((1-(4-(trifluoromethyl)phenyl)ethyl)amino)butanoate;
1,1-dioxidothietan-3-yl (S)-2-methylene-4-oxo-4-((1-(4-(trifluoromethyl)phenyl)ethyl)amino)butanoate;
3-methyloxetan-3-yl (S)-2-methylene-4-oxo-4-((1-(4-(trifluoromethyl)phenyl)ethyl)amino)butanoate;
2-morpholinoethyl 2-(diethoxyphosphoryl)-4-oxo-4-(((S)-1-(4-(trifluoromethyl)phenyl)ethyl)amino)butanoate;
(S)-2-methylene-4-oxo-4-((1-(5-(trifluoromethyl)pyridin-2-yl)ethyl)amino)butanoic acid;
1-(methylsulfonyl)piperidin-4-yl (S)-2-methylene-4-oxo-4-((1-(5-(trifluoromethyl)pyridin-2-yl)ethyl)amino)butanoate;
1-methylpiperidin-4-yl (S)-2-methylene-4-oxo-4-((1-(5-(trifluoromethyl)pyridin-2-yl)ethyl)amino)butanoate;
1,1-dioxidothietan-3-yl (S)-2-methylene-4-oxo-4-((1-(5-(trifluoromethyl)pyridin-2-yl)ethyl)amino)butanoate;
2-hydroxyethyl (S)-2-methylene-4-oxo-4-((1-(5-(trifluoromethyl)pyridin-2-yl)ethyl)amino)butanoate;
1-acetylpiperidin-4-yl (S)-2-methylene-4-oxo-4-((1-(5-(trifluoromethyl)pyridin-2-yl)ethyl)amino)butanoate;
1-acetylazetidin-3-yl (S)-2-methylene-4-oxo-4-((1-(5-(trifluoromethyl)pyridin-2-yl)ethyl)amino)butanoate;
1-(methylsulfonyl)azetidin-3-yl (S)-2-methylene-4-oxo-4-((1-(5-(trifluoromethyl)pyridin-2-yl)ethyl)amino)butanoate;
(S)-2-methylene-4-oxo-4-((1-(6-(trifluoromethyl)pyridin-3-yl)ethyl)amino)butanoic acid;
2-methylene-4-oxo-4-((1-(5-(trifluoromethyl)pyridin-2-yl)cyclobutyl)amino)butanoic acid;
2-methylene-4-oxo-4-((5-(trifluoromethyl)-2,3-dihydro-1H-inden-1-yl)amino)butanoic acid;
(S,Z)-2-(2-oxo-2-((1-(4-(trifluoromethyl)phenyl)ethyl)amino)ethyl)but-2-enoic acid; and
(S,E)-2-(2-oxo-2-((1-(4-(trifluoromethyl)phenyl)ethyl)amino)ethyl)but-2-enoic acid; (S,E)-2-(2-oxo-2-((1-(4-(trifluoromethyl)phenyl)ethyl)amino)ethyl)pent-2-enoic acid;
(S,E)-4-methyl-2-(2-oxo-2-((1-(4-(trifluoromethyl)phenyl)ethyl)amino)ethyl)pent-2-enoic acid;
(S,E)-5-methyl-2-(2-oxo-2-((1-(4-(trifluoromethyl)phenyl)ethyl)amino)ethyl)hex-2-enoic acid;
(S,E)-2-(2-oxo-2-((1-(4-(trifluoromethyl)phenyl)ethyl)amino)ethyl)-5-phenylpent-2-enoic acid;
(S,E)-2-benzylidene-4-oxo-4-((1-(4-(trifluoromethyl)phenyl)ethyl)amino)butanoic acid;
(S,Z)-2-benzylidene-4-oxo-4-((1-(4-(trifluoromethyl)phenyl)ethyl)amino)butanoic acid;
(S,E)-4-(dimethylamino)-2-(2-oxo-2-((1-(4-(trifluoromethyl)phenyl)ethyl)amino)ethyl)but-2-enoic acid;
(S)-3,3-dimethyl-2-methylene-4-oxo-4-((1-(4-(trifluoromethyl)phenyl)ethyl)amino)butanoic acid;
3-methyl-2-methylene-4-oxo-4-((2-(4-(trifluoromethyl)phenyl)propan-2-yl)amino)butanoic acid;
3-methyl-2-methylene-4-oxo-4-((2-(4-(trifluoromethyl)phenyl)propan-2-yl)amino)butanoic acid ISOMER 1;
3-methyl-2-methylene-4-oxo-4-((2-(4-(trifluoromethyl)phenyl)propan-2-yl)amino)butanoic acid ISOMER 2;
3-methyl-2-methylene-4-oxo-4-(((S)-1-(4-(trifluoromethyl)phenyl)ethyl)amino)butanoic acid ISOMER 1;
3-methyl-2-methylene-4-oxo-4-(((S)-1-(4-(trifluoromethyl)phenyl)ethyl)amino)butanoic acid;
(S)-2-methylene-4-oxo-4-((1-(4-(pentafluoro-A6-sulfaneyl)phenyl)ethyl)amino)butanoic acid
(S)-3-(1H-tetrazol-5-yl)-N-(1-(4-(trifluoromethyl)phenyl)ethyl)but-3-enamide;
   or a pharmaceutically acceptable salt and/or solvate of any one thereof.

Compounds of formula (I) may be synthesised as shown in the schemes below and as shown in the Example section.

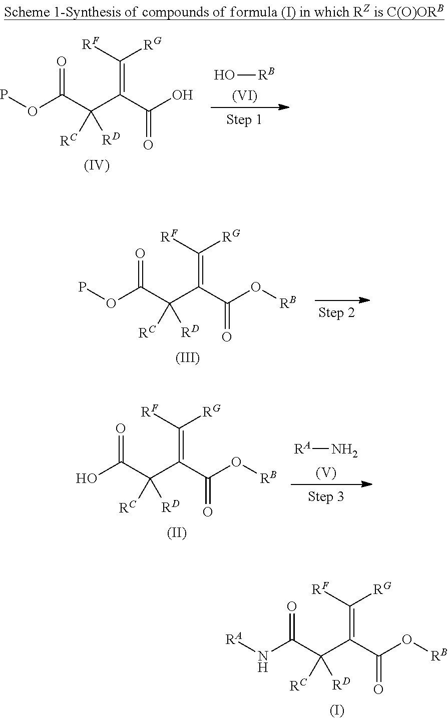

wherein R^{B} is as defined elsewhere herein except that it is not H, and R^{A}, R^{C} and R^{D} are defined elsewhere herein and P is a carboxylic acid protecting group, such as para-methoxybenzyl (PMB).
Step 1: Compounds of formula (IV) may be reacted with alcohols of formula (VI), using coupling conditions such as EDC·HCl/DMAP in the presence of a base such as DIPEA, to provide compounds of formula (III).
Step 2: Compounds of formula (III) may be converted to compounds of formula (II) by removal of protecting group P. For example, if P is PMB, P is removed using acidic conditions, such as TFA in DCM.
Step 3: Compounds of formula (II) may be converted to compounds of formula (I) by a coupling reaction using amines of formula (V) under standard coupling conditions, such as HATU and NMM.

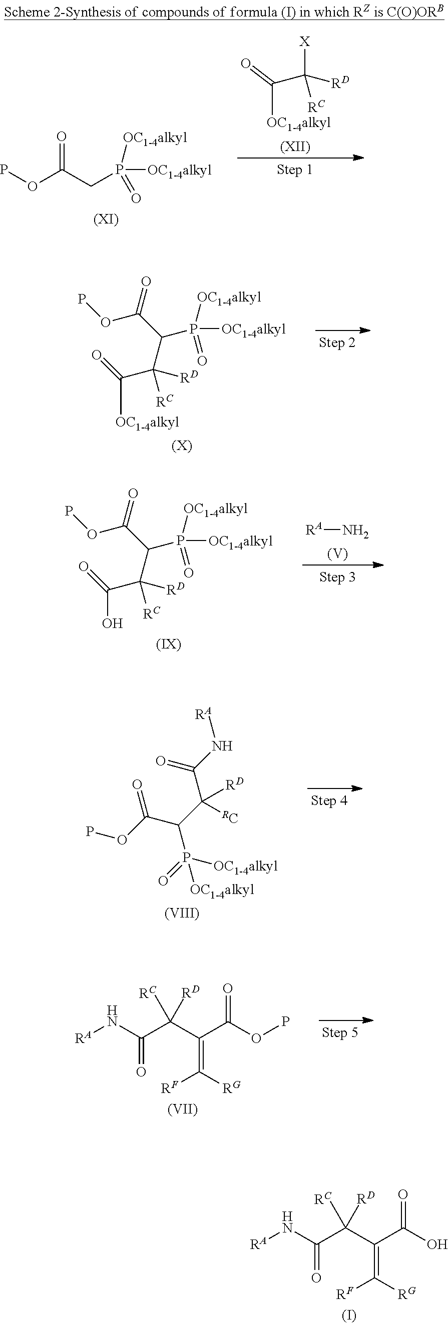

Wherein R^{B} is H, R^{A}, R^{C}, R^{D}, R^{F} and R^{G} are defined elsewhere herein, X is halo, such as Br, and P is a carboxylic acid protecting group such as C₁₋₄alkyl, e.g., tBu.
Step 1: Compounds of formula (XI) are reacted under basic conditions (such as NaH in THF) with esters of formula (XII) to give compounds of formula (X).
Step 2: The esters of formula (X) are hydrolysed under basic conditions (such as 1M NaOH in THF) to give carboxylic acids of formula (IX).
Step 3: Acids of formula (IX) may be converted to amides of formula (VIII) using amines of formula (V) under standard amide coupling conditions, such as with coupling agent HATU, and a base, such as DIPEA.
Step 4: Phosphonates of formula (VIII) may be converted to enoates of formula (VII) using the Horner-Wadsworth-Emmons modification of the Wittig reaction, such as by using aqueous C(R^{F})(R^{G})═O in the presence of a base such as K₂CO₃.
Step 5: Esters of formula (VII) may be converted to compounds of formula (I) by removal of protecting group P using conditions known to the skilled person. For example, if P is C₁₋₄alkyl, e.g., tBu, P may be removed under acidic conditions, such as TFA in DCM. Compounds of formula (I) in Scheme 2, wherein R^{B} is H, may be converted to compounds of formula (I) wherein R^{B} is other than H using analogous methods to those shown in Scheme 1, i.e., reacting the compound of formula (I) (R^{B}═H) with alcohol HO—R^{B} (VI), where in R^{B} is as defined above for formula (I) except that it is not H.

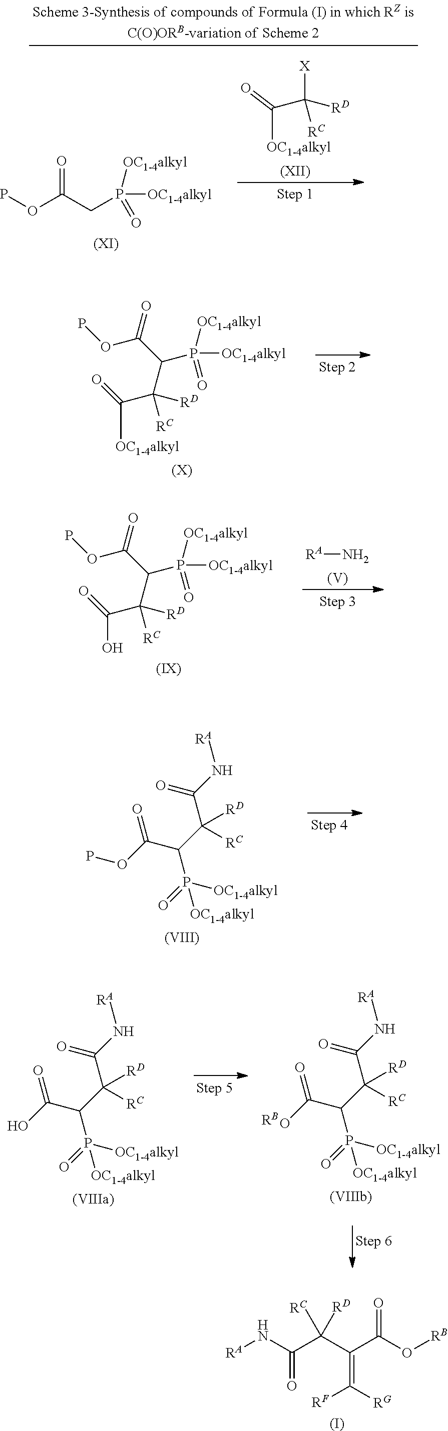

Wherein R^{B} is H, and X, P, R^{A}, R^{C}, R^{D}, R^{F} and R^{G} are defined elsewhere herein.

The route of Scheme 3 is suitable for the preparation of compounds of formula (I) in which R^{B} is other than H.
Steps 1-3: Identical to Steps 1-3 of Scheme 2.
Step 4: Compounds of formula (VIII) may be converted to compounds of formula (VIIIa) by removal of the protecting group P. For example, if P is C₁₋₄alkyl, e.g., tBu, P may be removed under acidic conditions, such as TFA in DCM.
Step 5: Compounds of formula (VIIIa) may be converted to compounds of formula (VIIIb) where R^{B} is other than H using analogous methods to those shown in Scheme 1, i.e., reacting the compound of formula (I) (R^{B}═H) with alcohol (VI).
Step 6: Compounds of formula (VIIIb) may be converted to compounds of formula (I) in which R^{B} is other than H using the Horner-Wadsworth-Emmons modification of the Wittig reaction, such as by using aqueous C(R^{F})(R^{G})═O in the presence of a base such as K₂CO₃. Alternatively, Step 5 may be omitted and the reaction of Step 6 may be used to convert compounds of formula (VIIIa) to compounds of formula (I) in which R^{B} is H.

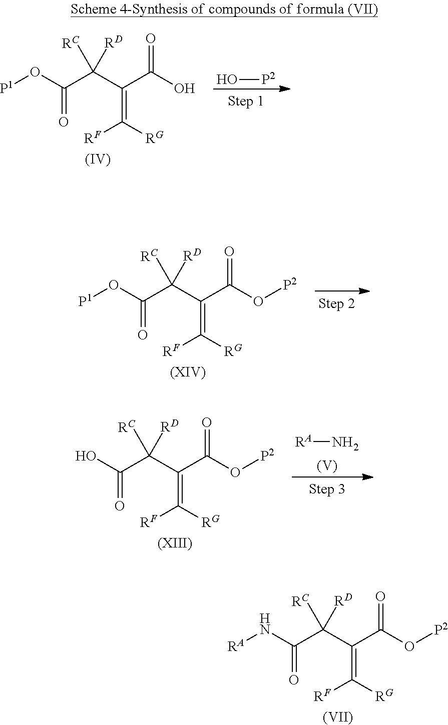

wherein P¹ and P² are orthogonal carboxylic acid protecting groups (for example, P¹ may be PMB and P² may be CH₂CCl₃) and R^{A}, R^{C}, R^{D}, R^{F} and R^{G} are as defined elsewhere herein.
Step 1: Compounds of formula (IV) may be condensed with HO-P² to introduce protecting group P₂ using coupling conditions, such as EDC·HCl/DMAP and a base such as DIPEA, to give compounds of formula (XIV).
Step 2: Compounds of formula (XIV) may be converted to compounds of formula (XIII) by removal of protecting group P¹ using conditions, known to the person skilled in the art, under which orthogonal protecting group P² is not labile. For example, if P¹ is PMB, this group may be removed using acidic conditions, such as TFA in DCM, which do not result in the removal of orthogonal protecting group P².
Step 3: Compounds of formula (XIII) are coupled with amines of formula (V) under standard amide coupling conditions (such as HATU in the presence of a base such as DIPEA) to give compounds of formula (VII). Compounds of formula (VII) may be converted to compounds of formula (I) as shown in Scheme 2. The CH₂CCl₃ protecting group may be removed using Zn and NH₄OAc in aqueous THF.

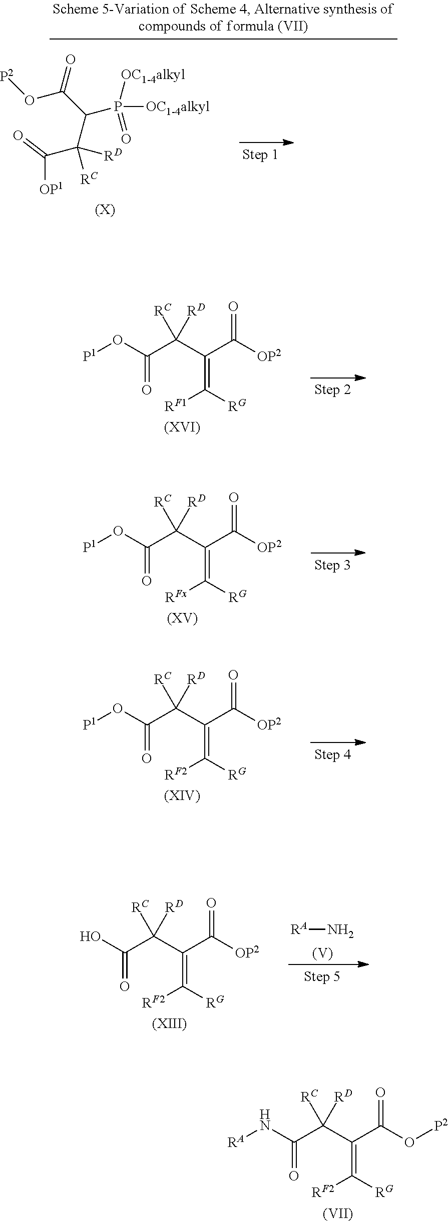

wherein R^{A}, R^{C}, R^{D} and R^{G} are defined elsewhere herein, P¹ and P² are orthogonal carboxylic acid protecting groups (for example, P¹ may be methyl and P² may be tert-butyl), R^{F1} and R^{F2} are both R^{F} groups as defined elsewhere herein and are different from one another, in particular R^{F1} is alkyl and R^{F2} is alkyl substituted with OR^{K} or N(R^{K})(R^{L}), wherein each of R^{K} and R^{L} is as defined elsewhere herein; and R^{Fx} is a precursor of R^{F2}, for example a haloalkyl group such as bromoethyl, which can be converted to a group R^{F2} by reaction with HOR^{K} or HN(R^{K})(R^{L}).
Step 1: Compounds of formula (X) may be converted to compounds of formula (XVI) using the Horner-Wadsworth-Emmons modification of the Wittig reaction, such as by using aqueous C(R^{Fx})(R^{G})═O in the presence of a base such as K₂CO₃.
Step 2: Compounds of formula (XVI) may be converted to compounds of formula (XV) by any known method. For example, when R^{Fx} is 2-bromoethyl, it can be prepared by bromination of the compound of formula (XVI) for example using N-bromosuccinimide.
Step 3: Compounds of formula (XV) may be converted to compounds of formula (XIV) by any known method, for example when R^{Fx} is 2-bromoethyl, the compound of formula (XV) may be reacted with an amine of formula HN(R^{K})(R^{L}) to give a compound of formula (XIV) in which R^{F2} is —CH₂CH₂—N(R^{K})(R^{L}). This is described in Example 49.
Steps 4 and 5: These steps correspond to Steps 2 and 3 of Scheme 4.

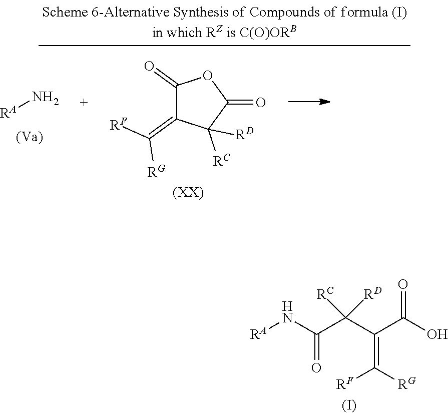

wherein:
R^{C}, R^{D}, R^{F} and R^{G} are as defined elsewhere herein; R^{B} is H, and R^{A} is CH₂(substituted phenyl) or CH₂(substituted pyridyl), wherein the CH₂ group is optionally substituted with one R^{A3} and the phenyl or pyridyl group is substituted with one or more R^{A4}, wherein R^{A3} and R^{A4} are as defined above

### Compounds of Formula (Va):

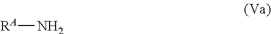

wherein R^{A} is CH₂(substituted phenyl) or CH₂(substituted pyridyl), wherein the CH₂ group is optionally substituted with one R^{A3} and the phenyl or pyridyl group is substituted with one or more R^{A4}, wherein R^{A3} and R^{A4} are as defined above for formula (I);
may be reacted with an itaconic anhydride derivative of formula (XX) in a solvent such as dichloroethane and at reduced temperature (typically at 0° C. to 10° C.) to give compounds of formula (I) in which R^{B} is H.

This method is more suitable for compounds in which R^{A} is CH₂(substituted phenyl) in which the CH₂ group is optionally substituted with a single R^{A3} substituent. It is particularly suitable for compounds of formula (I) in which the CH₂ group is substituted with a single methyl substituent.

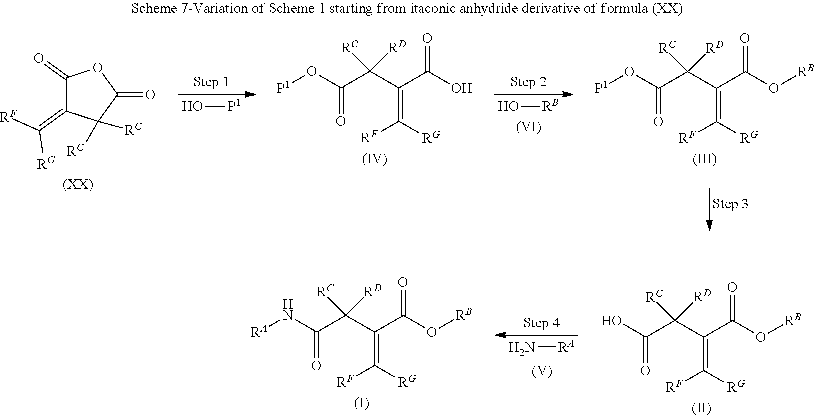

Wherein R^{A}, R^{B}, R^{C}, R^{D}, R^{F} and R^{G} are as defined elsewhere herein.
Step 1: The itaconic anhydride derivative of formula (XX) is reacted with an alcohol HO-P¹, where P¹ is a carboxylic acid protecting group such as 2,2,2-trichloroethyl, in the presence of a Lewis acid such as boron trifluoride diethyl etherate to give a compound of formula (IV).
Step 2: The compound of formula (IV) is reacted with a alcohol of formula (VI) using coupling conditions such as EDC·HCl/DMAP in the presence of a base such as DIPEA, to provide a compound of formula (III).
Step 3: The protecting group P¹ is removed to give a compound of formula (II). For example, if the protecting group is 2,2,2-trichloroethyl, it can be removed by treatment with zinc and ammonium acetate.
Step 4: Compounds of formula (II) are coupled with amines of formula (V) under standard amide coupling conditions (such as HATU in the presence of a base such as DIPEA) to give compounds of formula (I) in which R^{B} is other than H and R^{C} and R^{D} are both H.

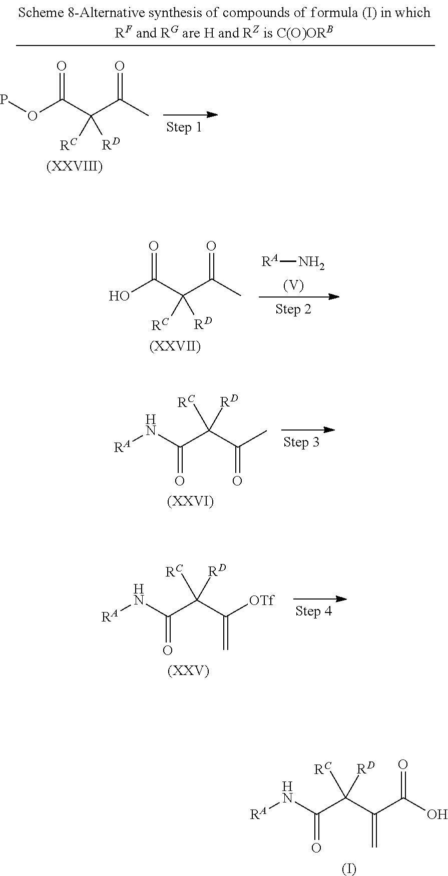

wherein R^{A}, R^{C} and R^{D} are as defined elsewhere herein, and P is a carboxylic acid protecting group such as C₁₋₆ alkyl.
Step 1: Compounds of formula (XXVIII) may be converted to compounds of formula (XXVII) by hydrolysis, for example alkaline hydrolysis using an alkali metal hydroxide such as lithium hydroxide in a suitable solvent, such as methanol/water.
Step 2: Compounds of formula (XXVII) are coupled with amines of formula (V) under standard amide coupling conditions (such as dicyclohexylcarbodiimide (DCC) in the presence of a base such as dimethylaminopyridine (DMAP) to give compounds of formula (XXVI).
Step 3: Compounds of formula (XXVI) may be converted to enol triflate compounds of formula (XXV) by treatment of triflating reagents such as 1,1,1-trifluoro-N-phenyl-N-((trifluoromethyl) sulfonyl)methanesulfonamide after deprotonation with a base such as lithium diisopropylamide (LDA).
Step 4: Compounds of formula (XXV) may be carbonylated by reaction with triphenylphosphine, palladium acetate and carbon monoxide, followed by reaction with triethylamine and acetic acid to give a compound of Formula (I).

Compounds of formula (I) in Scheme 8, wherein R^{B} is H, may be converted to compounds of formula (I) wherein R^{B} is other than H using analogous methods to those shown in Scheme 1, i.e., reacting the compound of formula (I) (R^{B}═H) with alcohol HO—R^{B} (VI), where in R^{B} is as defined above for formula (I) except that it is not H.

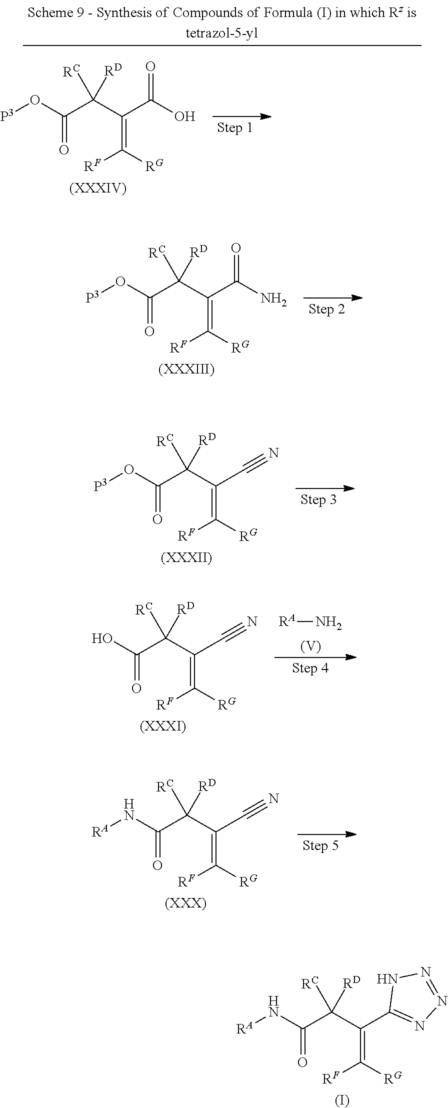

wherein P³ is suitably a C₁₋₆ alkyl or benzyl group or a carboxylic acid protecting group.
Step 1: A carboxylic acid of formula (XXXIV) is converted to an acid chloride by reaction with oxalyl chloride in the presence of catalytic amount of N,N-dimethylformamide and a solvent such as dichloromethane (DCM), following which, the acid chloride is reacted with aqueous ammonia to give an amide of formula (XXXIII).
Step 2: The amide of formula (XXXIII) may be converted to a nitrile of formula (XXXII) by reaction with trifluoroacetic anhydride (TFAA) and triethylamine (TEA) in a solvent such as DCM and at room temperature (15° to 25° C.).
Step 3: Hydrolysis of the ester compound (XXXII), for example alkaline hydrolysis using LiOH in THF/H₂O, yields an acid of formula (XXXI).
Step 4: The acid (XXXI) may be reacted with an amine of formula (V) to give a compound of formula (XXX). The reaction is suitably conducted in the presence of a coupling reagent such as N,N-dicyclohexyl carbodiimide (DCC) and under basic conditions, for example in the presence of dimethylaminopyridine (DMAP) using a reaction solvent such as DCM.
Step 5: The nitrile (XXX) may be converted to a tetrazole of formula (I) by reaction with trimethylsilyl azide (TMSN₃) in the presence of a catalyst such as dibutyltin oxide. The reaction may be conducted at elevated temperature, for example 80° to 100° C. in a solvent such as 1,4-dioxane.

In Schemes 1 to 9, compounds of formulae (IV), (V), (Va), (VI) (X), (XII), (XX), (XXVIII) and (XXXIV) are readily available or may be synthesised by known methods as described in the examples below.

The skilled person will appreciate that protecting groups may be used throughout the synthetic schemes described herein to give protected derivatives of any of the above compounds or generic formulae. Protective groups and the means for their removal are described in “Protective Groups in Organic Synthesis”, by Theodora W. Greene and Peter G. M. Wuts, published by John Wiley & Sons Inc; 4th Rev Ed., 2006, ISBN-10: 0471697540. Examples of nitrogen protecting groups include trityl (Tr), tert-butyloxycarbonyl (BOC), 9-fluorenylmethyloxycarbonyl (Fmoc), acetyl (Ac), benzyl (Bn) and para-methoxy benzyl (PMB). Examples of oxygen protecting groups include acetyl (Ac), methoxymethyl (MOM), para-methoxybenzyl (PMB), benzyl, tert-butyl, methyl, ethyl, tetrahydropyranyl (THP), and silyl ethers and esters (such as trimethylsilyl (TMS), tert-butyldimethylsilyl (TBDMS), tri-iso-propylsilyloxymethyl (TOM), and triisopropylsilyl (TIPS) ethers and esters). Specific examples of carboxylic acid protecting groups include alkyl esters (such as C₁₋₆ alkyl and C₁₋₆ haloalkyl e.g. C₁₋₄ alkyl esters and C₁₋₄ haloalkyl esters), benzyl esters (including substituted benzyl esters such as p-methoxybenzyl esters), and silyl esters.

In one embodiment, there is provided a process for preparing a compound of formula (I) in which R^{Z} is C(O)OR^{B}, or a salt, such as a pharmaceutically acceptable salt, thereof, which comprises reacting a compound of formula (II):

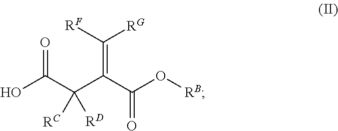

or a salt thereof;
with amine R^{A}—NH₂ (V), or a salt thereof;
wherein R^{B} is:

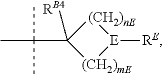

and R^{A}, R^{C}, R^{D}, R^{F} and R^{G} are defined elsewhere herein.

In another embodiment, there is provided a process for preparing a compound of formula (I) in which R^{Z} is C(O)OR^{B}, or a salt, such as a pharmaceutically acceptable salt, thereof, which comprises deprotecting a compound of formula (VII):

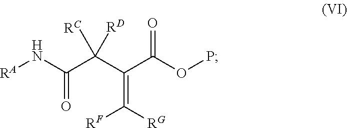

or a salt thereof;
wherein R^{A}, R^{C}, R^{D}, R^{F} and R^{G} are defined elsewhere herein and P is a carboxylic acid protecting group, such as C₁₋₄alkyl, e.g., tBu or CH₂CCl₃.

Thus, in one embodiment, there is provided a compound of formula (II):

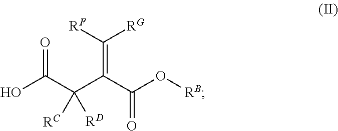

or a salt thereof;
wherein R^{B} is:

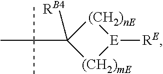

and R^{A}, R^{C}, R^{D}, R^{F} and R^{G} are defined elsewhere herein.

In another embodiment, there is provided a compound of formula (VII):

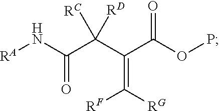

or a salt thereof;
wherein R^{A}, R^{C}, R^{D}, R^{F} and R^{G} are defined elsewhere herein and P is a carboxylic acid protecting group, such as C₁₋₄alkyl, e.g., tBu, or CH₂CCl₃.

In another embodiment, there is provided a process for preparing a compound of formula (I) in which R^{Z} is C(O)OR^{B}, or a salt, such as a pharmaceutically acceptable salt, thereof, by reacting a compound of formula (VIIIa) or (VIIIb):

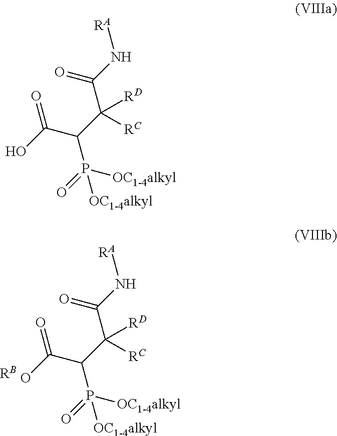

or a salt thereof;
wherein R^{A}, R^{C} and R^{D} are defined elsewhere herein;
with aqueous (R^{F})C(R^{G})═O in the presence of a base.

In another embodiment, there is provided a process for preparing a compound of formula (I) in which R^{Z} is C(O)OR^{B}, where R^{B} is H, or a salt, such as a pharmaceutically acceptable salt, thereof, which comprises reacting a compound of formula (Va):

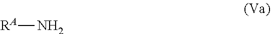

wherein R^{A} is CH₂(substituted phenyl) or CH₂(substituted phenyl), wherein the CH₂ group is optionally substituted with one or two R^{A3} and the phenyl or pyridyl group is substituted with one or more R^{A4}, wherein R^{A3} and R^{A4} are as defined above for formula (I);
with an itaconic anhydride derivative of formula (XX):

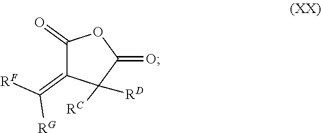

wherein R^{C}, R^{D}, R^{F} and R^{G} are defined elsewhere herein.

This method is particularly suitable for compounds in which R^{A} is CH₂(substituted phenyl) or CH₂(substituted pyridyl), especially CH₂(substituted phenyl), in which the CH₂ group is optionally substituted with a single R^{A3} substituent. It is particularly suitable when the CH₂ group is substituted with a single methyl substituent.

In a further embodiment, there is provided a process for preparing a compound of formula (I) in which R^{F} and R^{G} are both H and R^{Z} is C(O)OR^{B}, or a salt, such as a pharmaceutically acceptable salt, thereof, which comprises reacting a compound of formula (XXV):

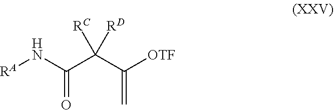

wherein R^{A}, R^{C} and R^{D} are defined elsewhere herein;
with triphenylphosphine, palladium acetate and carbon monoxide, followed by with a base such as triethylamine and a carboxylic acid such as acetic acid;
to give a compound of formula (I) in which R^{B} is H; and optionally reacting a compound of formula (I) in which R^{Z} is C(O)OH with a compound of formula (VI):

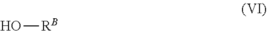

where in R^{B} is as defined above for formula (I) except that it is not H;
to give a compound of formula (I) in which R^{Z} is C(O)OR^{B}, where in R^{B} is as defined above for formula (I) except that it is not H.

In a further embodiment, there is provided a process for preparing a compound of formula (I) in which R^{Z} is tetrazol-5-yl, or a salt, such as a pharmaceutical acceptable salt, thereof, which comprises reacting a compound of formula (XXXI):

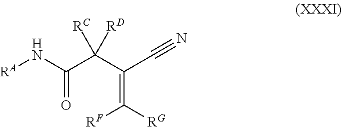

or a salt thereof
wherein R^{A}, R^{C}, R^{D}, R^{F} and R^{G} are defined elsewhere herein;
with trimethylsilyl azide (TMSN₃) in the presence of a catalyst. Suitable catalysts include dibutyl tin oxide.

In another embodiment, there is provided a compound of formula (VIIIa) or (VIIIb):

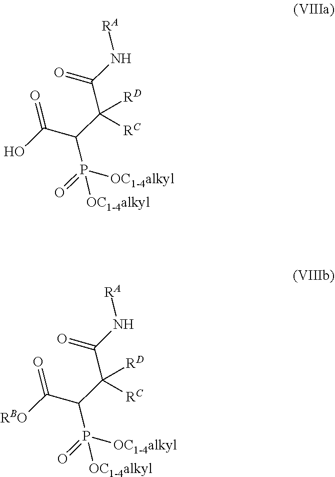

or a salt thereof;
wherein R^{A}, R^{C} and R^{D} are defined elsewhere herein;

In a further embodiment, there is provided a compound of formula (XXXI):

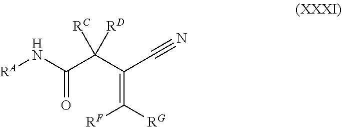

or a salt thereof
wherein R^{A}, R^{C}, R^{D}, R^{F} and R^{G} are defined elsewhere herein.

In a further embodiment, there is provided a compound of formula (XXV):

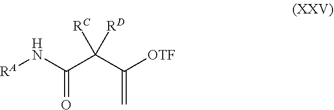

wherein R^{A}, R^{C} and R^{D} are defined elsewhere herein.

It will be appreciated that for use in therapy the salts of the compounds of formula (I) should be pharmaceutically acceptable. Suitable pharmaceutically acceptable salts will be apparent to those skilled in the art. Pharmaceutically acceptable salts include acid addition salts, suitably salts of compounds of the invention comprising a basic group such as an amino group, formed with inorganic acids, e.g., hydrochloric acid, hydrobromic acid, sulfuric acid, nitric acid or phosphoric acid. Also included are salts formed with organic acids, e.g., succinic acid, maleic acid, acetic acid, fumaric acid, citric acid, tartaric acid, benzoic acid, p-toluenesulfonic acid, methanesulfonic acid, naphthalenesulfonic acid and 1,5-naphthalenedisulfonic acid. Other salts, e.g., oxalates or formates, may be used, for example in the isolation of compounds of formula (I) and are included within the scope of this invention, as are basic addition salts such as sodium, potassium, calcium, aluminium, zinc, magnesium and other metal salts.

Pharmaceutically acceptable salts may also be formed with organic bases such as basic amines, e.g., with ammonia, meglumine, tromethamine, piperazine, arginine, choline, diethylamine, benzathine or lysine. Thus, in one embodiment there is provided a compound of formula (I) in the form of a pharmaceutically acceptable salt. Alternatively, there is provided a compound of formula (I) in the form of a free acid. When the compound contains a basic group as well as the free acid it may be Zwitterionic.

Suitably, the compound of formula (I) is not a salt, e.g., is not a pharmaceutically acceptable salt.

Suitably, where the compound of formula (I) is in the form of a salt, the pharmaceutically acceptable salt is a basic addition salt such as a carboxylate salt formed with a group 1 metal (e.g., a sodium or potassium salt), a group 2 metal (e.g., a magnesium or calcium salt) or an ammonium salt of a basic amine (e.g., an NH₄⁺ salt), such as a sodium salt.

The compounds of formula (I) may be prepared in crystalline or non-crystalline form and, if crystalline, may optionally be solvated, e.g., as the hydrate. This invention includes within its scope stoichiometric solvates (e.g., hydrates) as well as compounds containing variable amounts of solvent (e.g., water). Suitably, the compound of formula (I) is not a solvate.

The invention extends to a pharmaceutically acceptable derivative thereof, such as a pharmaceutically acceptable prodrug of compounds of formula (I). Typical prodrugs of compounds of formula (I) which comprise a carboxylic acid include ester (e.g. C₁₋₆ alkyl e.g. C₁₋₄ alkyl ester) derivatives thereof. Thus, in one embodiment, the compound of formula (I) is provided as a pharmaceutically acceptable prodrug. In another embodiment, the compound of formula (I) is not provided as a pharmaceutically acceptable prodrug.

Certain compounds of formula (I) may metabolise under certain conditions. Without wishing to be bound by theory, formation of an active metabolite (such as in vivo) of a compound of formula (I) may be beneficial by contributing to the biological activity observed of the compound of formula (I). Thus, in one embodiment, there is provided an active metabolite of the compound of formula (I) and its use as a pharmaceutical e.g. for the treatment or prevention of the diseases mentioned herein.

It is to be understood that the present invention encompasses all isomers of compounds of formula (I) including all geometric, tautomeric and optical forms, and mixtures thereof (e.g. racemic mixtures). In particular, the invention extends to all tautomeric forms of the compounds of formula (I). Where additional chiral centres are present in compounds of formula (I), the present invention includes within its scope all possible diastereoisomers, including mixtures thereof. The different isomeric forms may be separated or resolved one from the other by conventional methods, or any given isomer may be obtained by conventional synthetic methods or by stereospecific or asymmetric syntheses.

The present invention also includes all isotopic forms of the compounds provided herein, whether in a form (i) wherein all atoms of a given atomic number have a mass number (or mixture of mass numbers) which predominates in nature (referred to herein as the “natural isotopic form”) or (ii) wherein one or more atoms are replaced by atoms having the same atomic number, but a mass number different from the mass number of atoms which predominates in nature (referred to herein as an “unnatural variant isotopic form”). It is understood that an atom may naturally exists as a mixture of mass numbers. The term “unnatural variant isotopic form” also includes embodiments in which the proportion of an atom of given atomic number having a mass number found less commonly in nature (referred to herein as an “uncommon isotope”) has been increased relative to that which is naturally occurring e.g. to the level of >20%, >50%, >75%, >90%, >95% or >99% by number of the atoms of that atomic number (the latter embodiment referred to as an “isotopically enriched variant form”). The term “unnatural variant isotopic form” also includes embodiments in which the proportion of an uncommon isotope has been reduced relative to that which is naturally occurring. Isotopic forms may include radioactive forms (i.e. they incorporate radioisotopes) and non-radioactive forms. Radioactive forms will typically be isotopically enriched variant forms.

An unnatural variant isotopic form of a compound may thus contain one or more artificial or uncommon isotopes such as deuterium (²H or D), carbon-11 (¹¹C), carbon-13 (¹³C), carbon-14 (¹⁴C), nitrogen-13 (¹³N), nitrogen-15 (¹⁵N), oxygen-15 (¹⁵O), oxygen-17 (¹⁷O), oxygen-18 (¹⁸O), phosphorus-32 (³²P), sulphur-35 (³⁵S), chlorine-36 (³⁶Cl), chlorine-37 (³¹Cl), fluorine-18 (¹⁸F) iodine-123 (¹²³I), iodine-125 (¹²⁵I) in one or more atoms or may contain an increased proportion of said isotopes as compared with the proportion that predominates in nature in one or more atoms.

Unnatural variant isotopic forms comprising radioisotopes may, for example, be used for drug and/or substrate tissue distribution studies. The radioactive isotopes tritium, i.e. ³H, and carbon-14, i.e. ¹⁴C, are particularly useful for this purpose in view of their ease of incorporation and ready means of detection. Unnatural variant isotopic forms which incorporate deuterium i.e. ²H or D may afford certain therapeutic advantages resulting from greater metabolic stability, for example, increased in vivo half-life or reduced dosage requirements, and hence may be preferred in some circumstances. Further, unnatural variant isotopic forms may be prepared which incorporate positron emitting isotopes, such as ¹¹C, ¹⁸F, ¹⁵O and ¹³N, and would be useful in positron emission topography (PET) studies for examining substrate receptor occupancy.

In one embodiment, the compounds of formula (I) are provided in a natural isotopic form. In one embodiment, the compounds of formula (I) are provided in an unnatural variant isotopic form. In a specific embodiment, the unnatural variant isotopic form is a form in which deuterium (i.e. ²H or D) is incorporated where hydrogen is specified in the chemical structure in one or more atoms of a compound of formula (I). In one embodiment, the atoms of the compounds of formula (I) are in an isotopic form which is not radioactive. In one embodiment, one or more atoms of the compounds of formula (I) are in an isotopic form which is radioactive. Suitably radioactive isotopes are stable isotopes. Suitably the unnatural variant isotopic form is a pharmaceutically acceptable form.

In one embodiment, a compound of formula (I) is provided whereby a single atom of the compound exists in an unnatural variant isotopic form. In another embodiment, a compound of formula (I) is provided whereby two or more atoms exist in an unnatural variant isotopic form.

Unnatural isotopic variant forms can generally be prepared by conventional techniques known to those skilled in the art or by processes described herein e.g. processes analogous to those described in the accompanying Examples for preparing natural isotopic forms. Thus, unnatural isotopic variant forms could be prepared by using appropriate isotopically variant (or labelled) reagents in place of the normal reagents employed in the Examples. Since the compounds of formula (I) are intended for use in pharmaceutical compositions it will readily be understood that they are each preferably provided in substantially pure form, for example at least 60% pure, more suitably at least 75% pure and preferably at least 85%, especially at least 98% pure (% are on a weight for weight basis). Impure preparations of the compounds may be used for preparing the purer forms used in the pharmaceutical compositions.

### Therapeutic Indications

Compounds of formula (I) are of use in therapy, particularly for treating or preventing an inflammatory disease or a disease associated with an undesirable immune response. As shown in Biological Example 1 below, example compounds of formula (I) reduced cytokine release more effectively than dimethyl itaconate, as demonstrated by lower IC₅₀ values. Cytokines are important mediators of inflammation and immune-mediated disease as evidenced by the therapeutic benefit delivered by antibodies targeting them.

Thus, in a further aspect, the present invention provides a compound of formula (I) or a pharmaceutically acceptable salt and/or solvate thereof as defined herein, for use as a medicament. Also provided is a pharmaceutical composition comprising a compound of formula (I) or a pharmaceutically acceptable salt and/or solvate thereof as defined herein. Such a pharmaceutical composition contains the compound of formula (I) and a pharmaceutically acceptable carrier or excipient.

In a further aspect, the present invention provides a compound of formula (I) or a pharmaceutically acceptable salt and/or solvate thereof as defined herein, for use in treating or preventing an inflammatory disease or a disease associated with an undesirable immune response. In a further aspect, the present invention provides the use of a compound of formula (I) or a pharmaceutically acceptable salt and/or solvate thereof as defined herein, in the manufacture of a medicament for treating or preventing an inflammatory disease or a disease associated with an undesirable immune response. In a further aspect, the present invention provides a method of treating or preventing an inflammatory disease or a disease associated with an undesirable immune response, which comprises administering a compound of formula (I) or a pharmaceutically acceptable salt and/or solvate thereof as defined herein.

For all aspects of the invention, suitably the compound is administered to a subject in need thereof, wherein the subject is suitably a human subject.

In one embodiment is provided a compound of formula (I) or a pharmaceutically acceptable salt and/or solvate thereof as defined herein, for use in treating an inflammatory disease or disease associated with an undesirable immune response. In one embodiment of the invention is provided the use of a compound of formula (I) or a pharmaceutically acceptable salt and/or solvate thereof as defined herein, in the manufacture of a medicament for treating an inflammatory disease or a disease associated with an undesirable immune response. In one embodiment of the invention is provided a method of treating an inflammatory disease or a disease associated with an undesirable immune response, which comprises administering a compound of formula (I) or a pharmaceutically acceptable salt and/or solvate thereof as defined herein.

In one embodiment is provided a compound of formula (I) or a pharmaceutically acceptable salt and/or solvate thereof as defined herein, for use in preventing an inflammatory disease or a disease associated with an undesirable immune response. In one embodiment of the invention is provided the use of a compound of formula (I) or a pharmaceutically acceptable salt and/or solvate thereof as defined herein, in the manufacture of a medicament for preventing an inflammatory disease or a disease associated with an undesirable immune response. In one embodiment of the invention is provided a method of preventing an inflammatory disease or a disease associated with an undesirable immune response, which comprises administering a compound of formula (I) or a pharmaceutically acceptable salt and/or solvate thereof as defined herein.

In one embodiment is provided a compound of formula (I) or a pharmaceutically acceptable salt and/or solvate thereof as defined herein, for use in treating or preventing an inflammatory disease. In one embodiment of the invention is provided the use of a compound of formula (I) or a pharmaceutically acceptable salt and/or solvate thereof as defined herein, in the manufacture of a medicament for treating or preventing an inflammatory disease. In one embodiment of the invention is provided a method of treating or preventing an inflammatory disease, which comprises administering a compound of formula (I) or a pharmaceutically acceptable salt and/or solvate thereof as defined herein.

In one embodiment is provided a compound of formula (I) or a pharmaceutically acceptable salt and/or solvate thereof as defined herein, for use in treating or preventing a disease associated with an undesirable immune response. In one embodiment of the invention is provided the use of a compound of formula (I) or a pharmaceutically acceptable salt and/or solvate thereof as defined herein, in the manufacture of a medicament for treating or preventing a disease associated with an undesirable immune response. In one embodiment of the invention is provided a method of treating or preventing a disease associated with an undesirable immune response, which comprises administering a compound of formula (I) or a pharmaceutically acceptable salt and/or solvate thereof as defined herein.

An undesirable immune response will typically be an immune response which gives rise to a pathology i.e. is a pathological immune response or reaction.

In one embodiment, the inflammatory disease or disease associated with an undesirable immune response is an auto-immune disease.

In one embodiment, the inflammatory disease or disease associated with an undesirable immune response is, or is associated with, a disease selected from the group consisting of: psoriasis (including chronic plaque, erythrodermic, pustular, guttate, inverse and nail variants), asthma, chronic obstructive pulmonary disease (COPD, including chronic bronchitis and emphysema), heart failure (including left ventricular failure), myocardial infarction, angina pectoris, other atherosclerosis and/or atherothrombosis-related disorders (including peripheral vascular disease and ischaemic stroke), a mitochondrial and neurodegenerative disease (such as Parkinson's disease, Alzheimer's disease, Huntington's disease, amyotrophic lateral sclerosis, retinitis pigmentosa or mitochondrial encephalomyopathy), autoimmune paraneoplastic retinopathy, transplantation rejection (including antibody-mediated and T cell-mediated forms), multiple sclerosis, transverse myelitis, ischaemia-reperfusion injury (e.g. during elective surgery such as cardiopulmonary bypass for coronary artery bypass grafting or other cardiac surgery, following percutaneous coronary intervention, following treatment of acute ST-elevation myocardial infarction or ischaemic stroke, organ transplantation, or acute compartment syndrome), AGE-induced genome damage, an inflammatory bowel disease (e.g. Crohn's disease or ulcerative colitis), primary sclerosing cholangitis (PSC), PSC-autoimmune hepatitis overlap syndrome, non-alcoholic fatty liver disease (non-alcoholic steatohepatitis), rheumatica, granuloma annulare, cutaneous lupus erythematosus (CLE), systemic lupus erythematosus (SLE), lupus nephritis, drug-induced lupus, autoimmune myocarditis or myopericarditis, Dressler's syndrome, giant cell myocarditis, post-pericardiotomy syndrome, drug-induced hypersensitivity syndromes (including hypersensitivity myocarditis), eczema, sarcoidosis, erythema nodosum, acute disseminated encephalomyelitis (ADEM), neuromyelitis optica spectrum disorders, MOG (myelin oligodendrocyte glycoprotein) antibody-associated disorders (including MOG-EM), optic neuritis, CLIPPERS (chronic lymphocytic inflammation with pontine perivascular enhancement responsive to steroids), diffuse myelinoclastic sclerosis, Addison's disease, alopecia areata, ankylosing spondylitis, other spondyloarthritides (including peripheral spondyloarthritis, that is associated with psoriasis, inflammatory bowel disease, reactive arthritis or juvenile onset forms), antiphospholipid antibody syndrome, autoimmune hemolytic anaemia, autoimmune hepatitis, autoimmune inner ear disease, pemphigoid (including bullous pemphigoid, mucous membrane pemphigoid, cicatricial pemphigoid, herpes gestationis or pemphigoid gestationis, ocular cicatricial pemphigoid), linear IgA disease, Behçet's disease, celiac disease, Chagas disease, dermatomyositis, diabetes mellitus type I, endometriosis, Goodpasture's syndrome, Graves' disease, Guillain-Barre syndrome and its subtypes (including acute inflammatory demyelinating polyneuropathy, AIDP, acute motor axonal neuropathy (AMAN), acute motor and sensory axonal neuropathy (AMSAN), pharyngeal-cervical-brachial variant, Miller-Fisher variant and Bickerstaff's brainstem encephalitis), progressive inflammatory neuropathy, Hashimoto's disease, hidradenitis suppurativa, inclusion body myositis, necrotising myopathy, Kawasaki disease, IgA nephropathy, Henoch-Schonlein purpura, idiopathic thrombocytopenic purpura, thrombotic thrombocytopenic purpura (TTP), Evans' syndrome, interstitial cystitis, mixed connective tissue disease, undifferentiated connective tissue disease, morphea, myasthenia gravis (including MuSK antibody positive and seronegative variants), narcolepsy, neuromyotonia, pemphigus vulgaris, pernicious anaemia, psoriatic arthritis, polymyositis, primary biliary cholangitis (also known as primary biliary cirrhosis), rheumatoid arthritis, palindromic rheumatism, schizophrenia, autoimmune (meningo-)encephalitis syndromes, scleroderma, Sjogren's syndrome, stiff person syndrome, polymylagia rheumatica, giant cell arteritis (temporal arteritis), Takayasu arteritis, polyarteritis nodosa, Kawasaki disease, granulomatosis with polyangitis (GPA; formerly known as Wegener's granulomatosis), eosinophilic granulomatosis with polyangiitis (EGPA; formerly known as Churg-Strauss syndrome), microscopic polyarteritis/polyangiitis, hypocomplementaemic urticarial vasculitis, hypersensitivity vasculitis, cryoglobulinemia, thromboangiitis obliterans (Buerger's disease), vasculitis, leukocytoclastic vasculitis, vitiligo, acute disseminated encephalomyelitis, adrenoleukodystrophy, Alexander's disease, Alper's disease, balo concentric sclerosis or Marburg disease, cryptogenic organising pneumonia (formerly known as bronchiolitis obliterans organizing pneumonia), Canavan disease, central nervous system vasculitic syndrome, Charcot-Marie-Tooth disease, childhood ataxia with central nervous system hypomyelination, chronic inflammatory demyelinating polyneuropathy (CIDP), diabetic retinopathy, globoid cell leukodystrophy (Krabbe disease), graft-versus-host disease (GVHD) (including acute and chronic forms, as well as intestinal GVHD), hepatitis C (HCV) infection or complication, herpes simplex viral infection or complication, human immunodeficiency virus (HIV) infection or complication, lichen planus, monomelic amyotrophy, cystic fibrosis, pulmonary arterial hypertension (PAH, including idiopathic PAH), lung sarcoidosis, idiopathic pulmonary fibrosis, paediatric asthma, atopic dermatitis, allergic dermatitis, contact dermatitis, allergic rhinitis, rhinitis, sinusitis, conjunctivitis, allergic conjunctivitis, keratoconjunctivitis sicca, dry eye, xerophthalmia, glaucoma, macular oedema, diabetic macular oedema, central retinal vein occlusion (CRVO), macular degeneration (including dry and/or wet age related macular degeneration, AMD), post-operative cataract inflammation, uveitis (including posterior, anterior, intermediate and pan uveitis), iridocyclitis, scleritis, corneal graft and limbal cell transplant rejection, gluten sensitive enteropathy (coeliac disease), dermatitis herpetiformis, eosinophilic esophagitis, achalasia, autoimmune dysautonomia, autoimmune encephalomyelitis, autoimmune oophoritis, autoimmune orchitis, autoimmune pancreatitis, aortitis and periaortitis, autoimmune retinopathy, autoimmune urticaria, (idiopathic) Castleman's disease, Cogan's syndrome, IgG4-related disease, retroperitoneal fibrosis, juvenile idiopathic arthritis including systemic juvenile idiopathic arthritis (Still's disease), adult-onset Still's disease, ligneous conjunctivitis, Mooren's ulcer, pityriasis lichenoides et varioliformis acuta (PLEVA, also known as Mucha-Habermann disease), multifocal motor neuropathy (MMN), paediatric acute-onset neuropsychiatric syndrome (PANS) (including paediatric autoimmune neuropsychiatric disorders associated with streptococcal infections (PANDAS)), paraneoplastic syndromes (including paraneoplastic cerebellar degeneration, Lambert-Eaton myaesthenic syndrome, limbic encephalitis, brainstem encephalitis, opsoclonus myoclonus ataxia syndrome, anti-NMDA receptor encephalitis, thymoma-associated multiorgan autoimmunity), perivenous encephalomyelitis, reflex sympathetic dystrophy, relapsing polychondritis, sperm & testicular autoimmunity, Susac's syndrome, Tolosa-Hunt syndrome, Vogt-Koyanagi-Harada Disease, anti-synthetase syndrome, autoimmune enteropathy, immune dysregulation polyendocrinopathy enteropathy X-linked (IPEX), microscopic colitis, autoimmune lymphoproliferative syndrome (ALPS), autoimmune polyendocrinopathy-candidiasis-ectodermal dystrophy syndrome (APEX), gout, pseudogout, amyloid (including AA or secondary amyloidosis), eosinophilic fasciitis (Shulman syndrome) progesterone hypersensitivity (including progesterone dermatitis), familial Mediterranean fever (FMF), tumour necrosis factor (TNF) receptor-associated periodic fever syndrome (TRAPS), hyperimmunoglobulinaemia D with periodic fever syndrome (HIDS), PAPA (pyogenic arthritis, pyoderma gangrenosum, severe cystic acne) syndrome, deficiency of interleukin-1 receptor antagonist (DIRA), deficiency of the interleukin-36-receptor antagonist (DITRA), cryopyrin-associated periodic syndromes (CAPS) (including familial cold autoinflammatory syndrome [FCAS], Muckle-Wells syndrome, neonatal onset multisystem inflammatory disease [NOMID]), NLRP12-associated autoinflammatory disorders (NLRP12AD), periodic fever aphthous stomatitis (PFAPA), chronic atypical neutrophilic dermatosis with lipodystrophy and elevated temperature (CANDLE), Majeed syndrome, Blau syndrome (also known as juvenile systemic granulomatosis), macrophage activation syndrome, chronic recurrent multifocal osteomyelitis (CRMO), familial cold autoinflammatory syndrome, mutant adenosine deaminase 2 and monogenic interferonopathies (including Aicardi-Goutières syndrome, retinal vasculopathy with cerebral leukodystrophy, spondyloenchondrodysplasia, STING [stimulator of interferon genes]-associated vasculopathy with onset in infancy, proteasome associated autoinflammatory syndromes, familial chilblain lupus, dyschromatosis symmetrica hereditaria), Schnitzler syndrome; familial cylindromatosis, congenital B cell lymphocytosis, OTULIN-related autoinflammatory syndrome, type 2 diabetes mellitus, insulin resistance and the metabolic syndrome (including obesity-associated inflammation), atherosclerotic disorders (e.g. myocardial infarction, angina, ischaemic heart failure, ischaemic nephropathy, ischaemic stroke, peripheral vascular disease, aortic aneurysm), renal inflammatory disorders (e.g. diabetic nephropathy, membranous nephropathy, minimal change disease, crescentic glomerulonephritis, acute kidney injury, renal transplantation).

In one embodiment, the inflammatory disease or disease associated with an undesirable immune response is, or is associated with, a disease selected from the following autoinflammatory diseases: familial Mediterranean fever (FMF), tumour necrosis factor (TNF) receptor-associated periodic fever syndrome (TRAPS), hyperimmunoglobulinaemia D with periodic fever syndrome (HIDS), PAPA (pyogenic arthritis, pyoderma gangrenosum, and severe cystic acne) syndrome, deficiency of interleukin-1 receptor antagonist (DIRA), deficiency of the interleukin-36-receptor antagonist (DITRA), cryopyrin-associated periodic syndromes (CAPS) (including familial cold autoinflammatory syndrome [FCAS], Muckle-Wells syndrome, and neonatal onset multisystem inflammatory disease [NOMID]), NLRP12-associated autoinflammatory disorders (NLRP12AD), periodic fever aphthous stomatitis (PFAPA), chronic atypical neutrophilic dermatosis with lipodystrophy and elevated temperature (CANDLE), Majeed syndrome, Blau syndrome (also known as juvenile systemic granulomatosis), macrophage activation syndrome, chronic recurrent multifocal osteomyelitis (CRMO), familial cold autoinflammatory syndrome, mutant adenosine deaminase 2 and monogenic interferonopathies (including Aicardi-Goutières syndrome, retinal vasculopathy with cerebral leukodystrophy, spondyloenchondrodysplasia, STING [stimulator of interferon genes]-associated vasculopathy with onset in infancy, proteasome associated autoinflammatory syndromes, familial chilblain lupus, dyschromatosis symmetrica hereditaria) and Schnitzler syndrome.

In one embodiment, the inflammatory disease or disease associated with an undesirable immune response is, or is associated with, a disease selected from the following diseases mediated by excess NF-κB or gain of function in the NF-κB signalling pathway or in which there is a major contribution to the abnormal pathogenesis therefrom (including non-canonical NF-κB signalling): familial cylindromatosis, congenital B cell lymphocytosis, OTULIN-related autoinflammatory syndrome, type 2 diabetes mellitus, insulin resistance and the metabolic syndrome (including obesity-associated inflammation), atherosclerotic disorders (e.g. myocardial infarction, angina, ischaemic heart failure, ischaemic nephropathy, ischaemic stroke, peripheral vascular disease, aortic aneurysm), renal inflammatory disorders (e.g. diabetic nephropathy, membranous nephropathy, minimal change disease, crescentic glomerulonephritis, acute kidney injury, renal transplantation), asthma, COPD, type 1 diabetes mellitus, rheumatoid arthritis, multiple sclerosis, inflammatory bowel disease (including ulcerative colitis and Crohn's disease), and SLE.

In one embodiment, the disease is selected from the group consisting of rheumatoid arthritis, psoriatic arthritis, ankylosing spondylitis, systemic lupus erythematosus, multiple sclerosis, psoriasis, Crohn's disease, ulcerative colitis, uveitis, cryopyrin-associated periodic syndromes, Muckle-Wells syndrome, juvenile idiopathic arthritis, chronic obstructive pulmonary disease and asthma.

In one embodiment, the disease is multiple sclerosis.

In one embodiment, the disease is psoriasis.

In one embodiment, the disease is asthma.

In one embodiment, the disease is chronic obstructive pulmonary disease.

In one embodiment, the disease is systemic lupus erythematosus.

### Administration

The compound of formula (I) is usually administered as a pharmaceutical composition. Thus, in one embodiment, is provided a pharmaceutical composition comprising a compound of formula (I) and one or more pharmaceutically acceptable diluents or carriers.

The compound of formula (I) may be administered by any convenient method, e.g. by oral, parenteral, buccal, sublingual, nasal, rectal, intrathecal or transdermal administration, and the pharmaceutical compositions adapted accordingly.

The compound of formula (I) may be administered topically to the target organ e.g. topically to the eye, lung, nose or skin. Hence the invention provides a pharmaceutical composition comprising a compound of formula (I) optionally in combination with one or more topically acceptable diluents or carriers.

A compound of formula (I) which is active when given orally can be formulated as a liquid or solid, e.g. as a syrup, suspension, emulsion, tablet, capsule or lozenge.

A liquid formulation will generally consist of a suspension or solution of the compound of formula (I) in a suitable liquid carrier(s). Suitably the carrier is non-aqueous e.g. polyethylene glycol or an oil. The formulation may also contain a suspending agent, preservative, flavouring and/or colouring agent.

A composition in the form of a tablet can be prepared using any suitable pharmaceutical carrier(s) routinely used for preparing solid formulations, such as magnesium stearate, starch, lactose, sucrose and cellulose.

A composition in the form of a capsule can be prepared using routine encapsulation procedures, e.g. pellets containing the active ingredient can be prepared using standard carriers and then filled into a hard gelatine capsule; alternatively, a dispersion or suspension can be prepared using any suitable pharmaceutical carrier(s), e.g. aqueous gums, celluloses, silicates or oils and the dispersion or suspension then filled into a soft gelatine capsule.

Typical parenteral compositions consist of a solution or suspension of the compound of formula (I) in a sterile aqueous carrier or parenterally acceptable oil, e.g. polyethylene glycol, polyvinyl pyrrolidone, lecithin, arachis oil or sesame oil. Alternatively, the solution can be lyophilised and then reconstituted with a suitable solvent just prior to administration.

Compositions for nasal administration may conveniently be formulated as aerosols, drops, gels and powders. Aerosol formulations typically comprise a solution or fine suspension of the compound of formula (I) in a pharmaceutically acceptable aqueous or non-aqueous solvent and are usually presented in single or multidose quantities in sterile form in a sealed container which can take the form of a cartridge or refill for use with an atomising device. Alternatively, the sealed container may be a disposable dispensing device such as a single dose nasal inhaler or an aerosol dispenser fitted with a metering valve. Where the dosage form comprises an aerosol dispenser, it will contain a propellant which can be a compressed gas e.g. air, or an organic propellant such as a chlorofluorocarbon (CFC) or a hydrofluorocarbon (HFC). Aerosol dosage forms can also take the form of pump-atomisers.

Topical administration to the lung may be achieved by use of an aerosol formulation. Aerosol formulations typically comprise the active ingredient suspended or dissolved in a suitable aerosol propellant, such as a chlorofluorocarbon (CFC) or a hydrofluorocarbon (HFC).

Topical administration to the lung may also be achieved by use of a non-pressurised formulation such as an aqueous solution or suspension. These may be administered by means of a nebuliser e.g. one that can be hand-held and portable or for home or hospital use (i.e. non-portable). The formulation may comprise excipients such as water, buffers, tonicity adjusting agents, pH adjusting agents, surfactants and co-solvents.

Topical administration to the lung may also be achieved by use of a dry-powder formulation. The formulation will typically contain a topically acceptable diluent such as lactose, glucose or mannitol (preferably lactose).

The compound of the invention may also be administered rectally, for example in the form of suppositories or enemas, which include aqueous or oily solutions as well as suspensions and emulsions and foams. Such compositions are prepared following standard procedures, well known by those skilled in the art. For example, suppositories can be prepared by mixing the active ingredient with a conventional suppository base such as cocoa butter or other glycerides. In this case, the drug is mixed with a suitable non-irritating excipient which is solid at ordinary temperatures but liquid at the rectal temperature and will therefore melt in the rectum to release the drug. Such materials are cocoa butter and polyethylene glycols.

Generally, for compositions intended to be administered topically to the eye in the form of eye drops or eye ointments, the total amount of the compound of the present invention will be about 0.0001 to less than 4.0% (w/w).

Preferably, for topical ocular administration, the compositions administered according to the present invention will be formulated as solutions, suspensions, emulsions and other dosage forms.

The compositions administered according to the present invention may also include various other ingredients, including, but not limited to, tonicity agents, buffers, surfactants, stabilizing polymer, preservatives, co-solvents and viscosity building agents. Suitable pharmaceutical compositions of the present invention include a compound of the invention formulated with a tonicity agent and a buffer. The pharmaceutical compositions of the present invention may further optionally include a surfactant and/or a palliative agent and/or a stabilizing polymer.

Various tonicity agents may be employed to adjust the tonicity of the composition, preferably to that of natural tears for ophthalmic compositions. For example, sodium chloride, potassium chloride, magnesium chloride, calcium chloride, simple sugars such as dextrose, fructose, galactose, and/or simply polyols such as the sugar alcohols mannitol, sorbitol, xylitol, lactitol, isomaltitol, maltitol, and hydrogenated starch hydrolysates may be added to the composition to approximate physiological tonicity. Such an amount of tonicity agent will vary, depending on the particular agent to be added. In general, however, the compositions will have a tonicity agent in an amount sufficient to cause the final composition to have an ophthalmically acceptable osmolality (generally about 150-450 mOsm, preferably 250-350 mOsm and most preferably at approximately 290 mOsm). In general, the tonicity agents of the invention will be present in the range of 2 to 4% w/w. Preferred tonicity agents of the invention include the simple sugars or the sugar alcohols, such as D-mannitol.

An appropriate buffer system (e.g. sodium phosphate, sodium acetate, sodium citrate, sodium borate or boric acid) may be added to the compositions to prevent pH drift under storage conditions. The particular concentration will vary, depending on the agent employed. Preferably however, the buffer will be chosen to maintain a target pH within the range of pH 5 to 8, and more preferably to a target pH of pH 5 to 7.

Surfactants may optionally be employed to deliver higher concentrations of compound of the present invention. The surfactants function to solubilise the compound and stabilise colloid dispersion, such as micellar solution, microemulsion, emulsion and suspension. Examples of surfactants which may optionally be used include polysorbate, poloxamer, polyosyl 40 stearate, polyoxyl castor oil, tyloxapol, Triton, and sorbitan monolaurate. Preferred surfactants to be employed in the invention have a hydrophile/lipophile/balance “HLB” in the range of 12.4 to 13.2 and are acceptable for ophthalmic use, such as TritonX114 and tyloxapol.

Additional agents that may be added to the ophthalmic compositions of compounds of the present invention are demulcents which function as a stabilising polymer. The stabilizing polymer should be an ionic/charged example with precedence for topical ocular use, more specifically, a polymer that carries negative charge on its surface that can exhibit a zeta-potential of (−)10-50 mV for physical stability and capable of making a dispersion in water (i.e. water soluble). A preferred stabilising polymer of the invention would be polyelectrolyte, or polyelectrolytes if more than one, from the family of cross-linked polyacrylates, such as carbomers and Pemulen®, specifically Carbomer 974p (polyacrylic acid), at 0.1-0.5% w/w.

Other compounds may also be added to the ophthalmic compositions of the compound of the present invention to increase the viscosity of the carrier. Examples of viscosity enhancing agents include, but are not limited to: polysaccharides, such as hyaluronic acid and its salts, chondroitin sulfate and its salts, dextrans, various polymers of the cellulose family; vinyl polymers; and acrylic acid polymers.

Topical ophthalmic products are typically packaged in multidose form. Preservatives are thus required to prevent microbial contamination during use. Suitable preservatives include: benzalkonium chloride, chlorobutanol, benzododecinium bromide, methyl paraben, propyl paraben, phenylethyl alcohol, edentate disodium, sorbic acid, polyquaternium-1, or other agents known to those skilled in the art. Such preservatives are typically employed at a level of from 0.001 to 1.0% w/v. Unit dose compositions of the present invention will be sterile, but typically unpreserved. Such compositions, therefore, generally will not contain preservatives.

Compositions suitable for buccal or sublingual administration include tablets, lozenges and pastilles where the compound of formula (I) is formulated with a carrier such as sugar and acacia, tragacanth, or gelatine and glycerine.

Compositions suitable for transdermal administration include ointments, gels and patches.

The composition may contain from 0.1% to 100% by weight, for example from 10 to 60% by weight, of the compound of formula (I), depending on the method of administration. The composition may contain from 0% to 99% by weight, for example 40% to 90% by weight, of the carrier, depending on the method of administration. The composition may contain from 0.05 mg to 1000 mg, for example from 1.0 mg to 500 mg, such as from 1.0 mg to 50 mg, e.g. about 10 mg of the compound of formula (I), depending on the method of administration. The composition may contain from 50 mg to 1000 mg, for example from 100 mg to 400 mg of the carrier, depending on the method of administration. The dose of the compound used in the treatment of the aforementioned disorders will vary in the usual way with the seriousness of the disorders, the weight of the sufferer, and other similar factors. However, as a general guide suitable unit doses may be 0.05 to 1000 mg, more suitably 1.0 to 500 mg, such as from 1.0 mg to 50 mg, e.g. about 10 mg and such unit doses may be administered more than once a day, for example two or three times a day. Such therapy may extend for a number of weeks or months.

In one embodiment of the invention, the compound of formula (I) is used in combination with a further therapeutic agent or agents. When the compound of formula (I) is used in combination with other therapeutic agents, the compounds may be administered either sequentially or simultaneously by any convenient route. Alternatively, the compounds may be administered separately.

Therapeutic agents which may be used in combination with the present invention include: corticosteroids (glucocorticoids), retinoids (e.g. acitretin, isotretinoin, tazarotene), anthralin, vitamin D analogues (e.g. cacitriol, calcipotriol), calcineurin inhibitors (e.g. tacrolimus, pimecrolimus), phototherapy or photochemotherapy (e.g. psoralen ultraviolet irradiation, PUVA) or other form of ultraviolet light irradiation therapy, ciclosporine, thiopurines (e.g. azathioprine, 6-mercaptopurine), methotrexate, anti-TNFα agents (e.g. infliximab, etanercept, adalimumab, certolizumab, golimumab and biosimilars), phosphodiesterase-4 (PDE4) inhibition (e.g. apremilast, crisaborole), anti-IL-17 agents (e.g. brodalumab, ixekizumab, secukinumab), anti-IL12/IL-23 agents (e.g. ustekinumab, briakinumab), anti-IL-23 agents (e.g. guselkumab, tildrakizumab), JAK (Janus Kinase) inhibitors (e.g. tofacitinib, ruxolitinib, baricitinib, filgotinib, upadacitinib), plasma exchange, intravenous immune globulin (IVIG), cyclophosphamide, anti-CD20 B cell depleting agents (e.g. rituximab, ocrelizumab, ofatumumab, obinutuzumab), anthracycline analogues (e.g. mitoxantrone), cladribine, sphingosine 1-phosphate receptor modulators or sphingosine analogues (e.g. fingolimod, siponimod, ozanimod, etrasimod), interferon beta preparations (including interferon beta 1b/1a), glatiramer, anti-CD3 therapy (e.g. OKT3), anti-CD52 targeting agents (e.g. alemtuzumab), leflunomide, teriflunomide, gold compounds, laquinimod, potassium channel blockers (e.g. dalfampridine/4-aminopyridine), mycophenolic acid, mycophenolate mofetil, purine analogues (e.g. pentostatin), mTOR (mechanistic target of rapamycin) pathway inhibitors (e.g. sirolimus, everolimus), anti-thymocyte globulin (ATG), IL-2 receptor (CD25) inhibitors (e.g. basiliximab, daclizumab), anti-IL-6 receptor or anti-IL-6 agents (e.g. tocilizumab, siltuximab), Bruton's tyrosine kinase (BTK) inhibitors (e.g. ibrutinib), tyrosine kinase inhibitors (e.g. imatinib), ursodeoxycholic acid, hydroxychloroquine, chloroquine, B cell activating factor (BAFF, also known as BLyS, B lymphocyte stimulator) inhibitors (e.g. belimumab, blisibimod), other B cell targeted therapy including fusion proteins targeting both APRIL (A PRoliferation-Inducing Ligand) and BLyS (e.g. atacicept), PI3K inhibitors including pan-inhibitors or those targeting the p110δ and/or p110γ containing isoforms (e.g. idelalisib, copanlisib, duvelisib), interferon α receptor inhibitors (e.g. anifrolumab, sifalimumab), T cell co-stimulation blockers (e.g. abatacept, belatacept), thalidomide and its derivatives (e.g. lenalidomide), dapsone, clofazimine, leukotriene antagonists (e.g. montelukast), theophylline, anti-IgE therapy (e.g. omalizumab), anti-IL-5 agents (e.g. mepolizumab, reslizumab), long-acting muscarinic agents (e.g. tiotropium, aclidinium, umeclidinium), PDE4 inhibitors (e.g. roflumilast), riluzole, free radical scavengers (e.g. edaravone), proteasome inhibitors (e.g. bortezomib), complement cascade inhibitors including those directed against C5 (e.g. eculizumab), immunoadsor, antithymocyte globulin, 5-aminosalicylates and their derivatives (e.g. sulfasalazine, balsalazide, mesalamine), anti-integrin agents including those targeting α4β1 and/or α4β7 integrins (e.g. natalizumab, vedolizumab), anti-CD11-α agents (e.g. efalizumab), non-steroidal anti-inflammatory drugs (NSAIDs) including the salicylates (e.g. aspirin), propionic acids (e.g. ibuprofen, naproxen), acetic acids (e.g. indomethacin, diclofenac, etodolac), oxicams (e.g. meloxicam) and fenamates (e.g. mefenamic acid), selective or relatively selective COX-2 inhibitors (e.g. celecoxib, etroxicoxib, valdecoxib and etodolac, meloxicam, nabumetone), colchicine, IL-4 receptor inhibitors (e.g. dupilumab), topical/contact immunotherapy (e.g. diphenylcyclopropenone, squaric acid dibutyl ester), anti-IL-1 receptor therapy (e.g. anakinra), IL-1β inhibitor (e.g. canakinumab), IL-1 neutralising therapy (e.g. rilonacept), chlorambucil, specific antibiotics with immunomodulatory properties and/or ability to modulate NRF2 (e.g. tetracyclines including minocycline, clindamycin, macrolide antibiotics), anti-androgenic therapy (e.g. cyproterone, spironolactone, finasteride), pentoxifylline, ursodeoxycholic acid, obeticholic acid, fibrate, cystic fibrosis transmembrane conductance regulator (CFTR) modulators, VEGF (vascular endothelial growth factor) inhibitors (e.g. bevacizumab, ranibizumab, pegaptanib, aflibercept), pirfenidone, and mizoribine.

Compounds of formula (I) may display one or more of the following desirable properties:
low IC₅₀ values for inhibiting release of cytokines, e.g., IL-1β, from cells;
low EC₅₀ and/or high Eₘₐₓ values for activating the NRF2 pathway;
high EC₅₀ and/or low Eₘₐₓ values for activating the NRF2 pathway;
enhanced efficacy through improved hydrolytic stability;
reduced dose and dosing frequency through improved pharmacokinetics;
improved oral systemic bioavailability;
reduced plasma clearance following intravenous dosing;
improved metabolic stability, e.g., as demonstrated by improved stability in plasma and/or hepatocytes;
augmented cell permeability;
enhanced aqueous solubility;
good tolerability, for example, by limiting the flushing and/or gastrointestinal side effects provoked by oral DMF (Hunt T. et al., 2015; WO2014/152494A1, incorporated herein by reference), possibly by reducing or eliminating HCA2 activity;
low toxicity at the relevant therapeutic dose;
distinct anti-inflammatory profiles resulting from varied electrophilicities, leading to differential targeting of the cysteine proteome (van der Reest J. et al., 2018) and, therefore, modified effects on gene activation;
insensitivity of biological effects to added glutathione;
avoiding the oncometabolite fumaric acid (Kulkarni R. A. et al., 2019);
decreased plasma protein binding.

The invention is further defined by the following clauses:

Clause 1. A compound of formula (I):

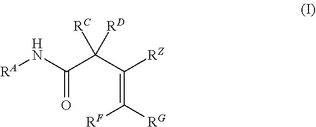

wherein:
R^{A} is selected from the group consisting of C₆₋₁₀ cycloalkyl optionally fused to phenyl, CH₂(phenyl) and CH₂(pyridyl);
wherein when R^{A} is C₆₋₁₀ cycloalkyl optionally fused to phenyl, the cycloalkyl ring is optionally substituted on an available carbon atom by one or more R^{A2}, wherein R^{A2} is independently selected from the group consisting of methyl, halo and trifluoromethyl, and/or two R^{A2} are attached to the same carbon atom and join to form a C₃₋₆ cycloalkyl or a 4-6 membered heterocyclic ring; and the fused phenyl ring is optionally substituted on an available carbon atom by one or more substituents selected from the group consisting of C₁₋₄ haloalkyl and halo;
wherein when R^{A} is CH₂(phenyl) or CH₂(pyridyl):
   the CH₂ group is optionally substituted by one or two R^{A3} wherein each R^{A3} is independently selected from the group consisting of C₁₋₄ alkyl, C₃₋₅ cycloalkyl, C₁₋₄ haloalkyl and C₁₋₂ hydroxyalkyl, or two R^{A3} groups are attached to the CH₂ carbon atom and join to form a C₃₋₆ cycloalkyl or a 4- or 5-membered heterocyclic ring; and
   the phenyl or pyridyl group is optionally substituted by one or more R^{A4}, wherein each R^{A4} is independently selected from the group consisting of C₁₋₄ haloalkyl, halo, SF₅ or SfC₁₋₄ haloalkyl;
   R^{Z} is selected from the group consisting of C(O)OR^{B} and tetrazol-5-yl;
   R^{B} is selected from the group consisting of H; C₂₋₃ alkyl substituted with OH or N(R^{B2})(R^{B3}); and

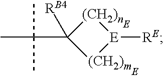

wherein:
   when R^{B} is C₂₋₃ alkyl substituted with N(R^{B2})(R^{B3}), R^{B2} is selected from H and methyl and R^{B3} is selected from methyl and SO₂(C₁₋₂ alkyl) or R^{B2} and R^{B3} together with the nitrogen atom to which they are attached, combine to form a 4- to 6-membered heterocyclic ring optionally containing a further heteroatom selected from O and N;
   wherein, when R^{B} is

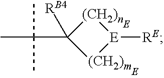

R^{B4} is H or methyl;
E is N, O or SO₂;
wherein when E is N, R^{E} is selected from the group consisting of R^{9B}, C(O)R^{9B} and SO₂R^{9B}; wherein R^{9B} is C₁₋₄ alkyl; and when E is O or SO₂, R^{E} is absent;
nE and mE are independently 1 or 2;
R^{C} and R^{D} are each independently H, C₁₋₂ alkyl, hydroxy, methoxy or fluoro;
each of R^{F} and R^{G} is independently selected from H, phenyl and C₁₋₄ alkyl optionally substituted with phenyl, OR^{K} or N(R^{K})(R^{L}), wherein each of R^{K} and R^{L} is independently selected from H, methyl and ethyl, provided that at least one of R^{F} and R^{G} is H; and
   wherein when the CH₂ of the CH₂(substituted phenyl) or CH₂(substituted pyridyl) of R^{A} is substituted by one R^{A3} the stereochemistry of the carbon to which R^{A3} is attached is as follows:

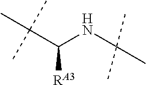

wherein the dashed lines indicate attachment to the remainder of the compound of formula (I); and wherein when R^{A4} is Cl, the CH₂ group is unsubstituted or is substituted by one R^{A3}; or a pharmaceutically acceptable salt and/or solvate thereof.

Clause 2. The compound or salt and/or solvate thereof according to Clause 1, which is a compound of formula (Ia):

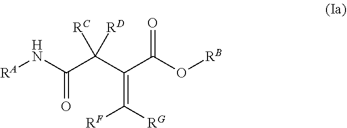

wherein:
R^{A} is selected from the group consisting of C₆₋₁₀ cycloalkyl optionally fused to phenyl, CH₂(phenyl) and CH₂(pyridyl);
wherein when R^{A} is C₆₋₁₀ cycloalkyl optionally fused to phenyl, the cycloalkyl ring is optionally substituted on an available carbon atom by one or more R^{A2}, wherein R^{A2} is selected from the group consisting of methyl, halo and trifluoromethyl, and/or two R^{A2} are attached to the same carbon atom and join to form a C₃₋₆ cycloalkyl or a 4-6 membered heterocyclic ring; and the fused phenyl ring is optionally substituted on an available carbon atom by one or more substituents selected from the group consisting of C₁₋₄ haloalkyl and halo;
wherein when R^{A} is CH₂(phenyl) or CH₂(pyridyl):
   the CH₂ group is optionally substituted by one or two R^{A3} wherein R^{A3} is selected from the group consisting of C₁₋₄ alkyl, C₃₋₅ cycloalkyl, C₁₋₄ haloalkyl and C₁₋₂ hydroxyalkyl, or two R^{A3} groups are attached to the CH₂ carbon atom and join to form a C₃₋₆ cycloalkyl or a 4- or 5-membered heterocyclic ring; and
   the phenyl or pyridyl group is optionally substituted by one or more R^{A4}, wherein R^{A4} is selected from the group consisting of C₁₋₄ haloalkyl, halo, or SC₁₋₄ haloalkyl;
R^{B} is selected from the group consisting of H; C₂₋₃ alkyl substituted with OH or N(R^{B2})(R^{B3}); and

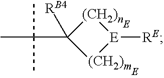

wherein:
   when R^{B} is C₂₋₃ alkyl substituted with N(R^{B2})(R^{B3}), R^{B2} is selected from H and methyl and R^{B3} is selected from methyl and SO₂(C₁₋₂ alkyl) or R^{B2} and R^{B3}, together with the nitrogen atom to which they are attached, combine to form a 4- to 6-membered heterocyclic ring optionally containing a further heteroatom selected from O and N;
   wherein, when R^{B} is

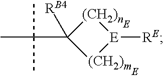

R^{B4} is H or methyl;
E is N, O or SO₂;
wherein when E is N, R^{E} is selected from the group consisting of R^{9B}, C(O)R^{9B} and SO₂R^{9B}; wherein R^{9B} is C₁₋₄ alkyl; and when E is O or SO₂, R^{E} is absent;
nE and mE are independently 1 or 2;
R^{C} and R^{D} are each independently H, C₁₋₂ alkyl, hydroxy, methoxy or fluoro;
each of R^{F} and R^{G} is independently H or methyl, provided that at least one of R^{F} and R^{G} is H; and
   wherein when the CH₂ of the CH₂(substituted phenyl) or CH₂(substituted pyridyl) of R^{A} is substituted by one R^{A3} the stereochemistry of the carbon to which R^{A3} is attached is as follows:

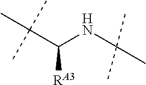

wherein the dashed lines indicate attachment to the remainder of the compound of formula (I); and wherein when R^{A4} is Cl, the CH₂ group is unsubstituted or is substituted by one R^{A3}; and
wherein, when R^{B} is H, R^{A} is not unsubstituted cyclohexyl;
or a pharmaceutically acceptable salt and/or solvate thereof.

Clause 3. The compound or salt and/or solvate thereof according to clause 1 or clause 2, which is a compound of formula (Ib):

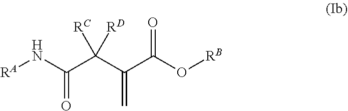

wherein:
R^{A} is selected from the group consisting of C₆₋₁₀ cycloalkyl and CH₂phenyl;
wherein when R^{A} is C₆₋₁₀ cycloalkyl, R^{A} is optionally substituted on an available carbon atom by one or more R^{A2}, wherein R^{A2} is selected from the group consisting of methyl, halo and trifluoromethyl, and/or two R^{A2} are attached to the same carbon atom and join to form a C₃₋₆ cycloalkyl or a 4-6 membered heterocyclic ring;
wherein when R^{A} is CH₂phenyl:
   the CH₂ group is optionally substituted by one or two R^{A3} wherein R^{A3} is selected from the group consisting of C₁₋₄ alkyl, C₃₋₅ cycloalkyl, C₁₋₄ haloalkyl and C₁₋₂ hydroxyalkyl, or two R^{A3} groups are attached to the CH₂ carbon atom and join to form a C₃₋₆ cycloalkyl or a 4-6 membered heterocyclic ring; and
   the phenyl group is optionally substituted by one or more R^{A4}, wherein one R^{A4} is in the 4-position, wherein R^{A4} is selected from the group consisting of C₁₋₄ haloalkyl, halo, or SC₁₋₄ haloalkyl;
R^{B} is selected from the group consisting of H and

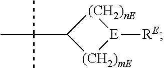

wherein:
   E is N or O;
   wherein when E is N, R^{E} is SO₂R^{9B}; wherein R^{9B} is C₁₋₄ alkyl; and when E is O, R^{E} is absent;
   nE and mE are independently 1 or 2;
R^{C} and R^{D} are each independently H, C₁₋₂ alkyl, hydroxy, methoxy or fluoro; and
   wherein when the CH₂ of the CH₂phenyl is substituted by one R^{A3} the stereochemistry of the carbon to which R^{A3} is attached is as follows:

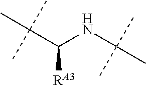

wherein the dashed lines indicate attachment to the remainder of the compound of formula (I); and wherein when R^{A4} is Cl, the CH₂ group is unsubstituted or is substituted by one R^{A3};
or a pharmaceutically acceptable salt and/or solvate thereof.

Clause 4. The compound or salt and/or solvate thereof according to any one of clauses 1 to 3, wherein R^{A} is unsubstituted C₆₋₁₀ cycloalkyl.

Clause 5. The compound or salt and/or solvate thereof according to any one of clauses 1 to 4, wherein R^{A} is unsubstituted C₇₋₁₀ cycloalkyl

Clause 6. The compound or salt and/or solvate thereof according to any one of clauses 1 to 5, wherein R^{A} is cyclooctyl optionally substituted on an available carbon atom by one or more R^{A2}.

Clause 7. The compound or salt and/or solvate thereof according to any one of clauses 1 to 4, wherein R^{A} is C₆₋₁₀ cycloalkyl substituted on an available carbon atom by one or more (such as one, two or three e.g. one) R^{A2}.

Clause 8. The compound or salt and/or solvate thereof according to clause 1 or clause 2 wherein R^{A} is C₆₋₁₀ cycloalkyl fused to phenyl, wherein the cycloalkyl ring is optionally substituted on an available carbon atom by one or more R^{A2}; and wherein the phenyl ring is optionally substituted on an available carbon atom by one or more substituents selected from the group consisting of C₁₋₄ haloalkyl and halo, for example, the phenyl ring is substituted on an available carbon atom by a trifluoromethyl substituent.

Clause 9. The compound or salt and/or solvate thereof according to any one of clauses 6 to 8, wherein R^{A2} is methyl.

Clause 10. The compound or salt and/or solvate thereof according to any one of clauses 6 8 10, wherein R^{A2} is halo.

Clause 11. The compound or salt and/or solvate thereof according to any one of clauses 6 to 8, wherein R^{A2} is trifluoromethyl.

Clause 12. The compound or salt and/or solvate thereof according to any one of clauses 6 to 11, wherein two R^{A2} are attached to the same carbon atom and join to form a C₃₋₆ cycloalkyl or a 4- or 5-membered heterocyclic ring.

Clause 13. The compound or salt and/or solvate thereof according to clause 12, wherein two R^{A2} are attached to the same carbon atom and join to form a C₃₋₆ cycloalkyl ring.

Clause 14. The compound or salt and/or solvate thereof according to clause 12, wherein two R^{A2} are attached to the same carbon atom and join to form a 4-6 membered heterocyclic ring.

Clause 15. The compound or salt and/or solvate thereof according to any one of clauses 1 to 3, wherein R^{A} is CH₂(phenyl) wherein the phenyl is substituted by one or more (such as one, two or three e.g. one) R^{A4}.

Clause 16. The compound or salt and/or solvate thereof according to clause 15, wherein one R^{A4} is in the 4-position.

Clause 17. The compound or salt and/or solvate thereof according to any one of clauses 1 to 3, wherein R^{A} is CH₂(pyridyl) wherein the pyridyl group is substituted by one or more (such as one, two or three e.g. one) R^{A4}.

Clause 18. The compound or salt and/or solvate thereof according to clause 17, wherein one R^{A4} is in the para-position with respect to the linkage to the remainder of the molecule.

Clause 19. The compound or salt and/or solvate thereof according to clause 18, wherein the pyridyl group is a 2-pyridyl group.

Clause 20. The compound or salt and/or solvate thereof according to clause 18, wherein the pyridyl group is a 3-pyridyl group.

Clause 21. The compound or salt and/or solvate thereof according to any one of clauses 15 to 21 wherein R^{A} is CH₂(substituted phenyl) or CH₂(substituted pyridyl) and the phenyl or pyridyl group is substituted by two R^{A4}, and wherein the first R^{A4} substituent is in the para position with respect to the linkage to the remainder of the molecule and the second R^{A4} substituent is in the meta position with respect to the linkage to the remainder of the molecule.

Clause 22. The compound or salt and/or solvate thereof according to any one of clauses 15 to 21, wherein R^{A4} is C₁₋₄ haloalkyl.

Clause 23. The compound or salt and/or solvate thereof according to clause 22, wherein R^{A4} is CHF₂, CF₃CF₂ or CF₃.

Clause 24. The compound or salt and/or solvate thereof according to clause 23 wherein R^{A4} is CF₃.

Clause 25. The compound or salt and/or solvate thereof according to any one of clauses 15 to 21, wherein R^{A4} is halo such as fluoro or bromo.

Clause 26. The compound or salt and/or solvate thereof according to any one of clauses 15 to 21, wherein R^{A4} is SC₁₋₄ haloalkyl or SF₅.

Clause 27. The compound or salt and/or solvate thereof according to any one of clauses 15 to 26, wherein the CH₂ group is not substituted.

Clause 28. The compound or salt and/or solvate thereof according to any one of clauses 15 to 26, wherein the CH₂ group is substituted by one R^{A3}.

Clause 29. The compound or salt and/or solvate thereof according to any one of clauses 15 to 26, wherein the CH₂ group is substituted by two R^{A3}.

Clause 30. The compound or salt and/or solvate thereof according to clause 29, wherein R^{A3} is C₁₋₄ alkyl.

Clause 31. The compound or salt and/or solvate thereof according to clause 30, wherein R^{A3} is methyl or ethyl.

Clause 32. The compound or salt and/or solvate thereof according to clause 29, wherein each R^{A3} is C₁₋₄alkyl.

Clause 33. The compound or salt and/or solvate thereof according to clause 32, wherein each R^{A3} is methyl.

Clause 34. The compound or salt and/or solvate thereof according to clause 28 or clause 29, wherein R^{A3} is C₃₋₅cycloalkyl.

Clause 35. The compound or salt and/or solvate thereof according to clause 34, wherein R^{A3} is cyclopropyl.

Clause 36. The compound or salt and/or solvate thereof according to clause 28 or clause 29, wherein R^{A3} is C₁₋₄ haloalkyl.

Clause 37. The compound or salt and/or solvate thereof according to clause 36, wherein R^{A3} is trifluoromethyl.

Clause 38. The compound or salt and/or solvate thereof according to clause 28 or clause 29, wherein R^{A3} is C₁₋₂hydroxyalkyl.

Clause 39. The compound or salt and/or solvate thereof according to clause 38, wherein R^{A3} is CH₂OH.

Clause 40. The compound or salt and/or solvate thereof according to any one of clauses 15 to 26, wherein two R^{A3} groups are attached to the CH₂ carbon atom and join to form a C₃₋₆ cycloalkyl or a 4- or 5-membered heterocyclic ring.

Clause 41. The compound or salt and/or solvate thereof according to clause 40, wherein two R^{A3} groups are attached to the CH₂ carbon atom and join to form a C₃₋₆ cycloalkyl ring.

Clause 42. The compound or salt and/or solvate thereof according to clause 41, wherein two R^{A3} groups join to form a cyclopropyl or cyclobutyl ring.

Clause 43. The compound or salt and/or solvate thereof according to clause 40, wherein two R^{A3} groups are attached to the CH₂ carbon atom and join to form a 4- or 5-membered heterocyclic ring.

Clause 44. The compound or salt and/or solvate thereof according to clause 43, wherein two R^{A3} groups join to form an oxetanyl ring.

The compound or salt and/or solvate thereof according Clause 45. The compound or salt and/or solvate thereof according to any one of clauses 1 to 44, wherein R^{Z} is C(O)OR^{B}.

Clause 46. The compound or salt and/or solvate thereof according to clause 45, wherein R^{B} is H.

Clause 47. The compound or salt and/or solvate thereof according to clause 45, wherein R^{B} is C₂₋₃ alkyl substituted with OH or N(R^{B2})(R^{B3}).

Clause 48. The compound or salt and/or solvate thereof according to clause 47, wherein R^{B2} is H and R^{B3} is SO₂(C₁₋₂ alkyl).

Clause 49. The compound or salt and/or solvate thereof according to clause 47, wherein R^{B2} is H or methyl, especially methyl, and R^{B3} is methyl.

Clause 50. The compound or salt and/or solvate thereof according to clause 47, wherein R^{B2} and R^{B3}, together with the nitrogen atom to which they are attached, combine to form a 4- to 6-membered heterocyclic ring, especially a 6-membered heterocyclic ring optionally containing a further heteroatom selected from O and N.

Clause 51. The compound or salt and/or solvate thereof according to claim 50, wherein R^{B2} and R^{B3}, together with the nitrogen atom to which they are attached, combine to form a morpholine ring.

Clause 52. The compound or salt and/or solvate thereof according to any one of clauses 1 to 46, wherein R^{B} is:

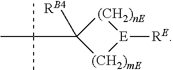

wherein R^{B4}, mE, nE, E and R^{E} are as defined in clause 1.

Clause 53. The compound or salt and/or solvate thereof according to clause 52, wherein R^{B4} is H.

Clause 54. The compound or salt and/or solvate thereof according to clause 52, wherein R^{B4} is methyl.

Clause 55. The compound or salt and/or solvate thereof according to any one of clauses 52 to 54, wherein E is N.

Clause 56. The compound or salt and/or solvate thereof according to clause 55, wherein R^{E} is SO₂R^{9B}.

Clause 57. The compound or salt and/or solvate thereof according to clause 55, wherein R^{E} is R^{9B}.

Clause 58. The compound or salt and/or solvate thereof according to clause 55, wherein R^{E} is C(O)R^{9B}.

Clause 59. The compound or salt and/or solvate thereof according to any one of clauses 56 to 58, wherein R^{9B} is C₁₋₄ alkyl.

Clause 60. The compound or salt and/or solvate thereof according to clause 59, wherein R^{9B} is methyl.

Clause 61. The compound or salt and/or solvate thereof according to any one of clauses 52 to 54, wherein E is O.

Clause 62. The compound or salt and/or solvate thereof according to any one of clauses 52 to 54, wherein E is SO₂

Clause 63. The compound or salt and/or solvate thereof according to clause 61 or clause 62, wherein R^{E} is absent.

Clause 64. The compound or salt and/or solvate thereof according to any one of clauses 52 to 63, wherein nE is 1 or 2.

Clause 65. The compound or salt and/or solvate thereof according to any one of clauses 52 to 64, wherein mE is 1 or 2.

Clause 66. The compound or salt and/or solvate thereof according to clause 52, wherein:
nE is 1 or 2, mE is 1 or 2, E is N and R^{E} is SO₂R^{9B}, wherein R^{9B} is methyl; or
nE is 2, mE is 2, E is N and R^{E} is R^{9B} wherein R^{9B} is methyl; or
nE is 1 or 2, mE is 1 or 2, E is N and R^{E} is C(O)R^{9B}, wherein R^{9B} is methyl; or
nE is 1, mE is 1 and E is O or SO₂.

Clause 67. The compound or salt and/or solvate thereof according to clause 53 wherein E is N or O; and
when E is N, R^{E} is SO₂R^{9B}; wherein R^{9B} is C₁₋₄ alkyl; and
when E is O, R^{E} is absent.

Clause 68. The compound or salt and/or solvate thereof according to any one of clauses 1 or 4 to 44, wherein R^{Z} is tetrazol-5-yl.

Clause 69. The compound or salt and/or solvate thereof according to any one of clauses 1 to 68, wherein R^{C} is H.

Clause 70. The compound or salt and/or solvate thereof according to any one of clauses 1 to 68, wherein R^{C} is C₁₋₂alkyl.

Clause 71. The compound or salt and/or solvate thereof according to any one of clauses 1 to 68, wherein R^{C} is hydroxy.

Clause 72. The compound or salt and/or solvate thereof according to any one of clauses 1 to 68, wherein R^{C} is methoxy.

Clause 73. The compound or salt and/or solvate thereof according to any one of clauses 1 to 68, wherein R^{C} is fluoro.

Clause 74. The compound or salt and/or solvate thereof according to any one of clauses 1 to 73, wherein R^{D} is H.

Clause 75. The compound or salt and/or solvate thereof according to any one of clauses 1 to 73, wherein R^{D} is C₁₋₂alkyl.

Clause 76. The compound or salt and/or solvate thereof according to any one of clauses 1 to 73, wherein R^{D} is hydroxy.

Clause 77. The compound or salt and/or solvate thereof according to any one of clauses 1 to 73, wherein R^{D} is methoxy.

Clause 78. The compound or salt and/or solvate thereof according to any one of clauses 1 to 73, wherein R^{D} is fluoro.

Clause 79. The compound or salt and/or solvate thereof according to any one of clauses 1 to 78, wherein R^{F} is H and R^{G} is H.

Clause 80. The compound or salt and/or solvate thereof according to any one of clauses 1, 2 or 4 to 78, wherein each of R^{F} and R^{G} is independently H or methyl, provided that at least one of R^{F} and R^{G} is H.

Clause 81. The compound or salt and/or solvate thereof according to any one of clauses 1 or 4 to 78, which is a compound of formula (Ic):

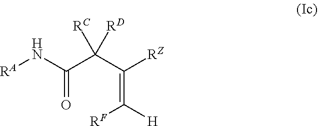

wherein R^{F} is phenyl or C₁₋₄ alkyl optionally substituted with phenyl, OR^{K} or N(R^{K})(R^{L}) and R^{G} is H, wherein R^{K} and R^{L}, R^{A}, R^{C}, R^{D} and R^{Z} are as defined above for Formula (I) in clause 1.

Clause 82. The compound or salt and/or solvate thereof according to any one of clauses 1 or 4 to 78, wherein R^{F} is H and R^{G} is phenyl or C₁₋₄ alkyl optionally substituted with phenyl, OR^{K} or N(R^{K})(R^{L}), wherein R^{K} and R^{L} are as defined above for Formula (I) in clause 1.

Clause 83. The compound or salt and/or solvate thereof according to clause 81 or clause 82, wherein R^{K} and R^{L} are each independently H or methyl.

Clause 84. The compound or salt and/or solvate thereof according to clause 81 wherein R^{Z} is C(O)OR^{B}, R^{F} is phenyl or C₁₋₄ alkyl optionally substituted with phenyl, OR^{K} or N(R^{K})(R^{L}), R^{G} is H and the compound is a compound of formula (Id):

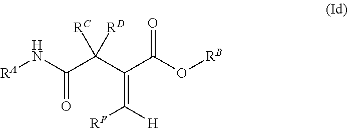

wherein R^{F} is phenyl or C₁₋₄ alkyl optionally substituted with phenyl, OR^{K} or N(R^{K})(R^{L}) and R^{A}, R^{C}, R^{D} and R^{B} are as defined for formula (I) in clause 1.

Clause 85. The compound or salt and/or solvate thereof according to any one of clauses 1 to 84 wherein when the CH₂ of the CH₂(substituted phenyl) is substituted by one R^{A3} the stereochemistry of the carbon to which R^{A3} is attached is as follows:

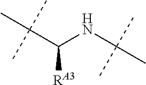

wherein the dashed lines indicate attachment to the remainder of the compound of formula (I).

Clause 86. The compound or salt and/or solvate thereof according to any one of clauses 1 to 87 wherein when R^{A4} is Cl, the CH₂ group is unsubstituted or is substituted by one R^{A3}.

Clause 88. The compound according to clause 1, which is selected from the list consisting of:
1-(methylsulfonyl)piperidin-4-yl 4-((4-chlorobenzyl)amino)-2-methylene-4-oxobutanoate;
1-(methylsulfonyl)piperidin-4-yl 4-(cyclooctylamino)-2-methylene-4-oxobutanoate;
(S)-2-methylene-4-oxo-4-((1-(4-(trifluoromethyl)phenyl)ethyl)amino)butanoic acid;
2-methylene-4-oxo-4-((2-(4-(trifluoromethyl)phenyl)propan-2-yl)amino)butanoic acid;
2-methylene-4-oxo-4-((1-(4-(trifluoromethyl)phenyl)cyclobutyl)amino)butanoic acid;
2-methylene-4-oxo-4-((4-(trifluoromethyl)benzyl)amino)butanoic acid;
2-methylene-4-oxo-4-((2,2,2-trifluoro-1-(4-(trifluoromethyl)phenyl)ethyl)amino)butanoic acid;
(S)-2-methylene-4-oxo-4-((1-(4-(trifluoromethyl)phenyl)propyl)amino)butanoic acid;
1-(methylsulfonyl)piperidin-4-yl 2-methylene-4-oxo-4-((1-(4-(trifluoromethyl)phenyl)cyclobutyl) amino)butanoate;
4-((cyclopropyl(4-(trifluoromethyl)phenyl)methyl)amino)-2-methylene-4-oxobutanoic acid;
2-methylene-4-oxo-4-((1-(4-(trifluoromethyl)phenyl)cyclopropyl)amino)butanoic acid;
(R)-4-((2-hydroxy-1-(4-(trifluoromethyl)phenyl)ethyl)amino)-2-methylene-4-oxobutanoic acid;
2-methylene-4-oxo-4-((3-(4-(trifluoromethyl)phenyl)oxetan-3-yl)amino)butanoic acid;
2-methylene-4-oxo-4-((1-(4-(trifluoromethyl)phenyl)cyclopentyl)amino)butanoic acid;
(S)-4-((1-(4-chlorophenyl)ethyl)amino)-2-methylene-4-oxobutanoic acid;
(S)-4-((1-(4-bromophenyl)ethyl)amino)-2-methylene-4-oxobutanoic acid;
(S)-4-((1-(4-bromophenyl)ethyl)amino)-2-methylene-4-oxobutanoic acid;
(S)-4-((1-(4-(difluoromethyl)phenyl)ethyl)amino)-2-methylene-4-oxobutanoic acid;
(S)-4-((1-(3-fluoro-4-(trifluoromethyl)phenyl)ethyl)amino)-2-methylene-4-oxobutanoic acid;
(S)-2-methylene-4-oxo-4-((1-(4-(perfluoroethyl)phenyl)ethyl)amino)butanoic acid;
(R)-2-methylene-4-oxo-4-((2,2,2-trifluoro-1-(4-(trifluoromethyl)phenyl)ethyl)amino)butanoic acid;
1-(methylsulfonyl)piperidin-4-yl (S)-2-methylene-4-oxo-4-((1-(4-(trifluoromethyl)phenyl)ethyl)amino)butanoate;
4-((cyclopropyl(4-(trifluoromethyl)phenyl)methyl)amino)-2-methylene-4-oxobutanoic acid;
1-methylpiperidin-4-yl (S)-2-methylene-4-oxo-4-((1-(4-(trifluoromethyl)phenyl)ethyl)amino)butanoate;
2-(methylsulfonamido)ethyl (S)-2-methylene-4-oxo-4-((1-(4-(trifluoromethyl)phenyl)ethyl)amino)butanoate;
2-(dimethylamino)ethyl (S)-2-methylene-4-oxo-4-((1-(4-(trifluoromethyl)phenyl)ethyl)amino)butanoate;
1,1-dioxidothietan-3-yl (S)-2-methylene-4-oxo-4-((1-(4-(trifluoromethyl)phenyl)ethyl)amino)butanoate;
3-methyloxetan-3-yl (S)-2-methylene-4-oxo-4-((1-(4-(trifluoromethyl)phenyl)ethyl)amino)butanoate;
2-morpholinoethyl 2-(diethoxyphosphoryl)-4-oxo-4-(((S)-1-(4-(trifluoromethyl)phenyl)ethyl)amino)butanoate;
(S)-2-methylene-4-oxo-4-((1-(5-(trifluoromethyl)pyridin-2-yl)ethyl)amino)butanoic acid;
1-(methylsulfonyl)piperidin-4-yl (S)-2-methylene-4-oxo-4-((1-(5-(trifluoromethyl)pyridin-2-yl)ethyl)amino)butanoate;
1-methylpiperidin-4-yl (S)-2-methylene-4-oxo-4-((1-(5-(trifluoromethyl)pyridin-2-yl)ethyl)amino)butanoate;
1,1-dioxidothietan-3-yl (S)-2-methylene-4-oxo-4-((1-(5-(trifluoromethyl)pyridin-2-yl)ethyl)amino)butanoate;
2-hydroxyethyl (S)-2-methylene-4-oxo-4-((1-(5-(trifluoromethyl)pyridin-2-yl)ethyl)amino)butanoate;
1-acetylpiperidin-4-yl (S)-2-methylene-4-oxo-4-((1-(5-(trifluoromethyl)pyridin-2-yl)ethyl)amino)butanoate;
1-acetylazetidin-3-yl (S)-2-methylene-4-oxo-4-((1-(5-(trifluoromethyl)pyridin-2-yl)ethyl)amino)butanoate;
1-(methylsulfonyl)azetidin-3-yl (S)-2-methylene-4-oxo-4-((1-(5-(trifluoromethyl)pyridin-2-yl)ethyl)amino)butanoate;
(S)-2-methylene-4-oxo-4-((1-(6-(trifluoromethyl)pyridin-3-yl)ethyl)amino)butanoic acid;
2-methylene-4-oxo-4-((1-(5-(trifluoromethyl)pyridin-2-yl)cyclobutyl)amino)butanoic acid;
2-methylene-4-oxo-4-((5-(trifluoromethyl)-2,3-dihydro-1H-inden-1-yl)amino)butanoic acid;
(S,Z)-2-(2-oxo-2-((1-(4-(trifluoromethyl)phenyl)ethyl)amino)ethyl)but-2-enoic acid; and
(S,E)-2-(2-oxo-2-((1-(4-(trifluoromethyl)phenyl)ethyl)amino)ethyl)but-2-enoic acid;
(S,E)-2-(2-oxo-2-((1-(4-(trifluoromethyl)phenyl)ethyl)amino)ethyl)pent-2-enoic acid;
(S,E)-4-methyl-2-(2-oxo-2-((1-(4-(trifluoromethyl)phenyl)ethyl)amino)ethyl)pent-2-enoic acid;
(S,E)-5-methyl-2-(2-oxo-2-((1-(4-(trifluoromethyl)phenyl)ethyl)amino)ethyl)hex-2-enoic acid;
(S,E)-2-(2-oxo-2-((1-(4-(trifluoromethyl)phenyl)ethyl)amino)ethyl)-5-phenylpent-2-enoic acid;
(S,E)-2-benzylidene-4-oxo-4-((1-(4-(trifluoromethyl)phenyl)ethyl)amino)butanoic acid;
(S,Z)-2-benzylidene-4-oxo-4-((1-(4-(trifluoromethyl)phenyl)ethyl)amino)butanoic acid;
(S,E)-4-(dimethylamino)-2-(2-oxo-2-((1-(4-(trifluoromethyl)phenyl)ethyl)amino)ethyl)but-2-enoic acid;
(S)-3,3-dimethyl-2-methylene-4-oxo-4-((1-(4-(trifluoromethyl)phenyl)ethyl)amino)butanoic acid;
3-methyl-2-methylene-4-oxo-4-((2-(4-(trifluoromethyl)phenyl)propan-2-yl)amino)butanoic acid;
3-methyl-2-methylene-4-oxo-4-((2-(4-(trifluoromethyl)phenyl)propan-2-yl)amino)butanoic acid ISOMER 1;
3-methyl-2-methylene-4-oxo-4-((2-(4-(trifluoromethyl)phenyl)propan-2-yl)amino)butanoic acid ISOMER 2;
3-methyl-2-methylene-4-oxo-4-(((S)-1-(4-(trifluoromethyl)phenyl)ethyl)amino)butanoic acid ISOMER 1;
3-methyl-2-methylene-4-oxo-4-(((S)-1-(4-(trifluoromethyl)phenyl)ethyl)amino)butanoic acid ISOMER 2;
(S)-2-methylene-4-oxo-4-((1-(4-(pentafluoro-A6-sulfaneyl)phenyl)ethyl)amino)butanoic acid;
(S)-3-(1H-tetrazol-5-yl)-N-(1-(4-(trifluoromethyl)phenyl)ethyl)but-3-enamide;
   or a pharmaceutically acceptable salt and/or solvate of any one thereof.

Clause 89. A pharmaceutical composition comprising a compound according to any one of clauses 1 to 88 or a pharmaceutically acceptable salt and/or solvate thereof.

Clause 90. A compound or salt and/or solvate thereof according to any one of clauses 1 to 88 or a pharmaceutical composition according to clause 89 for use as a medicament.

Clause 91. A compound or salt and/or solvate thereof according to any one of clauses 1 to 88 or a pharmaceutical composition according to clause 89 for use in treating or preventing an inflammatory disease or a disease associated with an undesirable immune response.

Clause 92. Use of a compound or salt and/or solvate thereof according to any one of clauses 1 to 88 or a pharmaceutical composition according to clause 89 in the manufacture of a medicament for treating or preventing an inflammatory disease or a disease associated with an undesirable immune response.

Clause 93. A method of treating or preventing an inflammatory disease or a disease associated with an undesirable immune response, which comprises administering a compound or salt and/or solvate thereof according to any one of clauses 1 to 88 or a pharmaceutical composition according to clause 89.

Clause 94. The compound or salt and/or solvate thereof, pharmaceutical composition, compound or salt and/or solvate thereof for use, use or method according to any one of clauses 1 to 93, for treating an inflammatory disease or a disease associated with an undesirable immune response.

Clause 95. The compound or salt and/or solvate thereof, pharmaceutical composition, compound or salt and/or solvate thereof for use, use or method according to any one of clauses 1 to 93, for preventing an inflammatory disease or a disease associated with an undesirable immune response.

Clause 96. The compound or salt and/or solvate thereof, pharmaceutical composition, compound or salt and/or solvate thereof for use, use or method according to any one of clauses 1 to 93, for treating or preventing an inflammatory disease.

Clause 97. The compound or salt and/or solvate thereof, pharmaceutical composition, compound or salt and/or solvate thereof for use, use or method according to any one of clauses 1 to 93, for treating or preventing a disease associated with an undesirable immune response.

Clause 98. The compound or salt and/or solvate thereof, pharmaceutical composition, compound or salt and/or solvate thereof for use, use or method according to any one of clauses 1 to 93, wherein the inflammatory disease or disease associated with an undesirable immune response is, or is associated with, a disease selected from the group consisting of: psoriasis (including chronic plaque, erythrodermic, pustular, guttate, inverse and nail variants), asthma, chronic obstructive pulmonary disease (COPD, including chronic bronchitis and emphysema), heart failure (including left ventricular failure), myocardial infarction, angina pectoris, other atherosclerosis and/or atherothrombosis-related disorders (including peripheral vascular disease and ischaemic stroke), a mitochondrial and neurodegenerative disease (such as Parkinson's disease, Alzheimer's disease, Huntington's disease, amyotrophic lateral sclerosis, retinitis pigmentosa or mitochondrial encephalomyopathy), autoimmune paraneoplastic retinopathy, transplantation rejection (including antibody-mediated and T cell-mediated forms), multiple sclerosis, transverse myelitis, ischaemia-reperfusion injury (e.g. during elective surgery such as cardiopulmonary bypass for coronary artery bypass grafting or other cardiac surgery, following percutaneous coronary intervention, following treatment of acute ST-elevation myocardial infarction or ischaemic stroke, organ transplantation, or acute compartment syndrome), AGE-induced genome damage, an inflammatory bowel disease (e.g. Crohn's disease or ulcerative colitis), primary sclerosing cholangitis (PSC), PSC-autoimmune hepatitis overlap syndrome, non-alcoholic fatty liver disease (non-alcoholic steatohepatitis), rheumatica, granuloma annulare, cutaneous lupus erythematosus (CLE), systemic lupus erythematosus (SLE), lupus nephritis, drug-induced lupus, autoimmune myocarditis or myopericarditis, Dressler's syndrome, giant cell myocarditis, post-pericardiotomy syndrome, drug-induced hypersensitivity syndromes (including hypersensitivity myocarditis), eczema, sarcoidosis, erythema nodosum, acute disseminated encephalomyelitis (ADEM), neuromyelitis optica spectrum disorders, MOG (myelin oligodendrocyte glycoprotein) antibody-associated disorders (including MOG-EM), optic neuritis, CLIPPERS (chronic lymphocytic inflammation with pontine perivascular enhancement responsive to steroids), diffuse myelinoclastic sclerosis, Addison's disease, alopecia areata, ankylosing spondylitis, other spondyloarthritides (including peripheral spondyloarthritis, that is associated with psoriasis, inflammatory bowel disease, reactive arthritis or juvenile onset forms), antiphospholipid antibody syndrome, autoimmune hemolytic anaemia, autoimmune hepatitis, autoimmune inner ear disease, pemphigoid (including bullous pemphigoid, mucous membrane pemphigoid, cicatricial pemphigoid, herpes gestationis or pemphigoid gestationis, ocular cicatricial pemphigoid), linear IgA disease, Behçet's disease, celiac disease, Chagas disease, dermatomyositis, diabetes mellitus type I, endometriosis, Goodpasture's syndrome, Graves' disease, Guillain-Barre syndrome and its subtypes (including acute inflammatory demyelinating polyneuropathy, AIDP, acute motor axonal neuropathy (AMAN), acute motor and sensory axonal neuropathy (AMSAN), pharyngeal-cervical-brachial variant, Miller-Fisher variant and Bickerstaff's brainstem encephalitis), progressive inflammatory neuropathy, Hashimoto's disease, hidradenitis suppurativa, inclusion body myositis, necrotising myopathy, Kawasaki disease, IgA nephropathy, Henoch-Schonlein purpura, idiopathic thrombocytopenic purpura, thrombotic thrombocytopenic purpura (TTP), Evans' syndrome, interstitial cystitis, mixed connective tissue disease, undifferentiated connective tissue disease, morphea, myasthenia gravis (including MuSK antibody positive and seronegative variants), narcolepsy, neuromyotonia, pemphigus vulgaris, pernicious anaemia, psoriatic arthritis, polymyositis, primary biliary cholangitis (also known as primary biliary cirrhosis), rheumatoid arthritis, palindromic rheumatism, schizophrenia, autoimmune (meningo-)encephalitis syndromes, scleroderma, Sjogren's syndrome, stiff person syndrome, polymylagia rheumatica, giant cell arteritis (temporal arteritis), Takayasu arteritis, polyarteritis nodosa, Kawasaki disease, granulomatosis with polyangitis (GPA; formerly known as Wegener's granulomatosis), eosinophilic granulomatosis with polyangiitis (EGPA; formerly known as Churg-Strauss syndrome), microscopic polyarteritis/polyangiitis, hypocomplementaemic urticarial vasculitis, hypersensitivity vasculitis, cryoglobulinemia, thromboangiitis obliterans (Buerger's disease), vasculitis, leukocytoclastic vasculitis, vitiligo, acute disseminated encephalomyelitis, adrenoleukodystrophy, Alexander's disease, Alper's disease, balo concentric sclerosis or Marburg disease, cryptogenic organising pneumonia (formerly known as bronchiolitis obliterans organizing pneumonia), Canavan disease, central nervous system vasculitic syndrome, Charcot-Marie-Tooth disease, childhood ataxia with central nervous system hypomyelination, chronic inflammatory demyelinating polyneuropathy (CIDP), diabetic retinopathy, globoid cell leukodystrophy (Krabbe disease), graft-versus-host disease (GVHD) (including acute and chronic forms, as well as intestinal GVHD), hepatitis C (HCV) infection or complication, herpes simplex viral infection or complication, human immunodeficiency virus (HIV) infection or complication, lichen planus, monomelic amyotrophy, cystic fibrosis, pulmonary arterial hypertension (PAH, including idiopathic PAH), lung sarcoidosis, idiopathic pulmonary fibrosis, paediatric asthma, atopic dermatitis, allergic dermatitis, contact dermatitis, allergic rhinitis, rhinitis, sinusitis, conjunctivitis, allergic conjunctivitis, keratoconjunctivitis sicca, dry eye, xerophthalmia, glaucoma, macular oedema, diabetic macular oedema, central retinal vein occlusion (CRVO), macular degeneration (including dry and/or wet age related macular degeneration, AMD), post-operative cataract inflammation, uveitis (including posterior, anterior, intermediate and pan uveitis), iridocyclitis, scleritis, corneal graft and limbal cell transplant rejection, gluten sensitive enteropathy (coeliac disease), dermatitis herpetiformis, eosinophilic esophagitis, achalasia, autoimmune dysautonomia, autoimmune encephalomyelitis, autoimmune oophoritis, autoimmune orchitis, autoimmune pancreatitis, aortitis and periaortitis, autoimmune retinopathy, autoimmune urticaria, (idiopathic) Castleman's disease, Cogan's syndrome, IgG4-related disease, retroperitoneal fibrosis, juvenile idiopathic arthritis including systemic juvenile idiopathic arthritis (Still's disease), adult-onset Still's disease, ligneous conjunctivitis, Mooren's ulcer, pityriasis lichenoides et varioliformis acuta (PLEVA, also known as Mucha-Habermann disease), multifocal motor neuropathy (MMN), paediatric acute-onset neuropsychiatric syndrome (PANS) (including paediatric autoimmune neuropsychiatric disorders associated with streptococcal infections (PANDAS)), paraneoplastic syndromes (including paraneoplastic cerebellar degeneration, Lambert-Eaton myaesthenic syndrome, limbic encephalitis, brainstem encephalitis, opsoclonus myoclonus ataxia syndrome, anti-NMDA receptor encephalitis, thymoma-associated multiorgan autoimmunity), perivenous encephalomyelitis, reflex sympathetic dystrophy, relapsing polychondritis, sperm & testicular autoimmunity, Susac's syndrome, Tolosa-Hunt syndrome, Vogt-Koyanagi-Harada Disease, anti-synthetase syndrome, autoimmune enteropathy, immune dysregulation polyendocrinopathy enteropathy X-linked (IPEX), microscopic colitis, autoimmune lymphoproliferative syndrome (ALPS), autoimmune polyendocrinopathy-candidiasis-ectodermal dystrophy syndrome (APEX), gout, pseudogout, amyloid (including AA or secondary amyloidosis), eosinophilic fasciitis (Shulman syndrome) progesterone hypersensitivity (including progesterone dermatitis), amilial Mediterranean fever (FMF), tumour necrosis factor (TNF) receptor-associated periodic fever syndrome (TRAPS), hyperimmunoglobulinaemia D with periodic fever syndrome (HIDS), PAPA (pyogenic arthritis, pyoderma gangrenosum, severe cystic acne) syndrome, deficiency of interleukin-1 receptor antagonist (DIRA), deficiency of the interleukin-36-receptor antagonist (DITRA), cryopyrin-associated periodic syndromes (CAPS) (including familial cold autoinflammatory syndrome [FCAS], Muckle-Wells syndrome, neonatal onset multisystem inflammatory disease [NOMID]), NLRP12-associated autoinflammatory disorders (NLRP12AD), periodic fever aphthous stomatitis (PFAPA), chronic atypical neutrophilic dermatosis with lipodystrophy and elevated temperature (CANDLE), Majeed syndrome, Blau syndrome (also known as juvenile systemic granulomatosis), macrophage activation syndrome, chronic recurrent multifocal osteomyelitis (CRMO), familial cold autoinflammatory syndrome, mutant adenosine deaminase 2 and monogenic interferonopathies (including Aicardi-Goutières syndrome, retinal vasculopathy with cerebral leukodystrophy, spondyloenchondrodysplasia, STING [stimulator of interferon genes]-associated vasculopathy with onset in infancy, proteasome associated autoinflammatory syndromes, familial chilblain lupus, dyschromatosis symmetrica hereditaria), Schnitzler syndrome; familial cylindromatosis, congenital B cell lymphocytosis, OTULIN-related autoinflammatory syndrome, type 2 diabetes mellitus, insulin resistance and the metabolic syndrome (including obesity-associated inflammation), atherosclerotic disorders (e.g. myocardial infarction, angina, ischaemic heart failure, ischaemic nephropathy, ischaemic stroke, peripheral vascular disease, aortic aneurysm), renal inflammatory disorders (e.g. diabetic nephropathy, membranous nephropathy, minimal change disease, crescentic glomerulonephritis, acute kidney injury, renal transplantation).

Clause 99. The compound or salt and/or solvate thereof, pharmaceutical composition, compound or salt and/or solvate thereof for use, use or method according to clause 98, wherein the inflammatory disease or disease associated with an undesirable immune response is selected from the group consisting of rheumatoid arthritis, psoriatic arthritis, ankylosing spondylitis, systemic lupus erythematosus, multiple sclerosis, psoriasis, Crohn's disease, ulcerative colitis, uveitis, cryopyrin-associated periodic syndromes, Muckle-Wells syndrome, juvenile idiopathic arthritis, chronic obstructive pulmonary disease and asthma.

Clause 100. The compound or salt and/or solvate thereof, pharmaceutical composition, compound or salt and/or solvate thereof for use, use or method according to clause 99, wherein the inflammatory disease or disease associated with an undesirable immune response is multiple sclerosis.

Clause 101. The compound or salt and/or solvate thereof, pharmaceutical composition, compound or salt and/or solvate thereof for use, use or method according to clause 99, wherein the inflammatory disease or disease associated with an undesirable immune response is psoriasis.

Clause 102. The compound or salt and/or solvate thereof, pharmaceutical composition, compound or salt and/or solvate thereof for use, use or method according to clause 99, wherein the inflammatory disease or disease associated with an undesirable immune response is asthma.

Clause 103. The compound or salt and/or solvate thereof, pharmaceutical composition, compound or salt and/or solvate thereof for use, use or method according to clause 99, wherein the inflammatory disease or disease associated with an undesirable immune response is chronic obstructive pulmonary disease.

Clause 104. The compound or salt and/or solvate thereof, pharmaceutical composition, compound or salt and/or solvate thereof for use, use or method according to clause 99, wherein the inflammatory disease or disease associated with an undesirable immune response is systemic lupus erythematosus.

Clause 105. The compound, pharmaceutical composition, compound or salt and/or solvate thereof for use, use or method according to any one of clauses 1 to 96, wherein the compound is for administration to a human subject.

Clause 106. The compound or salt and/or solvate thereof, pharmaceutical composition, compound or salt and/or solvate thereof for use, use or method according to any one of clauses 1 to 105, for use in combination with a further therapeutic agent, such as a corticosteroid (glucocorticoid), retinoid (e.g. acitretin, isotretinoin, tazarotene), anthralin, vitamin D analogue (e.g. cacitriol, calcipotriol), calcineurin inhibitors (e.g. tacrolimus, pimecrolimus), phototherapy or photochemotherapy (e.g. psoralen ultraviolet irradiation, PUVA) or other form of ultraviolet light irradiation therapy, ciclosporine, a thiopurine (e.g. azathioprine, 6-mercaptopurine), methotrexate, an anti-TNFα agents (e.g. infliximab, etanercept, adalimumab, certolizumab, golimumab or a biosimilar), phosphodiesterase-4 (PDE4) inhibition (e.g. apremilast, crisaborole), anti-IL-17 agent (e.g. brodalumab, ixekizumab, secukinumab), anti-IL12/IL-23 agent (e.g. ustekinumab, briakinumab), anti-IL-23 agent (e.g. guselkumab, tildrakizumab), JAK (Janus Kinase) inhibitor (e.g. tofacitinib, ruxolitinib, baricitinib, filgotinib, upadacitinib), plasma exchange, intravenous immune globulin (IVIG), cyclophosphamide, anti-CD20 B cell depleting agent (e.g. rituximab, ocrelizumab, ofatumumab, obinutuzumab), anthracycline analogue (e.g. mitoxantrone), cladribine, sphingosine 1-phosphate receptor modulator or sphingosine analogue (e.g. fingolimod, siponimod, ozanimod, etrasimod), interferon beta preparation (including interferon beta 1b/1a), glatiramer, anti-CD3 therapy (e.g. OKT3), anti-CD52 targeting agent (e.g. alemtuzumab), leflunomide, teriflunomide, gold compound, laquinimod, potassium channel blocker (e.g. dalfampridine/4-aminopyridine), mycophenolic acid, mycophenolate mofetil, purine analogue (e.g. pentostatin), mTOR (mechanistic target of rapamycin) pathway inhibitor (e.g. sirolimus, everolimus), anti-thymocyte globulin (ATG), IL-2 receptor (CD25) inhibitor (e.g. basiliximab, daclizumab), anti-IL-6 receptor or anti-IL-6 agent (e.g. tocilizumab, siltuximab), Bruton's tyrosine kinase (BTK) inhibitor (e.g. ibrutinib), tyrosine kinase inhibitor (e.g. imatinib), ursodeoxycholic acid, hydroxychloroquine, chloroquine, B cell activating factor (BAFF, also known as BLyS, B lymphocyte stimulator) inhibitor (e.g. belimumab, blisibimod), other B cell targeted therapy including a fusion protein targeting both APRIL (A PRoliferation-Inducing Ligand) and BLyS (e.g. atacicept), PI3K inhibitor including pan-inhibitor or one targeting the p110δ and/or p110γ containing isoforms (e.g. idelalisib, copanlisib, duvelisib), an interferon α receptor inhibitor (e.g. anifrolumab, sifalimumab), T cell co-stimulation blocker (e.g. abatacept, belatacept), thalidomide and its derivatives (e.g. lenalidomide), dapsone, clofazimine, a leukotriene antagonist (e.g. montelukast), theophylline, anti-IgE therapy (e.g. omalizumab), an anti-IL-5 agent (e.g. mepolizumab, reslizumab), a long-acting muscarinic agent (e.g. tiotropium, aclidinium, umeclidinium), a PDE4 inhibitor (e.g. roflumilast), riluzole, a free radical scavenger (e.g. edaravone), a proteasome inhibitor (e.g. bortezomib), a complement cascade inhibitor including one directed against C5 (e.g. eculizumab), immunoadsor, antithymocyte globulin, 5-aminosalicylates and their derivatives (e.g. sulfasalazine, balsalazide, mesalamine), an anti-integrin agent including one targeting α4β1 and/or α4β7 integrins (e.g. natalizumab, vedolizumab), an anti-CD11-α agent (e.g. efalizumab), a non-steroidal anti-inflammatory drug (NSAID) including a salicylate (e.g. aspirin), a propionic acid (e.g. ibuprofen, naproxen), an acetic acid (e.g. indomethacin, diclofenac, etodolac), an oxicam (e.g. meloxicam) a fenamate (e.g. mefenamic acid), a selective or relatively selective COX-2 inhibitor (e.g. celecoxib, etroxicoxib, valdecoxib and etodolac, meloxicam, nabumetone), colchicine, an IL-4 receptor inhibitor (e.g. dupilumab), topical/contact immunotherapy (e.g. diphenylcyclopropenone, squaric acid dibutyl ester), anti-IL-1 receptor therapy (e.g. anakinra), IL-1P inhibitor (e.g. canakinumab), IL-1 neutralising therapy (e.g. rilonacept), chlorambucil, a specific antibiotic with immunomodulatory properties and/or ability to modulate NRF2 (e.g. tetracyclines including minocycline, clindamycin, macrolide antibiotics), anti-androgenic therapy (e.g. cyproterone, spironolactone, finasteride), pentoxifylline, ursodeoxycholic acid, obeticholic acid, fibrate, a cystic fibrosis transmembrane conductance regulator (CFTR) modulator, a VEGF (vascular endothelial growth factor) inhibitor (e.g. bevacizumab, ranibizumab, pegaptanib, aflibercept), pirfenidone or mizoribine.

Clause 107. A process for preparing a compound of formula (I) in which R^{Z} is C(O)OR^{B}, or a salt such as a pharmaceutical acceptable salt thereof, which comprises reacting a compound of formula (II):

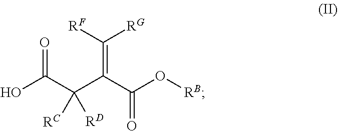

or a salt thereof;
with amine R^{A}—NH₂ (V), or a salt thereof;
wherein R^{B} is:

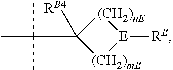

and R^{A}, R^{C} and R^{D} are defined in clause 1.

Clause 108. A process for preparing a compound of formula (I) in which R^{Z} is C(O)OR^{B}, or a salt, such as a pharmaceutical acceptable salt, thereof, which comprises deprotecting a compound of formula (VII):

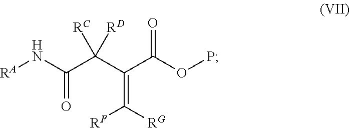

or a salt thereof;
wherein R^{A}, R^{C}, R^{D} R^{F} and R^{G} are defined in clause 1 and P is a carboxylic acid protecting group, such as C₁₋₄alkyl, e.g., tBu, or CH₂CCl₃.

Clause 109. A process for preparing a compound of formula (I) in which R^{Z} is C(O)OR^{B}, or a salt, such as a pharmaceutically acceptable salt, thereof, by reacting a compound of formula (VIIIa) or (VIIIb):

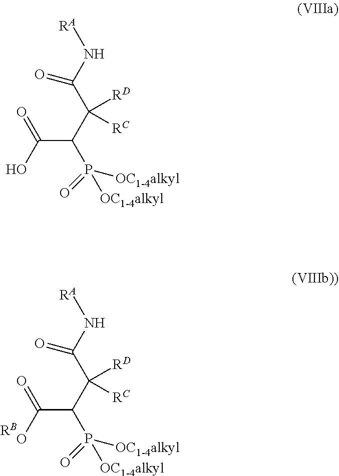

or a salt thereof;
wherein R^{A}, R^{C}, R^{D}, R^{F} and R^{G} are defined elsewhere herein;
with aqueous C(R^{F})(R^{G})═O in the presence of a base.

Clause 110. A process for preparing a compound of formula (I) in which R^{Z} is C(O)OR^{B}, where R^{B} is H, or a salt, such as a pharmaceutically acceptable salt, thereof, which comprises reacting a compound of formula (Va):

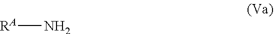

wherein R^{A} is CH₂(substituted phenyl) or CH₂(substituted phenyl), wherein the CH₂ group is optionally substituted with one or two R^{A3} and the phenyl or pyridyl group is substituted with one or more R^{A4}, wherein R^{A3} and R^{A4} are as defined above for formula (I) in clause 1;
with an itaconic anhydride derivative of formula (XX):

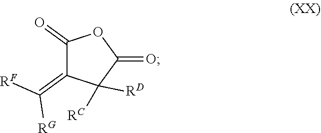

wherein R^{C}, R^{D}, R^{F} and R^{G} are defined in clause 1.

Clause 111. A process for preparing a compound of formula (I) in which R^{F} and R^{G} are both H and R^{Z} is C(O)OR^{B}, or a salt, such as a pharmaceutically acceptable salt, thereof, which comprises reacting a compound of formula (XXV):

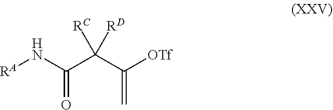

with triphenylphosphine, palladium acetate and carbon monoxide, followed by with a base such as triethylamine and a carboxylic acid such as acetic acid to give a compound of formula (I) in which R^{B} is H; and optionally
reacting a compound of formula (I) in which R^{Z} is C(O)OH with a compound of formula (VI):

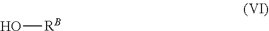

where in R^{B} is as defined in clause 1 except that it is not H;
to give a compound of formula (I) in which R^{Z} is C(O)OR^{B}, where in R^{B} is as defined in clause 1 except that it is not H.

Clause 112. A process for preparing a compound of formula (I) in which R^{Z} is tetrazol-5-yl, or a salt, such as a pharmaceutical acceptable salt, thereof, which comprises reacting a compound of formula (XXXI):

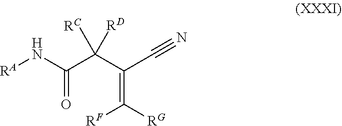

or a salt thereof
wherein R^{A}, R^{C}, R^{D}, R^{F} and R^{G} are defined elsewhere herein;
with trimethylsilyl azide (TMSN₃) in the presence of a catalyst.

Clause 113. A compound of formula (II):

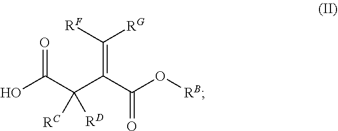

or a salt thereof;
wherein R^{B} is:

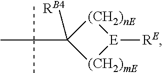

and R^{C}, R^{D}, R^{F} and R^{G} are as defined in clause 1.

Clause 114. A compound of formula (VII):

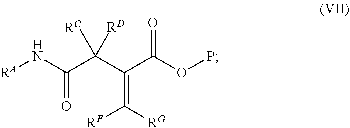

or a salt thereof;
wherein R^{A}, R^{C} and R^{D} are defined in clause 1 and P is a carboxylic acid protecting group, such as C₁₋₄alkyl, e.g., tBu, or CH₂CCl₃.

Clause 115. A compound of formula (VIIIa) or (VIIIb):

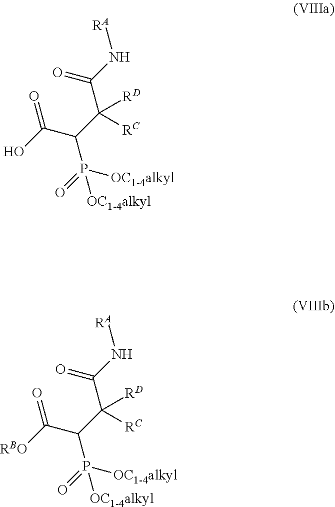

or a salt thereof;
wherein R^{A}, R^{B}, R^{C} and R^{D} are defined in clause 1.

Clause 116. A compound of formula (XXXI):

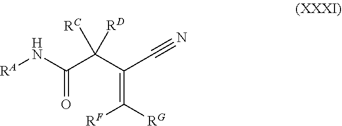

or a salt thereof
wherein R^{A}, R^{C}, R^{D}, R^{F} and R^{G} are defined in clause 1.

Clause 117. A compound of formula (XXV):

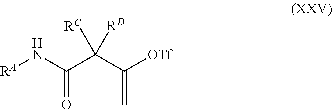

wherein R^{A}, R^{C} and R^{D} are defined in clause 1.

### Abbreviations

Ac acetyl
br broad singlet
BBFO broadband fluorine observe
BEH ethylene bridged hybrid
CSH charged surface hybrid
d doublet
DAD diode array detector
DCC N,N′-dicyclohexylcarbodiimide
DCM dichloromethane
DIPEA N,N-diisopropylethylamine
DMAP 4-dimethylaminopyridine
DMF dimethyl fumarate
DMSO dimethyl sulfoxide
EDC 1-ethyl-3-(3-dimethylaminopropyl)carbodiimide
Et ethyl
ES⁺ electrospray
FBS fetal bovine serum
g gram(s)
GSH glutathione
h hour(s)
HATU 1-[bis(dimethylamino)methylene]-1H-1,2,3-triazolo[4,5-b]pyridinium 3-oxide hexafluorophosphate
HCl hydrochloric acid
HPLC high-performance liquid chromatography
IL interleukin
IPA isopropyl alcohol
LDA lithium diisopropylamide
LCMS liquid chromatography-mass spectrometry
m multiplet
M molar concentration/molar mass
m/z mass to charge ratio
MHz (mega)hertz
min(s) minute(s)
mL millilitres
mmol millimole
MS mass spectrometry
MTBE methyl tertiary-butyl ether
NMM 4-methylmorpholine
NMR nuclear magnetic resonance
NMP N-methyl-2-pyrrolidone
p quintet
PBS phosphate buffered saline
PDA photodiode array
PMB para-methoxybenzyl
q quartet
rpm revolutions per minute
RT room temperature
s singlet
t triplet
TBTU 2-(1H-benzotriazole-1-yl)-1,1,3,3-tetramethylaminium tetrafluoroborate
Tf trifluoromethanesulfonyl, i.e., CF₃SO₂—
TFA trifluoroacetic acid
THF tetrahydrofuran
μL microlitre
μM micromolar
UPLC ultra performance liquid chromatography
wt weight
° C. degrees centigrade

### EXAMPLES

### Analytical Equipment

Thin layer chromatography (TLC) was performed on silica gel plates (GF254, glass, silica gel size: 400-600 mesh). Spots were visualized by UV light (214 and 254 nm) or color reagents (iodine, KMnO₄ aq.).

Bruker 400 MHz Avance Ill spectrometer fitted with a BBFO 5 mm probe, or a Bruker 500 MHz Avance Ill HD spectrometer equipped with a Bruker 5 mm SmartProbe™. ¹H chemical shifts are reported in 5 values in ppm with the deuterated solvent as the internal standard. Data are reported as follows: chemical shift, multiplicity (s=singlet, d=doublet, t=triplet, q=quartet, br=broad, m=multiplet), coupling constant (Hz), integration.

UPLC/MS analysis was carried out on a Waters Acquity UPLC system using either a Waters Acquity CSH C18 or BEH C18 column (2.1×30 mm) maintained at a temperature of 40° C. and eluted with a linear acetonitrile gradient appropriate for the lipophilicity of the compound over 3 or 10 minutes at a constant flow rate of 0.77 mL/min. The aqueous portion of the mobile phase was either 0.1% Formic Acid (CSH C18 column) or 10 mM Ammonium Bicarbonate (BEH C18 column). LC-UV chromatograms were recorded using a Waters Acquity PDA detector between 210 and 400 nm. Mass spectra were recorded using a Waters Acquity Qda detector with electrospray ionisation switching between positive and negative ion mode. Sample concentration was adjusted to give adequate UV response.

LCMS analysis was carried out on a Agilent LCMS system using either a Waters Acquity CSH C18 or BEH C18 column (4.6×30 mm) maintained at a temperature of 40° C. and eluted with a linear acetonitrile gradient appropriate for the lipophilicity of the compound over 4 or 15 minutes at a constant flow rate of 2.5 mL/min. The aqueous portion of the mobile phase was either 0.1% Formic Acid (CSH C18 column) or 10 mM Ammonium Bicarbonate (BEH C18 column). LC-UV chromatograms were recorded using an Agilent VWD or DAD detector at 254 nm. Mass spectra were recorded using an Agilent MSD detector with electrospray ionisation switching between positive and negative ion mode. Sample concentration was adjusted to give adequate UV response.

Alternatively, the analytical LCMS equipment and methods set out in the following table were also used:

LCMS Instrument Details

| System | Instrument Name | LC Detector | ELS detector | Mass detector |
|---|---|---|---|---|
| 1 (basic) | Agilent LCMS 1200 | G1315D DAD | 380 ELSD | Agilent G6120B |
| 2 (acidic) | Agilent LCMS 1200 | G1315C DAD | 380 ELSD | Agilent G6110A |

LCMS/HPLC Method Details

| | | | | | | | Flow |
|---|---|---|---|---|---|---|---|
| | | | | | | Column | Rate |
| Method | Solvent | | | UV | Mass | Temp. | ml/ |
| Name | System | Column | Gradient | range | Range | ° C. | min |
| A (basic | A) water + 10 | Waters X- | From 95:5 to 0:100 | 190-400 | 100-1800 | 40 | 2.0 |
| LCMS) | mM NH₄HCO₃ | Bridge C18 | in 1.6 min, 0:100 for | nm | amu | | |
| | B) acetonitrile | (50 mm × 4.6 | 1.4 min, from 0:100 to | | | | |
| | | mm × 3.5 μm) | 95:5 in 0.1 min, | | | | |
| | | | 95:5 for 0.7 min. | | | | |
| B (acidic | A) water + | Waters X- | From 95:5 to 0:100 | 190-400 | 100-1100 | 40 | 2.0 |
| LCMS) | 0.05% TFA | Bridge C18 | in 1.6 min, 0:100 | nm | amu | | |
| | B) acetonitrile + | (50 mm × 4.6 | for 1.4 min, from 0:100 | | | | |
| | 0.05% TFA | mm × 3.5 μm) | to 95:5 in 0.05 min, | | | | |
| | | | 95:5 for 0.7 min | | | | |
| C (acidic | A) water + | Halo C18 | From 95:5 to 0:100 in | 190-400 | 100-1100 | 40 | 3.0 |
| LCMS) | 0.05% TFA | (30 mm × 4.6 | 0.8 min, 0:100 | nm | amu | | |
| | B) acetonitrile + | mm × 2.7 μm) | for 0.4 min, from 0:100 | | | | |
| | 0.05% TFA | | to 95:5 in 0.01 min, | | | | |
| | | | 95:5 for 0.2 min | | | | |

### Commercial Materials

4-((4-methoxybenzyl)oxy)-2-methylene-4-oxobutanoic acid is commercially available, for example from Combi-Blocks. Dimethyl itaconate was purchased from Sigma-Aldrich (product number: 109533). 4-Octyl itaconate was purchased from BOC biosciences (product number: B0001-007866).

### Other Materials

Reference Example 2, referred to in Table 4, corresponds with Example 49 in WO2020/222011 (Sitryx Therapeutics, 2020), and has the following structure:

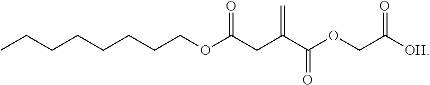 [SMILES: C=C(CC(=O)OCCCCCCCC)C(=O)OCC(=O)O]

### Synthesis of Intermediates

### Intermediate 1: 3-(((1-(methylsulfonyl)piperidin-4-yl)oxy)carbonyl)but-3-enoic acid

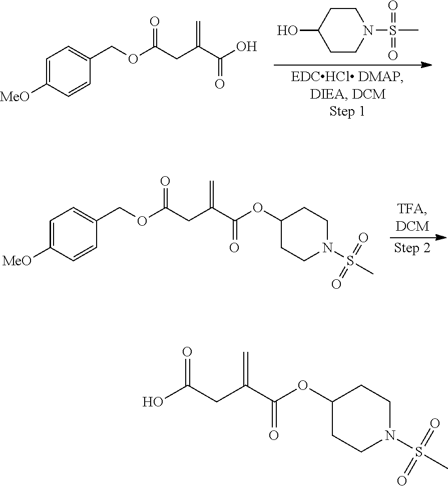 [SMILES: C=C(CC(=O)OCc1ccc(OC)cc1)C(=O)O ; CS(=O)(=O)N1CCC(O)CC1 ; C=C(CC(=O)OCc1ccc(OC)cc1)C(=O)OC1CCN(S(C)(=O)=O)CC1 ; C=C(CC(=O)O)C(=O)OC1CCN(S(C)(=O)=O)CC1]

### Step 1

To a solution of 4-((4-methoxybenzyl)oxy)-2-methylene-4-oxobutanoic acid (1.4 g, 5.6 mmol), DIPEA (1.5 mL, 8.4 mmol), 1-(methylsulfonyl)piperidin-4-ol (1.00 g, 5.6 mmol) and DMAP (0.068 g, 0.56 mmol) in DCM (12 mL) at 0° C. was added EDC·HCl (1.61 g, 8.4 mmol) in DCM (12 mL) dropwise. The mixture was allowed to warm to RT and stirred for 16 h. The mixture was poured into 1 M HCl (50 mL) and extracted with EtOAc (3×50 mL). The combined organic extracts were washed with brine (80 mL), dried (Na₂SO₄) and concentrated. The crude product was purified by chromatography on silica gel (0-100% EtOAc/isohexane) to afford 4-(4-methoxybenzyl) 1-(1-(methylsulfonyl)piperidin-4-yl) 2-methylenesuccinate (0.895 g, 2.18 mmol) as a yellow oil. ¹H NMR (400 MHz, DMSO-d6) δ 7.34-7.23 (m, 2H), 6.96-6.89 (m, 2H), 6.24 (d, J=1.3 Hz, 1H), 5.85 (d, J=1.3 Hz, 1H), 5.03 (s, 2H), 4.92-4.83 (m, 1H), 3.76 (s, 3H), 3.43 (s, 2H), 3.22-3.08 (m, 4H), 2.87 (s, 3H), 1.90-1.80 (m, 2H), 1.68-1.55 (m, 2H). LCMS m/z 433.9 (M+Na)⁺ (ES⁺).

### Step 2

To a solution of 4-(4-methoxybenzyl) 1-(1-(methylsulfonyl)piperidin-4-yl) 2-methylenesuccinate (0.895 g, 2.18 mmol) in DCM (25 mL) at 0° C. was added TFA (3.4 mL, 44 mmol) dropwise. The mixture was allowed to warm to RT and stirred for 30 min. The mixture was concentrated and the residue co-evaporated with toluene (2×50 mL). The crude product was purified by chromatography on silica gel (0-10% MeOH/DCM) to afford 3-(((1-(methylsulfonyl)piperidin-4-yl)oxy)carbonyl)but-3-enoic acid (0.370 g, 1.23 mmol) as a white solid. ¹H NMR (400 MHz, DMSO-d6) δ 12.37 (s, 1H), 6.22 (d, J=1.5 Hz, 1H), 5.79 (d, J=1.3 Hz, 1H), 4.99-4.91 (m, 1H), 3.31 (s, 2H), 3.26-3.12 (m, 4H), 2.89 (s, 3H), 1.96-1.85 (m, 2H), 1.77-1.65 (m, 2H). LCMS m/z 292.0 (M+H)⁺ (ES⁺).

### Intermediate 2: 4-(tert-butoxy)-3-(diethoxyphosphoryl)-4-oxobutanoic acid

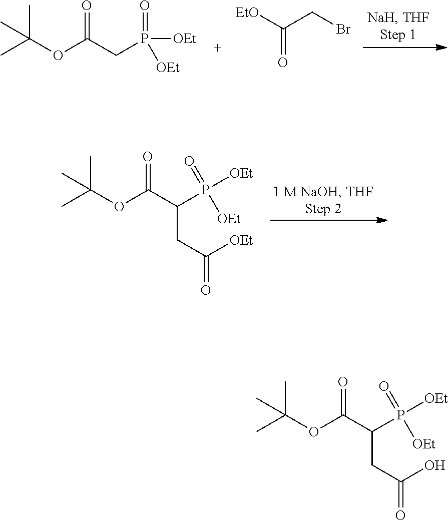 [SMILES: CCOP(=O)(CC(=O)OC(C)(C)C)OCC ; CCOC(=O)CBr ; CCOC(=O)CC(C(=O)OC(C)(C)C)P(=O)(OCC)OCC ; CCOP(=O)(OCC)C(CC(=O)O)C(=O)OC(C)(C)C]

### Step 1

Sodium hydride (60 wt % dispersion in mineral oil, 9.00 g, 225 mmol) was added portionwise to a solution of tert-butyl 2-(diethoxyphosphoryl)acetate (50 mL, 213 mmol) in THF (500 mL) at 0° C.

The mixture was stirred for 15 min before ethyl bromoacetate (23 mL, 210 mmol) was added dropwise. The mixture was stirred for 1 h then quenched with sat. aq. NH₄Cl (100 mL) and extracted with EtOAc (3×100 mL). The combined organic phases were washed with brine (300 mL), dried (MgSO₄) and concentrated to afford 1-(tert-butyl) 4-ethyl 2-(diethoxyphosphoryl)succinate (77.1 g, 182 mmol, 80% purity) as a colourless oil. LCMS m/z 361.2 (M+Na)⁺ (ES⁺). ¹H NMR (400 MHz, DMSO-d6) δ 4.13-4.01 (m, 6H), 3.28 (ddd, J=23.8, 11.3, 3.9 Hz, 1H), 2.78 (ddd, J=17.2, 11.3, 8.2 Hz, 1H), 2.64 (ddd, J=17.1, 8.5, 4.0 Hz, 1H), 1.40 (s, 9H), 1.28-1.21 (m, 6H), 1.18 (t, J=7.1 Hz, 3H).

### Step 2

An aqueous solution of sodium hydroxide (1 M, 250 mL, 250 mmol) was added to a solution of 1-(tert-butyl) 4-ethyl 2-(diethoxyphosphoryl)succinate (77.1 g, 182 mmol, 80% purity) in THF (250 mL). The mixture was stirred at RT for 16 h. The mixture was partially concentrated to ca. 250 mL, then extracted with EtOAc (3×100 mL). The aqueous phase was acidified to pH 1 with conc. HCl and extracted with EtOAc (3×100 mL). The combined organic phases were washed with brine (250 mL), dried (MgSO₄) and concentrated. The residue was triturated with hexane (300 mL) and the resulting solid collected by filtration to afford 4-(tert-butoxy)-3-(diethoxyphosphoryl)-4-oxobutanoic acid (53.00 g, 0.15 mol, 90% purity) as a white solid. LCMS m/z 333.2 (M+Na)⁺ (ES⁺). ¹H NMR (400 MHz, DMSO-d6) δ 12.44 (s, 1H), 4.11-3.99 (m, 4H), 3.22 (ddd, J=23.7, 11.5, 3.7 Hz, 1H), 2.73 (ddd, J=17.3, 11.5, 7.6 Hz, 1H), 2.56 (ddd, J=17.3, 8.6, 3.7 Hz, 1H), 1.40 (s, 9H), 1.25 (dt, J=8.3, 7.0 Hz, 6H). ³¹P NMR (162 MHz, DMSO-d6) δ 21.88.

### Intermediate 3: 3-((2,2,2-trichloroethoxy)carbonyl)but-3-enoic acid

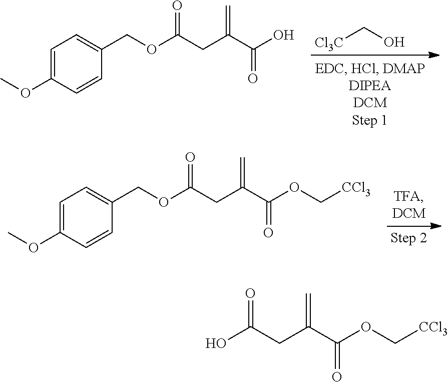

### Step 1

To a solution of 4-((4-methoxybenzyl)oxy)-2-methylene-4-oxobutanoic acid (30.0 g, 120 mmol), 2,2,2-trichloroethan-1-ol (19.7 g, 132 mmol), DMAP (11.7 g, 96 mmol) and DIPEA (46.4 g, 360 mmol) in DCM (500 mL) at 0° C. was added EDC·HCl (34.6 g, 180 mmol), and the resulting pale-yellow mixture was stirred at room temperature overnight. The mixture was quenched with dilute aqueous HCl (0.5 M), the phases were separated and the aqueous layer was extracted with DCM (3×500 mL). The combined organic phases were washed with brine, dried over Na₂SO₄ and filtered. The filtrate was concentrated under reduced pressure at 30° C., and the residue was purified by flash column chromatography (120 g silica, 0-20% MTBE/petroleum ether) to give 4-(4-methoxybenzyl) 1-(2,2,2-trichloroethyl) 2-methylenesuccinate (35 g, 91.7 mmol, 76%) as a colorless oil. LCMS: (System 2, Method C) m/z 402.8/404.8 (M+Na)⁺ (ES⁺).

### Step 2

A solution of 4-(4-methoxybenzyl) 1-(2,2,2-trichloroethyl) 2-methylenesuccinate (35.0 g, 91.7 mmol) in TFA (40 mL) and DCM (80 mL) was stirred at room temperature for 16 h. The mixture was concentrated under reduced pressure at 30° C. and the residue was purified by reversed phase column chromatography (330 g C18 silica; flow rate: 60 mL/min; 60-80% MeCN/10 mM formic acid/water; collection wavelength: 214 nm). The collected fractions were concentrated under reduced pressure at 30° C. to remove MeCN, and the residue was lyophilized to give 3-((2,2,2-trichloroethoxy)carbonyl)but-3-enoic acid (23.0 g, 88.0 mmol, 96%) as a colorless oil. LCMS: (System 2, Method C) m/z 282.8/284.8 (M+Na)⁺ (ES⁺).

### Intermediate 4: (S)-1-(4-((trifluoromethyl)thio)phenyl)ethan-1-amine

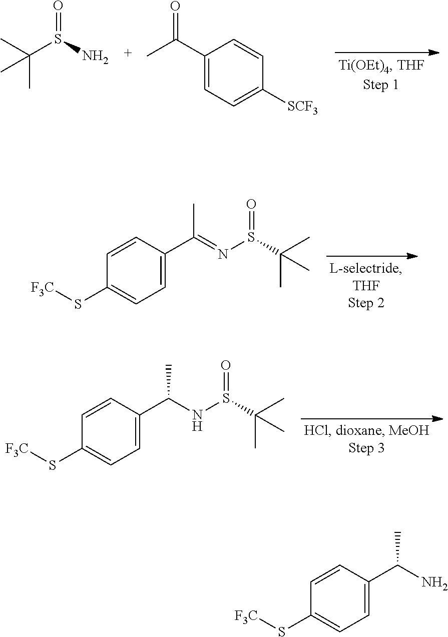

### Step 1

(R)-2-Methylpropane-2-sulfinamide (2.30 g, 19.0 mmol) was added portionwise to a solution of 1-(4-((trifluoromethyl)thio)phenyl)ethan-1-one (5.01 g, 22.8 mmol) and titanium(IV) ethoxide (8.0 mL, 38 mmol) in THF (100 mL) at RT. The mixture was heated to 65° C. and stirred for 16 h. The mixture was concentrated and the residue was taken up in EtOAc (30 mL). Water was added slowly and the resulting precipitate was filtered and the solid washed with additional EtOAc (50 mL). The phases were separated and the aqueous layer was further extracted with EtOAc (2×30 mL). The combined organic layers were washed with brine (50 mL), dried (Na₂SO₄) and concentrated. The crude product was purified by chromatography on silica gel (0-100% MTBE/isohexane) to afford (R,E)-2-methyl-N-(1-(4-((trifluoromethyl)thio)phenyl)ethylidene)propane-2-sulfinamide (5.61 g, 17 mmol) as a yellow oil. ¹H NMR (400 MHz, DMSO-d6) δ 8.02 (d, J=8.3 Hz, 2H), 7.87-7.80 (m, 2H), 2.75 (s, 3H), 1.23 (s, 9H). LCMS m/z 324.0 (M+H)⁺ (ES⁺).

### Step 2

A solution of L-selectride (1 M in THF, 18.6 mL, 18.6 mmol) was added dropwise to a solution of (R,E)-2-methyl-N-(1-(4-((trifluoromethyl)thio)phenyl)ethylidene)propane-2-sulfinamide (2.00 g, 6.18 mmol) in THF (22 mL) at 0° C. The mixture was stirred at 0° C. for 30 min and then warmed to RT and stirred for 2 h. The mixture was slowly quenched with water (˜10 mL), then 1 M HCl (30 mL) was added. The mixture was extracted with EtOAc (3×20 mL) and the combined organic layers were washed with brine (40 mL), dried (Na₂SO₄) and concentrated. The crude product was purified by chromatography on silica gel (0-10% MeOH/DCM), then further purified by chromatography on silica gel (0-100% MTBE/isohexane then 10% MeOH/DCM) to afford (R)-2-methyl-N—((S)-1-(4-((trifluoromethyl)thio)phenyl)ethyl)propane-2-sulfinamide (1.77 g, 5.30 mmol) as a white solid. ¹H NMR (400 MHz, DMSO-d6) δ 7.70-7.64 (m, 2H), 7.55-7.48 (m, 2H), 5.49 (d, J=5.6 Hz, 1H), 4.53-4.43 (m, 1H), 1.47 (d, J=6.8 Hz, 3H), 1.11 (s, 9H). LCMS m/z 326.1 (M+H)⁺ (ES⁺).

### Step 3

HCl (4 N in 1,4-dioxane, 2.7 mL, 10.8 mmol) was added dropwise to a solution of (R)-2-methyl-N—((S)-1-(4-((trifluoromethyl)thio)phenyl)ethyl)propane-2-sulfinamide (1.77 g, 5.30 mmol) in MeOH (20 mL) 0° C. The mixture was warmed to RT and stirred for 2 h. The mixture was concentrated and the residue was dissolved in water (30 mL) and extracted with DCM (2×20 mL). The aqueous layer was basified with sat. aq. Na₂CO₃ (˜15 mL) until pH-10, then extracted with DCM (3×15 mL). The combined organic layers were dried (Na₂SO₄) and concentrated to afford (S)-1-(4-((trifluoromethyl)thio)phenyl)ethan-1-amine (1.02 g, 4.6 mmol) as a colourless oil which partially solidified on standing. ¹H NMR (400 MHz, DMSO-d6) δ 7.67-7.61 (m, 2H), 7.58-7.50 (m, 2H), 4.03 (q, J=6.6 Hz, 1H), 1.91 (s, 2H), 1.24 (d, J=6.6 Hz, 3H). LCMS m/z 222.1 (M+H)⁺ (ES⁺).

### Intermediate 5: (S)-1-(4-(perfluoroethyl)phenyl)ethan-1-amine, HCl

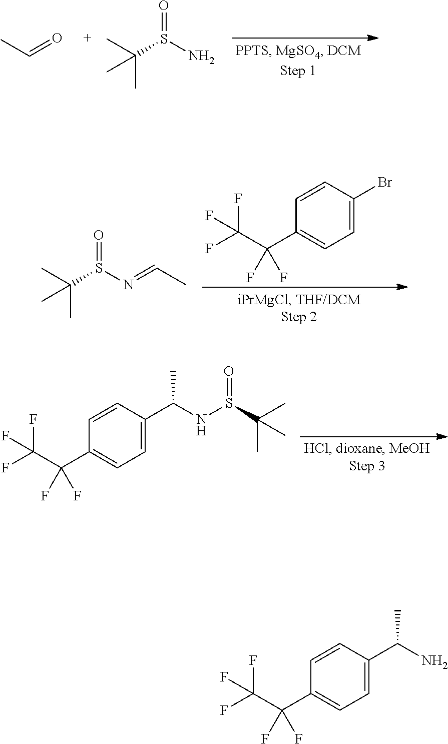

### Step 1

PPTS (518 mg, 2.06 mmol), MgSO₄ (25.5 g, 206 mmol) and acetaldehyde (4.6 mL, 83 mmol) are added to a solution of (S)-2-methylpropane-2-sulfinamide (5.00 g, 41.3 mmol) in DCM (70 mL). The mixture was stirred for 18 h at RT, then filtered through celite, washing with DCM (100 mL). The filtrate was concentrated. The crude product was purified by chromatography on silica gel (0-100% DCM/isohexane) to afford (S,E)-N-ethylidene-2-methylpropane-2-sulfinamide (4.61 g, 30 mmol) as a clear colourless oil. ¹H NMR (400 MHz, DMSO-d6) δ 7.95 (q, J=5.1 Hz, 1H), 2.20 (d, J=5.1 Hz, 3H), 1.10 (s, 9H).

### Step 2

1-bromo-4-(perfluoroethyl)benzene (1.00 g, 3.64 mmol) was added dropwise to a solution of isopropylmagnesium chloride (2 M in THF, 2.0 mL, 4.0 mmol) diluted with THF (4.7 mL), maintaining the internal temperature between 15-25° C. The mixture was stirred at RT for 2 h, then added dropwise to a solution of (S,E)-N-ethylidene-2-methylpropane-2-sulfinamide (535 mg, 3.64 mmol) in DCM (20 mL) at 0° C. The mixture was warmed to RT and stirred for 3 h. Water (50 mL) was added and the mixture was extracted with DCM (3×15 mL). The combined organic layers were washed with brine (40 mL), dried (Na₂SO₄) and concentrated. The crude product was purified by chromatography on silica gel (0-100% MTBE/isohexane) then further purified by chromatography on RP Flash C18 (0-100% (0.1% Formic acid in MeCN)/(0.1% Formic Acid in Water)) to afford (S)-2-methyl-N—((S)-1-(4-(perfluoroethyl)phenyl)ethyl)propane-2-sulfinamide (68 mg, 0.19 mmol) as a light tan solid. ¹H NMR (400 MHz, DMSO-d6) δ 7.66 (s, 4H), 5.84 (d, J=7.5 Hz, 1H), 4.54-4.43 (m, 1H), 1.41 (d, J=6.8 Hz, 3H), 1.12 (s, 9H). LCMS m/z 344.1 (M+H)⁺ (ES⁺).

### Step 3

To a solution of (S)-2-methyl-N—((S)-1-(4-(perfluoroethyl)phenyl)ethyl)propane-2-sulfinamide (68 mg, 0.19 mmol) in MeOH (0.75 mL) was added HCl (4 N in 1,4-dioxane, 0.1 mL, 0.4 mmol) dropwise at 0° C. The reaction was warmed to RT and stirred for 2 h. The mixture was concentrated to afford (S)-1-(4-(perfluoroethyl)phenyl)ethan-1-amine, HCl (52 mg, 0.19 mmol) as a colourless oil that was used without purification. ¹H NMR (400 MHz, DMSO-d6) δ 8.51 (s, 3H), 7.80 (d, J=8.5 Hz, 2H), 7.76 (d, J=8.7 Hz, 2H), 4.54 (q, J=6.9 Hz, 1H), 1.53 (d, J=6.8 Hz, 3H). LCMS m/z 240.1 (M+H)⁺ (ES⁺).

### Intermediate 6: (S)-cyclopropyl(4-(trifluoromethyl)phenyl)methanamine hydrochloride

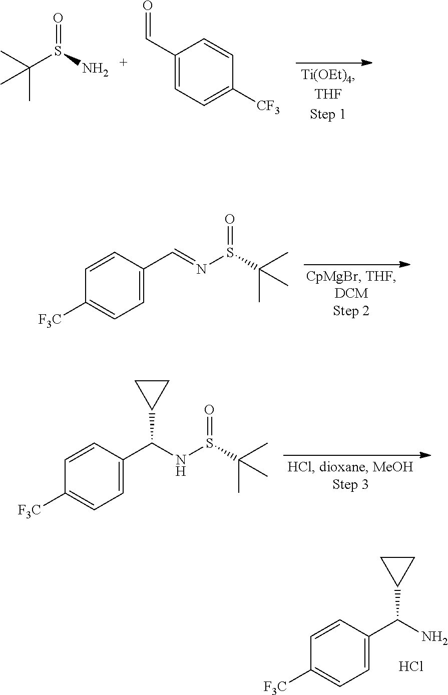

### Step 1

To a solution of 4-(trifluoromethyl)benzaldehyde (2.35 mL, 17.2 mmol) and titanium (IV) ethoxide (7.95 mL, 37.9 mmol) in THF (35 mL) was added (R)-2-methylpropane-2-sulfinamide (2.09 g, 17.2 mmol) portionwise at RT. The mixture was stirred at RT for 16 h. The mixture was concentrated and the residue was re-dissolved in EtOAc (30 mL). Water (10 mL) was added slowly, and the resulting precipitate was filtered and the solid was washed with EtOAc (50 mL). The resulting mixture was separated and the aqueous layer was extracted with EtOAc (2×30 mL). The combined organic layers were washed with brine (50 mL), dried (Na₂SO₄) and concentrated. The crude product was purified by chromatography on silica gel (0-100% MTBE/isohexane) to afford (R,E)-2-methyl-N-(4-(trifluoromethyl)benzylidene)propane-2-sulfinamide (4.20 g, 15 mmol) as a yellow oil. ¹H NMR (400 MHz, DMSO-d6) δ 8.67 (s, 1H), 8.16 (d, J=8.1 Hz, 2H), 7.91 (d, J=8.1 Hz, 2H), 1.20 (s, 9H). LCMS m/z 278.0 (M+H)⁺ (ES⁺).

### Step 2

Cyclopropylmagnesium bromide (0.5 M in THF, 45 mL, 22.5 mmol) was added dropwise over 1 h to a solution of (R,E)-2-methyl-N-(4-(trifluoromethyl)benzylidene)propane-2-sulfinamide (4.20 g, 15.1 mmol) in DCM (90 mL) at −78° C. The mixture was stirred at −78° C. for 1 h, then warmed to RT. The mixture was stirred for 2 h, then quenched with sat. aq NH₄Cl (100 mL). The mixture was separated and the aqueous layer was extracted with DCM (3×75 mL). The combined organic layers were washed with water (250 mL), brine (250 mL), dried (Na₂SO₄) and concentrated. The crude product was purified by chromatography on silica gel (0-50% EtOAc/isohexane) to afford (R)—N—((S)-cyclopropyl(4-(trifluoromethyl)phenyl)methyl)-2-methylpropane-2-sulfinamide (1.19 g, 3.5 mmol) as a colourless oil which solidified on standing. ¹H NMR (400 MHz, DMSO-d6) δ 7.69 (d, J=8.2 Hz, 2H), 7.62 (d, J=8.2 Hz, 2H), 5.50 (d, J=5.7 Hz, 1H), 3.62 (dd, J=9.1, 5.7 Hz, 1H), 1.17 (t, J=7.1 Hz, 1H), 1.10 (s, 9H), 0.65-0.56 (m, 1H), 0.51-0.38 (m, 3H). LCMS m/z 320.0 (M+H)⁺ (ES⁺).

### Step 3

To a solution of (R)—N—((S)-cyclopropyl(4-(trifluoromethyl)phenyl)methyl)-2-methylpropane-2-sulfinamide (1.19 g, 3.5 mmol) in MeOH (14 mL) was added HCl (4 N in dioxane, 1.86 mL, 7.5 mmol) dropwise at 0° C. The mixture was warmed to RT and stirred for 2 h, then concentrated to afford (S)-cyclopropyl(4-(trifluoromethyl)phenyl)methanamine hydrochloride (918 mg, 3.5 mmol) as a white solid. ¹H NMR (400 MHz, DMSO-d6) δ 8.69 (s, 3H), 7.83 (d, J=8.2 Hz, 2H), 7.77 (d, J=8.2 Hz, 2H), 3.71 (d, J=9.8 Hz, 1H), 1.36-1.25 (m, 1H), 0.75-0.58 (m, 2H), 0.56-0.47 (m, 1H), 0.45-0.36 (m, 1H).

### Intermediate 7: (S)-1-(5-(trifluoromethyl)pyridin-2-yl)ethan-1-amine dihydrochloride

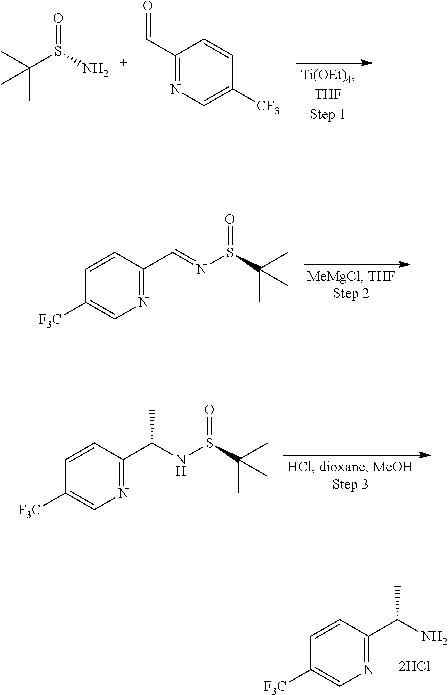

### Step 1

Titanium(IV) ethoxide (8.9 mL, 43 mmol) was added to a mixture of 5-(trifluoromethyl)picolinaldehyde (5.00 g, 28.6 mmol) and (S)-2-methylpropane-2-sulfinamide (5.19 g, 42.8 mmol) in THF (100 mL). The mixture was heated to 75° C. and stirred for 3 days. The mixture was cooled to RT and diluted with water (50 mL) and stirred rapidly for 10 min. The mixture was then filtered through celite, eluting with EtOAc (200 mL). The filtrate was concentrated and the residue was dissolved in DCM (10 mL), dried (MgSO₄) and concentrated. The crude product was purified by chromatography on silica gel (0-30% MTBE/isohexane) to afford (S,E)-2-methyl-N-((5-(trifluoromethyl)pyridin-2-yl)methylene)propane-2-sulfinamide (4.73 g, 14 mmol, 85% purity) as a white solid. ¹H NMR (400 MHz, DMSO-d6) δ 9.19-9.16 (m, 1H), 8.56 (s, 1H), 8.44-8.39 (m, 1H), 8.31-8.22 (m, 1H), 1.22 (s, 9H).

### Step 2

Methylmagnesium chloride (3 M in THF, 4.0 mL, 12 mmol) was added dropwise to a solution of (S,E)-2-methyl-N-((5-(trifluoromethyl)pyridin-2-yl)methylene)propane-2-sulfinamide (2.00 g, 6.11 mmol, 85% purity) in THF (25 mL) at −78° C. The mixture was stirred for 3 h, then quenched with sat. aq. NH₄Cl (10 mL). The mixture was separated and the aqueous phase was extracted with EtOAc (3×50 mL). The combined organic phases were washed with brine (100 mL), dried (MgSO₄) and concentrated. The crude product was purified by chromatography on silica gel (0-100% EtOAc/isohexane) to afford (S)-2-methyl-N—((S)-1-(5-(trifluoromethyl)pyridin-2-yl)ethyl)propane-2-sulfinamide (1.68 g, 5.7 mmol) as a pale yellow solid. ¹H NMR (400 MHz, DMSO-d6) δ 8.97-8.83 (m, 1H), 8.28-8.17 (m, 1H), 7.79 (d, J=8.3 Hz, 1H), 5.90 (d, J=8.1 Hz, 1H), 4.63-4.42 (m, 1H), 1.45 (d, J=6.9 Hz, 3H), 1.13 (s, 9H). LCMS m/z 295.1 (M+H)⁺ (ES⁺).

### Step 3

A mixture of (S)-2-methyl-N—((S)-1-(5-(trifluoromethyl)pyridin-2-yl)ethyl)propane-2-sulfinamide (1.68 g, 5.7 mmol) and HCl (4 M in 1,4-dioxane, 6 mL, 24 mmol) in MeOH (15 mL) was stirred for 18 h at RT. The mixture was concentrated and the residue triturated with MTBE (30 mL). The resulting solid was isolated by filtration to afford (S)-1-(5-(trifluoromethyl)pyridin-2-yl)ethan-1-amine, 2HCl (1.44 g, 5.4 mmol) as a pale yellow solid. ¹H NMR (400 MHz, DMSO-d6) δ 9.04-9.01 (m, 1H), 8.68 (br. s, 3H), 8.34 (ddd, J=8.3, 2.4, 0.8 Hz, 1H), 8.11 (br. s, 1H), 7.82 (d, J=8.3 Hz, 1H), 4.73-4.54 (m, 1H), 1.53 (d, J=6.8 Hz, 3H). LCMS m/z 191.2 (M+H)⁺ (ES⁺).

### Intermediate 8: (S)-1-(6-(trifluoromethyl)pyridin-3-yl)ethan-1-amine hydrochloride

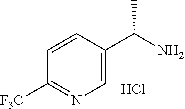

Prepared by an analogous method to Intermediate 5 starting from 6-(trifluoromethyl)nicotinaldehyde (4.22 g, 24.1 mmol). Yield: 737 mg, 3.2 mmol. White solid. ¹H NMR (400 MHz, DMSO) δ 8.92 (d, J=2.2 Hz, 1H), 8.77 (br. s, 3H), 8.29 (dd, J=8.2, 2.2 Hz, 1H), 8.02 (dd, J=8.2, 0.8 Hz, 1H), 4.72-4.52 (m, 1H), 1.58 (d, J=6.9 Hz, 3H). LCMS m/z 191.2 (M+H)⁺ (ES⁺).

### Intermediate 9: 1-(5-(trifluoromethyl)pyridin-2-yl)cyclobutan-1-amine

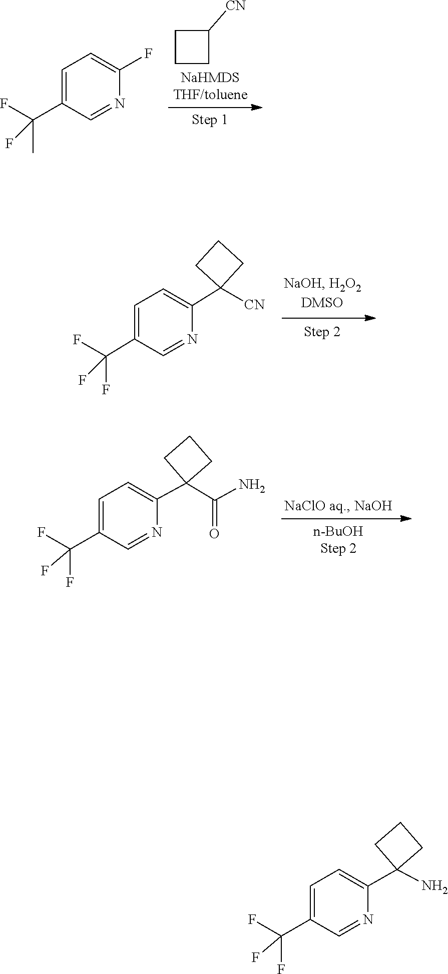

### Step 1

To the solution of cyclobutanecarbonitrile (1.47 g, 18.2 mmol) in toluene (30 mL) was added NaHMDS (18.2 mL, 18.2 mmol, 1M in THF solution) at 0° C., followed by 2-fluoro-5-(trifluoromethyl)pyridine (3.00 g, 18.17 mmol). The resulting dark brown mixture was stirred at room temperature for 3 days. The mixture was quenched with saturated aqueous NH₄Cl (20 mL), the organic phase separated and the aqueous phase extracted with EtOAc (3×10 mL). The combined organic layers were concentrated under reduced pressure, and the residue was purified by flash column chromatography on silica (0-10% tert-butyl methyl ether/petroleum ether) to give 1-(5-(trifluoromethyl)pyridin-2-yl)cyclobutane-1-carbonitrile (950 mg, 23% yield) as a colorless oil. LCMS m/z 227.2 (M+H)⁺ (ES⁺).

### Step 2

To the mixture of 1-(5-(trifluoromethyl)pyridin-2-yl)cyclobutane-1-carbonitrile (380 mg, 1.7 mmol) and 2N NaOH aqueous (1.7 mL, 3.4 mmol) in DMSO (2 mL) was added hydrogen peroxide aqueous solution (381 mg, 3.4 mmol, 30% wt) at 0° C. The mixture was stirred at 25° C. for 1 hour. The reaction mixture was extracted with EtOAc (3×3 mL). The EtOAc layer was washed with brine, dried over Na₂SO₄, and the filtrate was concentrated under reduced pressure to give 1-(5-(trifluoromethyl)pyridin-2-yl)cyclobutane-1-carboxamide (330 mg, 81% yield) as white solid. LCMS m/z 245.2 (M+H)⁺ (ES⁺).

### Step 3

A mixture of 1-(5-(trifluoromethyl)pyridin-2-yl)cyclobutane-1-carboxamide (330 mg, 1.3 mmol), sodium hydroxide (108 mg, 2.7 mmol) and sodium hypochlorite (2.14 g, 4.0 mmol, 14 wt % aqueous solution) in 1-butanol (5 mL) was stirred at 25° C. for 16 hours. The reaction mixture was quenched with H₂O (5 mL), and extracted with EtOAc (2×3 mL). The EtOAc layer was washed by brine, dried over Na₂SO₄, and the filtrate was concentrated under reduced pressure The residue was purified by flash column chromatography on silica (0-25% tert-butyl methyl ether/petroleum ether) to give 1-(5-(trifluoromethyl)pyridin-2-yl)cyclobutan-1-amine (120 mg, 41% yield) as light-brown solid. ¹H NMR (400 MHz, CDCl₃) δ: 8.84 (s, 1H), 7.93 (dd, J=8.4, 6.4 Hz, 1H), 7.63 (d, J=8.0 Hz, 1H), 2.71-2.27 (m, 2H), 2.26-2.16 (m, 2H), 2.09 (s, 2H), 1.93-1.84 (m, 2H), 2.11-2.05 (m, 2H). LCMS m/z 217.2 (M+H)⁺ (ES⁺).

### Intermediate 10: 3-(((9H-fluoren-9-yl)methoxy)carbonyl)but-3-enoic acid

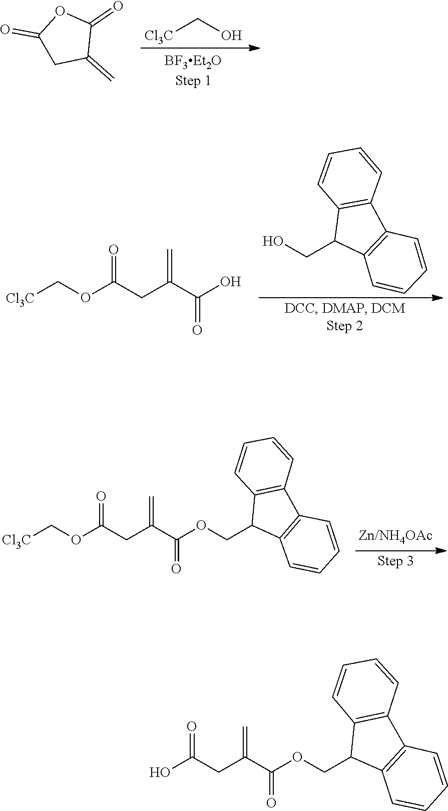

### Step 1

To a mixture of 3-methylenedihydrofuran-2,5-dione (10.0 g, 89.2 mmol) and 2,2,2-trichloroethanol (20.0 g, 133.8 mmol) was added boron trifluoride diethyl etherate (1.27 g, 8.9 mmol), and the mixture was allowed to stir at 75° C. for 40 minutes. The mixture was cooled to room temperature, quenched with methanol (4 mL), diluted with EtOAc (100 mL) and water (20 mL). The organic phase was separated and aqueous phase extracted with EtOAc (2×50 mL). The separated organics were washed with brine and dried over Na₂SO₄. The filtrate was concentrated under reduced pressure and the residue was purified by reversed column chromatography (Column: Boston ODS 120 g Flash; Flow Rate: 40 mL/min; solvent system: MeCN/(10 mmol/L HCl/water); MeCN gradient: 60-80%; collection wavelength: 214 nm). The fractions were concentrated under reduced pressure to remove MeCN, and the residue was lyophilized to give 2-methylene-4-oxo-4-(2,2,2-trichloroethoxy)butanoic acid (18.0 g, 77% yield) as white solid, containing 5% regioisomer. The solid was triturated in n-hexane (150 mL)/tert-butyl methyl ether (20 mL), stirred at room temperature overnight and recovered by filtration. The wet cake was dried at 40° C. under reduced pressure to give 2-methylene-4-oxo-4-(2,2,2-trichloroethoxy)butanoic acid (16.0 g, 68% yield). LCMS m/z 261.2 (M+H)⁺ (ES⁺).

### Step 2

A mixture of 2-methylene-4-oxo-4-(2,2,2-trichloroethoxy)butanoic acid (13.0 g, 49.7 mmol), (9H-fluoren-9-yl)methanol (9.76 g, 49.7 mmol), DCC (15.36 g, 74.6 mmol) and DMAP (910 mg, 7.5 mmol) in DCM (200 mL) was stirred at room temperature for 30 minutes. The mixture was filtered, and the filtrate was concentrated under reduced pressure. The residue was purified by flash column chromatography on silica (0-20% tert-butyl methyl ether/petroleum ether) to give 1-((9H-fluoren-9-yl)methyl) 4-(2,2,2-trichloroethyl) 2-methylenesuccinatee (12.0 g, 55% yield) as a white solid. LCMS m/z 461.0 (M+Na)⁺ (ES⁺).

### Step 3

A mixture of 1-((9H-fluoren-9-yl)methyl) 4-(2,2,2-trichloroethyl) 2-methylenesuccinatee (12.0 g, 27.3 mmol), zinc powder (5.32 g, 81.9 mmol) and NH₄OAc (10.50 g, 136.5 mol) in THF (80 mL) and H₂O (20 mL) was stirred at 25° C. for 2 hours. The reaction mixture was filtered, and the filtrate was extracted with tert-butyl methyl ether (2×5 mL). The organic phase was washed with brine, dried over Na₂SO₄ and filtrate was concentrated under reduced pressure. The residue was purified by flash column chromatography on silica (20-40% tert-butyl methyl ether/petroleum ether)) to give 3-(((9H-fluoren-9-yl)methoxy)carbonyl)but-3-enoic acid (8.41 g, 73% yield) as white solid. LCMS m/z 331.0 (M+Na)⁺ (ES⁺).

### Intermediate 11: 5-(trifluoromethyl)-2,3-dihydro-1H-inden-1-amine

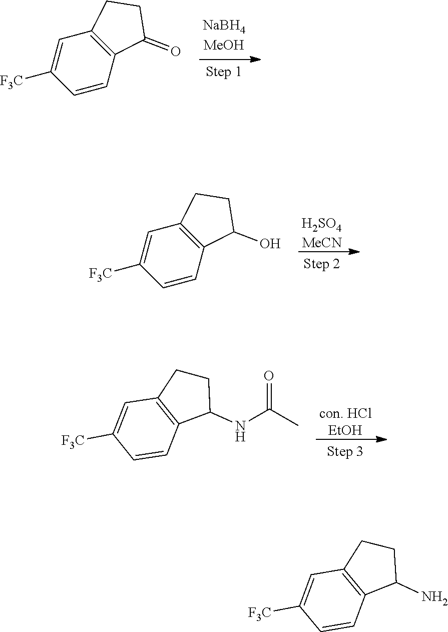

### Step 1

To a solution of 5-(trifluoromethyl)-2,3-dihydro-1H-inden-1-one (2.00 g, 9.99 mmol) in MeOH (50 mL) was added sodium borohydride (759 mg, 19.98 mmol) at 0° C., and the mixture was allowed to stir at room temperature for 0.5 hour. The mixture was concentrated under reduced pressure then quenched with water (50 mL) and extracted with EtOAc (2×60 mL). The organic layer was washed with brine, dried over Na₂SO₄. The filtrate was concentrated under reduced pressure, and the residue was purified by flash column chromatography on silica (0-20% tert-Butyl methyl ether/diethyl ether) to give 5-(trifluoromethyl)-2,3-dihydro-1H-inden-1-ol (1.90 g, 94% yield) as white solid. ¹H NMR (400 MHz, CDCl₃) δ: 7.50 (s, 3H), 5.30-2.25 (m, 1H), 3.13-3.06 (m, 1H), 2.90-2.82 (m, 1H), 2.60-2.52 (m, 1H), 2.03-1.96 (m, 1H), 1.79 (d, J=7.2 Hz, 1H).

### Step 2

To the solution of 5-(trifluoromethyl)-2,3-dihydro-1H-inden-1-ol (250 mg, 1.24 mmol) in acetonitrile (2 mL) was added concentrated H₂SO₄ (607 mg, 6.20 mmol) at 0° C., and the mixture was stirred at room temperature for 1 hour. The reaction mixture was quenched with water (3 mL) and concentrated under reduced pressure to remove acetonitrile. The residue was extracted with EtOAc (3×2 mL) and the combined organic layers were washed by brine, dried over Na₂SO₄, concentrated under reduced pressure to give N-(5-(trifluoromethyl)-2,3-dihydro-1H-inden-1-yl)acetamide (300 mg, 98% yield) as off-white solid. LCMS m/z 244.2 (M+H)⁺ (ES⁺).

### Step 3

A mixture of N-(5-(trifluoromethyl)-2,3-dihydro-1H-inden-1-yl)acetamide (300 mg, 1.23 mmol) in ethanol (2 mL) and concentrated HCl (1 mL) was stirred at 95° C. for 48 hours. The solvent was removed under reduced pressure, and the residue was partitioned between EtOAc (3 mL) and 2N NaOH (to final pH=8) and extracted with EtOAc (2×2 mL). The organic layer was washed with brine, dried over Na₂SO₄ and concentrated under reduced pressure to give 5-(trifluoromethyl)-2,3-dihydro-1H-inden-1-amine (230 mg, 93% yield) as white solid. LCMS m/z 202.2 (M+H)⁺ (ES⁺).

### Intermediate 12: (S)-1-(4-(pentafluoro-A6-sulfaneyl)phenyl)ethan-1-amine hydrochloride

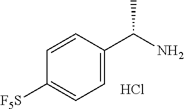

Prepared by an analogous method to Intermediate 6 starting from 4-(pentafluoro-A6-sulfaneyl)benzaldehyde (1.00 g, 4.31 mmol) and using methylmagnesium chloride solution in place of cyclopropylmagnesium bromide in Step 2. Yield: 275 mg, 0.92 mmol. White solid. ¹H NMR (400 MHz, DMSO) δ 8.80 (s, 3H), 8.02-7.94 (m, 2H), 7.79 (d, J=8.5 Hz, 2H), 4.57-4.46 (m, 1H), 1.53 (d, J=6.8 Hz, 3H). LCMS m/z 248.1 (M+H)⁺ (ES⁺).

### Synthesis of Examples

In the Examples, compounds described as ISOMER 1 and ISOMER 2, are enantiomers or diasteromers which have been separated by chromatography and characterised by NMR but for which the absolute stereochemistry has been arbitrarily assigned.

### Example 1: 1-(methylsulfonyl)piperidin-4-yl 4-((4-chlorobenzyl)amino)-2-methylene-4-oxobutanoate

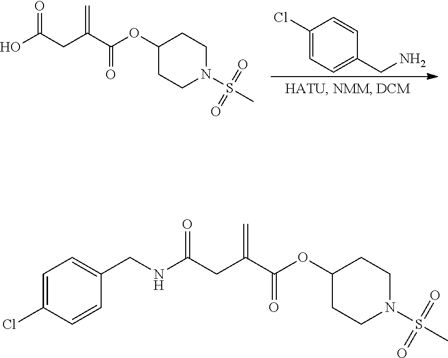 [SMILES: C=C(CC(=O)O)C(=O)OC1CCN(S(C)(=O)=O)CC1 ; NCc1ccc(Cl)cc1 ; C=C(CC(=O)NCc1ccc(Cl)cc1)C(=O)OC1CCN(S(C)(=O)=O)CC1]

HATU (0.26 g, 0.68 mmol) was added to a solution of 3-(((1-(methylsulfonyl)piperidin-4-yl)oxy)carbonyl)but-3-enoic acid (Intermediate 1, 0.180 g, 0.62 mmol), 4-methylmorpholine (0.10 ml, 0.93 mmol) and (4-chlorophenyl)methanamine (0.075 mL, 0.62 mmol) in DCM (3 mL) at 0° C. The mixture was warmed to RT and stirred for 1 h, then diluted with EtOAc (10 mL) and water (10 mL). The phases were separated and the aqueous layer was extracted with EtOAc (3×10 mL). The combined organic phases were washed with brine (2×20 mL), dried (MgSO₄) and concentrated. The crude product was purified by chromatography on silica gel (0-10% MeOH/DCM), then triturated with MTBE and isolated by filtration to afford 1-(methylsulfonyl)piperidin-4-yl 4-((4-chlorobenzyl)amino)-2-methylene-4-oxobutanoate (0.256 g, 0.49 mmol) as a white solid. ¹H NMR (500 MHz, DMSO-d6) δ 8.44 (t, J=6.0 Hz, 1H), 7.41-7.35 (m, 2H), 7.30-7.25 (m, 2H), 6.21 (d, J=1.5 Hz, 1H), 5.75 (d, J=1.4 Hz, 1H), 4.93-4.86 (m, 1H), 4.25 (d, J=5.9 Hz, 2H), 3.24 (s, 2H), 3.23-3.18 (m, 2H), 3.19-3.12 (m, 2H), 2.88 (s, 3H), 1.92-1.84 (m, 2H), 1.71-1.61 (m, 2H). LCMS m/z 415.2 (M+H)⁺ (ES⁺).

### Example 2: 1-(methylsulfonyl)piperidin-4-yl 4-(cyclooctylamino)-2-methylene-4-oxobutanoate

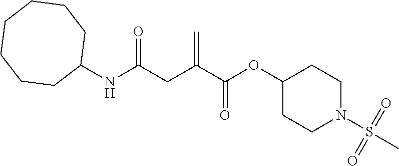 [SMILES: C=C(CC(=O)NC1CCCCCCC1)C(=O)OC1CCN(S(C)(=O)=O)CC1]

Prepared by an analogous method to Example 1 starting from cyclooctanamine (0.079 g, 0.62 mmol). Yield: 314 mg, 0.54 mmol. White solid. ¹H NMR (500 MHz, DMSO-d6) δ 7.80 (d, J=7.8 Hz, 1H), 6.17 (d, J=1.6 Hz, 1H), 5.69 (d, J=1.4 Hz, 1H), 4.96-4.84 (m, 1H), 3.78-3.68 (m, 1H), 3.29-3.22 (m, 2H), 3.21-3.14 (m, 2H), 3.13 (s, 2H), 2.89 (s, 3H), 1.94-1.86 (m, 2H), 1.73-1.58 (m, 6H), 1.57-1.37 (m, 10H). LCMS m/z 401.3 (M+H)⁺ (ES⁺).

### Example 3: (S)-2-methylene-4-oxo-4-((1-(4-(trifluoromethyl)phenyl)ethyl)amino)butanoic acid

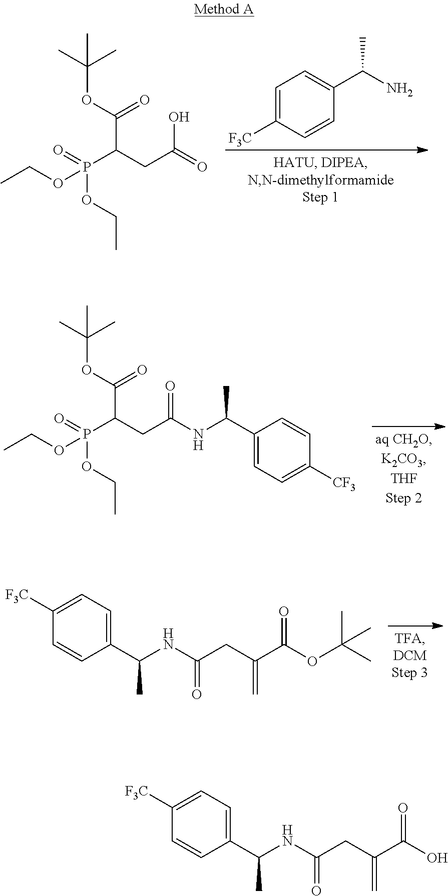 [SMILES: CCOP(=O)(OCC)C(CC(=O)O)C(=O)OC(C)(C)C ; C[C@H](N)c1ccc(C(F)(F)F)cc1 ; CCOP(=O)(OCC)C(CC(=O)N[C@@H](C)c1ccc(C(F)(F)F)cc1)C(=O)OC(C)(C)C ; C=C(CC(=O)N[C@@H](C)c1ccc(C(F)(F)F)cc1)C(=O)OC(C)(C)C ; C=C(CC(=O)N[C@@H](C)c1ccc(C(F)(F)F)cc1)C(=O)O]

### Step 1

HATU (2.41 g, 6.34 mmol) was added to a mixture of (S)-1-(4-(trifluoromethyl)phenyl)ethan-1-amine (1.00 g, 5.29 mmol), 4-(tert-butoxy)-3-(diethoxyphosphoryl)-4-oxobutanoic acid (Intermediate 2, 1.72 g, 5.55 mmol) and DIPEA (2.8 mL, 16 mmol) in N,N-dimethylformamide (20 mL). The mixture was stirred at RT for 16 h, before water (150 mL) was added. The mixture was extracted with EtOAc (3×50 mL). The combined organic phases were washed with 1 M HCl (200 mL), sat. aq. NaHCO₃ (200 mL), brine (3×200 mL), dried (MgSO₄) and concentrated to afford tert-butyl 2-(diethoxyphosphoryl)-4-oxo-4-(((S)-1-(4-(trifluoromethyl)phenyl)ethyl)amino)butanoate (2.88 g, 5.4 mmol, 91% purity) as a brown oil. ¹H NMR (400 MHz, DMSO) δ 8.55 (t, J=7.4 Hz, 1H), 7.71-7.59 (m, 2H), 7.57-7.44 (m, 2H), 4.98-4.85 (m, 1H), 4.11-3.95 (m, 4H), 3.28-3.13 (m, 1H), 2.85-2.71 (m, 1H), 2.48-2.42 (m, 1H), 1.40-1.20 (m, 18H). LCMS m/z 426.3 (M-tBu+H)⁺ (ES⁺).

### Step 2

Formaldehyde (37% aqueous, 2.0 mL, 27 mmol) was added to a suspension of tert-butyl 2-(diethoxyphosphoryl)-4-oxo-4-(((S)-1-(4-(trifluoromethyl)phenyl)ethyl)amino)butanoate (2.88 g, 5.4 mmol, 91% purity) and potassium carbonate (1.13 g, 8.15 mmol) in THF (25 mL). The mixture was stirred at RT for 4 h. The mixture was diluted with water (100 mL) and extracted with EtOAc (3×50 mL). The combined organic phases were washed with brine (100 mL), dried (MgSO₄) and concentrated. The crude product was purified by chromatography on silica gel (0-100% MTBE/isohexane) to afford tert-butyl (S)-2-methylene-4-oxo-4-((1-(4-(trifluoromethyl)phenyl)ethyl)amino)butanoate (1.75 g, 4.5 mmol, 92% purity) as a colourless oil. ¹H NMR (400 MHz, DMSO) δ 8.43 (d, J=7.7 Hz, 1H), 7.66 (d, J=8.1 Hz, 2H), 7.53 (d, J=8.1 Hz, 2H), 6.02 (d, J=1.8 Hz, 1H), 5.58 (d, J=1.6 Hz, 1H), 5.00-4.90 (m, 1H), 3.13 (s, 2H), 1.40-1.34 (m, 12H). LCMS m/z 302.3 (M-tBu+H)⁺ (ES⁺).

### Step 3

A mixture of tert-butyl (S)-2-methylene-4-oxo-4-((1-(4-(trifluoromethyl)phenyl)ethyl)amino)butanoate (1.75 g, 4.5 mmol, 92% purity) and TFA (5 mL, 60 mmol) in DCM (10 mL) was stirred at RT for 1 h, then concentrated. The residue was co-evaporated with toluene (2×10 mL), then triturated with MTBE (50 mL) to afford (S)-2-methylene-4-oxo-4-((1-(4-(trifluoromethyl)phenyl)ethyl)amino)butanoic acid (878 mg, 2.9 mmol) as a white solid. ¹H NMR (400 MHz, DMSO) δ 12.46 (s, 1H), 8.45 (d, J=7.7 Hz, 1H), 7.66 (d, J=8.1 Hz, 2H), 7.52 (d, J=8.1 Hz, 2H), 6.09 (d, J=1.7 Hz, 1H), 5.63 (q, J=1.4 Hz, 1H), 5.08-4.85 (m, 1H), 3.16 (s, 2H), 1.35 (d, J=7.1 Hz, 3H). LCMS m/z 302.2 (M+H)⁺ (ES⁺).

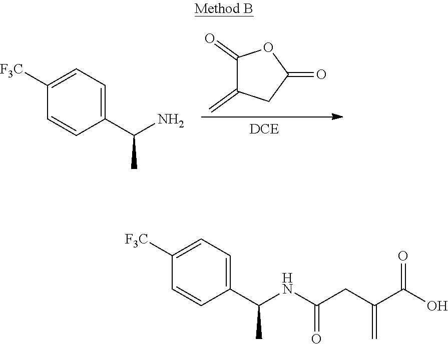

To an ice cold solution of 3-methylenedihydrofuran-2,5-dione (13.09 g, 116.82 mmol) in 1,2-dichloroethane (400 mL), a solution of (S)-1-(4-(trifluoromethyl)phenyl)ethan-1-amine (17.00 g, 89.86 mmol) in 1,2-dichloroethane (100 mL) was dropwise added at 5° C. over 20 minutes. The resulting mixture was stirred at 5° C. for 30 minutes, then at 25° C. for 4 hours. The solvent was removed under reduced pressure and the residue was suspended in ethyl acetate (170 mL) and stirred at 80° C. for 20 minutes until the solid was completely dissolved, then cooled to 25° C. n-heptane (170 mL) was dropwise added over 30 minutes and the resulting white suspension was stirred at 25° C. for 1.5 hours. The solid was recovered by filtration and dried under reduced pressure to give (S)-2-methylene-4-oxo-4-((1-(4-(trifluoromethyl)phenyl)ethyl)amino)butanoic acid (21.50 g, 79% yield) as a white solid. LCMS m/z 302.1 (M+H)⁺ (ES⁺).

### Example 4: 2-methylene-4-oxo-4-((2-(4-(trifluoromethyl)phenyl)propan-2-yl)amino)butanoic acid

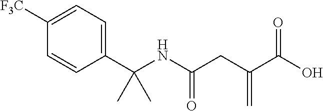 [SMILES: C=C(CC(=O)NC(C)(C)c1ccc(C(F)(F)F)cc1)C(=O)O]

Prepared by an analogous method to Example 3, Method A starting from 2-(4-(trifluoromethyl)phenyl)propan-2-amine, HCl (527 mg, 2.20 mmol), except that an extra equivalent of DIPEA was used in Step 1. Yield: 215 mg, 0.68 mmol. White solid. ¹H NMR (400 MHz, DMSO) δ 12.46 (s, 1H), 8.27 (s, 1H), 7.60 (d, J=8.4 Hz, 2H), 7.54 (d, J=8.4 Hz, 2H), 6.06 (d, J=1.8 Hz, 1H), 5.60 (d, J=1.7 Hz, 1H), 3.16 (s, 2H), 1.54 (s, 6H). LCMS m/z 316.3 (M+H)⁺ (ES⁺).

### Example 5: 2-methylene-4-oxo-4-((1-(4-(trifluoromethyl)phenyl)cyclobutyl)amino)butanoic acid

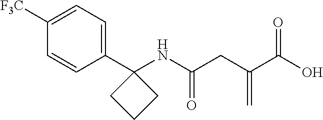 [SMILES: C=C(CC(=O)NC1(c2ccc(C(F)(F)F)cc2)CCC1)C(=O)O]

Prepared by an analogous method to Example 3, Method A starting from 1-(4-(trifluoromethyl)phenyl)cyclobutan-1-amine, HCl (250 mg, 0.99 mmol), except that an extra equivalent of DIPEA was used in Step 1. Yield: 115 mg, 0.35 mmol. White solid. ¹H NMR (400 MHz, DMSO) δ 12.46 (s, 1H), 8.69 (s, 1H), 7.64 (d, J=8.5 Hz, 2H), 7.60 (d, J=8.6 Hz, 2H), 6.07 (d, J=1.8 Hz, 1H), 5.60 (d, J=1.7 Hz, 1H), 3.14 (s, 2H), 2.43 (t, J=7.8 Hz, 4H), 2.11-1.96 (m, 1H), 1.93-1.79 (m, 1H). LCMS m/z 328.3 (M+H)⁺ (ES⁺).

### Example 6: 2-methylene-4-oxo-4-((4-(trifluoromethyl)benzyl)amino)butanoic acid

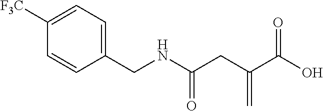 [SMILES: C=C(CC(=O)NCc1ccc(C(F)(F)F)cc1)C(=O)O]

Prepared by an analogous method to Example 3, Method A starting from (4-(trifluoromethyl)phenyl)methanamine (0.250 g, 1.43 mmol). Yield: 67 mg, 0.23 mmol. White solid. ¹H NMR (400 MHz, DMSO) δ 12.51 (s, 1H), 8.47 (t, J=6.1 Hz, 1H), 7.66 (d, J=8.0 Hz, 2H), 7.47 (d, J=8.0 Hz, 2H), 6.13 (d, J=1.7 Hz, 1H), 5.69 (d, J=1.7 Hz, 1H), 4.34 (d, J=5.9 Hz, 2H), 3.19 (s, 2H). LCMS m/z 288.2 (M+H)⁺ (ES⁺).

### Example 7: 2-methylene-4-oxo-4-((2,2,2-trifluoro-1-(4-(trifluoromethyl)phenyl)ethyl)amino)butanoic acid

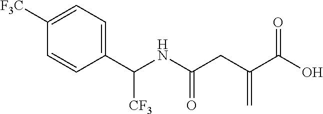 [SMILES: C=C(CC(=O)NC(c1ccc(C(F)(F)F)cc1)C(F)(F)F)C(=O)O]

Prepared by an analogous method to Example 3, Method A starting from 2,2,2-trifluoro-1-(4-(trifluoromethyl)phenyl)ethan-1-amine (0.250 g, 1.03 mmol). Yield: 147 mg, 0.41 mmol. White solid. ¹H NMR (400 MHz, DMSO) δ 12.48 (s, 1H), 9.31 (d, J=9.5 Hz, 1H), 7.94-7.74 (m, 4H), 6.11 (d, J=1.7 Hz, 1H), 5.99-5.87 (m, 1H), 5.67 (d, J=1.6 Hz, 1H), 3.38-3.17 (m, 2H). LCMS m/z 356.3 (M+H)⁺ (ES⁺).

### Example 8: (S)-2-methylene-4-oxo-4-((1-(4-(trifluoromethyl)phenyl)propyl)amino)butanoic acid

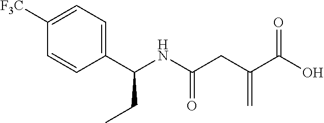 [SMILES: C=C(CC(=O)N[C@@H](CC)c1ccc(C(F)(F)F)cc1)C(=O)O]

Prepared by an analogous method to Example 3, Method A starting from (S)-1-(4-(trifluoromethyl)phenyl)propan-1-amine (0.100 g, 0.49 mmol). Yield: 48 mg, 0.15 mmol. White solid. ¹H NMR (400 MHz, DMSO) δ 12.45 (s, 1H), 8.49-8.34 (m, 1H), 7.66 (d, J=8.1 Hz, 2H), 7.51 (d, J=8.1 Hz, 2H), 6.07 (d, J=1.8 Hz, 1H), 5.61 (s, 1H), 4.73 (app q, J=7.6 Hz, 1H), 3.19 (d, J=15.5 Hz, 1H), 3.14 (d, J=15.5 Hz, 1H), 1.68 (m, 2H), 0.86 (t, J=7.3 Hz, 3H). LCMS m/z 316.2 (M+H)⁺ (ES⁺).

### Example 9: 1-(methylsulfonyl)piperidin-4-yl 2-methylene-4-oxo-4-((1-(4-(trifluoromethyl)phenyl)cyclobutyl)amino)butanoate

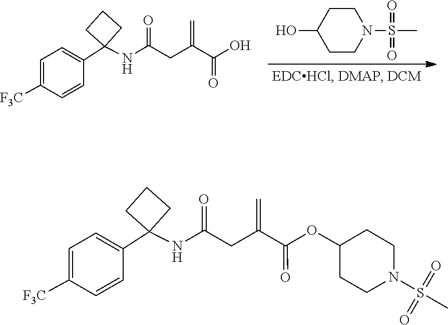 [SMILES: C=C(CC(=O)NC1(c2ccc(C(F)(F)F)cc2)CCC1)C(=O)O ; CS(=O)(=O)N1CCC(O)CC1 ; C=C(CC(=O)NC1(c2ccc(C(F)(F)F)cc2)CCC1)C(=O)OC1CCN(S(C)(=O)=O)CC1]

EDC·HCl (51 mg, 0.27 mmol) was added portionwise to a mixture of 2-methylene-4-oxo-4-((1-(4-(trifluoromethyl)phenyl)cyclobutyl)amino)butanoic acid (Example 5, 73 mg, 0.22 mmol), 1-(methylsulfonyl)piperidin-4-ol (60 mg, 0.33 mmol), DMAP (33 mg, 0.27 mmol) in DCM (1.2 mL) at 0° C. The mixture was warmed to RT and stirred for 18 h. The mixture was diluted with 1 M HCl (20 mL) and DCM (20 mL) and the phases were separated. The aqueous phase was extracted with DCM (2×20 mL). The combined organic phases were washed with brine (50 mL), dried (MgSO₄) and concentrated. The crude product was purified by chromatography on silica gel (0-10% MeOH/DCM) to afford 1-(methylsulfonyl)piperidin-4-yl 2-methylene-4-oxo-4-((1-(4-(trifluoromethyl)phenyl)cyclobutyl)amino)butanoate (7 mg, 0.01 mmol) as a white solid. ¹H NMR (400 MHZ, DMSO) δ 8.71 (s, 1H), 7.65 (d, J=8.3 Hz, 2H), 7.59 (d, J=8.3 Hz, 2H), 6.15 (d, J=1.5 Hz, 1H), 5.67 (d, J=1.4 Hz, 1H), 4.88-4.81 (m, 1H), 3.29-3.21 (m, 2H), 3.19 (s, 2H), 3.11 (ddd, J=11.8, 7.5, 3.7 Hz, 2H), 2.87 (s, 3H), 2.47-2.39 (m, 4H), 2.09-1.97 (m, 1H), 1.90-1.79 (m, 3H), 1.67-1.55 (m, 2H). LCMS m/z 489.3 (M+H)⁺ (ES⁺).

### Example 10: 4-((cyclopropyl(4-(trifluoromethyl)phenyl)methyl)amino)-2-methylene-4-oxobutanoic acid

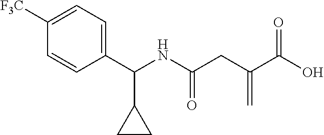 [SMILES: C=C(CC(=O)NC(c1ccc(C(F)(F)F)cc1)C1CC1)C(=O)O]

Prepared by an analogous method to Example 3, Method A starting from cyclopropyl(4-(trifluoromethyl)phenyl)methanamine (250 mg, 1.16 mmol), except that an extra equivalent of DIPEA was used in Step 1. Yield: 219 mg, 0.66 mmol. White solid. ¹H NMR (400 MHz, DMSO) δ 12.45 (s, 1H), 8.59 (s, 1H), 7.67 (d, J=8.2 Hz, 2H), 7.58 (d, J=8.1 Hz, 2H), 6.08 (d, J=1.8 Hz, 1H), 5.62 (s, 1H), 4.27 (t, J=8.5 Hz, 1H), 3.19-3.12 (m, 2H), 1.18-1.02 (m, 1H), 0.57-0.46 (m, 2H), 0.45-0.37 (m, 1H), 0.37-0.31 (m, 1H). LCMS m/z 328.3 (M+H)⁺ (ES⁺).

### Example 11: 2-methylene-4-oxo-4-((1-(4-(trifluoromethyl)phenyl)cyclopropyl)amino)butanoic acid

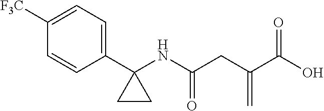 [SMILES: C=C(CC(=O)NC1(c2ccc(C(F)(F)F)cc2)CC1)C(=O)O]

Prepared by an analogous method to Example 3, Method A starting from 1-(4-(trifluoromethyl)phenyl)cyclopropan-1-amine (250 mg, 1.24 mmol), except that an extra equivalent of DIPEA was used in Step 1. Yield: 150 mg, 0.46 mmol. White solid. ¹H NMR (400 MHz, DMSO) δ 12.53 (s, 1H), 8.71 (s, 1H), 7.57 (d, J=8.0 Hz, 2H), 7.34 (d, J=8.1 Hz, 2H), 6.11 (s, 1H), 5.67 (s, 1H), 3.16 (s, 2H), 1.30-1.23 (m, 2H), 1.23-1.15 (m, 2H). LCMS m/z 314.3 (M+H)⁺ (ES⁺).

### Example 12: (R)-4-((2-hydroxy-1-(4-(trifluoromethyl)phenyl)ethyl)amino)-2-methylene-4-oxobutanoic acid

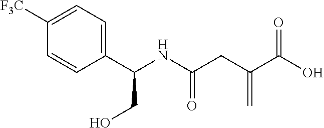 [SMILES: C=C(CC(=O)N[C@@H](CO)c1ccc(C(F)(F)F)cc1)C(=O)O]

Prepared by an analogous method to Example 3, Method A starting from (R)-2-amino-2-(4-(trifluoromethyl)phenyl)ethan-1-ol (240 mg, 1.17 mmol), except that an extra equivalent of DIPEA was used in Step 1. Yield: 108 mg, 0.33 mmol. White solid. ¹H NMR (400 MHz, DMSO) δ 12.44 (s, 1H), 8.38 (d, J=8.0 Hz, 1H), 7.66 (d, J=8.1 Hz, 2H), 7.53 (d, J=8.1 Hz, 2H), 6.09 (d, J=1.8 Hz, 1H), 5.64 (d, J=1.7 Hz, 1H), 4.98-4.76 (m, 2H), 3.58 (t, J=6.0 Hz, 2H), 3.25-3.14 (m, 2H). LCMS m/z 318.3 (M+H)⁺ (ES⁺).

### Example 13: 2-methylene-4-oxo-4-((3-(4-(trifluoromethyl)phenyl)oxetan-3-yl)amino)butanoic acid

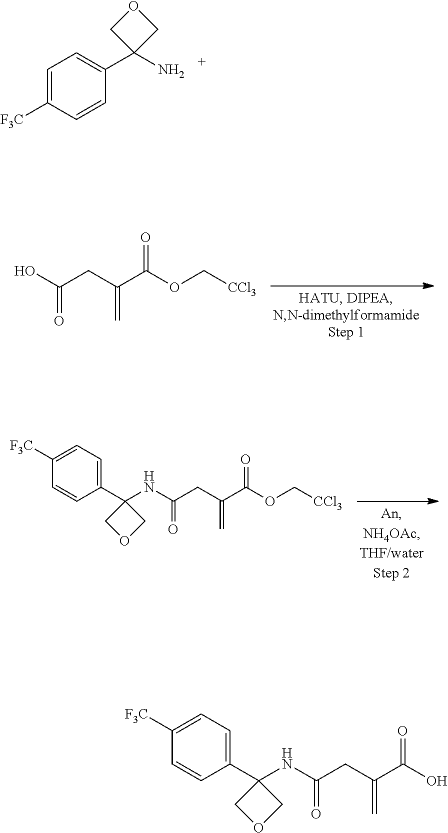

### Step 1

HATU (409 mg, 1.1 mmol) was added to a mixture of 3-(4-(trifluoromethyl) phenyl)oxetan-3-amine, HCl (0.250 g, 0.99 mol), 3-((2,2,2-trichloroethoxy)carbonyl)but-3-enoic acid (Intermediate 3, 234 mg, 0.90 mmol) and DIPEA (0.50 mL, 2.9 mmol) in N,N-dimethylformamide (5 mL). The mixture was stirred at RT for 1 h, then water (20 mL) was added. The mixture was extracted with EtOAc (3×20 mL). The combined organic phases were washed with sat. aq. NaHCO₃ (50 mL), 1 M HCl (50 mL), brine (3×50 mL), dried (MgSO₄) and concentrated. The crude product was purified by chromatography on silica gel (0-100% MTBE/isohexane) to afford 2,2,2-trichloroethyl 2-methylene-4-oxo-4-((3-(4-(trifluoromethyl)phenyl)oxetan-3-yl)amino)butanoate (193 mg, 0.39 mmol, 92% purity) as a white solid. ¹H NMR (400 MHz, DMSO) δ 9.24 (s, 1H), 7.83-7.65 (m, 4H), 6.33 (d, J=1.2 Hz, 1H), 5.96 (d, J=1.2 Hz, 1H), 4.96 (s, 2H), 4.85 (d, J=6.8 Hz, 2H), 4.71 (d, J=6.8 Hz, 2H), 3.38 (s, 2H). LCMS m/z 460.1/462.1 (M+H)⁺ (ES⁺).

### Step 2

Zinc (100 mg, 1.53 mmol) was added to a mixture of 2,2,2-trichloroethyl 2-methylene-4-oxo-4-((3-(4-(trifluoromethyl)phenyl)oxetan-3-yl)amino)butanoate (193 mg, 0.39 mmol, 92% purity) and ammonium acetate (240 mg, 3.11 mmol) in THF (2.4 mL) and water (0.8 mL). The mixture was stirred at RT for 3 h, then filtered and acidified to pH ˜3 with 1 M HCl. The mixture was extracted with EtOAc (3×15 mL). The combined organic phases were washed with brine (25 mL), dried (MgSO₄) and concentrated. The crude product was purified by chromatography on silica gel (0-100% MTBE/isohexane) to afford impure product. Impure product was dissolved in DMSO (1 mL), filtered and purified by reversed phase preparative HPLC on a Waters X-Select CSH C18 ODB prep column, 130 Å, 5 μm, 30 mm×100 mm, flow rate 40 mL min-1 eluting with a 0.1% formic acid in water-MeCN gradient over 17.5 mins using UV detection across all wavelengths with PDA as well as a QDA and ELS detector. At-column dilution pump gives 2 mL min-1 MeCN over the entire method, which is included in the following MeCN percentages. Gradient information: 0.0-0.5 min, 10% MeCN; 0.5-10.5 min, ramped from 10% MeCN to 40% MeCN; 10.5-10.6 min, ramped from 40% MeCN to 100% MeCN; 10.6-12.5 min, held at 100% MeCN to afford 2-methylene-4-oxo-4-((3-(4-(trifluoromethyl)phenyl)oxetan-3-yl)amino)butanoic acid (30 mg, 0.90 mmol) as a white solid. ¹H NMR (400 MHz, DMSO) δ 12.57 (s, 1H), 9.15 (s, 1H), 7.76 (d, J=8.5 Hz, 2H), 7.72 (d, J=8.5 Hz, 2H), 6.13 (d, J=1.7 Hz, 1H), 5.70 (d, J=1.7 Hz, 1H), 4.84 (d, J=6.7 Hz, 2H), 4.69 (d, J=6.7 Hz, 2H), 3.26 (s, 2H). LCMS m/z 330.3 (M+H)⁺ (ES⁺).

### Example 14: 2-methylene-4-oxo-4-((1-(4-(trifluoromethyl)phenyl)cyclopentyl)amino) butanoic acid

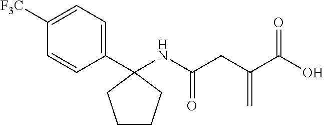 [SMILES: C=C(CC(=O)NC1(c2ccc(C(F)(F)F)cc2)CCCC1)C(=O)O]

Prepared by an analogous method to Example 3, Method A starting from 1-(4-(trifluoromethyl)phenyl)cyclopentan-1-amine (250 mg, 1.09 mmol). Yield: 215 mg, 0.60 mmol. White solid. ¹H NMR (400 MHz, DMSO) δ 12.47 (s, 1H), 8.20 (s, 1H), 7.57 (q, J=8.6 Hz, 4H), 6.10-6.00 (m, 1H), 5.61-5.55 (m, 1H), 3.14 (s, 2H), 2.33-2.21 (m, 2H), 1.94-1.66 (m, 6H). LCMS m/z 342.0 (M+H)⁺ (ES⁺).

### Example 15: (S)-4-((1-(4-chlorophenyl)ethyl)amino)-2-methylene-4-oxobutanoic acid

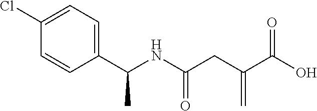 [SMILES: C=C(CC(=O)N[C@@H](C)c1ccc(Cl)cc1)C(=O)O]

Prepared by an analogous method to Example 3, Method A starting from (S)-1-(4-chlorophenyl)ethan-1-amine (288 mg, 1.85 mmol), except TBTU was used in place of HATU for Step 1. Yield: 241 mg, 0.89 mmol. White solid. ¹H NMR (400 MHz, DMSO) δ 12.45 (s, 1H), 8.35 (d, J=7.9 Hz, 1H), 7.39-7.29 (m, 4H), 6.08 (d, J=1.8 Hz, 1H), 5.62 (d, J=1.7 Hz, 1H), 4.94-4.78 (m, 1H), 3.13 (s, 2H), 1.32 (d, J=7.0 Hz, 3H). LCMS m/z 268.1 (M+H)⁺ (ES⁺).

### Example 16: (S)-4-((1-(4-bromophenyl)ethyl)amino)-2-methylene-4-oxobutanoic acid

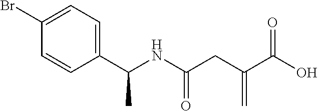 [SMILES: C=C(CC(=O)N[C@@H](C)c1ccc(Br)cc1)C(=O)O]

Prepared by an analogous method to Example 3, Method A starting from (S)-1-(4-bromophenyl)ethan-1-amine (0.250 g, 1.25 mmol), except TBTU was used in place of HATU for Step 1. Yield: 136 mg, 0.43 mmol. White solid. ¹H NMR (400 MHz, DMSO) δ 12.45 (s, 1H), 8.35 (d, J=7.9 Hz, 1H), 7.59-7.40 (m, 2H), 7.35-7.18 (m, 2H), 6.08 (d, J=1.8 Hz, 1H), 5.62 (d, J=1.7 Hz, 1H), 4.91-4.79 (m, 1H), 3.13 (s, 2H), 1.32 (d, J=7.0 Hz, 3H). LCMS m/z 312.0/314.0 (M+H)⁺ (ES⁺).

### Example 17: (S)-4-((1-(4-bromophenyl)ethyl)amino)-2-methylene-4-oxobutanoic acid

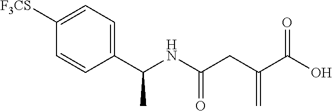

Prepared by an analogous method to Example 3, Method A starting from (S)-1-(4-((trifluoromethyl)thio)phenyl)ethan-1-amine (Intermediate 4, 0.300 g, 1.36 mmol), except that an extra equivalent of DIPEA was used in Step 1. Yield: 283 mg, 0.84 mmol. White solid. ¹H NMR (400 MHz, DMSO) δ 12.47 (s, 1H), 8.42 (d, J=7.8 Hz, 1H), 7.71-7.61 (m, 2H), 7.51-7.41 (m, 2H), 6.09 (d, J=1.7 Hz, 1H), 5.63 (d, J=1.6 Hz, 1H), 5.00-4.88 (m, 1H), 3.15 (s, 2H), 1.35 (d, J=7.1 Hz, 3H). LCMS m/z 334.0 (M+H)⁺ (ES⁺).

### Example 18: (S)-4-((1-(4-(difluoromethyl)phenyl)ethyl)amino)-2-methylene-4-oxobutanoic acid

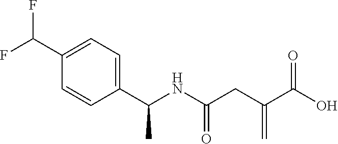 [SMILES: C=C(CC(=O)N[C@@H](C)c1ccc(C(F)F)cc1)C(=O)O]

Prepared by an analogous method to Example 3, Method A starting from (S)-1-(4-(difluoromethyl)phenyl)ethan-1-amine (0.100 g, 0.58 mmol), except TBTU was used in place of HATU for Step 1. Yield: 43 mg, 0.15 mmol. White solid. ¹H NMR (400 MHz, DMSO) δ 12.46 (s, 1H), 8.39 (d, J=7.8 Hz, 1H), 7.50 (d, J=8.0 Hz, 2H), 7.44 (d, J=8.1 Hz, 2H), 6.99 (t, J=56.0 Hz, 1H), 6.08 (d, J=1.8 Hz, 1H), 5.63 (d, J=1.6 Hz, 1H), 4.99-4.86 (m, 1H), 3.15 (s, 2H), 1.35 (d, J=7.0 Hz, 3H). LCMS m/z 284.1 (M+H)⁺ (ES⁺).

### Example 19: (S)-4-((1-(3-fluoro-4-(trifluoromethyl)phenyl)ethyl)amino)-2-methylene-4-oxobutanoic acid

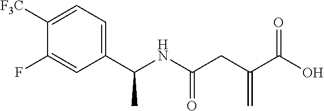 [SMILES: C=C(CC(=O)N[C@@H](C)c1ccc(C(F)(F)F)c(F)c1)C(=O)O]

Prepared by an analogous method to Example 3, Method A starting from (S)-1-(3-fluoro-4-(trifluoromethyl)phenyl)ethan-1-amine (0.250 g, 1.21 mmol), except TBTU was used in place of HATU for Step 1. Yield: 124 mg, 0.38 mmol. White solid. ¹H NMR (400 MHz, DMSO) δ 12.48 (s, 1H), 8.47 (d, J=7.6 Hz, 1H), 7.71 (app t, J=7.9 Hz, 1H), 7.42 (d, J=12.2 Hz, 1H), 7.35 (d, J=8.1 Hz, 1H), 6.09 (d, J=1.7 Hz, 1H), 5.65 (d, J=1.6 Hz, 1H), 4.97-4.89 (m, 1H), 3.24-3.08 (m, 2H), 1.35 (d, J=7.1 Hz, 3H). LCMS m/z 320.0 (M+H)⁺ (ES⁺).

### Example 20: (S)-2-methylene-4-oxo-4-((1-(4-(perfluoroethyl)phenyl)ethyl)amino)butanoic acid

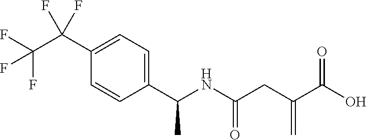 [SMILES: C=C(CC(=O)N[C@@H](C)c1ccc(C(F)(F)C(F)(F)F)cc1)C(=O)O]

Prepared by an analogous method to Example 3, Method A starting from (S)-1-(4-(perfluoroethyl)phenyl)ethan-1-amine, HCl (Intermediate 5, 52 mg, 0.19 mmol), except that an extra 2 equivalents of DIPEA were used in Step 1. Yield: 26 mg, 0.075 mmol. White solid. ¹H NMR (400 MHz, DMSO) δ 12.46 (s, 1H), 8.47 (d, J=7.8 Hz, 1H), 7.63 (d, J=8.3 Hz, 2H), 7.56 (d, J=8.2 Hz, 2H), 6.09 (d, J=1.8 Hz, 1H), 5.63 (d, J=1.7 Hz, 1H), 5.01-4.91 (m, 1H), 3.16 (s, 2H), 1.36 (d, J=7.1 Hz, 3H). LCMS m/z 352.1 (M+H)⁺ (ES⁺).

### Example 21: (R)-2-methylene-4-oxo-4-((2,2,2-trifluoro-1-(4-(trifluoromethyl)phenyl)ethyl) amino)butanoic acid

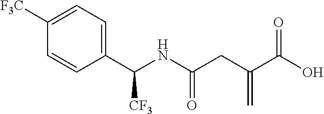 [SMILES: C=C(CC(=O)N[C@H](c1ccc(C(F)(F)F)cc1)C(F)(F)F)C(=O)O]

Prepared by an analogous method to Example 3, Method A starting from (R)-2,2,2-trifluoro-1-(4-(trifluoromethyl)phenyl)ethan-1-amine hydrochloride (0.30 g, 1.07 mmol). Yield: 279 mg, 0.78 mmol. White solid. ¹H NMR (400 MHz, DMSO) δ 12.48 (s, 1H), 9.31 (d, J=9.5 Hz, 1H), 7.82 (s, 4H), 6.12 (d, J=1.7 Hz, 1H), 6.01-5.89 (m, 1H), 5.67 (d, J=1.6 Hz, 1H), 3.29 (d, J=5.2 Hz, 2H). LCMS m/z 356.3 (M+H)⁺ (ES⁺).

### Example 22: 1-(methylsulfonyl)piperidin-4-yl (S)-2-methylene-4-oxo-4-((1-(4-(trifluoromethyl)phenyl)ethyl)amino)butanoate

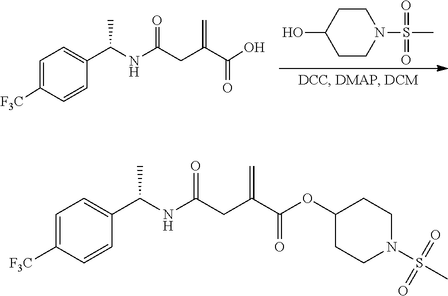 [SMILES: C=C(CC(=O)N[C@@H](C)c1ccc(C(F)(F)F)cc1)C(=O)O ; CS(=O)(=O)N1CCC(O)CC1 ; C=C(CC(=O)N[C@@H](C)c1ccc(C(F)(F)F)cc1)C(=O)OC1CCN(S(C)(=O)=O)CC1]

DCC (103 mg, 0.5 mmol) was added to a mixture of (S)-2-methylene-4-oxo-4-((1-(4-(trifluoromethyl)phenyl)ethyl)amino)butanoic acid (Example 3, 100 mg, 0.33 mmol), 1-(methylsulfonyl)piperidin-4-ol (60 mg, 0.33 mmol) and DMAP (6 mg, 50 μmol) in DCM (1.4 mL) at RT. The mixture was stirred for 18 h. The mixture was filtered and concentrated. The crude product was purified by chromatography on silica gel (0-10% MeOH/DCM) to afford impure product. The mixture was triturated with MTBE (10 mL) and filtered through a syringe filter. The filtrate was discarded and the syringe filter was washed with DCM. The filtrate was concentrated to afford 1-(methylsulfonyl)piperidin-4-yl (S)-2-methylene-4-oxo-4-((1-(4-(trifluoromethyl)phenyl)ethyl)amino)butanoate (7 mg, 0.01 mmol) as a white solid. ¹H NMR (400 MHz, DMSO) δ 8.48 (d, J=7.7 Hz, 1H), 7.68 (d, J=8.1 Hz, 2H), 7.52 (d, J=8.1 Hz, 2H), 6.17 (d, J=1.5 Hz, 1H), 5.71 (d, J=1.4 Hz, 1H), 4.98-4.91 (m, 1H), 4.89-4.80 (m, 1H), 3.25-3.17 (m, 4H), 3.18-3.07 (m, 2H), 2.86 (s, 3H), 1.96-1.78 (m, 2H), 1.69-1.56 (m, 2H), 1.36 (d, J=7.0 Hz, 3H). LCMS m/z 463.2 (M+H)⁺ (ES⁺).

### Example 23: 4-((cyclopropyl(4-(trifluoromethyl)phenyl)methyl)amino)-2-methylene-4-oxobutanoic acid

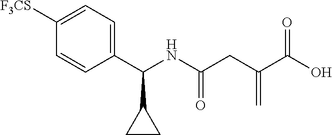

Prepared by an analogous method to Example 3, Method A starting from (S)-cyclopropyl(4-(trifluoromethyl)phenyl)methanamine hydrochloride (Intermediate 6, 0.50 g, 1.74 mmol), except that an extra equivalent of DIPEA was used in Step 1. Yield: 405 mg, 1.2 mmol. White solid. ¹H NMR (400 MHz, DMSO) δ 12.46 (s, 1H), 8.57 (d, J=8.0 Hz, 1H), 7.67 (d, J=8.1 Hz, 2H), 7.58 (d, J=8.1 Hz, 2H), 6.09 (d, J=1.8 Hz, 1H), 5.63 (d, J=1.7 Hz, 1H), 4.27 (t, J=8.5 Hz, 1H), 3.21-3.11 (m, 2H), 1.18-1.07 (m, 1H), 0.56-0.45 (m, 2H), 0.45-0.37 (m, 1H), 0.37-0.30 (m, 1H). LCMS m/z 328.0 (M+H)⁺ (ES⁺).

### Example 24: 1-methylpiperidin-4-yl (S)-2-methylene-4-oxo-4-((1-(4-(trifluoromethyl) phenyl)ethyl)amino)butanoate

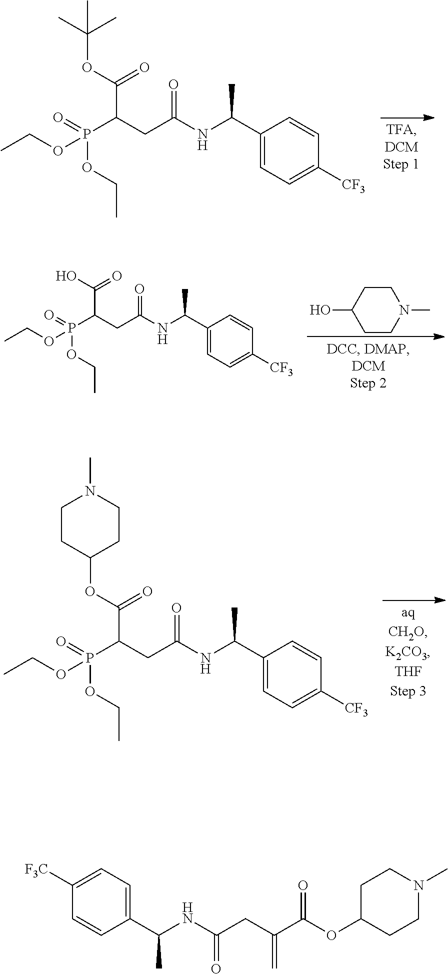

### Step 1

A mixture of tert-butyl 2-(diethoxyphosphoryl)-4-oxo-4-(((S)-1-(4-(trifluoromethyl)phenyl)ethyl)amino)butanoate (Example 3, Step 1, 1.24 g, 90% Wt, 2.31 mmol) and TFA (3.0 mL, 39 mmol) in DCM (6 mL) was stirred at RT for 3 h. The mixture was concentrated and co-evaporated with toluene (3×5 mL) to afford 2-(diethoxyphosphoryl)-4-oxo-4-(((S)-1-(4-(trifluoromethyl)phenyl)ethyl)amino)butanoic acid (1.30 g, 2.3 mmol, 75% purity) as a pale yellow gum. ¹H NMR (400 MHz, DMSO) δ 8.55 (dd, J=7.7, 2.8 Hz, 1H), 7.71-7.60 (m, 2H), 7.55-7.48 (m, 2H), 4.99-4.85 (m, 1H), 4.13-3.95 (m, 4H), 3.36-3.19 (m, 1H), 2.87-2.76 (m, 1H), 2.55-2.42 (m, 1H), 1.34 (d, J=7.1 Hz, 3H), 1.28-1.17 (m, 6H) (exchangeable proton not visible). LCMS m/z 426.1 (M+H)⁺ (ES⁺).

### Step 2

DCC (0.11 g, 0.53 mmol) was added to a mixture of 2-(diethoxyphosphoryl)-4-oxo-4-(((S)-1-(4-(trifluoromethyl)phenyl)ethyl)amino)butanoic acid (0.25 g, 0.44 mmol, 75% purity), 1-methylpiperidin-4-ol (53 mg, 0.46 mmol) and DMAP (8 mg, 70 μmol) in DCM (3.0 mL) at RT. The mixture was stirred for 72 h. The mixture was filtered and the solid was washed with DCM (5 mL). The mixture was concentrated and redissolved in DCM (2 mL), filtered through a syringe filter and concentrated to afford 1-methylpiperidin-4-yl 2-(diethoxyphosphoryl)-4-oxo-4-(((S)-1-(4-(trifluoromethyl)phenyl)ethyl)amino)butanoate (299 mg, 0.46 mmol, 80% purity) as a brown gum that was used without further purification. LCMS m/z 523.3 (M+H)⁺ (ES⁺).

### Step 3

Formaldehyde (37% aqueous, 0.17 mL, 2.3 mmol) was added to a suspension of 1-methylpiperidin-4-yl 2-(diethoxyphosphoryl)-4-oxo-4-(((S)-1-(4-(trifluoromethyl)phenyl)ethyl)amino)butanoate (299 mg, 0.46 mmol, 80% purity) and potassium carbonate (127 mg, 0.92 mmol) in THF (2.0 mL). The mixture was stirred at RT for 18 h, then diluted with water (20 mL) and extracted with EtOAc (3×20 mL). The combined organic phases were washed with brine (50 mL), dried (MgSO₄) and concentrated. The crude product was purified by chromatography on silica gel (0-10% MeOH/DCM then 10%(0.7 M ammonia/MeOH)/DCM) to afford 1-methylpiperidin-4-yl (S)-2-methylene-4-oxo-4-((1-(4-(trifluoromethyl)phenyl)ethyl)amino)butanoate (86 mg, 0.21 mmol) as a white solid. ¹H NMR (400 MHz, DMSO) δ 8.47 (d, J=7.7 Hz, 1H), 7.67 (d, J=8.2 Hz, 2H), 7.52 (d, J=8.1 Hz, 2H), 6.12 (d, J=1.6 Hz, 1H), 5.66 (d, J=1.5 Hz, 1H), 5.00-4.88 (m, 1H), 4.74-4.63 (m, 1H), 3.25-3.14 (m, 2H), 2.49-2.39 (m, 2H), 2.24-2.10 (m, 5H), 1.86-1.66 (m, 2H), 1.61-1.43 (m, 2H), 1.36 (d, J=7.1 Hz, 3H). LCMS m/z 399.2 (M+H)⁺ (ES⁺).

### Example 25: 2-(methylsulfonamido)ethyl (S)-2-methylene-4-oxo-4-((1-(4-(trifluoromethyl) phenyl)ethyl)amino)butanoate

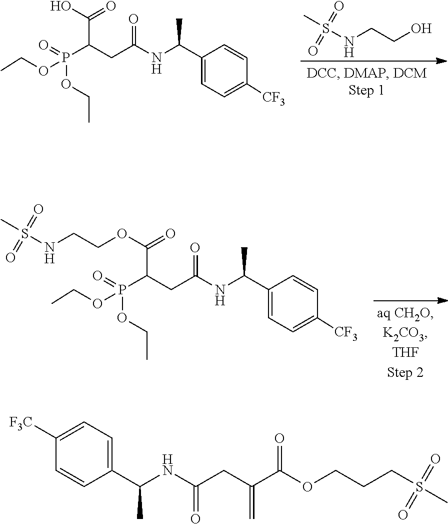

### Step 1

DCC (0.11 g, 0.53 mmol) was added to a mixture of 2-(diethoxyphosphoryl)-4-oxo-4-(((S)-1-(4-(trifluoromethyl)phenyl)ethyl)amino)butanoic acid (Example 24, Step 1, 0.25 g, 0.44 mmol), N-(2-hydroxyethyl)methanesulfonamide (64 mg, 0.46 mmol) and DMAP (8 mg, 70 μmol) in DCM (3.0 mL) at RT. The mixture was stirred for 72 h. The mixture was filtered and the solid was washed with DCM (5 mL). The mixture was concentrated and redissolved in DCM (2 mL), filtered through a syringe filter and concentrated to afford 2-(methylsulfonamido)ethyl 2-(diethoxyphosphoryl)-4-oxo-4-(((S)-1-(4-(trifluoromethyl)phenyl)ethyl)amino)butanoate (0.343 g, 0.44 mmol, 70% purity) as a brown gum that was used without further purification. LCMS m/z 547.0 (M+H)⁺ (ES⁺).

### Step 2

Formaldehyde (37% aqueous, 0.17 mL, 2.3 mmol) was added to a suspension of 2-(methylsulfonamido)ethyl 2-(diethoxyphosphoryl)-4-oxo-4-(((S)-1-(4-(trifluoromethyl)phenyl)ethyl)amino)butanoate (0.343 g, 0.44 mmol, 70% purity) and potassium carbonate (122 mg, 0.89 mmol) in THF (2.0 mL). The mixture was stirred at RT for 18 h, then diluted with water (20 mL) and extracted with EtOAc (3×20 mL). The combined organic phases were washed with brine (50 mL), dried (MgSO₄) and concentrated. The crude product was purified by chromatography on silica gel (0-10% MeOH/DCM) to afford impure product. The crude product was dissolved in DMSO (2 mL), filtered and purified by reversed phase preparative HPLC (Waters X-Select CSH C18 ODB prep column, 130 Å, 5 μm, 30 mm×100 mm, flow rate 40 mL min-1 eluting with a 0.1% formic acid in water-MeCN gradient over 12.5 mins using UV detection across all wavelengths with PDA as well as a QDA and ELS detector. At-column dilution pump gives 2 mL min-1 MeCN over the entire method, which is included in the following MeCN percentages. Gradient information: 0.0-0.5 min, 20% MeCN; 0.5-10.5 min, ramped from 20% MeCN to 50% MeCN; 10.5-10.6 min, ramped from 50% MeCN to 100% MeCN; 10.6-12.5 min, held at 100% MeCN). The clean fractions were evaporated in a Genevac to afford 2-(methylsulfonamido)ethyl (S)-2-methylene-4-oxo-4-((1-(4-(trifluoromethyl)phenyl)ethyl)amino)butanoate (12 mg, 28 μmol) as a colourless gum. ¹H NMR (400 MHz, DMSO) δ 8.49 (d, J=7.6 Hz, 1H), 7.68 (d, J=8.2 Hz, 2H), 7.52 (d, J=8.1 Hz, 2H), 7.21 (t, J=6.0 Hz, 1H), 6.22 (d, J=1.5 Hz, 1H), 5.73 (d, J=1.3 Hz, 1H), 4.99-4.90 (m, 1H), 4.17-4.03 (m, 2H), 3.25-3.16 (m, 4H), 2.90 (s, 3H), 1.36 (d, J=7.0 Hz, 3H). LCMS m/z 423.2 (M+H)⁺ (ES⁺).

### Example 26: 2-(dimethylamino)ethyl (S)-2-methylene-4-oxo-4-((1-(4-(trifluoromethyl) phenyl)ethyl)amino)butanoate

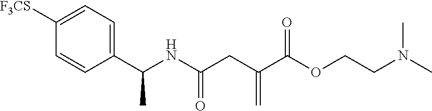

Prepared by an analogous method to Example 25 starting from 2-(dimethylamino)ethan-1-ol (53 μL, 0.53 mmol) except EDC HCl was used instead of DCC in Step 1. Yield: 7 mg, 0.02 mmol. White solid. ¹H NMR (400 MHz, DMSO) δ 8.48 (d, J=7.7 Hz, 1H), 7.68 (d, J=8.2 Hz, 2H), 7.52 (d, J=8.1 Hz, 2H), 6.12 (d, J=1.6 Hz, 1H), 5.69 (d, J=1.4 Hz, 1H), 5.03-4.85 (m, 1H), 4.17-4.03 (m, 2H), 3.20 (s, 2H), 2.44 (t, J=5.9 Hz, 2H), 2.14 (s, 6H), 1.36 (d, J=7.0 Hz, 3H). LCMS m/z 373.2 (M+H)⁺ (ES⁺).

### Example 27: 1,1-dioxidothietan-3-yl (S)-2-methylene-4-oxo-4-((1-(4-(trifluoromethyl) phenyl)ethyl)amino)butanoate

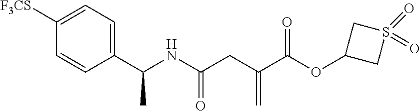

Prepared by an analogous method to Example 22 starting from 3-hydroxythietane 1,1-dioxide (110 mg, 0.90 mmol) Yield: 109 mg, 0.27 mmol. White solid. ¹H NMR (400 MHz, CDCl₃) δ: 7.59 (d, J=8.0 Hz, 2H), 7.42 (d, J=8.4 Hz, 2H), 6.41 (s, 1H), 6.02 (d, J=6.8 Hz, 1H), 5.92 (s, 1H), 5.35-5.30 (m, 1H), 5.16-5.11 (m, 1H), 4.59-4.50 (m, 2H), 4.19-4.06 (m, 2H), 3.24 (d, J=2.0 Hz, 2H), 1.52 (d, J=5.6 Hz, 3H). LCMS m/z 406.1 (M+H)⁺ (ES⁺).

### Example 28: 3-methyloxetan-3-yl (S)-2-methylene-4-oxo-4-((1-(4-(trifluoromethyl)phenyl) ethyl)amino)butanoate

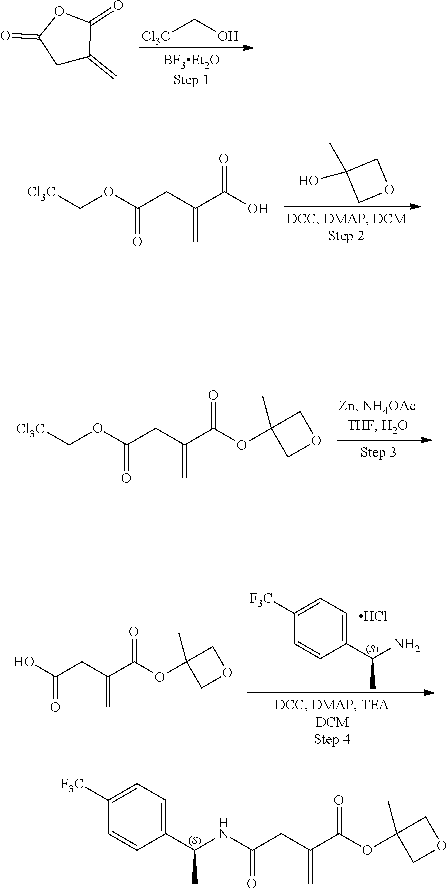

### Step 1

To a mixture of 3-methylenedihydrofuran-2,5-dione (10.0 g, 89.2 mmol) and 2,2,2-trichloroethanol (20.0 g, 133.8 mmol) was added boron trifluoride diethyl etherate (1.27 g, 8.92 mmol), and the mixture was allowed to stir at 75° C. for 40 minutes. The mixture was cooled to room temperature, quenched with methanol (4 mL), diluted with EtOAc (100 mL) and water (20 mL). The organic phase was separated, and the aqueous phase extracted with EtOAc (2×50 mL). The combined organic layers were washed with brine, dried over Na₂SO₄ and concentrated under reduced pressure. The residue was purified by reversed column chromatography (Column: Boston ODS 120 g Flash; Flow Rate: 40 mL/min; solvent system: MeCN/(10 mmol/L HCl/water); MeCN: gradient: 60-80%; collection wavelength: 214 nm). The clean fractions were concentrated under reduced pressure to remove MeCN, and the residue was lyophilized to give 2-methylene-4-oxo-4-(2,2,2-trichloroethoxy)butanoic acid (18.0 g, 77% yield) as white solid, containing 5% regioisomer as measured by ¹H NMR. The solid was triturated with n-hexane (150 mL)/tert-butyl methyl ether (20 mL), stirred at room temperature overnight, recovered by filtration and dried at 40° C. under reduced pressure to give 2-methylene-4-oxo-4-(2,2,2-trichloroethoxy)butanoic acid (16.0 g, 68% yield). ¹H NMR (400 MHz, DMSO-d6) δ 12.74 (br, 1H), 6.22 (s, 1H), 5.86 (s, 1H), 4.89 (s, 2H), 3.49 (s, 2H). LCMS m/z 261.2 (M+H)⁺ (ES⁺).

### Step 2

A mixture of 2-methylene-4-oxo-4-(2,2,2-trichloroethoxy)butanoic acid (520 mg, 2.00 mmol), 3-methyloxetan-3-ol (175 mg, 2.0 mmol), DCC (618 mg, 3.0 mmol) and DMAP (49 mg, 0.4 mmol) in DCM (10 mL) was stirred at room temperature for 30 minutes. The mixture was filtered and the filtrate was concentrated under reduced pressure. The residue was purified by flash column chromatography on silica (0-20% tert-butyl methyl ether/petroleum ether) to give 1-(3-methyloxetan-3-yl) 4-(2,2,2-trichloroethyl) 2-methylenesuccinate (420 mg, 63% yield) as yellow oil. LCMS m/z 331.0 (M+H)⁺ (ES⁺).

### Step 3

A mixture of 1-(3-methyloxetan-3-yl) 4-(2,2,2-trichloroethyl) 2-methylenesuccinate (420 mg, 1.27 mmol), zinc powder (165 mg, 2.54 mmol) and NH₄OAc (293 mg, 3.81 mmol) in THF (4 mL)/water (1 mL) was stirred at room temperature for 2 hours. The reaction mixture was filtered and the filtrate was acidified with 0.5 N HCl until pH=5-6, and extracted with ethyl acetate (3×5 mL). The organic layer was washed by brine, dried over Na₂SO₄. The filtrate was concentrated under reduced pressure to give 3-((3-methyloxetan-3-yloxy)carbonyl)but-3-enoic acid (250 mg, 98% yield) as yellow solid. The product was used to the next step without further purification. LCMS m/z 201.2 (M+H)⁺ (ES⁺).

### Step 4

A solution of (S)-1-(4-(trifluoromethyl)phenyl)ethanamine hydrochloride (282 mg, 1.25 mmol) and TEA (126 mg, 1.25 mmol) in DCM (6 mL) was stirred at room temperature for 10 minutes, and to the mixture was added 3-((3-methyloxetan-3-yloxy)carbonyl)but-3-enoic acid (250 mg, 1.25 mmol), DCC (344 mg, 1.62 mmol) and DMAP (30 mg, 0.25 mmol). The reaction mixture was stirred at room temperature for 2 hours then the solid was filtered, and the filtrate was concentrated under reduced pressure. The residue was purified by prep-HPLC (Column: Waters X-bridge Prep C18 OBD 10 μm 19×250 mm; Flow Rate: 20 mL/min; solvent system: MeCN/(0.2% formic acid/water); MeCN gradient: 35-65%; collection wavelength: 214 nm). The fractions were concentrated under reduced pressure to remove MeCN, and the residue was lyophilized to give (S)-3-methyloxetan-3-yl 2-methylene-4-oxo-4-(1-(4-(trifluoromethyl) phenyl)ethylamino)butanoate (68.2 mg, 14% yield) as white solid. ¹H NMR (400 MHz, DMSO-d6) δ 8.50 (d, 1H), 7.67 (d, J=8.4 Hz, 2H), 7.52 (d, J=8.4 Hz, 2H), 6.14 (s, 1H), 5.72 (s, 1H), 4.97-4.93 (m, 1H), 4.59-4.53 (m, 2H), 4.40 (t, J=7.2 Hz, 2H), 3.21 (s, 2H), 1.52 (s, 3H), 1.36 (d, J=7.2 Hz, 3H). LCMS m/z 372.2 (M+H)⁺ (ES⁺).

### Example 29: 2-morpholinoethyl 2-(diethoxyphosphoryl)-4-oxo-4-(((S)-1-(4-(trifluoromethyl)phenyl)ethyl)amino)butanoate

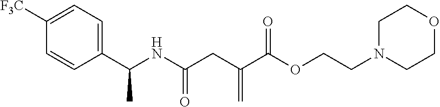 [SMILES: C=C(CC(=O)N[C@@H](C)c1ccc(C(F)(F)F)cc1)C(=O)OCCN1CCOCC1]

Prepared by an analogous method to Example 25 starting from 2-(dimethylamino)ethan-1-ol (53 μL, 0.53 mmol) except EDC HCl was used instead of DCC in Step 1. Yield: 86 mg, 0.20 mmol. White solid. ¹H NMR (400 MHz, DMSO) δ 8.48 (d, J=7.7 Hz, 1H), 7.68 (d, J=8.1 Hz, 2H), 7.53 (d, J=8.1 Hz, 2H), 6.12 (d, J=1.5 Hz, 1H), 5.70 (d, J=1.5 Hz, 1H), 5.01-4.89 (m, 1H), 4.22-4.09 (m, 2H), 3.58-3.51 (m, 4H), 3.21 (s, 2H), 2.43-2.34 (m, 4H), 1.37 (d, J=7.1 Hz, 3H) [2 protons obscured by DMSO peak]. LCMS m/z 415.3 (M+H)⁺ (ES⁺).

### Example 30: (S)-2-methylene-4-oxo-4-((1-(5-(trifluoromethyl)pyridin-2-yl)ethyl)amino) butanoic acid

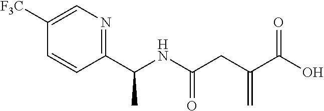 [SMILES: C=C(CC(=O)N[C@@H](C)c1ccc(C(F)(F)F)cn1)C(=O)O]

### Method A

Prepared by an analogous method to Example 3, Method A starting from (S)-1-(5-(trifluoromethyl)pyridin-2-yl)ethan-1-amine, 2HCl (Intermediate 7, 0.60 g, 2.3 mmol), except that an extra equivalent of DIPEA was used in Step 1 and TBTU was used in place of HATU. Yield: 133 mg, 0.32 mmol. White solid (obtained as TFA salt). ¹H NMR (400 MHz, DMSO) δ 12.47 (s, 1H), 8.89 (d, J=2.3 Hz, 1H), 8.50 (d, J=7.4 Hz, 1H), 8.14 (dd, J=8.3, 2.4 Hz, 1H), 7.58 (d, J=8.3 Hz, 1H), 6.10 (d, J=1.7 Hz, 1H), 5.65 (d, J=1.6 Hz, 1H), 5.05-4.86 (m, 1H), 3.19 (s, 2H), 1.39 (d, J=7.1 Hz, 3H). LCMS m/z 303.3 (M+H)⁺ (ES⁺).

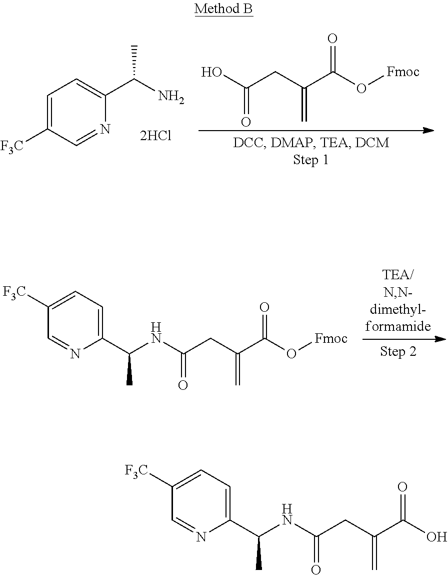

### Step 1

A mixture of (S)-1-(5-(trifluoromethyl)pyridin-2-yl)ethan-1-amine dihydrochloride (Intermediate 7, 1.00 g, 3.83 mmol), 3-(((9H-fluoren-9-yl)methoxy)carbonyl)but-3-enoic acid (Intermediate 10, 1.35 g, 3.83 mmol), triethylamine (774 mg, 7.66 mmol), DCC (1.19 g, 5.74 mmol) and DMAP (47 mg, 0.38 mmol) in dichloromethane (20 mL) was stirred at 25° C. for 1 hour. The mixture was filtered and concentrated under reduced pressure, and the residue was purified by flash column chromatography on silica (10-30% tert-butyl methyl ether/petroleum ether) to give (S)-((9H-fluoren-9-yl)methyl carbonic) (S)-2-methylene-4-oxo-4-((1-(5-(trifluoromethyl)pyridin-2-yl)ethyl)amino)butanoic anhydride (1.30 g, 65% yield) as white solid. LCMS m/z 481.2 (M+H)⁺ (ES⁺).

### Step 2

A mixture of (S)-((9H-fluoren-9-yl)methyl carbonic) (S)-2-methylene-4-oxo-4-((1-(5-(trifluoromethyl)pyridin-2-yl)ethyl)amino)butanoic anhydride (1.30 g, 2.48 mmol) and triethylamine (751 mg, 7.44 mmol) in N,N-dimethylformamide (5 mL) was stirred at 25° C. for 2 hours. The mixture was acidified with 0.5N HCl to pH 5-6, and extracted with EtOAc (3×10 mL). The combined organic layers were washed by brine, dried over Na₂SO₄, and concentrated under reduced pressure. The residue was purified by flash column chromatography on silica (10-50% tert-butyl methyl ether/petroleum ether) to give (S)-2-methylene-4-oxo-4-((1-(5-(trifluoromethyl)pyridin-2-yl)ethyl)amino)butanoic acid (700 mg, 83% yield) as white solid. LCMS m/z 303.2 (M+H)⁺

### Example 31: 1-(methylsulfonyl)piperidin-4-yl (S)-2-methylene-4-oxo-4-((1-(5-(trifluoromethyl)pyridin-2-yl)ethyl)amino)butanoate

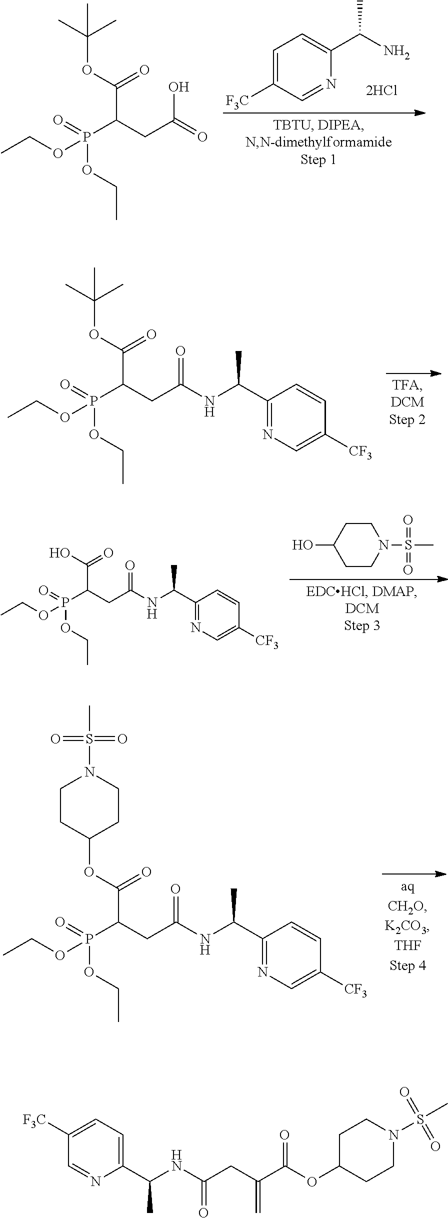

### Step 1

TBTU (0.88 g, 2.7 mmol) was added to a mixture of (S)-1-(5-(trifluoromethyl)pyridin-2-yl)ethan-1-amine 2HCl (Intermediate 7, 0.60 g, 2.30 mmol), 4-(tert-butoxy)-3-(diethoxyphosphoryl)-4-oxobutanoic acid (Intermediate 2, 0.74 g, 2.4 mmol) and DIPEA (2.0 mL, 11 mmol) in N,N-dimethylformamide (10 mL). The mixture was stirred at RT for 1 h. 1 M NH₄Cl (50 mL) was added. The mixture was extracted with EtOAc (3×50 mL). The combined organic phases were washed with sat. aq. NaHCO₃ (50 mL), brine (3×50 mL), dried (MgSO₄) and concentrated to afford tert-butyl 2-(diethoxyphosphoryl)-4-oxo-4-(((S)-1-(5-(trifluoromethyl)pyridin-2-yl)ethyl)amino)butanoate (1.19 g, 2.0 mmol, 80% purity) as a brown oil. ¹H NMR (400 MHz, DMSO) δ 8.95-8.85 (m, 1H), 8.62 (dd, J=16.5, 7.3 Hz, 1H), 8.22-8.07 (m, 1H), 7.55 (dd, J=17.9, 8.3 Hz, 1H), 4.95 (h, J=7.0 Hz, 1H), 4.12-3.94 (m, 4H), 3.30-3.16 (m, 1H), 2.81 (ddt, J=15.3, 11.6, 7.5 Hz, 1H), 2.56-2.46 (m, 1H), 1.41-1.20 (m, 18H). LCMS m/z 483.0 (M+H)⁺ (ES⁺).

### Step 2

A mixture of tert-butyl 2-(diethoxyphosphoryl)-4-oxo-4-(((S)-1-(5-(trifluoromethyl)pyridin-2-yl)ethyl)amino)butanoate (0.50 g, 0.83 mmol, 80% purity) and TFA (1.5 mL, 19 mmol) in DCM (3 mL) was stirred at RT for 3 h. The mixture was concentrated and co-evaporated with toluene (3×5 mL) to afford 2-(diethoxyphosphoryl)-4-oxo-4-(((S)-1-(5-(trifluoromethyl)pyridin-2-yl)ethyl)amino)butanoic acid, trifluoroacetic acid salt (597 mg, ca 80% purity) as a pale yellow gum that was used without further purification. ¹H NMR (400 MHz, DMSO) δ 8.89 (d, J=2.4 Hz, 1H), 8.62 (dd, J=13.5, 7.4 Hz, 1H), 8.29-8.06 (m, 1H), 7.55 (dd, J=15.0, 8.3 Hz, 1H), 5.05-4.89 (m, 1H), 4.12-3.97 (m, 4H), 3.28 (dddd, J=23.6, 11.1, 7.7, 3.5 Hz, 1H), 2.92-2.79 (m, 1H), 2.58-2.51 (m, 1H), 1.40-1.35 (m, 3H), 1.28-1.18 (m, 6H). (exchangeable proton not visible). LCMS m/z 427.1 (M+H)⁺ (ES⁺).

### Step 3

EDC (208 mg, 1.09 mmol) was added to a mixture of 2-(diethoxyphosphoryl)-4-oxo-4-(((S)-1-(5-(trifluoromethyl)pyridin-2-yl)ethyl)amino)butanoic acid, trifluoroacetic acid (0.59 g, 0.90 mmol, ca. 80% purity), 1-(methylsulfonyl)piperidin-4-ol (195 mg, 1.09 mmol) and DMAP (111 mg, 91 mmol) in DCM (5.0 mL) at RT. The mixture was stirred for 18 h. The mixture was diluted with sat. NH₄Cl (20 mL) and extracted with DCM (3×20 mL). The combined organic phases were washed with brine (50 mL) dried (MgSO₄) and concentrated. The crude product was purified by chromatography on silica gel (0-10% (0.7 M Ammonia/MeOH)/DCM) to afford 1-(methylsulfonyl)piperidin-4-yl 2-(diethoxyphosphoryl)-4-oxo-4-(((S)-1-(5-(trifluoromethyl)pyridin-2-yl)ethyl)amino)butanoate (0.29 g, 0.40 mmol, 80% purity) as a pale yellow oil. ¹H NMR (400 MHz, DMSO) δ 8.95-8.83 (m, 1H), 8.64 (t, J=7.6 Hz, 1H), 8.16 (ddd, J=8.6, 5.8, 2.2 Hz, 1H), 7.75-7.46 (m, 1H), 5.01-4.91 (m, 1H), 4.90-4.77 (m, 1H), 4.11-3.99 (m, 4H), 3.44-3.03 (m, 5H), 2.95-2.84 (m, 4H), 2.71-2.55 (m, 1H), 1.93-1.49 (m, 4H), 1.41-1.35 (m, 3H), 1.28-1.19 (m, 6H). LCMS m/z 588.3 (M+H)⁺ (ES⁺).

### Step 4

Formaldehyde (37% aqueous, 0.15 mL, 2.0 mmol) was added to a suspension of 1-(methylsulfonyl)piperidin-4-yl 2-(diethoxyphosphoryl)-4-oxo-4-(((S)-1-(5-(trifluoromethyl)pyridin-2-yl)ethyl)amino)butanoate (0.29 g, 0.40 mmol, 80% purity) and potassium carbonate (110 mg, 0.8 mmol) in THF (2.0 mL). The mixture was stirred at RT for 18 h, then diluted with water (20 mL) and extracted with EtOAc (3×20 mL). The combined organic phases were washed with brine (50 mL), dried (MgSO₄) and concentrated. The crude product was purified by chromatography on silica gel (0-10% MeOH/DCM) to afford 1-(methylsulfonyl)piperidin-4-yl (S)-2-methylene-4-oxo-4-((1-(5-(trifluoromethyl)pyridin-2-yl)ethyl)amino)butanoate (121 mg, 0.26 mmol) as a white solid. ¹H NMR (400 MHz, DMSO) δ 8.92-8.88 (m, 1H), 8.53 (d, J=7.4 Hz, 1H), 8.20-8.14 (m, 1H), 7.57 (d, J=8.3 Hz, 1H), 6.18 (d, J=1.5 Hz, 1H), 5.72 (d, J=1.5 Hz, 1H), 5.06-4.94 (m, 1H), 4.90-4.82 (m, 1H), 3.30-3.17 (m, 4H), 3.17-3.07 (m, 2H), 2.87 (s, 3H), 1.97-1.79 (m, 2H), 1.70-1.57 (m, 2H), 1.39 (d, J=7.1 Hz, 3H). LCMS m/z 464.3 (M+H)⁺ (ES⁺).

### Example 32: 1-methylpiperidin-4-yl (S)-2-methylene-4-oxo-4-((1-(5-(trifluoromethyl)pyridin-2-yl)ethyl)amino)butanoate

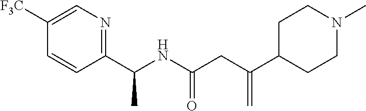 [SMILES: C=C(CC(=O)N[C@@H](C)c1ccc(C(F)(F)F)cn1)C1CCN(C)CC1]

Prepared by an analogous method to Example 31 starting from 1-methylpiperidin-4-ol (128 mg, 1.11 mmol). Yield: 21 mg, 0.05 mmol. Yellow gum. ¹H NMR (400 MHz, DMSO) δ 8.92-8.87 (m, 1H), 8.52 (d, J=7.4 Hz, 1H), 8.18 (dd, J=8.3, 2.4 Hz, 1H), 7.57 (d, J=8.3 Hz, 1H), 6.12 (d, J=1.6 Hz, 1H), 5.68 (d, J=1.5 Hz, 1H), 5.04-4.94 (m, 1H), 4.73-4.62 (m, 1H), 3.26-3.21 (m, 2H), 2.47-2.37 (m, 2H), 2.19-2.08 (m, 5H), 1.81-1.67 (m, 2H), 1.59-1.43 (m, 2H), 1.40 (d, J=7.1 Hz, 3H). LCMS m/z 400.4 (M+H)⁺ (ES⁺).

### Example 33: 1,1-dioxidothietan-3-yl (S)-2-methylene-4-oxo-4-((1-(5-(trifluoromethyl) pyridin-2-yl)ethyl)amino)butanoate

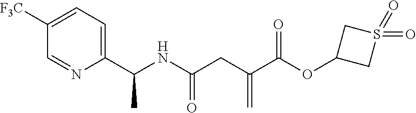 [SMILES: C=C(CC(=O)N[C@@H](C)c1ccc(C(F)(F)F)cn1)C(=O)OC1CS(=O)(=O)C1]

Prepared by an analogous method to Example 28 using 3-hydroxythietane 1,1-dioxide, (208 mg, 1.70 mmol) in Step 2 and (S)-1-(5-(trifluoromethyl)pyridin-2-yl)ethan-1-amine 2HCl (Intermediate 7,105 mg, 0.4 mmol) in Step 4. Yield: 10 mg, 0.026 mmol. White solid. ¹H NMR (400 MHz, DMSO) δ 8.92-8.87 (m, 1H), 8.58 (d, J=7.4 Hz, 1H), 8.18 (dd, J=8.3, 2.4 Hz, 1H), 7.55 (d, J=8.3 Hz, 1H), 6.22 (d, J=1.3 Hz, 1H), 5.81 (d, J=1.4 Hz, 1H), 5.29 (tt, J=7.8, 2.9 Hz, 1H), 4.98 (p, J=7.1 Hz, 1H), 4.75-4.66 (m, 2H), 4.24-4.16 (m, 2H), 3.27 (s, 2H), 1.40 (d, J=7.1 Hz, 3H). LCMS m/z 407.0 (M+H)⁺ (ES⁺).

### Example 34: 2-hydroxyethyl (S)-2-methylene-4-oxo-4-((1-(5-(trifluoromethyl)pyridin-2-yl)ethyl)amino)butanoate

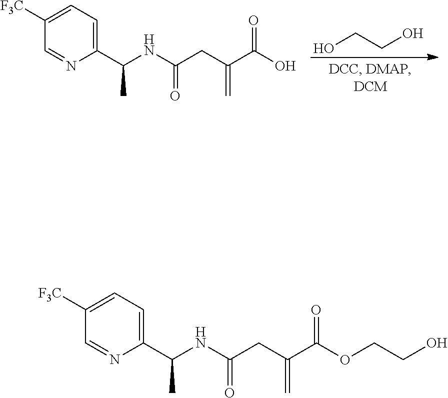

A mixture of (S)-2-methylene-4-oxo-4-((1-(5-(trifluoromethyl)pyridin-2-yl)ethyl)amino)butanoic acid (Example 30, 300 mg, 0.99 mmol), ethane-1,2-diol (123 mg, 2.00 mmol), DCC (309 mg, 1.50 mmol) and DMAP (12 mg, 0.10 mmol) in dichloromethane (3 mL) was stirred at 25° C. for 16 hours. The mixture was filtered and concentrated under reduced pressure. The residue was purified by prep-HPLC (Column: Waters SUNFIRE Prep C18 OBD 10 μm 19×250 mm; Flow Rate: 20 mL/min; solvent system: MeCN/(0.2% formic acid/water) MeCN gradient: 45-95%; collection wavelength: 214 nm). The fractions were concentrated under reduced pressure to remove MeCN, and the residue was lyophilized to give 2-hydroxyethyl (S)-2-methylene-4-oxo-4-((1-(5-(trifluoromethyl)pyridin-2-yl)ethyl)amino)butanoate (97 mg, 28% yield) as white solid. ¹H NMR (400 MHz, DMSO) δ 8.90 (s, 1H), 8.55 (d, J=7.2 Hz, 1H), 8.18 (dd, J=8.4, 2.4 Hz, 1H), 7.57 (d, J=8.4 Hz, 1H), 6.18 (d, J=1.2 Hz, 1H), 5.72 (d, J=1.2 Hz, 1H), 4.99-4.95 (m, 1H), 4.80 (t, J=5.6 Hz, 1H), 4.10-4.05 (m, 2H), 3.59-3.55 (m, 2H), 3.24 (s, 2H), 1.40 (d, J=6.8 Hz, 3H). LCMS m/z 347.2 (M+H)⁺ (ES⁺).

### Example 35: 1-acetylpiperidin-4-yl (S)-2-methylene-4-oxo-4-((1-(5-(trifluoromethyl)pyridin-2-yl)ethyl)amino)butanoate

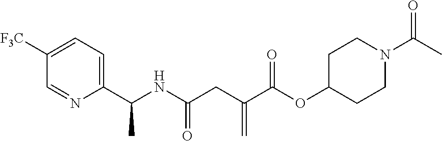 [SMILES: C=C(CC(=O)N[C@@H](C)c1ccc(C(F)(F)F)cn1)C(=O)OC1CCN(C(C)=O)CC1]

Prepared by an analogous method to Example 34 starting from 1-(4-hydroxypiperidin-1-yl)ethan-1-one (143 mg, 1.00 mmol) and (S)-2-methylene-4-oxo-4-((1-(5-(trifluoromethyl)pyridin-2-yl)ethyl)amino)butanoic acid (300 mg, 0.99 mmol). Yield: 41 mg, 0.1 mmol. White solid. ¹H NMR (400 MHz, DMSO) δ 8.90 (s, 1H), 8.55 (d, J=7.6 Hz, 1H), 8.17 (d, J=8.8 Hz, 1H), 7.57 (d, J=8.4 Hz, 1H), 6.17 (d, J=1.6 Hz, 1H), 5.70 (s, 1H), 5.01-4.92 (m, 2H), 3.45-3.38 (m, 4H), 3.26 (d, J=4.4 Hz, 2H), 1.97 (d, J=6.4 Hz, 3H), 1.82-1.61 (m, 2H), 1.55-1.43 (m, 2H), 1.38 (d, J=6.8 Hz, 3H). LCMS m/z 427.9 (M+H)⁺ (ES⁺).

### Example 36: 1-acetylazetidin-3-yl (S)-2-methylene-4-oxo-4-((1-(5-(trifluoromethyl)pyridin-2-yl)ethyl)amino)butanoate

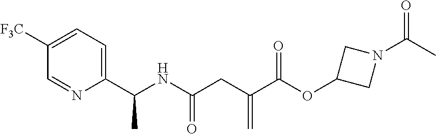 [SMILES: C=C(CC(=O)N[C@@H](C)c1ccc(C(F)(F)F)cn1)C(=O)OC1CN(C(C)=O)C1]

Prepared by an analogous method to Example 34 starting from 1-(3-hydroxyazetidin-1-yl)ethan-1-one (104 mg, 1.00 mmol) and (S)-2-methylene-4-oxo-4-((1-(5-(trifluoromethyl)pyridin-2-yl)ethyl)amino)butanoic acid (300 mg, 0.99 mmol). Yield: 104 mg, 0.26 mmol. White solid. ¹H NMR (400 MHz, DMSO) δ 8.90 (s, 1H), 8.58 (dd, J=7.2, 3.2 Hz, 1H), 8.18-8.15 (m, 1H), 7.56 (d, J=8.0 Hz, 1H), 6.21 (s, 1H), 5.78 (s, 1H), 5.17-5.12 (m, 1H), 4.99-4.96 (m, 1H), 4.46-4.40 (m, 1H), 4.17-4.12 (m, 1H), 4.00-3.98 (m, 1H), 3.75-3.71 (m, 1H), 3.27 (s, 2H), 1.74 (d, J=1.2 Hz, 3H), 1.39 (d, J=7.2 Hz, 3H). LCMS m/z 400.2 (M+H)⁺ (ES⁺).

### Example 37: 1-(methylsulfonyl)azetidin-3-yl (S)-2-methylene-4-oxo-4-((1-(5-(trifluoromethyl)pyridin-2-yl)ethyl)amino)butanoate

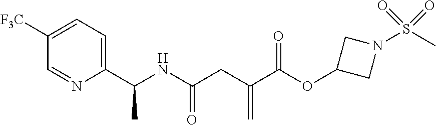 [SMILES: C=C(CC(=O)N[C@@H](C)c1ccc(C(F)(F)F)cn1)C(=O)OC1CN(S(C)(=O)=O)C1]

Prepared by an analogous method to Example 34 starting from (S)-2-methylene-4-oxo-4-((1-(5-(trifluoromethyl)pyridin-2-yl)ethyl)amino)butanoic acid (400 mg, 1.32 mmol) and 1-(methylsulfonyl)azetidin-3-ol (200 mg, 1.32 mmol. Yield: 105 mg, 0.24 mmol. White solid. ¹H NMR (400 MHz, DMSO) δ 8.90 (s, 1H), 8.59 (d, J=7.2 Hz, 1H), 8.18 (dd, J=8.0, 2.0 Hz, 1H), 7.56 (d, J=8.4 Hz, 1H), 6.22 (d, J=0.8 Hz, 1H), 5.79 (s, 1H), 5.17-5.14 (m, 1H), 5.00-4.97 (m, 1H), 4.23-4.18 (m, 2H), 3.87-3.83 (m, 2H), 3.27 (s, 2H), 3.03 (s, 3H), 1.40 (d, J=6.8 Hz, 3H). LCMS m/z 436.1 (M+H)⁺ (ES⁺).

### Example 38: (S)-2-methylene-4-oxo-4-((1-(6-(trifluoromethyl)pyridin-3-yl)ethyl)amino) butanoic acid

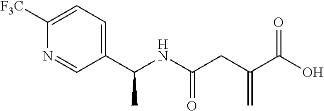 [SMILES: C=C(CC(=O)N[C@@H](C)c1ccc(C(F)(F)F)nc1)C(=O)O]

Prepared by an analogous method to Example 3, Method A starting from (S)-1-(6-(trifluoromethyl)pyridin-3-yl)ethan-1-amine, HCl (Intermediate 7, 0.150 g, 0.66 mmol). Yield: 98 mg, 0.32 mmol. White solid. ¹H NMR (400 MHz, DMSO) δ 12.48 (s, 1H), 8.71 (d, J=2.2 Hz, 1H), 8.53 (d, J=7.4 Hz, 1H), 7.98 (dd, J=8.1, 2.2 Hz, 1H), 7.84 (dd, J=8.2, 0.8 Hz, 1H), 6.09 (d, J=1.7 Hz, 1H), 5.65 (d, J=1.5 Hz, 1H), 5.05-4.93 (m, 1H), 3.23-3.10 (m, 2H), 1.40 (d, J=7.1 Hz, 3H). LCMS m/z 303.4 (M+H)⁺ (ES⁺).

### Example 39: 2-methylene-4-oxo-4-((1-(5-(trifluoromethyl)pyridin-2-yl)cyclobutyl)amino) butanoic acid

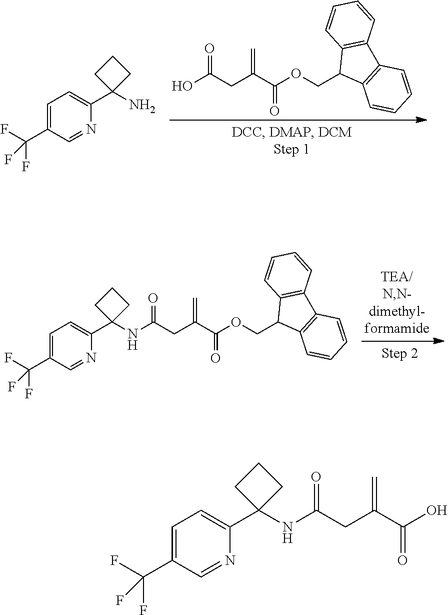 [SMILES: NC1(c2ccc(C(F)(F)F)cn2)CCC1 ; C=C(CC(=O)O)C(=O)OCC1c2ccccc2-c2ccccc21 ; C=C(CC(=O)NC1(c2ccc(C(F)(F)F)cn2)CCC1)C(=O)OCC1c2ccccc2-c2ccccc21 ; C=C(CC(=O)NC1(c2ccc(C(F)(F)F)cn2)CCC1)C(=O)O]

### Step 1

A mixture of 1-(5-(trifluoromethyl)pyridin-2-yl)cyclobutan-1-amine (Intermediate 9, 120 mg, 0.55 mmol), 3-(((9H-fluoren-9-yl)methoxy)carbonyl)but-3-enoic acid (Intermediate 10, 205 mg, 0.66 mmol), DCC (170 mg, 0.82 mmol) and DMAP (7 mg, 0.06 mmol) in DCM (5 mL) was stirred at room temperature for 1 hour. The mixture was filtered, and the filtrate was concentrated under reduced pressure. The residue was purified by flash column chromatography on silica (0-20% tert-butyl methyl ether/petroleum ether) to give (9H-fluoren-9-yl)methyl 2-methylene-4-oxo-4-((1-(5-(trifluoromethyl)pyridin-2-yl)cyclobutyl)amino)butanoate (150 mg, 54% yield) as a white solid. LCMS m/z 507.0 (M+H)⁺ (ES⁺).

### Step 2

A mixture of (9H-fluoren-9-yl)methyl 2-methylene-4-oxo-4-((1-(5-(trifluoromethyl)pyridin-2-yl)cyclobutyl)amino)butanoate (150 mg, 0.30 mmol) in N,N-Dimethylformamide (2 mL) and triethylamine (0.4 mL) was stirred at 25° C. for 3 hours. The reaction mixture was acidified with 0.5 N HCl until pH 6-7, and extracted with EtOAc (2×3 mL). The EtOAc layer was washed by brine, dried over Na₂SO₄, and the filtrate concentrated under reduced pressure. The residue was purified by prep-HPLC (Column: Waters X-Bridge C18 OBD 10 μm 19×250 mm; Flow Rate: 20 mL/min; solvent system: MeCN/(0.2% formic acid/water): MeCN gradient 55-95%; collection wavelength: 214 nm). The fractions were concentrated under reduced pressure to remove MeCN, and the residue lyophilized to give 2-methylene-4-oxo-4-((1-(5-(trifluoromethyl)pyridin-2-yl)cyclobutyl)amino)butanoic acid (53.4 mg, 55% yield) as white solid. ¹H NMR (400 MHz, DMSO) 12.51 (s, 1H), 8.94 (s, 1H), 8.84 (s, 1H), 8.03 (d, J=8.8, 6.4 Hz, 1H), 7.56 (d, J=8.0 Hz, 1H), 6.09 (s, 1H), 5.65 (s, 1H), 3.21 (s, 2H), 2.67-2.59 (m, 2H), 2.35-2.29 (m, 2H), 2.03-1.94 (m, 2H). LCMS m/z 329.2 (M+H)⁺ (ES⁺).

### Example 40: 2-methylene-4-oxo-4-((5-(trifluoromethyl)-2,3-dihydro-1H-inden-1-yl)amino) butanoic acid

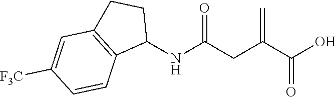 [SMILES: C=C(CC(=O)NC1CCc2cc(C(F)(F)F)ccc21)C(=O)O]

Prepared by an analogous method to Example 13 starting from 5-(trifluoromethyl)-2,3-dihydro-1H-inden-1-amine (Intermediate 11, 220 mg, 1.09 mmol) and 3-((2,2,2-trichloroethoxy)carbonyl)but-3-enoic acid (Intermediate 10, 272 mg, 1.04 mmol). Yield: 52 mg, 0.17 mmol. White solid. ¹H NMR (400 MHz, DMSO) δ 12.54 (br, 1H), 8.39 (d, J=8.3 Hz, 1H), 7.60 (s, 1H), 7.52 (d, J=8.4 Hz, 1H), 7.37 (d, J=8.0 Hz, 1H), 6.12 (d, J=1.2 Hz, 1H), 5.69 (s, 1H), 5.33 (q, J=8.3 Hz, 1H), 3.05-2.94 (m, 2H), 2.96-3.03 (m, 1H), 2.90-2.82 (m, 1H), 2.45-2.37 (m, 1H), 1.89-1.79 (m, 1H). LCMS m/z 314.0 (M+H)⁺ (ES⁺).

### Example 41: (S,Z)-2-(2-oxo-2-((1-(4-(trifluoromethyl)phenyl)ethyl)amino)ethyl)but-2-enoic acid and Example 42: (S,E)-2-(2-oxo-2-((1-(4-(trifluoromethyl)phenyl)ethyl) amino)ethyl)but-2-enoic acid

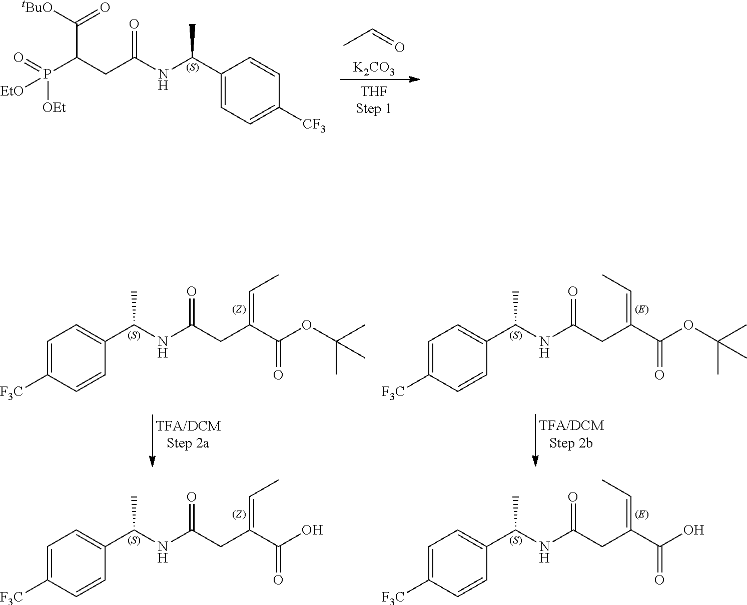

### Step 1

To the mixture of tert-butyl 2-(diethoxyphosphoryl)-4-oxo-4-(((S)-1-(4-(trifluoromethyl)phenyl)ethyl)amino)butanoate (Example 3 Step 1, 1.50 g, 3.12 mmol) and potassium carbonate (861 mg, 6.24 mmol) in THF (15 mL) and H₂O (3 mL) was added acetaldehyde (1.37 g, 31.16 mmol) at room temperature, and the reaction mixture was stirred at room temperature for 16 hours. The reaction mixture was quenched with water (10 mL) and extracted with MTBE (3×10 mL). The combined organic layers were washed with brine, dried over Na₂SO₄, and concentrated under reduced pressure. The residue was purified by flash column chromatography on silica (0-20% tert-Butyl methyl ether/petroleum ether) to give tert-butyl (S,E)-2-(2-oxo-2-((1-(4-(trifluoromethyl)phenyl)ethyl)amino)ethyl)but-2-enoate (250 mg, 22% yield) as colorless oil, followed by tert-butyl (S,Z)-2-(2-oxo-2-((1-(4-(trifluoromethyl)phenyl)ethyl)amino)ethyl)but-2-enoate (100 mg, 9% yield) as colorless oil.

E isomer ¹H NMR (400 MHz, CDCl₃) δ: 7.55 (d, J=8.0 Hz, 2H), 7.37 (d, J=8.4 Hz, 2H), 6.98 (q, J=7.2 Hz, 1H), 6.64 (d, J=7.2 Hz, 1H), 2.09-5.06 (m, 1H), 3.22 (s, 2H), 1.90 (d, J=7.2 Hz, 3H), 1.47 (s, 9H), 1.44 (d, J=7.2 Hz, 3H). LCMS m/z 394.3 (M+Na)⁺ (ES⁺).

Z isomer ¹H NMR (400 MHz, CDCl₃) δ: 7.56 (d, J=8.0 Hz, 2H), 7.40 (d, J=8.4 Hz, 2H), 6.45 (d, J=8.0 Hz, 1H), 6.27 (q, J=7.2 Hz, 1H), 5.11 (t, J=6.8 Hz, 1H), 3.11 (d, J=7.6 Hz, 2H), 2.02 (d, J=7.6 Hz, 3H), 1.47 (s, 9H), 1.46 (d, J=6.8 Hz, 3H). LCMS m/z 394.3 (M+Na)⁺ (ES⁺).

### Step 2a

A mixture of tert-butyl (S,Z)-2-(2-oxo-2-((1-(4-(trifluoromethyl)phenyl)ethyl)amino)ethyl)but-2-enoate (100 mg, 0.27 mmol) in dichloromethane (3 mL) and TFA (0.5 mL) was stirred at room temperature for 2 hours. The solvent was removed under reduced pressure and the residue was purified by prep-HPLC (Column: Waters SUNFIRE Prep C18 OBD 10 μm 19×250 mm; Flow Rate: 20 mL/min; solvent system: MeCN/(0.2% formic acid/water) MeCN gradient: 55-95%; collection wavelength: 214 nm). The fractions were concentrated under reduced pressure to remove MeCN, and the residue was lyophilized to give (S,Z)-2-(2-oxo-2-((1-(4-(trifluoromethyl)phenyl)ethyl)amino)ethyl)but-2-enoic acid (57.24 mg, 67% yield) as white solid.

¹H NMR (400 MHz, DMSO-d6) δ: 12.35 (br, 1H), 8.39 (d, J=7.6 Hz, 1H), 7.66 (d, J=8.0 Hz, 2H), 7.51 (d, J=8.0 Hz, 2H), 6.05 (q, J=7.2 Hz, 1H), 4.98-4.88 (m, 1H), 3.15-3.02 (m, 2H), 1.93 (d, J=7.2 Hz, 3H), 1.34 (d, J=7.2 Hz, 3H). LCMS m/z 316.1 (M+H)⁺ (ES⁺).

### Step 2b

A mixture of tert-butyl (S,E)-2-(2-oxo-2-((1-(4-(trifluoromethyl)phenyl)ethyl)amino)ethyl)but-2-enoate (250 mg, 0.67 mmol) in dichloromethane (3 mL) and TFA (0.5 mL) was stirred at room temperature for 2 hours The solvent was removed under reduced pressure and the residue was purified by prep-HPLC (Column: Waters SUNFIRE Prep C18 OBD 10 μm 19×250 mm; Flow Rate: 20 mL/min; solvent system: MeCN/(0.2% formic acid/water) MeCN gradient: 55-95%; collection wavelength: 214 nm). The fractions were concentrated under reduced pressure to remove MeCN, and the residue was lyophilized to give (S,E)-2-(2-oxo-2-((1-(4-(trifluoromethyl)phenyl)ethyl)amino)ethyl)but-2-enoic acid (145 mg, 68% yield) as white solid.

¹H NMR (400 MHz, DMSO-d6) δ: 12.14 (br, 1H), 8.41 (d, J=7.6 Hz, 1H), 7.66 (d, J=8.0 Hz, 2H), 7.52 (d, J=8.4 Hz, 2H), 6.84 (q, J=7.2 Hz, 1H), 4.97-4.87 (m, 1H), 3.24-3.11 (m, 2H), 1.73 (d, J=7.2 Hz, 3H), 1.35 (d, J=7.2 Hz, 3H). LCMS m/z 316.1 (M+H)⁺ (ES⁺).

### Example 43: (S,E)-2-(2-oxo-2-((1-(4-(trifluoromethyl)phenyl)ethyl)amino)ethyl)pent-2-enoic acid

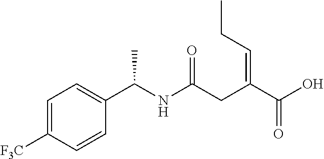 [SMILES: CC/C=C(\CC(=O)N[C@@H](C)c1ccc(C(F)(F)F)cc1)C(=O)O]

Prepared by an analogous method to Example 42 starting from tert-butyl 2-(diethoxyphosphoryl)-4-oxo-4-(((S)-1-(4-(trifluoromethyl)phenyl)ethyl)amino)butanoate (Example 3 Step 1, 900 mg, 1.87 mmol) and propionaldehyde (326 mg, 5.61 mmol). Yield: 66.7 mg, 0.20 mmol. White solid. ¹H NMR (400 MHz, DMSO-d6) δ 12.19 (s, 1H), 8.42 (d, J=7.6 Hz, 1H), 7.66 (d, J=8.0 Hz, 2H), 7.52 (d, J=8.4 Hz, 2H), 6.73 (t, J=7.5 Hz, 1H), 4.92 (p, J=7.2 Hz, 1H), 3.23-3.10 (m, 2H), 2.12 (pd, J=7.5, 1.6 Hz, 2H), 1.35 (d, J=7.1 Hz, 3H), 0.94 (t, J=7.5 Hz, 3H). LCMS m/z 330.3 (M+H)⁺ (ES⁺)

### Example 44: (S,E)-4-methyl-2-(2-oxo-2-((1-(4-(trifluoromethyl)phenyl)ethyl)amino)ethyl)pent-2-enoic acid

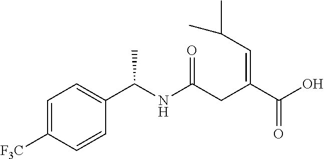 [SMILES: CC(C)/C=C(\CC(=O)N[C@@H](C)c1ccc(C(F)(F)F)cc1)C(=O)O]

Prepared by an analogous method to Example 42 starting from tert-butyl 2-(diethoxyphosphoryl)-4-oxo-4-(((S)-1-(4-(trifluoromethyl)phenyl)ethyl)amino)butanoate (Example 3 Step 1, 800 mg, 1.66 mmol) and isobutyraldehyde (359 mg, 4.98 mmol). Yield: 50.3 mg, 0.15 mmol. White solid. ¹H NMR (400 MHz, DMSO-d6) δ 12.20 (s, 1H), 8.43 (d, J=7.6 Hz, 1H), 7.65 (d, J=8.4 Hz, 2H), 7.52 (d, J=8.4 Hz, 2H), 6.54 (d, J=10.0 Hz, 1H), 4.94-4.90 (m, 1H), 3.24-3.10 (m, 2H), 2.60-2.55 (m, 1H), 1.35 (d, J=7.2 Hz, 3H), 0.93 (dd, J=16.8, 6.8 Hz, 6H). LCMS m/z 344.2 (M+H)⁺ (ES⁺)

### Example 45: (S,E)-5-methyl-2-(2-oxo-2-((1-(4-(trifluoromethyl)phenyl)ethyl)amino)ethyl)hex-2-enoic acid

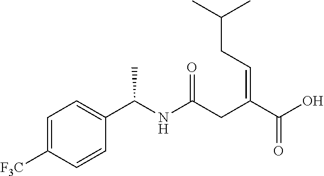 [SMILES: CC(C)C/C=C(\CC(=O)N[C@@H](C)c1ccc(C(F)(F)F)cc1)C(=O)O]

Prepared by an analogous method to Example 42 starting from tert-butyl 2-(diethoxyphosphoryl)-4-oxo-4-(((S)-1-(4-(trifluoromethyl)phenyl)ethyl)amino)butanoate (Example 3 Step 1, 800 mg, 1.66 mmol) and 3-methylbutanal (430 mg, 4.98 mmol). Yield: 118.0 mg, 0.33 mmol. White solid. ¹H NMR (400 MHz, DMSO-d6) δ 12.20 (br, 1H), 8.42 (d, J=7.6 Hz, 1H), 7.66 (d, J=8.4 Hz, 2H), 7.52 (d, J=8.4 Hz, 2H), 6.75 (t, J=7.6 Hz, 1H), 4.94-4.90 (m, 1H), 3.24-3.10 (m, 2H), 2.03-1.99 (m, 2H), 1.68-1.65 (m, 1H), 1.35 (d, J=7.2 Hz, 3H), 0.84 (d, J=6.8 Hz, 6H). LCMS m/z 358.3 (M+H)⁺ (ES⁺)

### Example 46: (S,E)-2-(2-oxo-2-((1-(4-(trifluoromethyl)phenyl)ethyl)amino)ethyl)-5-phenylpent-2-enoic acid

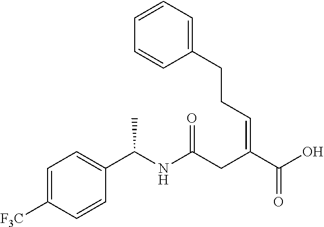 [SMILES: C[C@H](NC(=O)C/C(=C\CCc1ccccc1)C(=O)O)c1ccc(C(F)(F)F)cc1]

Prepared by an analogous method to Example 42 starting from tert-butyl 2-(diethoxyphosphoryl)-4-oxo-4-(((S)-1-(4-(trifluoromethyl)phenyl)ethyl)amino)butanoate (Example 3 Step 1, 800 mg, 1.66 mmol) and 3-phenylpropanal (446 mg, 3.32 mmol). Yield: 90.6 mg, 0.22 mmol. White solid. ¹H NMR (400 MHz, DMSO-d6) δ 12.22 (s, 1H), 8.43 (d, J=8.0 Hz, 1H), 7.62 (d, J=8.4 Hz, 2H), 7.51 (d, J=8.0 Hz, 2H), 7.29-7.25 (m, 2H), 7.20-7.16 (m, 3H), 6.77 (t, J=7.2 Hz, 1H), 4.95-4.88 (m, 1H), 3.23-3.09 (m, 2H, 2.67 (t, J=7.2 Hz, 2H), 2.46-2.39 (m, 2H), 1.34 (d, J=6.8 Hz, 3H). LCMS m/z 428.2 (M+Na)⁺ (ES⁺)

### Example 47: (S,E)-2-benzylidene-4-oxo-4-((1-(4-(trifluoromethyl)phenyl)ethyl)amino)butanoic acid and Example 48: (S,Z)-2-benzylidene-4-oxo-4-((1-(4-(trifluoromethyl)phenyl)ethyl)amino)butanoic acid

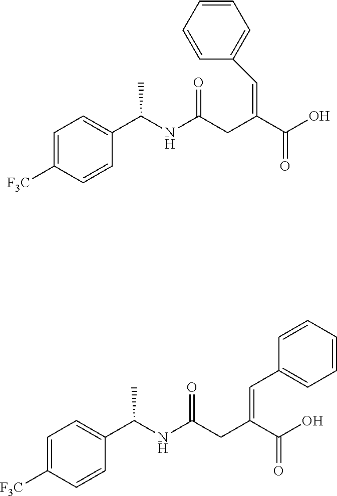 [SMILES: C[C@H](NC(=O)C/C(=C\c1ccccc1)C(=O)O)c1ccc(C(F)(F)F)cc1 ; C[C@H](NC(=O)C/C(=C/c1ccccc1)C(=O)O)c1ccc(C(F)(F)F)cc1]

Prepared by an analogous method to Examples 41 and 42 starting from tert-butyl 2-(diethoxyphosphoryl)-4-oxo-4-(((S)-1-(4-(trifluoromethyl)phenyl)ethyl)amino)butanoate (Example 3 Step 1, 800 mg, 1.66 mmol) and benzaldehyde (353 mg, 3.32 mmol).

E ISOMER. Yield: 106.2 mg, 0.28 mmol. White solid. ¹H NMR (400 MHz, DMSO-d6) δ 12.62 (br, 1H), 8.60 (d, J=8.0 Hz, 1H), 7.72 (s, 1H), 7.68 (d, J=8.4 Hz, 2H), 7.56 (d, J=8.0 Hz, 2H), 7.44-7.37 (m, 5H), 4.99-4.96 (m, 1H), 3.40-3.29 (m, 2H), 1.38 (d, J=7.2 Hz, 3H). LCMS m/z 378.1 (M+H)⁺ (ES⁺).

Z ISOMER. Yield: 75.2 mg, 0.20 mmol. White solid. ¹H NMR (400 MHz, DMSO-d6) δ 12.62 (s, 1H), 8.57 (d, J=7.6 Hz, 1H), 7.68 (d, J=8.4 Hz, 2H), 7.54 (d, J=8.4 Hz, 2H), 7.34-7.26 (m, 5H), 6.69 (s, 1H), 4.98-4.95 (m, 1H), 3.29 (s, 2H), 1.38 (d, J=2.8 Hz, 3H). LCMS m/z 378.3 (M+H)⁺ (ES⁺).

### Example 49: (S,E)-4-(dimethylamino)-2-(2-oxo-2-((1-(4-(trifluoromethyl)phenyl)ethyl)amino)ethyl)but-2-enoic acid

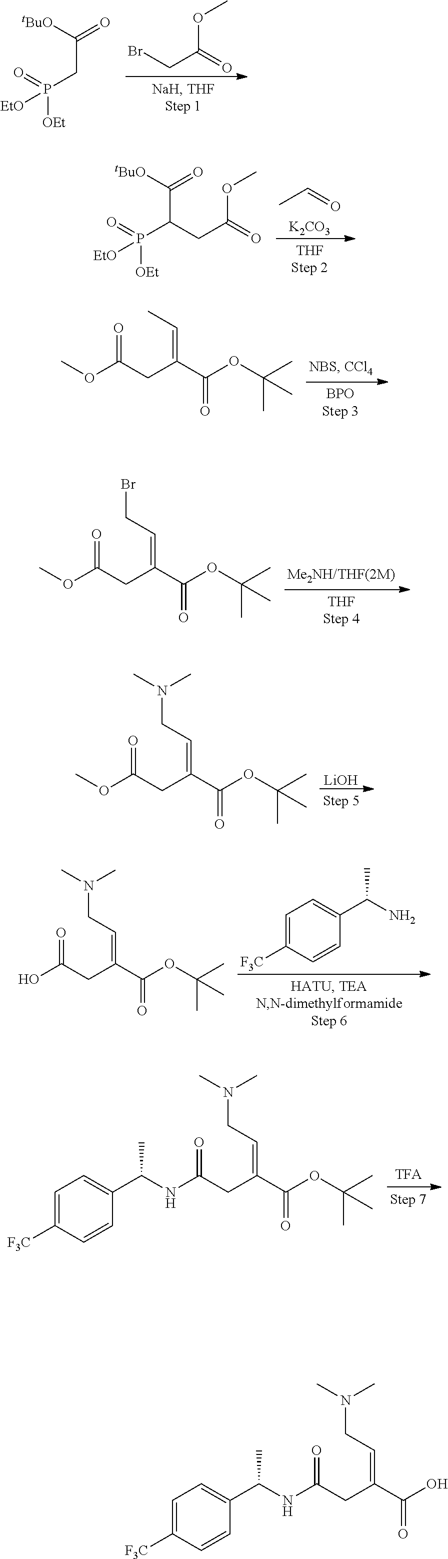

### Step 1

To the solution of tert-butyl 2-(diethoxyphosphoryl)acetate (1.0 g, 3.96 mmol) in THF (20 mL) was added sodium hydride (60% wt in mineral oil, 174 mg, 4.36 mmol) at 0° C., and the reaction mixture was stirred at 0° C. for 0.5 hour. Methyl 2-bromoacetate (667 mg, 4.36 mmol) was added and the reaction mixture was stirred at 0° C. for 3 hours. The reaction was quenched with 0.5N HCl (10 mL), and extracted with EtOAc (2×10 mL). The combined organic layers were washed with brine, dried over Na₂SO₄, filtered and concentrated under reduced pressure to give 1-(tert-butyl) 4-methyl 2-(diethoxyphosphoryl)succinate (1.20 g, 93% yield) as colorless oil. LCMS m/z 347.2 (M+Na)⁺ (ES⁺).

### Step 2

To the mixture of 1-(tert-butyl) 4-methyl 2-(diethoxyphosphoryl)succinate (1.20 g, 3.70 mmol) and potassium carbonate (766 mg, 5.55 mmol) in THF (15 mL) and water (3 mL) was added acetaldehyde (814 mg, 18.5 mmol) at room temperature, and the reaction mixture was stirred at room temperature for 24 hours. The reaction mixture was quenched with water (10 mL) and extracted with tert-butyl methyl ether (3×10 mL). The combined organic layers were washed with brine, dried over Na₂SO₄, and concentrated under reduced pressure. The residue was purified by flash column chromatography on silica (0-20% tert-butyl methyl ether/petroleum ether) to give 1-(tert-butyl) 4-methyl 2-ethylidenesuccinate (450 mg, 57% yield) as colorless oil. LCMS m/z 237.4 (M+Na)⁺ (ES⁺).

### Step 3

A mixture of 1-(tert-butyl) 4-methyl 2-ethylidenesuccinate (450 mg, 2.10 mmol), N-bromosuccinimide (336 mg, 1.89 mmol) and benzoyl peroxide (51 mg, 0.21 mmol) in carbon tetrachloride (10 mL) was stirred at 75° C. for 16 hours. The reaction mixture was concentrated under reduced pressure and the residue was purified by flash column chromatography on silica (0-7% tert-butyl methyl ether/petroleum ether) to give 1-(tert-butyl) 4-methyl 2-(2-bromoethylidene)succinate (350 mg, 57% yield) as light yellow oil. 1H NMR showed a E:Z ratio of 5/1. LCMS m/z 315.0 (M+Na)⁺ (ES⁺).

### Step 4

To a solution of 1-(tert-butyl) 4-methyl 2-(2-bromoethylidene)succinate (350 mg, 1.19 mmol) in THF (4 mL) was added dimethylamine (2 mol/L in THF solution, 0.6 mL, 1.20 mmol,), and the reaction mixture was stirred at 25° C. for 2 hours. The reaction mixture was concentrated under reduced pressure and the residue was purified by flash column chromatography on silica (0-20% methanol/dichloromethane) to give 1-(tert-butyl) 4-methyl 2-(2-(dimethylamino)ethylidene)succinate (250 mg, 81% yield) as colorless oil. 1H NMR showed a E:Z ratio of 6/1. LCMS m/z 258.4 (M+H)⁺ (ES⁺).

### Step 5

To a solution of 1-(tert-butyl) 4-methyl 2-(2-(dimethylamino)ethylidene)succinate (250 mg, 0.97 mmol) in THF (2 mL) was added 2N LiOH aqueous solution (0.5 mL, 1.00 mmol), and the reaction mixture was stirred at 25° C. for 8 hours. The reaction mixture was acidified with 2N HCl (0.5 mL), the solvent was removed under reduced pressure and the residue was suspended in THF (5 mL), dried over Na₂SO₄, concentrated under reduced pressure to give 3-(tert-butoxycarbonyl)-5-(dimethylamino)pent-3-enoic acid (200 mg, 85% yield) as colorless oil. The crude product was used in next step without purification. LCMS m/z 244.3 (M+H)⁺ (ES⁺).

### Step 6

To the solution of (S)-1-(4-(trifluoromethyl)phenyl)ethan-1-amine hydrocholoride (125 mg, 0.66 mmol), 3-(tert-butoxycarbonyl)-5-(dimethylamino)pent-3-enoic acid (200 mg, 0.82 mmol) and triethylamine (248 mg, 2.46 mmol) in N,N-dimethylformamide (5 mL) was added HATU (374 mg, 0.99 mmol) at 0° C.; and the mixture was stirred at room temperature for 3 hours, The reaction mixture was quenched with water (4 mL) and the aqueous layer was extracted with ethyl acetate (3×10 mL). The combined organic layers were washed by brine, dried over Na₂SO₄, and concentrated under reduced pressure. The residue was purified by flash column chromatography on silica (0-10% methanol/dichloromethane) to give tert-butyl (S)-4-(dimethylamino)-2-(2-oxo-2-((1-(4-(trifluoromethyl)phenyl)ethyl)amino)ethyl)but-2-enoate (300 mg) The product was further purified by prep-HPLC (Column: Waters SUNFIRE Prep C18 OBD 10 μm 19×250 mm; Flow Rate: 20 mL/min; solvent system: MeCN/(0.2% formic acid/water) gradient: 35-95% MeCN; collection wavelength: 214 nm). The fractions were concentrated under reduced pressure to remove MeCN, and the residue was lyophilized to give tert-butyl (S)-4-(dimethylamino)-2-(2-oxo-2-((1-(4-(trifluoromethyl)phenyl)ethyl)amino)ethyl)but-2-enoate (70 mg, 21% yield) as colorless oil. LCMS m/z 415.3 (M+H)⁺ (ES⁺).

### Step 7

A mixture of tert-butyl (S)-4-(dimethylamino)-2-(2-oxo-2-((1-(4-(trifluoromethyl)phenyl)ethyl)amino)ethyl)but-2-enoate (70 mg, 0.17 mmol) in dichloromethane (3 mL) and TFA (0.5 mL) was stirred at room temperature for 3 hours. The solvent was removed under reduced pressure and the residue was suspended in ethyl acetate (3 mL), saturated sodium bicarbonate was added to adjust pH to 6-7 then the solvent was removed under reduced pressure. The residue was purified by prep-HPLC (Column: Waters SUNFIRE Prep C18 OBD 10 μm 19×250 mm; Flow Rate: 20 mL/min; solvent system: MeCN/(0.2% formic acid/water) gradient: 35-95% MeCN; collection wavelength: 214 nm). The fractions were concentrated under reduced pressure to remove MeCN, and the residue was lyophilized to give (S)-4-(dimethylamino)-2-(2-oxo-2-((1-(4-(trifluoromethyl)phenyl)ethyl)amino)ethyl)but-2-enoic acid (26.7 mg, 44% yield) as colorless oil. 1H NMR showed a E:Z ratio of 2/1.

LCMS m/z 359.2 (M+H)⁺ (ES⁺). ¹H NMR (400 MHz, CD3OD) δ: 7.60 (d, J=8.0 Hz, 2H), 7.50 (d, J=8.0 Hz, 2H), 6.81 (t, J=7.6 Hz, 0.67H), 5.92 (d, J=7.6 Hz, 0.34H), 5.00 (q, J=6.8 Hz, 1H), 3.93-3.76 (m, 2H), 3.50-3.23 (m, 2H), 2.83 (s, 4H), 2.88-2.79 (m, 6H), 1.47-1.44 (m, 3H).

### Example 50: (S)-3,3-dimethyl-2-methylene-4-oxo-4-((1-(4-(trifluoromethyl)phenyl)ethyl)amino)butanoic acid

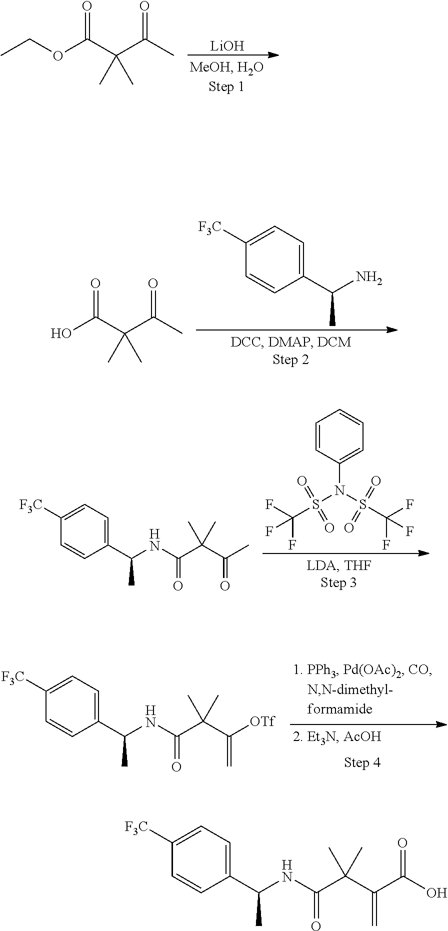

### Step 1

To a solution of ethyl 2,2-dimethyl-3-oxobutanoate (1.3 g, 8.22 mmol) in MeOH (40 mL) was added 2N LiOH aq. (12 mL, 24 mmol) at 0° C., and the reaction mixture was stirred at room temperature overnight. The reaction mixture was neutralized by 1 N HCl aq. to pH 3, concentrated under reduced pressure to remove MeOH and extracted with EtOAc (2×20 mL). The combined organic layer was dried over Na₂SO₄ and concentrated under reduced pressure to give 2,2-dimethyl-3-oxobutanoic acid (1.05 g, 98% yield) as light yellow oil. ¹H NMR (400 MHz, DMSO-d6) δ: 12.78 (br, 1H), 2.13 (s, 3H), 1.25 (s, 6H).

### Step 2

To the solution of 2,2-dimethyl-3-oxobutanoic acid (400 mg, 3.07 mmol), (S)-1-(4-(trifluoromethyl)phenyl)ethan-1-amine (581 mg, 3.07 mmol) in DCM (10 mL) was added DCC (948 mg, 4.60 mmol) and DMAP (38 mg, 0.31 mmol) at room temperature, and the mixture was stirred at room temperature for 16 hours. The reaction mixture was filtered, and the filtrate was concentrated under reduced pressure. The residue was purified by flash column chromatography on silica (20-35% tert-butyl methyl ether/petroleum ether) to give (S)-2,2-dimethyl-3-oxo-N-(1-(4-(trifluoromethyl)phenyl)ethyl)butanamide (460 mg, 50%) as white solid. LCMS m/z 302.2 (M+H)⁺ (ES⁺).

### Step 3

To the solution of (S)-2,2-dimethyl-3-oxo-N-(1-(4-(trifluoromethyl)phenyl)ethyl)butanamide (420 mg, 1.39 mmol) in THF (10 mL) was added LDA (2N in THF/heptanes/ethylbenzene, 1.00 mL, 2.00 mmol) at −20° C., and the reaction mixture was stirred at the same temperature for 20 minutes. 1,1,1-trifluoro-N-phenyl-N-((trifluoromethyl) sulfonyl)methanesulfonamide (498 mg, 1.39 mmol) was added and the reaction mixture was stirred at −10° C. for 2 hours. The reaction mixture was quenched by saturated NaHCO₃ (10 mL) and extracted with tert-butyl methyl ether (2×10 mL). The combined organic layers were dried over Na₂SO₄ and concentrated under reduced pressure. The residue was purified by flash column chromatography on silica (0-30% tert-butyl methyl ether/petroleum ether) to give (S)-3,3-dimethyl-4-oxo-4-((1-(4-(trifluoromethyl)phenyl)ethyl)amino)but-1-en-2-yl trifluoromethanesulfonate (450 mg, 60% purity, 45% yield) as colorless oil. The crude material was purified by reverse phase column chromatography (Column: Boston ODS 120 g Flash; Flow Rate: 30 mL/min; solvent system: MeCN/(0.2% formic acid/water) gradient: 65-85% MeCN: collection wavelength: 214 nm). The fractions were concentrated under reduced pressure to remove MeCN, and the residue was lyophilized to give (S)-3,3-dimethyl-4-oxo-4-((1-(4-(trifluoromethyl)phenyl)ethyl)amino)but-1-en-2-yl trifluoromethanesulfonate (150 mg, 22% yield) as white solid. LCMS m/z 434.2 (M+H)⁺ (ES⁺).

### Step 4

A mixture of (S)-3,3-dimethyl-4-oxo-4-((1-(4-(trifluoromethyl)phenyl)ethyl)amino)but-1-en-2-yl trifluoromethanesulfonate (150 mg, 0.34 mmol), triphenylphosphine (27 mg, 0.10 mmol), palladium(II) acetate (14 mg, 0.06 mmol) in N,N-dimethylformamide (4 mL) was saturated with CO gas (CO balloon). Meanwhile a solution of triethylamine (69 mg, 0.68 mmol) in N,N-dimethylformamide (2 mL) was cooled to 0° C. and AcOH (25 mg, 0.41 mmol) was added. This solution was added the first solution and the reaction mixture was allowed to stir at room temperature overnight with a CO balloon. The reaction mixture was diluted with NaHCO₃ (5 mL) and extracted with ethyl acetate (2×10 mL). The combined organic layers were washed with brine, dried over Na₂SO₄, and concentrated under reduced pressure. The residue was purified by prep-HPLC (Column: Waters SUNFIRE Prep C18 OBD 10 μm 19×250 mm; Flow Rate: 20 mL/min; solvent system: MeCN/(0.2% formic acid/water) gradient: 55-95% MeCN; collection wavelength: 214 nm). The fractions were concentrated under reduced pressure to remove MeCN, and the residue was lyophilized to give (S)-3,3-dimethyl-2-methylene-4-oxo-4-((1-(4-(trifluoromethyl)phenyl)ethyl)amino)butanoic acid (3.90 mg, 3% yield) as white solid. LCMS m/z 330.2 (M+H)⁺ (ES⁺). ¹H NMR (400 MHz, DMSO-d6) δ: 12.28 (br, 1H), 7.70 (br, 1H), 7.64 (d, J=8.0 Hz, 2H), 7.52 (d, J=8.0 Hz, 2H), 6.14 (s, 1H), 5.69 (s, 1H), 4.97-4.92 (m, 1H), 1.34 (d, J=6.8 Hz, 3H), 1.30 (d, J=3.6 Hz, 6H).

### Example 51: 3-methyl-2-methylene-4-oxo-4-((2-(4-(trifluoromethyl)phenyl)propan-2-yl)amino)butanoic acid

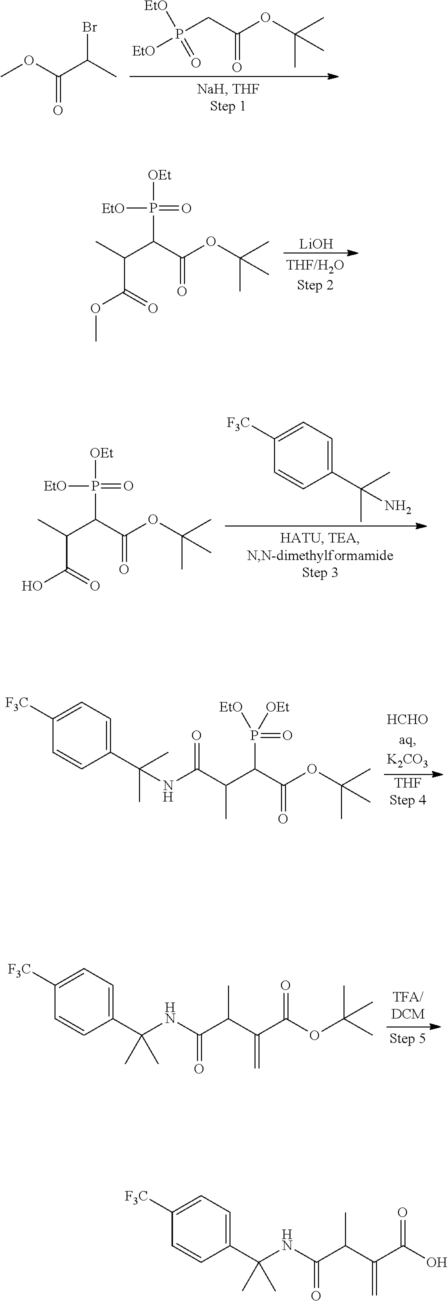

### Step 1

To a solution of tert-butyl 2-(diethoxyphosphoryl)acetate (2.00 g, 11.98 mmol) in THF (40 mL) was added sodium hydride (60% wt in mineral oil, 480 mg, 13.17 mmol) at 0° C., and the reaction mixture was stirred at 0° C. for 0.5 hour. Methyl 2-bromopropanoate (3.02 g, 11.98 mmol) was added and the reaction mixture was stirred at room temperature for 16 hours. The reaction mixture was quenched with saturated NH₄Cl (30 mL) and extracted with EtOAc (2×20 mL). The combined organic layers were washed with brine, dried over Na₂SO₄, concentrated under reduced pressure to give 1-(tert-butyl) 4-methyl 2-(diethoxyphosphoryl)-3-methylsuccinate (2.50 g, 61% yield) as colorless oil. LCMS m/z 361.3 (M+Na)⁺ (ES⁺).

### Step 2

To a solution of 1-(tert-butyl) 4-methyl 2-(diethoxyphosphoryl)-3-methylsuccinate (2.50 g, 7.39 mmol) in THF (30 mL) was added 2N LiOH aqueous solution (3.7 mL, 7.40 mmol), and the reaction mixture was stirred at 25° C. for 5 hours. The reaction mixture was acidified with 2N HCl to pH 4-5, the solvent was removed under reduced pressure and the residue was suspended in THF (5 mL), dried over Na₂SO₄, and concentrated under reduced pressure to give 4-(tert-butoxy)-3-(diethoxyphosphoryl)-2-methyl-4-oxobutanoic acid (2.00 g, 83% yield) as a colorless oil, which was used in next step without further purification. LCMS m/z 347.2 (M+Na)⁺ (ES⁺).

### Step 3

To a solution of 2-(4-(trifluoromethyl)phenyl)propan-2-amine (500 mg, 2.47 mmol) in N,N-dimethylformamide (12 mL) was added 4-(tert-butoxy)-3-(diethoxyphosphoryl)-2-methyl-4-oxobutanoic acid (800 mg, 2.47 mmol), triethylamine (749 mg, 7.41 mmol) and HATU (1.41 g, 3.70 mmol) at 0° C.; and the mixture was stirred at room temperature for 3 hours. The reaction mixture was quenched with 0.5N HCl (20 mL) and the aqueous layer was extracted with ethyl acetate (3×20 mL). The combined organic layers were washed by brine, dried over Na₂SO₄ and concentrated under reduced pressure. The residue was purified by flash column chromatography on silica (50-100% tert-butyl methyl ether/petroleum ether) to give tert-butyl 2-(diethoxyphosphoryl)-3-methyl-4-oxo-4-((2-(4-(trifluoromethyl)phenyl)propan-2-yl)amino)butanoate (1.00 g, 80% yield) as light-brown oil. LCMS m/z 510.2 (M+H)⁺ (ES⁺).

### Step 4

To a mixture of tert-butyl 2-(diethoxyphosphoryl)-3-methyl-4-oxo-4-((2-(4-(trifluoromethyl)phenyl)propan-2-yl)amino)butanoate (1.00 g, 1.96 mmol) and potassium carbonate (406 mg, 2.94 mmol) in THF (15 mL) and H₂O (3 mL) was added formaldehyde (37% aqueous solution, 476 mg, 5.88 mmol) at room temperature, and the reaction mixture was stirred at 65° C. for 16 hours. The reaction mixture was quenched with H₂O (10 mL) and extracted with tert-butyl methyl ether (3×10 mL). The combined organic layers were washed with brine, dried over Na₂SO₄ and concentrated under reduced pressure. The residue was purified by flash column chromatography on silica (0-20% tert-butyl methyl ether/petroleum ether) to give tert-butyl 3-methyl-2-methylene-4-oxo-4-((2-(4-(trifluoromethyl)phenyl)propan-2-yl)amino)butanoate (250 mg, 33% yield) as colorless oil. LCMS m/z 408.3 (M+Na)⁺ (ES⁺).

### Step 5

A mixture of tert-butyl 3-methyl-2-methylene-4-oxo-4-((2-(4-(trifluoromethyl)phenyl)propan-2-yl)amino)butanoate (250 mg, 0.65 mmol) in dichloromethane (3 mL) and TFA (1 mL) was stirred at room temperature for 3 hours. The solvent was removed under reduced pressure and the residue was triturated in n-heptane (5 mL), stirred at 25° C. for 1 hour and filtered. The filtrate was dried at 40° C. under reduced pressure to give 3-methyl-2-methylene-4-oxo-4-((2-(4-(trifluoromethyl)phenyl)propan-2-yl)amino)butanoic acid (150 mg, 70% yield). 30 mg of impure material were further purified by prep-HPLC (Column: Waters SUNFIRE Prep C18 OBD 10 μm 19×250 mm; Flow Rate: 20 mL/min; solvent system: MeCN/(0.2% formic acid/water) gradient: 55-95% MeCN; collection wavelength: 214 nm). The fractions were concentrated under reduced pressure to remove MeCN, and the residue was lyophilized to give 3-methyl-2-methylene-4-oxo-4-((2-(4-(trifluoromethyl)phenyl)propan-2-yl)amino)butanoic acid (6.76 mg) as white solid.

LCMS m/z 330.3 (M+H)⁺ (ES⁺). ¹H NMR (400 MHz, DMSO-d6) δ: 12.57 (br, 1H), 8.33 (s, 1H), 7.59 (d, J=8.4 Hz, 2H), 7.52 (d, J=8.4 Hz, 2H), 6.11 (s, 1H), 5.53 (s, 1H), 3.56-3.51 (m, 1H), 1.53 (d, J=16.8 Hz, 6H), 1.16 (d, J=6.8 Hz, 3H).

### Example 52: 3-methyl-2-methylene-4-oxo-4-((2-(4-(trifluoromethyl)phenyl)propan-2-yl)amino)butanoic acid ISOMER 1 and Example 53: 3-methyl-2-methylene-4-oxo-4-((2-(4-(trifluoromethyl)phenyl)propan-2-yl)amino)butanoic acid

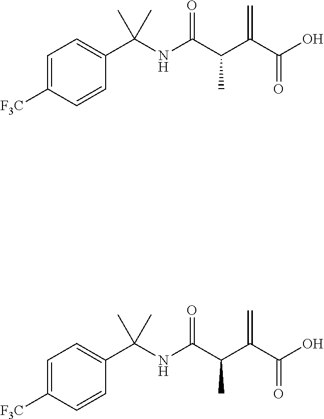 [SMILES: C=C(C(=O)O)[C@H](C)C(=O)NC(C)(C)c1ccc(C(F)(F)F)cc1 ; C=C(C(=O)O)[C@@H](C)C(=O)NC(C)(C)c1ccc(C(F)(F)F)cc1]

3-methyl-2-methylene-4-oxo-4-((2-(4-(trifluoromethyl)phenyl)propan-2-yl)amino)butanoic acid (Example 51, 120 mg, 0.36 mmol) was separated by SFC (Column: CHIRALPAK AD-5(30*250 mm 5 μm) (Daicel). Column temperature: 35° C. CO₂ flow Rate: 36 mL/min; co solvent flow rate: 9 mL/min; total flow rate: 45 mL/min. Co solvent: methanol. Gradient: methanol 20%. Collection wavelength: 215 nm). The SFC fractions were concentrated under reduced pressure to remove isopropanol to give 3-methyl-2-methylene-4-oxo-4-((2-(4-(trifluoromethyl)phenyl)propan-2-yl)amino)butanoic acid ISOMER 1 (36.4 mg, 100 ee %, 31% yield) and 3-methyl-2-methylene-4-oxo-4-((2-(4-(trifluoromethyl)phenyl)propan-2-yl)amino)butanoic acid ISOMER 2 (38.9 mg, 100 ee %, 33% yield). The absolute stereochemistry was arbitrarily assigned.

Chiral HPLC (Column: Kromasil (s,s)-Whelk-O-1 (4.6×100 mm); Flow Rate: 2 mL/min; Co-solvent: 15% methanol; collection wavelength: 200-400 nm): ISOMER 1 Rt=1.32 min; ISOMER 2: Rt=1.62 min.

ISOMER 1 LCMS m/z 330.3 (M+H)⁺ (ES⁺). ¹H NMR (400 MHz, DMSO-d6) δ: 12.56 (br, 1H), 8.30 (d, J=7.6 Hz, 1H), 7.59 (d, J=8.4 Hz, 2H), 7.52 (d, J=8.4 Hz, 2H), 6.12 (s, 1H), 5.55 (s, 1H), 3.54-3.53 (m, 1H), 1.53 (d, J=16.4 Hz, 6H), 1.16 (d, J=7.2 Hz, 3H).

ISOMER 2 LCMS m/z 330.3 (M+H)⁺ (ES⁺). ¹H NMR (400 MHz, DMSO-d6) δ: 12.54 (br, 1H), 8.46 (d, J=7.6 Hz, 1H), 7.59 (d, J=8.0 Hz, 2H), 7.52 (d, J=8.4 Hz, 2H), 6.12 (s, 1H), 5.55 (s, 1H), 3.56-3.51 (m, 1H), 1.52 (d, J=16.8 Hz, 6H), 1.16 (d, J=15.2 Hz, 3H).

### Example 54: 3-methyl-2-methylene-4-oxo-4-(((S)-1-(4-(trifluoromethyl)phenyl)ethyl)amino)butanoic acid ISOMER 1 and Example 55: 3-methyl-2-methylene-4-oxo-4-(((S)-1-(4-(trifluoromethyl)phenyl)ethyl)amino)butanoic acid ISOMER 2

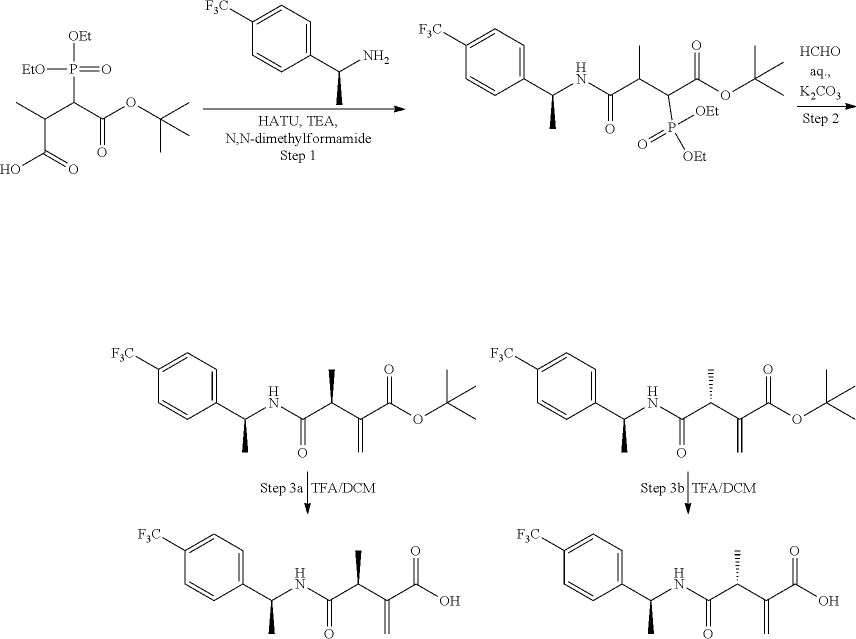

### Step 1

To the solution of (S)-1-(4-(trifluoromethyl)phenyl)ethan-1-amine (467 mg, 2.47 mmol) in N,N-dimethylformamide (12 mL) was added 4-(tert-butoxy)-3-(diethoxyphosphoryl)-2-methyl-4-oxobutanoic acid (800 mg, 2.47 mmol), triethylamine (749 mg, 7.41 mmol) and HATU (1.41 g, 3.70 mmol) at 0° C.; and the mixture was stirred at room temperature for 3 hours. The reaction mixture was quenched with 0.5N HCl (20 mL) and the aqueous layer was extracted with ethyl acetate (3×20 mL). The combined organic layers were washed by brine, dried over Na₂SO₄ and concentrated under reduced pressure. The residue was purified by flash column chromatography on silica (50-100% tert-butyl methyl ether/petroleum ether) to give tert-butyl 2-(diethoxyphosphoryl)-3-methyl-4-oxo-4-(((S)-1-(4-(trifluoromethyl)phenyl)ethyl)amino)butanoate (1.00 g, 82% yield) as light-brown oil. LCMS m/z 496.2 (M+H)⁺ (ES⁺).

### Step 2

To the mixture of tert-butyl 2-(diethoxyphosphoryl)-3-methyl-4-oxo-4-(((S)-1-(4-(trifluoromethyl)phenyl)ethyl)amino)butanoate (1.00 g, 2.02 mmol) and potassium carbonate (419 mg, 3.03 mmol) in THF (15 mL) and H₂O (3 mL) was added formaldehyde (491 mg, 6.06 mmol, 37% aqueous solution) at room temperature, and the reaction mixture was stirred at 65° C. for 16 hours. The reaction mixture was quenched with H₂O (10 mL) and extracted with tert-butyl methyl ether (3×10 mL). The combined organic layers were washed with brine, dried over Na₂SO₄ and concentrated under reduced pressure. The residue was purified by flash column chromatography on silica (0-20% tert-butyl methyl ether/petroleum ether) to give tert-butyl 3-methyl-2-methylene-4-oxo-4-(((S)-1-(4-(trifluoromethyl)phenyl)ethyl)amino)butanoate ISOMER 1 (300 mg, 40% yield) as colorless oil and tert-butyl 3-methyl-2-methylene-4-oxo-4-(((S)-1-(4-(trifluoromethyl)phenyl)ethyl)amino)butanoate (300 mg, 40% yield), as colorless oil.

ISOMER 1 LCMS m/z 394.2 (M+Na)⁺ (ES⁺). 1H NMR (400 MHz, CDCl3) δ: 7.58 (d, J=8.0 Hz, 2H), 7.41 (d, J=8.8 Hz, 2H), 6.47 (d, J=6.8 Hz, 1H), 6.28 (s, 1H), 5.77 (s, 1H), 5.10-5.03 (m, 1H), 3.53-3.48 (m, 1H), 1.50 (s, 9H), 1.42 (d, J=6.8 Hz, 3H), 1.31 (d, J=7.2 Hz, 3H).

ISOMER 2 LCMS m/z 394.3 (M+Na)⁺0.1H NMR (400 MHz, CDCl3) δ: 7.52 (d, J=8.0 Hz, 2H), 7.33 (d, J=8.4 Hz, 2H), 6.53 (d, J=7.6 Hz, 1H), 6.24 (s, 1H), 5.71 (s, 1H), 5.12-5.04 (m, 1H), 3.56-3.51 (m, 1H), 1.47 (d, J=7.2 Hz, 3H), 1.45 (s, 9H), 1.31 (d, J=6.8 Hz, 3H).

### Step 3a

A mixture of tert-butyl 3-methyl-2-methylene-4-oxo-4-(((S)-1-(4-(trifluoromethyl)phenyl)ethyl)amino)butanoate ISOMER 1 (300 mg, 0.80 mmol) in dichloromethane (3 mL) and TFA (1 mL) was stirred at room temperature for 3 hours. The solvent was removed under reduced pressure and the residue was purified by prep-HPLC (Column: Waters SUNFIRE Prep C18 OBD 10 μm 19×250 mm; Flow Rate: 20 mL/min; solvent system: MeCN/(0.2% formic acid/water) gradient: 55-95% MeCN; collection wavelength: 214 nm). The fractions were concentrated under reduced pressure to remove MeCN, and the residue was lyophilized to give 3-methyl-2-methylene-4-oxo-4-(((S)-1-(4-(trifluoromethyl)phenyl)ethyl)amino)butanoic acid ISOMER 1 (197 mg, 78% yield) as white solid. LCMS m/z 316.1 (M+H)⁺ (ES⁺). ¹H NMR (400 MHz, DMSO-d6) δ: 12.53 (br, 1H), 8.44 (d, J=7.6 Hz, 1H), 7.68 (d, J=8.0 Hz, 2H), 7.51 (d, J=8.0 Hz, 2H), 6.16 (s, 1H), 5.65 (s, 1H), 4.94-4.91 (m, 1H), 3.52-3.47 (m, 1H), 1.34 (d, J=6.8 Hz, 3H), 1.15 (d, J=7.2 Hz, 3H).

### Step 3b

A mixture of tert-butyl 3-methyl-2-methylene-4-oxo-4-(((S)-1-(4-(trifluoromethyl)phenyl)ethyl)amino)butanoate ISOMER 2 (300 mg, 0.80 mmol) in dichloromethane (3 mL) and TFA (1 mL) was stirred at room temperature for 3 hours. The solvent was removed under reduced pressure and the residue was purified by prep-HPLC (Column: Waters SUNFIRE Prep C18 OBD 10 μm 19×250 mm; Flow Rate: 20 mL/min; solvent system: MeCN/(0.2% formic acid/water) gradient: 55-95% MeCN; collection wavelength: 214 nm). The fractions were concentrated under reduced pressure to remove MeCN, and the residue was lyophilized to give 3-methyl-2-methylene-4-oxo-4-(((S)-1-(4-(trifluoromethyl)phenyl)ethyl)amino)butanoic acid ISOMER 2 (209 mg, 82% yield) as white solid. LCMS m/z 316.1 (M+H)⁺ (ES⁺). ¹H NMR (400 MHz, DMSO-d6) δ: 12.55 (s, 1H), 8.46 (d, J=7.6 Hz, 1H), 7.64 (d, J=8.4 Hz, 2H), 7.51 (d, J=8.0 Hz, 2H), 6.12 (s, 1H), 5.55 (s, 1H), 4.95-4.86 (m, 1H), 3.54-3.48 (m, 1H), 1.35 (d, J=6.8 Hz, 3H), 1.20 (d, J=7.2 Hz, 3H).

### Example 56: (S)-2-methylene-4-oxo-4-((1-(4-(pentafluoro-A6-sulfaneyl)phenyl)ethyl)amino)butanoic acid

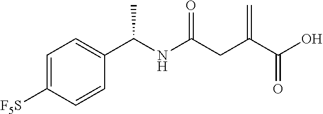

Prepared by an analogous method to Example 3, Method A starting from (S)-1-(4-(pentafluoro-λ6-sulfaneyl)phenyl)ethan-1-amine hydrochloride (Intermediate 12, 275 mg, 0.92 mmol), except that an extra equivalent of DIPEA was used in Step 1. Yield: 159 mg, 0.42 mmol. White solid. ¹H NMR (400 MHz, DMSO) δ 12.47 (s, 1H), 8.54-8.40 (m, 1H), 7.90-7.73 (m, 2H), 7.59-7.45 (m, 2H), 6.15-6.03 (m, 1H), 5.70-5.56 (m, 1H), 5.00-4.83 (m, 1H), 3.16 (s, 2H), 1.40-1.30 (m, 3H). LCMS m/z 360.0 (M+H)⁺ (ES⁺).

### Example 57: (S)-3-(1H-tetrazol-5-yl)-N-(1-(4-(trifluoromethyl)phenyl)ethyl)but-3-enamide

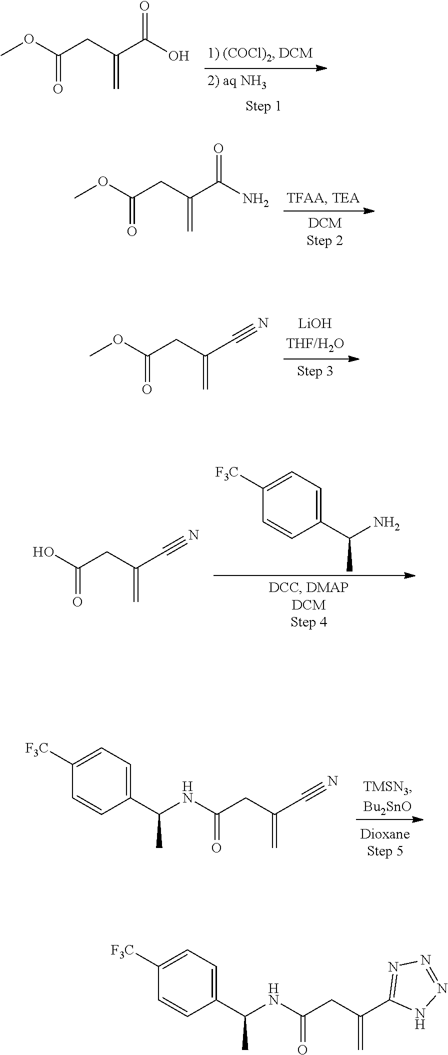

### Step 1

To the solution of 4-methoxy-2-methylene-4-oxobutanoic acid (1.40 g, 9.71 mmol) and N,N-dimethylformamide (0.1 mL) in DCM (20 mL) was added (COCl)₂ (1.85 g, 14.56 mmol) in portions at 0° C., and the reaction mixture was stirred at room temperature for 4 hours. Then mixture was concentrated under reduced pressure to give the crude acyl chloride. To a solution of acyl chloride in DCM (20 mL) was added aqueous ammonia (25% wt solution, 990 mg, 14.56 mmol,) at 0° C.; and the reaction mixture was stirred at room temperature for 1 hour. The mixture was concentrated under reduced pressure, and the residue was purified by flash column chromatography on silica (0-20% methanol/dichloromethane) to give methyl 3-carbamoylbut-3-enoate (1.00 g, 72% yield) as pale solid. ¹H NMR (400 MHz, CDCl₃) δ: 6.14 (br, 1H), 5.87 (s, 1H), 5.62 (br, 1H), 5.56 (s, 1H), 3.71 (s, 3H), 3.39 (s, 2H).

### Step 2

To a solution of methyl 3-carbamoylbut-3-enoate (1.00 g, 6.99 mmol) and triethylamine (1.42 g, 13.98 mmol) in DCM (10 mL) was added TFAA (2.20 g, 10.48 mmol) at 0° C., and the reaction mixture was stirred at room temperature for 1 hour. Then the mixture was quenched with water (5 mL) and extracted with Et₂O (2×10 mL). The combined organic layers were washed with brine, dried over Na₂SO₄ and filtered. The filtrate was concentrated under reduced pressure, and the residue was purified by flash column chromatography on silica (0-5% tert-butyl methyl ether/petroleum) to give methyl 3-cyanobut-3-enoate (500 mg, 57% yield) as colorless volatile oil. ¹H NMR (400 MHz, CDCl₃) δ: 6.08 (s, 1H), 5.92 (s, 1H), 3.77 (s, 3H), 3.29 (s, 2H).

### Step 3

To a solution of methyl 3-cyanobut-3-enoate (500 mg, 4.00 mmol) in THF (8 mL) was added 2N LiOH (4.0 mL, 8.00 mmol), and the reaction mixture was stirred at 25° C. for 2 hours. The reaction mixture was acidified with 2N HCl until pH 5 and extracted with Et₂O (3×5 mL). The combined organic layers were washed with brine, dried over Na₂SO₄ and filtered. The filtrate was concentrated under reduced pressure to give 3-cyanobut-3-enoic acid (450 mg, quant. yield) as colorless volatile oil. ¹H NMR (400 MHz, CDCl₃) δ: 6.08 (s, 1H), 5.95 (br, 1H), 5.92 (s, 1H), 3.30 (s, 2H).

### Step 4

A mixture of 3-cyanobut-3-enoic acid (450 mg, 4.05 mmol), (S)-1-(4-(trifluoromethyl)phenyl)ethan-1-amine (613 mg, 3.24 mmol), DCC (1.25 g, 6.07 mmol) and DMAP (49 mg, 0.40 mmol) in DCM (10 mL) was stirred at room temperature for 3 hours. The mixture was filtered, the filtrate was concentrated under reduced pressure, and the residue was purified flash column chromatography on silica (10-30% tert-butyl methyl ether/petroleum ether) to give (S)-3-cyano-N-(1-(4-(trifluoromethyl)phenyl)ethyl)but-3-enamide (750 mg, 61% yield) as white solid. ¹H NMR (400 MHz, DMSO-d₆) δ: 7.61 (d, J=8.4 Hz, 2H), 7.44 (d, J=8.0 Hz, 2H), 6.07 (s, 1H), 5.94 (s, 1H), 5.92 (d, J=8.0 Hz, 1H), 5.18-5.15 (m, 1H), 3.16 (s, 2H), 1.54 (d, J=6.8 Hz, 3H).

### Step 5

A mixture of (S)-3-cyano-N-(1-(4-(trifluoromethyl)phenyl)ethyl)but-3-enamide (200 mg, 0.71 mmol), azidotrimethylsilane (164 mg, 1.42 mmol) and dibutyltin oxide (18 mg, 0.07 mmol) in 1,4-dioxane (4 mL) was stirred at 90° C. for 2 hours under nitrogen atmosphere. The reaction mixture was filtered and the filtrate was concentrated at 40° C. under reduced pressure. The residue was purified by prep-HPLC (Column: Waters SUNFIRE Prep C18 OBD 10 μm 19×250 mm; Flow Rate: 20 mL/min; solvent system: MeCN/(0.05% formic acid/water) gradient: 65-95% MeCN; collection wavelength: 214 nm). The fractions were concentrated under reduced pressure to remove MeCN, and the residue was lyophilized to give (S)-3-(1H-tetrazol-5-yl)-N-(1-(4-(trifluoromethyl)phenyl)ethyl)but-3-enamide (174 mg, 76% yield) as white solid. LCMS m/z 326.2 (M+Na)⁺ (ES⁺). ¹H NMR (400 MHz, DMSO-d₆) δ: 16.52 (br, 1H), 8.67 (d, J=7.6 Hz, 1H), 7.65 (d, J=8.4 Hz, 2H), 7.53 (d, J=8.4 Hz, 2H), 6.06 (s, 1H), 5.63 (s, 1H), 4.95-4.88 (m, 1H), 3.55-3.46 (m, 2H), 1.37 (d, J=7.2 Hz, 1H).

### Biological Example 1—THP-1 AlphaLISA IL-1β and IL-6 Cytokine Assay

Measuring Inhibitory Effects on IL-1V Cytokine and IL-6 Output from THP-1s

The cytokine inhibition profiles of compounds of formula (I) were determined in a differentiated THP-1 cell assay. All assays were performed in RPMI-1640 growth medium (Gibco), supplemented with 10% fetal bovine serum (FBS; Gibco), 1% penicillin-streptomycin and 1% sodium pyruvate unless specified otherwise. The IL-1β and IL-6 cytokine inhibition assays were run in a background of differentiated THP-1 cells as described below. All reagents described were from Sigma-Aldrich unless specified otherwise. Compounds were prepared as 10 mM DMSO stocks.

### Assay Procedure

THP-1 cells were expanded as a suspension up to 80% confluence in appropriate growth medium. Cells were harvested, suspended, and treated with an appropriate concentration of phorbol 12-myristate 13-acetate (PMA) over a 72 hr period (37° C./5% CO₂).

Following 72 hrs of THP-1 cell incubation, cellular medium was removed and replaced with fresh growth media containing 1% of FBS. Working concentrations of compounds were prepared separately in 10% FBS treated growth medium and pre-incubated with the cells for 30 minutes (37° C./5% CO₂). Following the 30 minute compound pre-incubation, THP-1s were treated with an appropriate concentration of LPS and the THP-1s were subsequently incubated for a 24 hr period (37° C./5% CO₂). An appropriate final concentration of Nigericin was then dispensed into the THP-1 plates and incubated for 1 hour (37° C./5% CO₂) before THP-1 supernatants were harvested and collected in separate polypropylene 96-well holding plates.

Reagents from each of the IL-1β and IL-6 commercial kits (Perkin Elmer) were prepared and run according to the manufacturer's instructions. Subsequently, fluorescence signal detection in a microplate reader was measured (EnVision® Multilabel Reader, Perkin Elmer).

Percentage inhibition was calculated per cytokine by normalising the sample data to the high and low controls used within each plate (+/−LPS respectively). Percentage inhibition was then plotted against compound concentration and the 50% inhibitory concentration (IC₅₀) was determined from the resultant concentration-response curve.

Compounds of formula (I) were tested in this assay and the results of those compounds tested are shown in Table 1 below. Dimethyl itaconate and 4-octyl itaconate were included as comparator compounds.

TABLE 1
THP-1 cell IL-1β IC₅₀ values (μM)

| Compound | IL-1β (IC₅₀) | IL-6 (IC₅₀) |
|---|---|---|
| dimethyl itaconate | >100 | 100 |
| 4-octyl itaconate | 38.3 | 20.3 |
| Example 1 | 12.3 | |
| Example 2 | 8.5 | |
| Example 3 | 2.2 | >82.5 |
| | 4.0ª | |
| Example 4 | 3.4 | >63.1 |
| | 3.9ª | |
| Example 5 | 3.7 | >56.2 |
| | 5.6ª | |
| Example 6 | 25.3 | >100 |
| Example 7 | 8.2 | >100 |
| Example 8 | 43.5 | >100 |
| Example 9 | 8.2 | 7.27 |
| Example 10 | 16.6 | >100 |
| Example 11 | 8.3 | >100 |
| Example 12 | 26.6 | >100 |
| | 35.1ª | |
| Example 13 | 19.5 | >31.6 |
| Example 14 | 58.3 | >100 |
| Example 15 | 33.7 | >61.1 |
| | 32.6ª | |
| Example 16 | 3.8 | >56.2 |
| | 4.9ª | |
| Example 17 | 13.9 | >31.6 |
| dimethyl itaconate | >100 | 100 |
| 4-octyl itaconate | 38.3 | 20.3 |
| Example 18 | 56.0 | >31.6 |
| Example 19 | 8.8 | >31.6 |
| Example 20 | 17.4 | >100 |
| Example 21 | 4.0 | >100 |
| Example 22 | 7.1 | 3.6 |
| Example 23 | 13.4 | >100 |
| Example 24 | 4.7 | 4 |
| Example 25 | 4.1 | |
| Example 26 | 5.1 | 3.9 |
| Example 27 | 3.3 | 1.4 |
| Example 28 | 10.5 | 5.6 |
| Example 29 | 10.3 | 6.7 |
| Example 30 | 9.2 | >100 |
| Example 31 | 2.4 | 13.3 |
| Example 32 | 8.9 | |
| Example 33 | 1.82 | 3.64 |
| Example 34 | 14.8 | 25.8 |
| Example 35 | 42.7 | 11.9 |
| Example 36 | 3.9 | >100 |
| Example 37 | 1.7 | 7.29 |
| Example 38 | 29.6 | >100 |
| Example 39 | 15.4 | >100 |
| Example 40 | 26.9 | >100 |
| Example 41 | 24.3 | >31.6 |
| Example 42 | 2.3 | >68.1 |
| Example 43 | 2.1 | |
| Example 44 | 4.2 | |
| Example 45 | 2.1 | >31.6 |
| Example 46 | 2.5 | |
| Example 47 | >100 | >100 |
| Example 48 | 2.3 | >100 |
| Example 49 | 16.5 | >100 |
| Example 50 | 1.1 | >100 |
| dimethyl itaconate | >100 | 100 |
| 4-octyl itaconate | 38.3 | 20.3 |
| Example 51 | 10.2 | >100 |
| Example 52 | 18.7 | |
| Example 53 | 9.3 | >100 |
| Example 54 | 31.0 | >100 |
| Example 55 | 18.9 | >31.6 |
| Example 56 | 3.3 | >100 |
| Example 57 | 1.9 | >10 |

^{a}data from repeated experiments

Compounds of formula (I) that were tested in this assay exhibited improved cytokine-lowering potencies compared to dimethyl itaconate in respect of the cytokine IL-1p. Preferred compounds exhibited improved cytokine-lowering potencies compared to both dimethyl itaconate and 4-octyl itaconate in respect of the cytokines IL-1β and/or IL-6.

### Biological Example 2—NRF2+/−GSH Activation Assay

### Measuring Compound Activation Effects on the Anti-Inflammatory Transcription Factor NRF2 in DiscoverX PathHunter NRF2 Translocation Kit

Potency and efficacy of compounds of formula (I) against the target of interest to activate NRF2 (nuclear factor erythroid 2-related factor 2) were determined using the PathHunter NRF2 translocation kit (DiscoverX). The NRF2 translocation assay was run using an engineered recombinant cell line, utilising enzyme fragment complementation to determine activation of the Keap1-NRF2 protein complex and subsequent translocation of NRF2 into the nucleus. Enzyme activity was quantified using a chemiluminescent substrate consumed following the formation of a functional enzyme upon PK-tagged NRF2 translocation into the nucleus.

The assay was run under either +/−GSH (glutathione) conditions to determine the attenuating activities of GSH against target compounds.

Additionally, a defined concentration of dimethyl fumarate was used as the ‘High’ control to normalise test compound activation responses to.

### Assay Procedure

U2OS PathHunter eXpress cells were thawed from frozen prior to plating. Following plating, U2OS cells were incubated for 24 hrs (37° C./5% CO₂) in commercial kit provided cell medium.

Following 24 hrs of U2OS incubation, cells were directly treated with an appropriate final concentration of compound, for −GSH conditions, or for +GSH conditions, an intermediate plate containing 6× working concentrations of compound stocks was prepared in a 6 mM working concentration of GSH solution (solubilised in sterile PBS). Following a 30 minute compound-GSH pre-incubation (37° C./5% CO₂) for +GSH treatment, plated U2OS cells were incubated with an appropriate final concentration of compound and GSH.

Following compound (+/−GSH) treatment, the U2OS plates were incubated for a further 6 hours (37° C./5% CO₂) before detection reagent from the PathHunter NRF2 commercial kit was prepared and added to test plates according to the manufacturer's instructions. Subsequently, the luminescence signal detection in a microplate reader was measured (PHERAstar®, BMG Labtech).

Percentage activation was calculated by normalising the sample data to the high and low controls used within each plate (+/−DMF). Percentage activation/response was then plotted against compound concentration and the 50% activation concentration (EC₅₀) was determined from the plotted concentration-response curve.

A number of compounds of formula (I) were tested in this assay, and the results are shown in Table 2 below. Dimethyl itaconate and 4-octyl itaconate were included as comparator compounds.

TABLE 2
NRF2 activation

| | −GSH | | +GSH | |
|---|---|---|---|---|
| Compound | EC₅₀ (μM) | Eₘₐₓ (%) | EC₅₀ (μM) | Eₘₐₓ (%) |
| dimethyl itaconate | 21.4 | 137 | 56.4 | 77 |
| 4-octyl itaconate | 22.1 | 165 | 28.9 | 148 |
| Example 1 | 22.3 | 63 | >100 | 19 |
| Example 2 | 24.9 | 127 | 41.9 | 90 |
| Example 3 | >100 | 0 | >100 | 0 |
| Example 4 | >100 | 1 | >100 | 0 |
| dimethyl itaconate | 21.4 | 137 | 56.4 | 77 |
| 4-octyl itaconate | 22.1 | 165 | 28.9 | 148 |
| Example 5 | >100 | 17 | >100 | 0 |
| Example 9 | 19.2 | 109 | >100 | 55 |
| Example 22 | >100 | 3 | >100 | 8 |
| Example 24 | 9.05 | 78 | >100 | 30 |
| Example 27 | 6.3 | 84 | >100 | 10 |
| Example 29 | 22.7 | 86 | >100 | 22 |
| Example 31 | 43.3 | 82 | >100 | 27 |
| Example 32 | 46.2 | 58 | >100 | 12 |
| Example 34 | 5.7 | 81 | >100 | 16 |
| Example 36 | 15.9 | 65 | >100 | 25 |
| Example 37 | 15.8 | 90 | >100 | 11 |
| Example 42 | >100 | 8 | >100 | 3 |
| Example 57 | 46.8 | 55 | >100 | 48.5 |

Certain compounds of formula (I) tested in this assay showed activity in this assay (such as under −GSH conditions), as demonstrated by their EC₅₀ and/or Eₘₐₓ values for NRF2 activation, and thus may be expected to have utility in the treatment of diseases wherein such activity may be beneficial (such as multiple sclerosis, psoriasis and chronic obstructive pulmonary disease: Cuadrado et al., Nat. Rev. Drug Discov. 2019, 18, 295-317). Other compounds of formula (I) tested in this assay showed very little activity in this assay, as demonstrated by their EC₅₀ and/or Eₘₐₓ values for NRF2 activation, indicating that the IL1β-lowering effect is not a consequence of NRF2 activation. Such compounds may be expected to have utility in situations where activation of NRF2 could lead to toxicity or undesirable effects (He et al., J. Hepatol. 2020, 72, 1182-1195; Wu et al., Cancer Medicine. 2019, 8, 2252-2267).

### Biological Example 3—Hepatocyte Stability Assay

Defrosted cryo-preserved hepatocytes (viability>70%) were used to determine the metabolic stability of a compound via calculation of intrinsic clearance (CIᵢₙₜ; a measure of the removal of a compound from the liver in the absence of blood flow and cell binding). Clearance data are particularly important for in vitro work as they can be used in combination with in vivo data to predict the half-life and oral bioavailability of a drug.

The metabolic stability in hepatocytes assay involved a time-dependent reaction using both positive and negative controls. The cells must be pre-incubated at 37° C. then spiked with test compound (and positive control); samples taken at pre-determined time intervals were analysed to monitor the change in concentration of the initial drug compound over 60 minutes. A buffer incubation reaction (with no hepatocytes present) acted as a negative control and two cocktail solutions, containing compounds with known high and low clearance values (verapamil/7-hydroxycoumarin and propranolol/diltiazem), acted as positive controls.
1. The assay was run with a cell concentration of 0.5×10⁶ cells/mL in Leibovitz buffer.
2. All compounds and controls were run in duplicate.
3. Compound concentration was 10 μM.
4. All compounds and controls were incubated with both cells and buffer to show turnover is due to hepatic metabolism.
5. All wells on the incubation plate had 326.7 μL of either cells or buffer added.
6. Prior to assay, cell and buffer-only incubation plates were preincubated for 10 mins at 37° C.
7. The assay was initiated by adding compounds, 3.3 μL of 1 mM in 10% DMSO-90% Buffer; final DMSO concentration is 0.1%.
8. Samples were taken at regular timepoints (0, 5, 10, 20, 40, 60 min) until 60 mins.
9. Sample volume was 40 μL and added to 160 μL of crash solvent (acetonitrile with internal standard) and stored on ice.
10. At the end of the assay, the crash plates were centrifuged at 3500 rpm for 20 mins at 4° C.
11. 80 μL of clear supernatant was removed and mixed with 80 μL of deionised water before being analysed by LC-MS/MS.

Raw LC-MS/MS data were exported to, and analysed in, Microsoft Excel for determination of intrinsic clearance. The percentage remaining of a compound was monitored using the peak area of the initial concentration as 100%. Intrinsic clearance and half-life values were calculated using a graph of the natural log of percentage remaining versus the time of reaction in minutes. Half-life (min) and intrinsic clearance (CIᵢₙₜ in μL min⁻¹ 10⁻⁶ cells) values were calculated using the gradient of the graph (the elimination rate constant, k) and Equations 1 and 2.

$${t}_{\frac{1}{2}} = \frac{ln 2}{k} \qquad \{ Equation 1 \}$$

$${Cl}_{i n t} = ( \frac{ln 2}{{t}_{\frac{1}{2}}} ) \times ( \frac{350}{0.175} ) \qquad \{ Equation 2 \}$$

A number of compounds of formula (I) were tested in this assay, and the results are shown in Table 3 below. 4-Octyl itaconate was included as a comparator compound.

TABLE 3
Hepatocyte stability

| | | Clᵢₙₜ (μL min⁻¹ | |
|---|---|---|---|
| Compound | Species | 10⁻⁶ cells) | T½ (min) |
| 4-octyl itaconate | Human | 401 | 4 |
| | Mouse | 351 | 4 |
| Example 1 | Human | 12 | 117 |
| | | 31ª | 47 |
| | Mouse | 31 | 98 |
| | | 133 | 11ª |
| Example 2 | Human | 14 | 120 |
| | | 21ª | 77ª |
| | Mouse | 119 | 59 |
| | | 156ª | 9ª |
| Example 3 | Mouse | | 402 |
| | | <3 | >460ª |
| | Human | 4 | >460 |
| | | | 358ª |
| Example 4 | Mouse | 3 | >460 |
| | Human | | 390 |
| | | <3 | >460 |
| Example 5 | Mouse | 3 | 450 |
| | Human | <3 | >460 |
| Example 7 | Human | <3 | >460 |
| | Mouse | 21 | 75 |
| Example 9 | Human | 116 | 10 |
| | Mouse | 619 | 3.5 |
| Example 10 | Human | <3 | >460 |
| | Mouse | <3 | >460 |
| Example 11 | Human | <3 | >460 |
| | Mouse | <3 | >460 |
| Example 13 | Human | 3 | 430 |
| | Mouse | <3 | >460 |
| Example 14 | Human | <3 | >460 |
| 4-octyl itaconate | Human | 401 | 4 |
| | Mouse | 351 | 4 |
| | Mouse | 4 | 389 |
| Example 16 | Human | 6 | 281 |
| | Mouse | 7 | 246 |
| Example 17 | Human | 8 | 223 |
| | Mouse | 16 | 95 |
| Example 19 | Human | 4 | 437 |
| | Mouse | 4 | 402 |
| Example 20 | Human | <3 | >460 |
| | Mouse | 38 | 38 |
| Example 21 | Human | <3 | >460 |
| | Mouse | 76 | 22 |
| Example 22 | Human | 49 | 37 |
| | Mouse | 99 | 13 |
| Example 23 | Human | <3 | >460 |
| | Mouse | 4 | 355 |
| Example 24 | Human | 23 | 65 |
| | Mouse | 199 | 8 |
| Example 25 | Human | 21 | 70 |
| | Mouse | 153 | 11 |
| Example 26 | Human | 62 | 24 |
| | Mouse | >460 | <3 |
| Example 27 | Human | 126 | 12 |
| | Mouse | 215 | 6 |
| Example 28 | Human | 80 | 18 |
| | Mouse | >460 | <3 |
| Example 29 | Human | 39 | 38 |
| | Mouse | 130 | 13 |
| Example 30 | Human | <3 | >460 |
| | Mouse | <3 | >460 |
| Example 31 | Human | 11 | 138 |
| | Mouse | 34 | 35 |
| Example 32 | Human | 12 | 123 |
| | Mouse | 87 | 20 |
| 4-octyl itaconate | Human | 401 | 4 |
| | Mouse | 351 | 4 |
| Example 33 | Human | 151 | 11 |
| | Mouse | 205 | 10 |
| Example 34 | Human | 11 | 137 |
| | Mouse | 24 | 71 |
| Example 36 | Human | NT | NT |
| | Mouse | 61 | 22 |
| Example 37 | Human | 50 | 25 |
| | Mouse | 104 | 12 |
| Example 39 | Human | <3 | >460 |
| | Mouse | <3 | >460 |
| Example 40 | Human | <3 | >460 |
| | Mouse | <3 | >460 |
| Example 41 | Human | <3 | >460 |
| | Mouse | <3 | >460 |
| Example 42 | Human | <3 | >460 |
| | Mouse | 3 | 323 |
| Example 43 | Human | <3 | >460 |
| | Mouse | 9 | 163 |
| Example 44 | Human | <3 | >460 |
| | Mouse | 6 | 239 |
| Example 45 | Human | 8 | 230 |
| | Mouse | 30 | 47.6 |
| Example 46 | Human | 7 | 201 |
| | Mouse | 37 | 40 |
| Example 47 | Human | <3 | >460 |
| | Mouse | 12 | 132 |
| Example 48 | Human | 7 | 224 |
| | Mouse | 95 | 16 |
| Example 49 | Human | <3 | >460 |
| | Mouse | <3 | >460 |
| Example 50 | Human | <3 | >460 |
| | Mouse | 13 | 104 |
| Example 51 | Human | <3 | >460 |
| 4-octyl itaconate | Human | 401 | 4 |
| | Mouse | 351 | 4 |
| Example 52 | Muse | 4 | 431 |
| | Human | <3 | >460 |
| | Mouse | <3 | >460 |
| Example 53 | Human | <3 | >460 |
| | Mouse | 5 | 287 |
| Example 54 | Human | <3 | >460 |
| | Mouse | 4 | 400 |
| Example 55 | Human | <3 | >460 |
| | Mouse | 3 | 457 |
| Example 56 | Human | <3 | >460 |
| | Mouse | 11 | 166 |
| Example 57 | Human | <3 | >460 |
| | Mouse | <3 | >460 |

*NT = not tested in this assay
^{a}data from repeated experiments

The results indicate that the compounds of the invention, at least those of Table 3, are expected to have acceptable or improved metabolic stabilities, as shown by their intrinsic clearance (CIᵢₙₜ) and half-life (T_{1/2}) values, in this assay. All compounds in Table 3 were more stable, i.e., they exhibited lower intrinsic clearance (CIᵢₙₜ) and longer half-life (T_{1/2}) values compared with 4-octyl itaconate in at least human or mouse species. Preferred compounds exhibited lower intrinsic clearance (CIᵢₙₜ) and longer half-life (T_{1/2}) values compared with 4-octyl itaconate in both human and mouse species.

### Biological Example 4—Plasma Protein Binding

Equilibrium dialysis is an accurate and reliable method for determining protein binding affinities to chemical or biological substances of low molecular weight. The Rapid Equilibrium Dialysis (RED) plate is specifically designed and extensively validated for plasma protein binding assays. It is also designed for minimal non-specific binding (Thermo Fisher).

### Assay Preparation

Phosphate buffered saline (PBS) pH 7.4 was prepared using PBS tablets as per the manufacturer's instructions.
Compound and marker stocks were prepared at 10 mM in DMSO and further diluted to 100× the assay concentration in DMSO.
Plasma was thawed on the day of the assay and centrifuged. The supernatant was transferred to a fresh vessel and the pH adjusted to pH 7.4 using lactic acid or sodium hydroxide.

### Assay Procedure

Pre-prepared 100× assay compound stocks were spiked 1:100 into pH adjusted plasma. 200 μL of spiked plasma was added to the red compartment of the rapid equilibrium dialysis (RED) device for each replicate. 350 μL of PBS was added to the buffer compartment. The plate was sealed with a breathable lid and shaken on an orbital shaker in a 37° C. incubator, 5% CO₂ for 4 hours.

Standard final incubation conditions were 5 μM compound in plasma containing 1% (v/v) DMSO.

A sample of the plasma/compound mixture was taken, mixed with an equal volume of PBS and quenched 1:3 with ice-cold acetonitrile containing internal standard. Methanol was also added to matrix match with calibration curve samples. This was the t=0 sample.

Post-incubation, plasma and buffer were sampled from their respective RED device compartments into separate tubes containing an equal volume of the alternate matrix for each replicate. Samples were quenched 1:3 with ice-cold acetonitrile containing internal standard. Methanol was also added to matrix match with calibration curve samples.

Calibration curves were prepared in methanol and spiked such that the matrix was equivalent to the assay samples.

Quenched samples were mixed thoroughly and protein precipitated at −20° C. for a minimum of 12 hours. Samples were then centrifuged at 4° C. Supernatants were transferred to a fresh 96 well plate for analysis.

Positive control markers were included.

### Data Processing

Fraction unbound, percent bound and percent recovery were the standard assay parameters reported. They were calculated using the following equations:

$$Fraction unbound ( {f}_{u} ) = [ PBS ] / [ plasma ]$$

$$PPB ( \% ) - 100 \times ( 1 - {f}_{u} )$$

$$Recovery ( \% ) = \frac{( {C}_{PBS} \times {V}_{PBS} ) + ( {C}_{plasma} \times {V}_{plasma} )}{{C}_{0 plasma} \times {V}_{plasma}} \times 100 \%$$

$$\begin{matrix}{C}_{PBS} & Concentration in buffer compartment after incubation \\ {C}_{plasma} & Concentration in plasma compartment after incubation \\ {C}_{0 plasma} & Concentration in t = 0 sample \\ {V}_{PBS} & Volume in buffer side of incubation \\ {V}_{plasma} & Volume in plasma side of incubation\end{matrix}$$

An example compound of formula (I) was tested in this assay. Reference Example 2 (RE2) (which corresponds to Example 49 in WO2020/222011 (Sitryx Therapeutics, 2020)) was used as a comparator compound.

TABLE 4

| Compound | Species | Binding (%) |
|---|---|---|
| RE2 | Human | >99 |
| | Mouse | >99 |
| Example 3 | Human | 89.0 |
| | Mouse | 86.8 |
| Example 4 | Human | 89.2 |
| | Mouse | 83.7 |

The results in Table 4 show that Example 3 exhibits lower plasma protein binding (PPB) compared to a prior art ester compound. As such, at a given equivalent total drug in vivo blood concentration, amide compounds, such as the Examples provided here, would exhibit higher unbound drug concentrations compared to the equivalent ester compounds. In addition, these amides display PPB values less than 99%, making them technically more straightforward to measure.

### REFERENCES

The following publication cited in this specification are herein incorporated by reference in their entirety.
Ackermann et al. Proc. Soc. Exp. Bio. Med. 1949, 72(1), 1-9.
Andersen J. L. et al. Nat. Commun. 2018, 9, 4344.
Angiari S. and O'Neill L. A. Cell Res. 2018, 28, 613-615.
Bagavant G. et al. Indian J. Pharm. Sci. 1994, 56, 80-85.
Bambouskova M. et al. Nature 2018, 556, 501-504.
Blewett M. M. et al. Sci. Sign. 2016, 9 (445), rs10; 6.
Brennan M. S. et al. PLoS One 2015, 10, e0120254.
Brück J. et al. Exp. Dermatol. 2018, 27, 611-624.
Cocco M. et al. J. Med. Chem. 2014, 57, 10366-10382.
Cocco M. et al. J. Med. Chem. 2017, 60, 3656-3671.
Cordes T. et al. J. Biol. Chem. 2016, 291, 14274-14284.
Cordes T. et al. Mol. Metab. 2020, 32, 122-135.
Cuadrado et al., Nat. Rev. Drug Discov. 2019, 18, 295-317.
Daly R. et al. medRxiv 2019, 19001594; doi: https://doi.org/10.1101/19001594.
Daniels B. P. et al. Immunity 2019, 50(1), 64-76.e4.
Dibbert S. et al. Arch. Dermatol. Res. 2013, 305, 447-451.
ElAzzouny M. et al. J. Biol. Chem. 2017, 292, 4766-4769.
Gillard G. O. et al. J. Neuroimmunol. 2015, 283, 74-85.
Gu L. et al. Immunol. Cell Biol. 2020 doi:10.1111/imcb.12316.
Hanke T. et al. Pharmacol. Therapeut. 2016, 157, 163-187.
He et al., J. Hepatol. 2020, 72, 1182-1195.
Hunt T. et al. Consortium of Multiple Sclerosis Centers 2015 Annual Meeting, 27-30 May 2015, Indianapolis, IN, USA: Poster DX37.
Kornberg M. D. et al. Science 2018, 360, 449-453.
Kulkarni R. A. et al. Nat. Chem. Biol. 2019 10.1038/s41589-018-0217-y.
Lampropoulou V. et al. Cell Metab. 2016, 24, 158-166.
Lehmann J. C. U. et al. J. Invest. Dermatol. 2007, 127, 835-845.
Liao S.-T. et al. Nat. Commun. 2019, 10(1), 5091.
Liu H. et al. Cell Commun. Signal. 2018, 16, 81.
McGuire V. A. et al. Sci. Rep. 2016, 6, 31159.
Michelucci A. et al. Proc. Natl. Acad. Sci. USA 2013, 110, 7820-7825.
Mills E. A. et al. Front. Neurol. 2018, 9, 5.
Mills E. L. et al. Cell 2016, 167, 457-470.
Mills E. L. et al. Nature 2018, 556, 113-117.
Mrowietz U. et al. Trends Pharmacol. Sci. 2018, 39, 1-12.
Müller S. et al. J. Dermatol. Sci. 2017, 87, 246-251.
Murphy M. P. and O'Neill L. A. J. Cell 2018, 174, 780-784.
O'Neill L. A. J. and Artyomov M. N. Nat. Rev. Immunol. 2019 273-281.
Olagnier D. et al. Nat. Commun. 2018, 9, 3506.
Schmidt T. J. et al. Bioorg. Med. Chem. 2007, 15, 333-342.
Shan Q. et al. Biochem. Biophys. Res. Commun. 2019, 517, 538-544.
Straub R. H. and Schradin C. Evol. Med. Public Health 2016, 1, 37-51S.
Straub R. H. and Cutolo M. Rheumatology 2016, 55 (Suppl. 2), ii6-ii14.
Sun X. et al., FASEB J. 2019, 33, 12929-12940.
Tang C. et al. Cell Physiol. Biochem. 2018, 51, 979-990.
Tang C. et al. Biochem. Biophys. Res. Commun. 2019, 508, 921-927.
Tang H. et al. Biochem. Biophys. Res. Commun. 2008, 375, 562-565.
Tian et al. Eur. J. Pharmacol. 2020, 873, 172989.
van der Reest J. et al. Nat. Commun. 2018, 9, 1581.
von Glehn F. et al. Mult. Scler. Relat. Disord. 2018, 23, 46-50.
Wu et al., Cancer Medicine. 2019, 8, 2252-2267.
Yi F. et al. Hepatology 2020, 873, 172989.
Yu X.-H. et al. Immunol. Cell Biol. 2019, 97, 134-141.
Zhang S. et al. Bioorg. Med. Chem. 2012, 20, 6073-6079.
Zhang D. et al. Int. Immunopharmacol. 2019, 77, 105924.
Zhao C. et al. Microb. Pathogen. 2019, 133, 103541.
Zhao G. et al. Biochem. Biophys. Res. Commun. 2014, 448, 303-307.

### MISCELLANEOUS

All references referred to in this application, including patent and patent applications, are incorporated herein by reference to the fullest extent possible.

Throughout the specification and the claims which follow, unless the context requires otherwise, the word ‘comprise’, and variations such as ‘comprises’ and ‘comprising’, will be understood to imply the inclusion of a stated integer, step, group of integers or group of steps but not to the exclusion of any other integer, step, group of integers or group of steps.

The application, of which this description and claims form part, may be used as a basis for priority in respect of any subsequent application. The claims of such subsequent application may be directed to any feature or combination of features described herein. They may take the form of product, composition, process, or use claims and may include, by way of example and without limitation, the following claims.

1: A compound of formula (I): 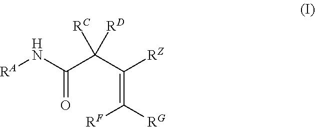
wherein:
R^{A} is selected from the group consisting of C₆₋₁₀ cycloalkyl optionally fused to phenyl, CH₂(phenyl) and CH₂(pyridyl);
wherein when R^{A} is C₆₋₁₀ cycloalkyl optionally fused to phenyl, the cycloalkyl ring s optionally substituted on an available carbon atom by one or more R^{A2}, wherein each R^{A2} is independently selected from the group consisting of methyl, halo and trifluoromethyl, and/or two R^{A2} are attached to the same carbon atom and join to form a C₃₋₆ cycloalkyl or a 4-6 membered heterocyclic ring; and the fused phenyl ring is optionally substituted on an available carbon atom by one or more substituents selected from the group consisting of C₁₋₄ haloalkyl and halo;
wherein when R^{A} is CH₂(phenyl) or CH₂(pyridyl):
the CH₂ group is optionally substituted by one or two R^{A3} wherein each R^{A3} is independently selected from the group consisting of C₁₋₄ alkyl, C₃₋₅ cycloalkyl, C₁₋₄ haloalkyl and C₁₋₂ hydroxyalkyl, or two R^{A3} groups are attached to the CH₂ carbon atom and join to form a C₃₋₆ cycloalkyl or a 4- or 5-membered heterocyclic ring; and
the phenyl or pyridyl group is optionally substituted by one or more R^{A4}, wherein each R^{A4} is independently selected from the group consisting of C₁₋₄ haloalkyl, halo, SF₅ or SC₁₋₄ haloalkyl;
R^{Z} is selected from the group consisting of C(O)OR^{B} and tetrazol-5-yl;
R^{B} is selected from the group consisting of H; C₂₋₃ alkyl substituted with OH or N(R^{B2})(R^{B3}); and 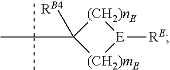
wherein:
when R^{B} is C₂₋₃ alkyl substituted with N(R^{B2})(R^{B3}), R^{B2} is selected from H and methyl and R^{B3} is selected from methyl and SO₂(C₁₋₂ alkyl) or R^{B2} and R^{B3}, together with the nitrogen atom to which they are attached, combine to form a 4- to 6-membered heterocyclic ring optionally containing a further heteroatom selected from O and N;
wherein, when R^{B} is 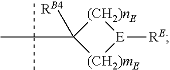
R^{B4} is H or methyl;
E is N, O or SO₂;
wherein when E is N, R^{E} is selected from the group consisting of R^{9B}, C(O)R^{9B} and SO₂R^{9B}; wherein R^{9B} is C₁₋₄ alkyl; and when E is O or SO₂, R^{E} is absent;
nE and mE are independently 1 or 2;
R^{C} and R^{D} are each independently H, C₁₋₂ alkyl, hydroxy, methoxy or fluoro;
each of R^{F} and R^{G} is independently selected from H, phenyl and C₁₋₄ alkyl optionally substituted with phenyl, OR^{K} or N(R^{K})(R^{L}), wherein each of R^{K} and R^{L} is independently selected from H, methyl and ethyl, provided that at least one of R^{F} and R^{G} is H; and
wherein when the CH₂ of the CH₂(substituted phenyl) or CH₂(substituted pyridyl) of R^{A} is substituted by one R^{A3} the stereochemistry of the carbon to which R^{A3} is attached is as follows: 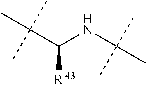
wherein the dashed lines indicate attachment to the remainder of the compound of formula (I);
and wherein when R^{A4} is Cl, the CH₂ group is unsubstituted or is substituted by one R^{A3};
or a pharmaceutically acceptable salt and/or solvate thereof.

2: The compound according to claim 1 which is a compound of formula (Ia): 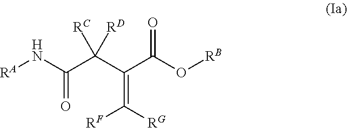
wherein:
R^{A} is selected from the group consisting of C₆₋₁₀ cycloalkyl optionally fused to phenyl, CH₂(phenyl) and CH₂(pyridyl);
wherein when R^{A} is C₆₋₁₀ cycloalkyl optionally fused to phenyl, the cycloalkyl ring is optionally substituted on an available carbon atom by one or more R^{A2}, wherein R^{A2} is selected from the group consisting of methyl, halo and trifluoromethyl, and/or two R^{A2} are attached to the same carbon atom and join to form a C₃₋₆ cycloalkyl or a 4-6 membered heterocyclic ring; and the fused phenyl ring is optionally substituted on an available carbon atom by one or more substituents selected from the group consisting of C₁₋₄ haloalkyl and halo;
wherein when R^{A} is CH₂(phenyl) or CH₂(pyridyl):
the CH₂ group is optionally substituted by one or two R^{A3} wherein R^{A3} is selected from the group consisting of C₁₋₄ alkyl, C₃₋₅ cycloalkyl, C₁₋₄ haloalkyl and C₁₋₂ hydroxyalkyl, or two R^{A3} groups are attached to the CH₂ carbon atom and join to form a C₃₋₆ cycloalkyl or a 4- or 5-membered heterocyclic ring; and
the phenyl or pyridyl group is optionally substituted by one or more R^{A4}, wherein R^{A4} is selected from the group consisting of C₁₋₄ haloalkyl, halo, or SC₁₋₄ haloalkyl;
R^{B} is selected from the group consisting of H; C₂₋₃ alkyl substituted with OH or N(R^{B2})(R^{B3}); and 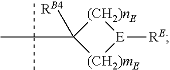
wherein:
when R^{B} is C₂₋₃ alkyl substituted with N(R^{B2})(R^{B3}), R^{B2} is selected from H and methyl and R^{B3} is selected from methyl and SO₂(C₁₋₂ alkyl) or R^{B2} and R^{B3}, together with the nitrogen atom to which they are attached, combine to form a 4- to 6-membered heterocyclic ring optionally containing a further heteroatom selected from O and N;
wherein, when R^{B} is 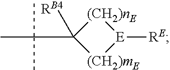
R^{B4} is H or methyl;
E is N, O or SO₂;
wherein when E is N, R^{E} is selected from the group consisting of R^{9B}, C(O)R^{9B} and SO₂R^{9B}; wherein R^{9B} is C₁₋₄ alkyl; and when E is O or SO₂, R^{E} is absent;
nE and mE are independently 1 or 2;
R^{C} and R^{D} are each independently H, C₁₋₂ alkyl, hydroxy, methoxy or fluoro;
each of R^{F} and R^{G} is independently H or methyl, provided that at least one of R^{F} and R^{G} is H; and
wherein when the CH₂ of the CH₂(substituted phenyl) or CH₂(substituted pyridyl) of R^{A} is substituted by one R^{A3} the stereochemistry of the carbon to which R^{A3} is attached is as follows: 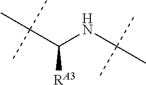
wherein the dashed lines indicate attachment to the remainder of the compound of formula (I);
and wherein when R^{A4} is Cl, the CH₂ group is unsubstituted or is substituted by one R^{A3}; and
wherein, when R^{B} is H, R^{A} is not unsubstituted cyclohexyl;
or a pharmaceutically acceptable salt and/or solvate thereof.

3-5. (canceled)

6: The compound or a pharmaceutically acceptable salt and/or solvate thereof according to claim 1, wherein R^{A} is CH₂(substituted phenyl) or CH₂(substituted pyridyl), wherein the CH₂ group is optionally substituted by one or two R^{A3} wherein each R^{A3} is independently as defined in claim 1.

7. (canceled)

8: The compound or a pharmaceutically acceptable salt and/or solvate thereof according to claim 6, wherein the CH₂ group is optionally substituted by one or two R^{A3} wherein R^{A3} is methyl, ethyl, cyclopropyl, trifluoromethyl or CH₂OH, or two R^{A3} groups are attached to the CH₂ carbon atom and join to form a cyclopropyl, cyclobutyl or cyclopentyl or oxetanyl group.

9-10. (canceled)

11: The compound or a pharmaceutically acceptable salt and/or solvate thereof according to claim 1 wherein:
the phenyl or pyridyl group is substituted by one R^{A4}, which is in the para-position with respect to the linkage to the remainder of the molecule; or
the phenyl or pyridyl group is substituted by two R^{A4}, wherein the first R^{A4} is in the para-position with respect to the linkage to the remainder of the molecule and the second R^{A4} is in the meta position with respect to the linkage to the remainder of the molecule, wherein each R^{A4} is independently selected from CHF₂, CF₃CF₂, CF₃, fluoro, chloro, bromo and SF₅.

12. (canceled)

13: The compound or a pharmaceutically acceptable salt and/or solvate thereof according to claim 1, wherein R^{Z} is C(O)OR^{B}.

14: The compound or a pharmaceutically acceptable salt and/or solvate thereof according to claim 13, wherein R^{B} is H or C₂₋₃ alkyl substituted with OH or N(R^{B2})(R^{B3}),
wherein:
R^{B2} is H and R^{B3} is SO₂(C₁₋₂ alkyl); or
R^{B2} is H or methyl, and R^{B3} is methyl; or
R^{B2} and R^{B3} together with the nitrogen atom to which they are attached combine to form a morpholine ring.

15-16. (canceled)

17: The compound or a pharmaceutically acceptable salt and/or solvate thereof according to claim 13, wherein R^{B} is 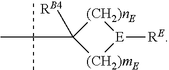
wherein R^{B4}, E, R^{E}, mE and nE are as defined in claim 1.

18: The compound or a pharmaceutically acceptable salt and/or solvate thereof according to claim 17, wherein E is O or SO₂ and R^{E} is absent.

19: The compound or a pharmaceutically acceptable salt and/or solvate thereof according to claim 17, wherein E is N and R^{E} is R^{9B}, C(O)R^{9B} or SO₂R^{9B}, wherein R^{9B} is methyl.

20-21. (canceled)

22: The compound or a pharmaceutically acceptable salt and/or solvate thereof according to claim 1, wherein R^{C} is H and R^{D} is H.

23: The compound or a pharmaceutically acceptable salt and/or solvate thereof according to claim 1, wherein:
R^{F} is H and R^{G} is H; or
each of R^{F} and R^{G} is independently H or methyl, provided that at least one of R^{F} and R^{G} is H; or
R^{F} is phenyl or C₁₋₄ alkyl optionally substituted with phenyl, OR^{K} or N(R^{K})(R^{L}) and R^{G} is H; or
R^{F} is H and R^{G} is phenyl or C₁₋₄ alkyl optionally substituted with phenyl, OR^{K} or N(R^{K})(R^{L}).

24: The compound or a pharmaceutically acceptable salt and/or solvate thereof according to claim 23 wherein:
the compound is a compound of formula (Ic): 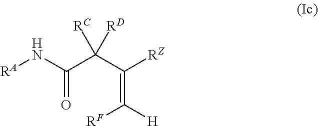
wherein R^{F} is phenyl or C₁₋₄ alkyl optionally substituted with phenyl, OR^{K} or N(R^{K})(R^{L}) and R^{A}, R^{C}, R^{D} and R^{Z} are as defined in claim 1; or
the compound is a compound of formula (Id): 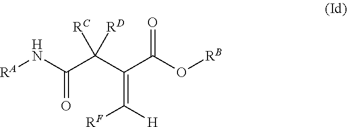
wherein R^{F} is phenyl or C₁₋₄ alkyl optionally substituted with phenyl, OR^{K} or N(R^{K})(R^{L}) and R^{A}, R^{C}, R^{D} and R^{B} are as defined for formula (I).

25: The compound according to claim 1 selected from:
1-(methylsulfonyl)piperidin-4-yl 4-((4-chlorobenzyl)amino)-2-methylene-4-oxobutanoate;
1-(methylsulfonyl)piperidin-4-yl 4-(cyclooctylamino)-2-methylene-4-oxobutanoate;
(S)-2-methylene-4-oxo-4-((1-(4-(trifluoromethyl)phenyl)ethyl)amino)butanoic acid;
2-methylene-4-oxo-4-((2-(4-(trifluoromethyl)phenyl)propan-2-yl)amino)butanoic acid;
2-methylene-4-oxo-4-((1-(4-(trifluoromethyl)phenyl)cyclobutyl)amino)butanoic acid;
2-methylene-4-oxo-4-((4-(trifluoromethyl)benzyl)amino)butanoic acid;
2-methylene-4-oxo-4-((2,2,2-trifluoro-1-(4-(trifluoromethyl)phenyl)ethyl)amino)butanoic acid;
(S)-2-methylene-4-oxo-4-((1-(4-(trifluoromethyl)phenyl)propyl)amino)butanoic acid;
1-(methylsulfonyl)piperidin-4-yl 2-methylene-4-oxo-4-((1-(4-(trifluoromethyl)phenyl)cyclobutyl) amino)butanoate;
4-((cyclopropyl(4-(trifluoromethyl)phenyl)methyl)amino)-2-methylene-4-oxobutanoic acid;
2-methylene-4-oxo-4-((1-(4-(trifluoromethyl)phenyl)cyclopropyl)amino)butanoic acid;
(R)-4-((2-hydroxy-1-(4-(trifluoromethyl)phenyl)ethyl)amino)-2-methylene-4-oxobutanoic acid;
2-methylene-4-oxo-4-((3-(4-(trifluoromethyl)phenyl)oxetan-3-yl)amino)butanoic acid;
2-methylene-4-oxo-4-((1-(4-(trifluoromethyl)phenyl)cyclopentyl)amino)butanoic acid;
(S)-4-((1-(4-chlorophenyl)ethyl)amino)-2-methylene-4-oxobutanoic acid;
(S)-4-((1-(4-bromophenyl)ethyl)amino)-2-methylene-4-oxobutanoic acid;
(S)-4-((1-(4-bromophenyl)ethyl)amino)-2-methylene-4-oxobutanoic acid;
(S)-4-((1-(4-(difluoromethyl)phenyl)ethyl)amino)-2-methylene-4-oxobutanoic acid;
(S)-4-((1-(3-fluoro-4-(trifluoromethyl)phenyl)ethyl)amino)-2-methylene-4-oxobutanoic acid;
(S)-2-methylene-4-oxo-4-((1-(4-(perfluoroethyl)phenyl)ethyl)amino)butanoic acid;
(R)-2-methylene-4-oxo-4-((2,2,2-trifluoro-1-(4-(trifluoromethyl)phenyl)ethyl)amino)butanoic acid;
1-(methylsulfonyl)piperidin-4-yl (S)-2-methylene-4-oxo-4-((1-(4-(trifluoromethyl)phenyl)ethyl)amino)butanoate;
4-((cyclopropyl(4-(trifluoromethyl)phenyl)methyl)amino)-2-methylene-4-oxobutanoic acid;
1-methylpiperidin-4-yl (S)-2-methylene-4-oxo-4-((1-(4-(trifluoromethyl)phenyl)ethyl)amino)butanoate;
2-(methylsulfonamido)ethyl (S)-2-methylene-4-oxo-4-((1-(4-(trifluoromethyl)phenyl)ethyl)amino)butanoate;
2-(dimethylamino)ethyl (S)-2-methylene-4-oxo-4-((1-(4-(trifluoromethyl)phenyl)ethyl)amino)butanoate;
1,1-dioxidothietan-3-yl (S)-2-methylene-4-oxo-4-((1-(4-(trifluoromethyl)phenyl)ethyl)amino)butanoate;
3-methyloxetan-3-yl (S)-2-methylene-4-oxo-4-((1-(4-(trifluoromethyl)phenyl)ethyl)amino)butanoate;
2-morpholinoethyl 2-(diethoxyphosphoryl)-4-oxo-4-(((S)-1-(4-(trifluoromethyl)phenyl)ethyl)amino)butanoate;
(S)-2-methylene-4-oxo-4-((1-(5-(trifluoromethyl)pyridin-2-yl)ethyl)amino)butanoic acid;
1-(methylsulfonyl)piperidin-4-yl (S)-2-methylene-4-oxo-4-((1-(5-(trifluoromethyl)pyridin-2-yl)ethyl)amino)butanoate;
1-methylpiperidin-4-yl (S)-2-methylene-4-oxo-4-((1-(5-(trifluoromethyl)pyridin-2-yl)ethyl)amino)butanoate;
1,1-dioxidothietan-3-yl (S)-2-methylene-4-oxo-4-((1-(5-(trifluoromethyl)pyridin-2-yl)ethyl)amino)butanoate;
2-hydroxyethyl (S)-2-methylene-4-oxo-4-((1-(5-(trifluoromethyl)pyridin-2-yl)ethyl)amino)butanoate;
1-acetylpiperidin-4-yl (S)-2-methylene-4-oxo-4-((1-(5-(trifluoromethyl)pyridin-2-yl)ethyl)amino)butanoate;
1-acetylazetidin-3-yl (S)-2-methylene-4-oxo-4-((1-(5-(trifluoromethyl)pyridin-2-yl)ethyl)amino)butanoate;
1-(methylsulfonyl)azetidin-3-yl (S)-2-methylene-4-oxo-4-((1-(5-(trifluoromethyl)pyridin-2-yl)ethyl)amino)butanoate;
(S)-2-methylene-4-oxo-4-((1-(6-(trifluoromethyl)pyridin-3-yl)ethyl)amino)butanoic acid;
2-methylene-4-oxo-4-((1-(5-(trifluoromethyl)pyridin-2-yl)cyclobutyl)amino)butanoic acid;
2-methylene-4-oxo-4-((5-(trifluoromethyl)-2,3-dihydro-1H-inden-1-yl)amino)butanoic acid;
(S,Z)-2-(2-oxo-2-((1-(4-(trifluoromethyl)phenyl)ethyl)amino)ethyl)but-2-enoic acid; and
(S,E)-2-(2-oxo-2-((1-(4-(trifluoromethyl)phenyl)ethyl)amino)ethyl)but-2-enoic acid;
(S,E)-2-(2-oxo-2-((1-(4-(trifluoromethyl)phenyl)ethyl)amino)ethyl)pent-2-enoic acid;
(S,E)-4-methyl-2-(2-oxo-2-((1-(4-(trifluoromethyl)phenyl)ethyl)amino)ethyl)pent-2-enoic acid;
(S,E)-5-methyl-2-(2-oxo-2-((1-(4-(trifluoromethyl)phenyl)ethyl)amino)ethyl)hex-2-enoic acid;
(S,E)-2-(2-oxo-2-((1-(4-(trifluoromethyl)phenyl)ethyl)amino)ethyl)-5-phenylpent-2-enoic acid;
(S,E)-2-benzylidene-4-oxo-4-((1-(4-(trifluoromethyl)phenyl)ethyl)amino)butanoic acid;
(S,Z)-2-benzylidene-4-oxo-4-((1-(4-(trifluoromethyl)phenyl)ethyl)amino)butanoic acid;
(S,E)-4-(dimethylamino)-2-(2-oxo-2-((1-(4-(trifluoromethyl)phenyl)ethyl)amino)ethyl)but-2-enoic acid;
(S)-3,3-dimethyl-2-methylene-4-oxo-4-((1-(4-(trifluoromethyl)phenyl)ethyl)amino)butanoic acid;
3-methyl-2-methylene-4-oxo-4-((2-(4-(trifluoromethyl)phenyl)propan-2-yl)amino)butanoic acid;
3-methyl-2-methylene-4-oxo-4-((2-(4-(trifluoromethyl)phenyl)propan-2-yl)amino)butanoic acid ISOMER 1;
3-methyl-2-methylene-4-oxo-4-((2-(4-(trifluoromethyl)phenyl)propan-2-yl)amino)butanoic acid ISOMER 2;
3-methyl-2-methylene-4-oxo-4-(((S)-1-(4-(trifluoromethyl)phenyl)ethyl)amino)butanoic acid ISOMER 1;
3-methyl-2-methylene-4-oxo-4-(((S)-1-(4-(trifluoromethyl)phenyl)ethyl)amino)butanoic acid;
(S)-2-methylene-4-oxo-4-((1-(4-(pentafluoro-λ6-sulfaneyl)phenyl)ethyl)amino)butanoic acid ISOMER 2;
(S)-3-(1H-tetrazol-5-yl)-N-(1-(4-(trifluoromethyl)phenyl)ethyl)but-3-enamide;
or a pharmaceutically acceptable salt and/or solvate of any one thereof.

26: A pharmaceutical composition comprising a compound or a pharmaceutically acceptable salt and/or solvate thereof according to claim 1.

27. (canceled)

28: A method of treating or preventing an inflammatory disease or a disease associated with an undesirable immune response comprising administering an effective amount of a compound or a pharmaceutically acceptable salt and/or solvate thereof according to claim 1.

29: The method according to claim 28 wherein the inflammatory disease or disease associated with an undesirable immune response is a disease selected from the group consisting of: psoriasis (including chronic plaque, erythrodermic, pustular, guttate, inverse and nail variants), asthma, chronic obstructive pulmonary disease (COPD, including chronic bronchitis and emphysema), heart failure (including left ventricular failure), myocardial infarction, angina pectoris, other atherosclerosis and/or atherothrombosis-related disorders (including peripheral vascular disease and ischaemic stroke), a mitochondrial and neurodegenerative disease (such as Parkinson's disease, Alzheimer's disease, Huntington's disease, amyotrophic lateral sclerosis, retinitis pigmentosa or mitochondrial encephalomyopathy), autoimmune paraneoplastic retinopathy, transplantation rejection (including antibody-mediated and T cell-mediated forms), multiple sclerosis, transverse myelitis, ischaemia-reperfusion injury (e.g. during elective surgery such as cardiopulmonary bypass for coronary artery bypass grafting or other cardiac surgery, following percutaneous coronary intervention, following treatment of acute ST-elevation myocardial infarction or ischaemic stroke, organ transplantation, or acute compartment syndrome), AGE-induced genome damage, an inflammatory bowel disease (e.g. Crohn's disease or ulcerative colitis), primary sclerosing cholangitis (PSC), PSC-autoimmune hepatitis overlap syndrome, non-alcoholic fatty liver disease (non-alcoholic steatohepatitis), rheumatica, granuloma annulare, cutaneous lupus erythematosus (CLE), systemic lupus erythematosus (SLE), lupus nephritis, drug-induced lupus, autoimmune myocarditis or myopericarditis, Dressler's syndrome, giant cell myocarditis, post-pericardiotomy syndrome, drug-induced hypersensitivity syndromes (including hypersensitivity myocarditis), eczema, sarcoidosis, erythema nodosum, acute disseminated encephalomyelitis (ADEM), neuromyelitis optica spectrum disorders, MOG (myelin oligodendrocyte glycoprotein) antibody-associated disorders (including MOG-EM), optic neuritis, CLIPPERS (chronic lymphocytic inflammation with pontine perivascular enhancement responsive to steroids), diffuse myelinoclastic sclerosis, Addison's disease, alopecia areata, ankylosing spondylitis, other spondyloarthritides (including peripheral spondyloarthritis, that is associated with psoriasis, inflammatory bowel disease, reactive arthritis or juvenile onset forms), antiphospholipid antibody syndrome, autoimmune hemolytic anaemia, autoimmune hepatitis, autoimmune inner ear disease, pemphigoid (including bullous pemphigoid, mucous membrane pemphigoid, cicatricial pemphigoid, herpes gestationis or pemphigoid gestationis, ocular cicatricial pemphigoid), linear IgA disease, Behçet's disease, celiac disease, Chagas disease, dermatomyositis, diabetes mellitus type I, endometriosis, Goodpasture's syndrome, Graves' disease, Guillain-Barre syndrome and its subtypes (including acute inflammatory demyelinating polyneuropathy, AIDP, acute motor axonal neuropathy (AMAN), acute motor and sensory axonal neuropathy (AMSAN), pharyngeal-cervical-brachial variant, Miller-Fisher variant and Bickerstaff s brainstem encephalitis), progressive inflammatory neuropathy, Hashimoto's disease, hidradenitis suppurativa, inclusion body myositis, necrotising myopathy, Kawasaki disease, IgA nephropathy, Henoch-Schonlein purpura, idiopathic thrombocytopenic purpura, thrombotic thrombocytopenic purpura (TTP), Evans' syndrome, interstitial cystitis, mixed connective tissue disease, undifferentiated connective tissue disease, morphea, myasthenia gravis (including MuSK antibody positive and seronegative variants), narcolepsy, neuromyotonia, pemphigus vulgaris, pernicious anaemia, psoriatic arthritis, polymyositis, primary biliary cholangitis (also known as primary biliary cirrhosis), rheumatoid arthritis, palindromic rheumatism, schizophrenia, autoimmune (meningo-)encephalitis syndromes, scleroderma, Sjogren's syndrome, stiff person syndrome, polymylagia rheumatica, giant cell arteritis (temporal arteritis), Takayasu arteritis, polyarteritis nodosa, Kawasaki disease, granulomatosis with polyangitis (GPA; formerly known as Wegener's granulomatosis), eosinophilic granulomatosis with polyangiitis (EGPA; formerly known as Churg-Strauss syndrome), microscopic polyarteritis/polyangiitis, hypocomplementaemic urticarial vasculitis, hypersensitivity vasculitis, cryoglobulinemia, thromboangiitis obliterans (Buerger's disease), vasculitis, leukocytoclastic vasculitis, vitiligo, acute disseminated encephalomyelitis, adrenoleukodystrophy, Alexander's disease, Alper's disease, balo concentric sclerosis or Marburg disease, cryptogenic organising pneumonia (formerly known as bronchiolitis obliterans organizing pneumonia), Canavan disease, central nervous system vasculitic syndrome, Charcot-Marie-Tooth disease, childhood ataxia with central nervous system hypomyelination, chronic inflammatory demyelinating polyneuropathy (CIDP), diabetic retinopathy, globoid cell leukodystrophy (Krabbe disease), graft-versus-host disease (GVHD) (including acute and chronic forms, as well as intestinal GVHD), hepatitis C (HCV) infection or complication, herpes simplex viral infection or complication, human immunodeficiency virus (HIV) infection or complication, lichen planus, monomelic amyotrophy, cystic fibrosis, pulmonary arterial hypertension (PAH, including idiopathic PAH), lung sarcoidosis, idiopathic pulmonary fibrosis, paediatric asthma, atopic dermatitis, allergic dermatitis, contact dermatitis, allergic rhinitis, rhinitis, sinusitis, conjunctivitis, allergic conjunctivitis, keratoconjunctivitis sicca, dry eye, xerophthalmia, glaucoma, macular oedema, diabetic macular oedema, central retinal vein occlusion (CRVO), macular degeneration (including dry and/or wet age related macular degeneration, AMD), post-operative cataract inflammation, uveitis (including posterior, anterior, intermediate and pan uveitis), iridocyclitis, scleritis, corneal graft and limbal cell transplant rejection, gluten sensitive enteropathy (coeliac disease), dermatitis herpetiformis, eosinophilic esophagitis, achalasia, autoimmune dysautonomia, autoimmune encephalomyelitis, autoimmune oophoritis, autoimmune orchitis, autoimmune pancreatitis, aortitis and periaortitis, autoimmune retinopathy, autoimmune urticaria, (idiopathic) Castleman's disease, Cogan's syndrome, IgG4-related disease, retroperitoneal fibrosis, juvenile idiopathic arthritis including systemic juvenile idiopathic arthritis (Still's disease), adult-onset Still's disease, ligneous conjunctivitis, Mooren's ulcer, pityriasis lichenoides et varioliformis acuta (PLEVA, also known as Mucha-Habermann disease), multifocal motor neuropathy (MMN), paediatric acute-onset neuropsychiatric syndrome (PANS) (including paediatric autoimmune neuropsychiatric disorders associated with streptococcal infections (PANDAS)), paraneoplastic syndromes (including paraneoplastic cerebellar degeneration, Lambert-Eaton myaesthenic syndrome, limbic encephalitis, brainstem encephalitis, opsoclonus myoclonus ataxia syndrome, anti-NMDA receptor encephalitis, thymoma-associated multiorgan autoimmunity), perivenous encephalomyelitis, reflex sympathetic dystrophy, relapsing polychondritis, sperm & testicular autoimmunity, Susac's syndrome, Tolosa-Hunt syndrome, Vogt-Koyanagi-Harada Disease, anti-synthetase syndrome, autoimmune enteropathy, immune dysregulation polyendocrinopathy enteropathy X-linked (IPEX), microscopic colitis, autoimmune lymphoproliferative syndrome (ALPS), autoimmune polyendocrinopathy-candidiasis-ectodermal dystrophy syndrome (APEX), gout, pseudogout, amyloid (including AA or secondary amyloidosis), eosinophilic fasciitis (Shulman syndrome) progesterone hypersensitivity (including progesterone dermatitis), amilial Mediterranean fever (FMF), tumour necrosis factor (TNF) receptor-associated periodic fever syndrome (TRAPS), hyperimmunoglobulinaemia D with periodic fever syndrome (HIDS), PAPA (pyogenic arthritis, pyoderma gangrenosum, severe cystic acne) syndrome, deficiency of interleukin-1 receptor antagonist (DIRA), deficiency of the interleukin-36-receptor antagonist (DITRA), cryopyrin-associated periodic syndromes (CAPS) (including familial cold autoinflammatory syndrome [FCAS], Muckle-Wells syndrome, neonatal onset multisystem inflammatory disease [NOMID]), NLRP12-associated autoinflammatory disorders (NLRP12AD), periodic fever aphthous stomatitis (PFAPA), chronic atypical neutrophilic dermatosis with lipodystrophy and elevated temperature (CANDLE), Majeed syndrome, Blau syndrome (also known as juvenile systemic granulomatosis), macrophage activation syndrome, chronic recurrent multifocal osteomyelitis (CRMO), familial cold autoinflammatory syndrome, mutant adenosine deaminase 2 and monogenic interferonopathies (including Aicardi-Goutières syndrome, retinal vasculopathy with cerebral leukodystrophy, spondyloenchondrodysplasia, STING [stimulator of interferon genes]-associated vasculopathy with onset in infancy, proteasome associated autoinflammatory syndromes, familial chilblain lupus, dyschromatosis symmetrica hereditaria), Schnitzler syndrome; familial cylindromatosis, congenital B cell lymphocytosis, OTULIN-related autoinflammatory syndrome, type 2 diabetes mellitus, insulin resistance and the metabolic syndrome (including obesity-associated inflammation), atherosclerotic disorders (e.g. myocardial infarction, angina, ischaemic heart failure, ischaemic nephropathy, ischaemic stroke, peripheral vascular disease, aortic aneurysm), renal inflammatory disorders (e.g. diabetic nephropathy, membranous nephropathy, minimal change disease, crescentic glomerulonephritis, acute kidney injury, renal transplantation).

30: The compound or a pharmaceutically acceptable salt and/or solvate thereof according to claim 1 for use in combination with a further therapeutic agent, such as a corticosteroid (glucocorticoid), retinoid (e.g. acitretin, isotretinoin, tazarotene), anthralin, vitamin D analogue (e.g. cacitriol, calcipotriol), calcineurin inhibitors (e.g. tacrolimus, pimecrolimus), phototherapy or photochemotherapy (e.g. psoralen ultraviolet irradiation, PUVA) or other form of ultraviolet light irradiation therapy, ciclosporine, a thiopurine (e.g. azathioprine, 6-mercaptopurine), methotrexate, an anti-TNFα agents (e.g. infliximab, etanercept, adalimumab, certolizumab, golimumab or a biosimilar), phosphodiesterase-4 (PDE4) inhibition (e.g. apremilast, crisaborole), anti-IL-17 agent (e.g. brodalumab, ixekizumab, secukinumab), anti-IL12/IL-23 agent (e.g. ustekinumab, briakinumab), anti-IL-23 agent (e.g. guselkumab, tildrakizumab), JAK (Janus Kinase) inhibitor (e.g. tofacitinib, ruxolitinib, baricitinib, filgotinib, upadacitinib), plasma exchange, intravenous immune globulin (IVIG), cyclophosphamide, anti-CD20 B cell depleting agent (e.g. rituximab, ocrelizumab, ofatumumab, obinutuzumab), anthracycline analogue (e.g. mitoxantrone), cladribine, sphingosine 1-phosphate receptor modulator or sphingosine analogue (e.g. fingolimod, siponimod, ozanimod, etrasimod), interferon beta preparation (including interferon beta 1b/1a), glatiramer, anti-CD3 therapy (e.g. OKT3), anti-CD52 targeting agent (e.g. alemtuzumab), leflunomide, teriflunomide, gold compound, laquinimod, potassium channel blocker (e.g. dalfampridine/4-aminopyridine), mycophenolic acid, mycophenolate mofetil, purine analogue (e.g. pentostatin), mTOR (mechanistic target of rapamycin) pathway inhibitor (e.g. sirolimus, everolimus), anti-thymocyte globulin (ATG), IL-2 receptor (CD25) inhibitor (e.g. basiliximab, daclizumab), anti-IL-6 receptor or anti-IL-6 agent (e.g. tocilizumab, siltuximab), Bruton's tyrosine kinase (BTK) inhibitor (e.g. ibrutinib), tyrosine kinase inhibitor (e.g. imatinib), ursodeoxycholic acid, hydroxychloroquine, chloroquine, B cell activating factor (BAFF, also known as BLyS, B lymphocyte stimulator) inhibitor (e.g. belimumab, blisibimod), other B cell targeted therapy including a fusion protein targeting both APRIL (A PRoliferation-Inducing Ligand) and BLyS (e.g. atacicept), PI3K inhibitor including pan-inhibitor or one targeting the p110δ and/or p110γ containing isoforms (e.g. idelalisib, copanlisib, duvelisib), an interferon α receptor inhibitor (e.g. anifrolumab, sifalimumab), T cell co-stimulation blocker (e.g. abatacept, belatacept), thalidomide and its derivatives (e.g. lenalidomide), dapsone, clofazimine, a leukotriene antagonist (e.g. montelukast), theophylline, anti-IgE therapy (e.g. omalizumab), an anti-IL-5 agent (e.g. mepolizumab, reslizumab), a long-acting muscarinic agent (e.g. tiotropium, aclidinium, umeclidinium), a PDE4 inhibitor (e.g. roflumilast), riluzole, a free radical scavenger (e.g. edaravone), a proteasome inhibitor (e.g. bortezomib), a complement cascade inhibitor including one directed against C5 (e.g. eculizumab), immunoadsor, antithymocyte globulin, 5-aminosalicylates and their derivatives (e.g. sulfasalazine, balsalazide, mesalamine), an anti-integrin agent including one targeting α4β1 and/or α4β7 integrins (e.g. natalizumab, vedolizumab), an anti-CD11-α agent (e.g. efalizumab), a non-steroidal anti-inflammatory drug (NSAID) including a salicylate (e.g. aspirin), a propionic acid (e.g. ibuprofen, naproxen), an acetic acid (e.g. indomethacin, diclofenac, etodolac), an oxicam (e.g. meloxicam) a fenamate (e.g. mefenamic acid), a selective or relatively selective COX-2 inhibitor (e.g. celecoxib, etroxicoxib, valdecoxib and etodolac, meloxicam, nabumetone), colchicine, an IL-4 receptor inhibitor (e.g. dupilumab), topical/contact immunotherapy (e.g. diphenylcyclopropenone, squaric acid dibutyl ester), anti-IL-1 receptor therapy (e.g. anakinra), IL-1β inhibitor (e.g. canakinumab), IL-1 neutralising therapy (e.g. rilonacept), chlorambucil, a specific antibiotic with immunomodulatory properties and/or ability to modulate NRF2 (e.g. tetracyclines including minocycline, clindamycin, macrolide antibiotics), anti-androgenic therapy (e.g. cyproterone, spironolactone, finasteride), pentoxifylline, ursodeoxycholic acid, obeticholic acid, fibrate, a cystic fibrosis transmembrane conductance regulator (CFTR) modulator, a VEGF (vascular endothelial growth factor) inhibitor (e.g. bevacizumab, ranibizumab, pegaptanib, aflibercept), pirfenidone or mizoribine.

31: A process for the preparation of a compound according to claim 1 or a salt and/or solvate thereof comprising:
A. for a compound in which R^{Z} is C(O)OR^{B}:
reacting a compound of formula (II): 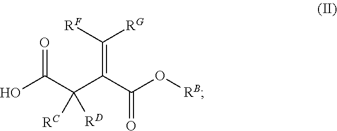
or a salt thereof;
with amine R^{A}—NH₂ (V), or a salt thereof;
wherein R^{B} is: 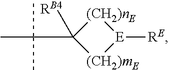
or
B. for a compound in which R^{Z} is C(O)OR^{B}:
deprotecting a compound of formula (VII): 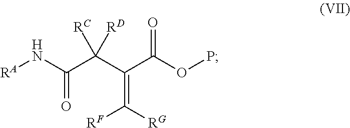
or a salt thereof,
wherein P is a carboxylic acid protecting group; or
C. for a compound in which R^{Z} is C(O)OR^{B}:
reacting a compound of formula (VIIIa) or (VIIIb): 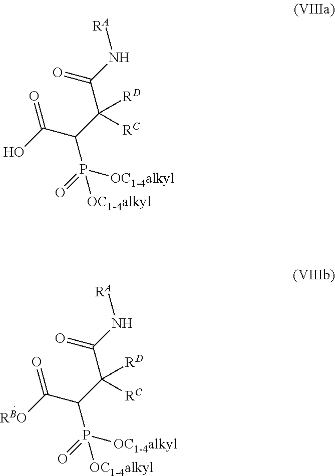
or a salt thereof;
with aqueous C(R^{F})(R^{G})═O in the presence of a base; or
D. for a compound in which R^{Z} is C(O)OR^{B}, where R^{B} is H and R^{A} is CH₂(substituted phenyl) or CH₂(substituted pyridyl), wherein the CH₂ group is optionally substituted with one R^{A3} and the phenyl or pyridyl group is substituted with one or more R^{A4}:
reacting a compound of formula (Va): 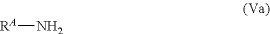
wherein R^{A} is CH₂(substituted phenyl) or CH₂(substituted phenyl), wherein the CH₂ group is optionally substituted with one or two R^{A3} and the phenyl or pyridyl group is substituted with one or more R^{A4};
with an itaconic anhydride derivative of formula (XX): 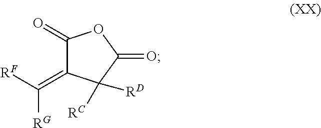
wherein R^{C}, R^{D}, R^{F} and R^{G} are defined in claim 1; or
E. for a compound in which R^{F} and R^{G} are both H and R^{Z} is C(O)OR^{B}:
reacting a compound of formula (XXV): 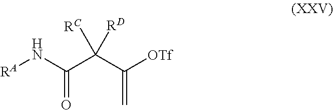
with triphenylphosphine, palladium acetate and carbon monoxide, followed by a base such as triethylamine and a carboxylic acid such as acetic acid to give a compound of formula (I) in which R^{B} is H; and optionally
reacting a compound of formula (I) in which R^{Z} is C(O)OH with a compound of formula (VI): 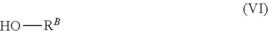
where in R^{B} is not H;
to give a compound of formula (I) in which R^{Z} is C(O)OR^{B}, where in R^{B} is as defined in claim 1 except that it is not H; or
F. for a compound in which R^{Z} is tetrazol-5-yl:
reacting a compound of formula (XXXI): 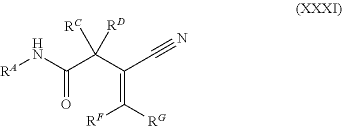
or a salt thereof;
with trimethylsilyl azide (TMSN₃) in the presence of a catalyst. Suitable catalysts include dibutyl tin oxide.

32. A compound of formula (II): 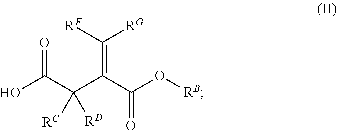
wherein
R^{C} and R^{D} are each independently H, C₁₋₂ alkyl, hydroxy, methoxy or fluoro;
each of R^{F} and R^{G} is independently selected from H, phenyl and C₁₋₄ alkyl optionally substituted with phenyl, OR^{K} or N(R^{K})(R^{L}), wherein each of R^{K} and R^{L} is independently selected from H, methyl and ethyl, provided that at least one of R^{F} and R^{G} is H;
R^{B} is: 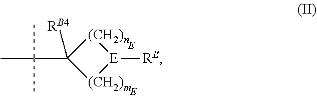
R^{B4} is H or methyl;
E is N, O or SO₂;
wherein when E is N, R^{E} is selected from the group consisting of R^{9B}, C(O)R^{9B} and SO₂R^{9B} wherein R^{9B} is C₁₋₄ alkyl; and when E is O or SO₂, R^{E} is absent;
nE and mE are independently 1 or 2; or
a compound of formula (VII): 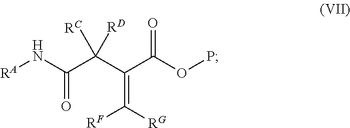
wherein R^{A} is selected from the group consisting of C₆₋₁₀ cycloalkyl optionally fused to phenyl, CH₂(phenyl) and CH₂(pyridyl);
wherein when R^{A} is C₆₋₁₀ cycloalkyl optionally fused to phenyl, the cycloalkyl ring s optionally substituted on an available carbon atom by one or more R^{A2}, wherein each R^{A2} is independently selected from the group consisting of methyl, halo and trifluoromethyl, and/or two R^{A2} are attached to the same carbon atom and join to form a C₃₋₆ cycloalkyl or a 4-6 membered heterocyclic ring; and the fused phenyl ring is optionally substituted on an available carbon atom by one or more substituents selected from the group consisting of C₁₋₄ haloalkyl and halo;
wherein when R^{A} is CH₂(phenyl) or CH₂(pyridyl):
the CH₂ group is optionally substituted by one or two R^{A3} wherein each R^{A3} is independently selected from the group consisting of C₁₋₄ alkyl, C₃₋₅ cycloalkyl, C₁₋₄ haloalkyl and C₁₋₂ hydroxyalkyl, or two R^{A3} groups are attached to the CH₂ carbon atom and join to form a C₃₋₆ cycloalkyl or a 4- or 5-membered heterocyclic ring; and
the phenyl or pyridyl group is optionally substituted by one or more R^{A4}, wherein each R^{A4} is independently selected from the group consisting of C₁₋₄ haloalkyl, halo, SF₅ or SC₁₋₄ haloalkyl;
R^{C} and R^{D} are each independently H, C₁₋₂ alkyl, hydroxy, methoxy or fluoro;
each of R^{F} and R^{G} is independently selected from H, phenyl and C₁₋₄ alkyl optionally substituted with phenyl, OR^{K} or N(R^{K})(R^{L}), wherein each of R^{K} and R^{L} is independently selected from H, methyl and ethyl, provided that at least one of R^{F} and R^{G} is H; and
wherein when the CH₂ of the CH₂(substituted phenyl) or CH₂(substituted pyridyl) of R^{A} is substituted by one R^{A3} the stereochemistry of the carbon to which R^{A3} is attached is as follows: 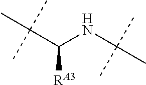
wherein the dashed lines indicate attachment to the remainder of the compound of formula (I):
and wherein when R^{A4} is Cl, the CH₂ group is unsubstituted or is substituted by one R^{A3}; and wherein P is a carboxylic acid protecting group; or
a compound of formula (VIIIa) or (VIIIb): 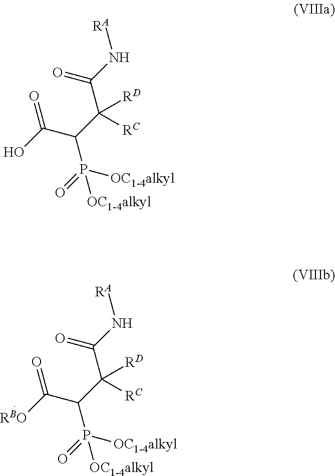
wherein R^{A} is selected from the group consisting of C₆₋₁₀ cycloalkyl optionally fused to phenyl, CH₂(phenyl) and CH₂(pyridyl):
wherein when R^{A} is C₆₋₁₀ cycloalkyl optionally fused to phenyl, the cycloalkyl ring s optionally substituted on an available carbon atom by one or more R^{A2}, wherein each R^{A2} is independently selected from the group consisting of methyl, halo and trifluoromethyl, and/or two R^{A2} are attached to the same carbon atom and join to form a C₃₋₆ cycloalkyl or a 4-6 membered heterocyclic ring; and the fused phenyl ring is optionally substituted on an available carbon atom by one or more substituents selected from the group consisting of C₁₋₄ haloalkyl and halo;
wherein when R^{A} is CH₂(phenyl) or CH₂(pyridyl);
the CH₂ group is optionally substituted by one or two R^{A3} wherein each R^{A3} is independently selected from the group consisting of C₁₋₄ alkyl, C₃₋₅ cycloalkyl, C₁₋₄ haloalkyl and C₁₋₂ hydroxyalkyl, or two R^{A3} groups are attached to the CH₂ carbon atom and join to form a C₃₋₆ cycloalkyl or a 4- or 5-membered heterocyclic ring; and
the phenyl or pyridyl group is optionally substituted by one or more R^{A4}, wherein each R^{A4} is independently selected from the group consisting of C₁₋₄ haloalkyl, halo, SF₅ or SC₁₋₄ haloalkyl;
R^{C} and R^{D} are each independently H, C₁₋₂ alkyl, hydroxy, methoxy or fluoro;
wherein when the CH₂ of the CH₂(substituted phenyl) or CH₂(substituted pyridyl) of R^{A} is substituted by one R^{A3} the stereochemistry of the carbon to which R^{A3} is attached is as follows: 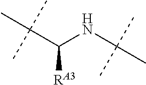
wherein the dashed lines indicate attachment to the remainder of the compound of formula (I);
and wherein when R^{A4} is Cl, the CH₂ group is unsubstituted or is substituted by one R^{A3}; or
a compound of formula (XXXI): 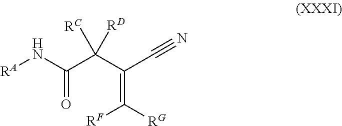
wherein R^{A} is selected from the group consisting of C₆₋₁₀ cycloalkyl optionally fused to phenyl, CH₂(phenyl) and CH₂(pyridyl);
wherein when R^{A} is C₆₋₁₀ cycloalkyl optionally fused to phenyl, the cycloalkyl ring s optionally substituted on an available carbon atom by one or more R^{A2}, wherein each R^{A2} is independently selected from the group consisting of methyl, halo and trifluoromethyl, and/or two R^{A2} are attached to the same carbon atom and join to form a C₃₋₆ cycloalkyl or a 4-6 membered heterocyclic ring; and the fused phenyl ring is optionally substituted on an available carbon atom by one or more substituents selected from the group consisting of C₁₋₄ haloalkyl and halo;
wherein when R^{A} is CH₂(phenyl) or CH₂(pyridyl):
the CH₂ group is optionally substituted by one or two R^{A3} wherein each R^{A3} is independently selected from the group consisting of C₁₋₄ alkyl, C₃₋₅ cycloalkyl, C₁₋₄ haloalkyl and C₁₋₂ hydroxyalkyl, or two R^{A3} groups are attached to the CH₂ carbon atom and join to form a C₃₋₆ cycloalkyl or a 4- or 5-membered heterocyclic ring; and
the phenyl or pyridyl group is optionally substituted by one or more R^{A4}, wherein each R^{A4} is independently selected from the group consisting of C₁₋₄ haloalkyl, halo, SF₅ or SC₁₋₄ haloalkyl;
R^{C} and R^{D} are each independently H, C₁₋₂ alkyl, hydroxy, methoxy or fluoro;
each of R^{F} and R^{G} is independently selected from H, phenyl and C₁₋₄ alkyl optionally substituted with phenyl, OR^{K} or N(R^{K})(R^{L}), wherein each of R^{K} and R^{L} is independently selected from H, methyl and ethyl, provided that at least one of R^{F} and R^{G} is H; and wherein when the CH₂ of the CH₂(substituted phenyl) or CH₂(substituted pyridyl) of R^{A} is substituted by one R^{A3} the stereochemistry of the carbon to which R^{A3} is attached is as follows: 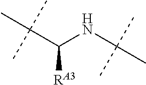
wherein the dashed lines indicate attachment to the remainder of the compound of formula (I);
and wherein when R^{A4} is Cl, the CH₂ group is unsubstituted or is substituted by one R^{A3}; or
a compound of formula (XXV): 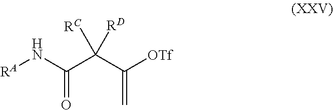
wherein R^{A} is selected from the group consisting of C₆₋₁₀ cycloalkyl optionally fused to phenyl, CH₂(phenyl) and CH₂(pyridyl);
wherein when R^{A} is C₆₋₁₀ cycloalkyl optionally fused to phenyl, the cycloalkyl ring s optionally substituted on an available carbon atom by one or more R^{A2}, wherein each R^{A2} is independently selected from the group consisting of methyl, halo and trifluoromethyl, and/or two R^{A2} are attached to the same carbon atom and join to form a C₃₋₆ cycloalkyl or a 4-6 membered heterocyclic ring; and the fused phenyl ring is optionally substituted on an available carbon atom by one or more substituents selected from the group consisting of C₁₋₄ haloalkyl and halo;
wherein when R^{A} is CH₂(phenyl) or CH₂(pyridyl):
the CH₂ group is optionally substituted by one or two R^{A3} wherein each R^{A3} is independently selected from the group consisting of C₁₋₄ alkyl, C₃₋₅ cycloalkyl, C₁₋₄ haloalkyl and C₁₋₂ hydroxyalkyl, or two R^{A3} groups are attached to the CH₂ carbon atom and join to form a C₃₋₆ cycloalkyl or a 4- or 5-membered heterocyclic ring; and
the phenyl or pyridyl group is optionally substituted by one or more R^{A4}, wherein each R^{A4} is independently selected from the group consisting of C₁₋₄ haloalkyl, halo, SF₅ or SC₁₋₄ haloalkyl:
R^{C} and R^{D} are each independently H, C₁₋₂ alkyl, hydroxy, methoxy or fluoro;
wherein when the CH₂ of the CH₂(substituted phenyl) or CH₂(substituted pyridyl) of R^{A} is substituted by one R^{A3} the stereochemistry of the carbon to which R^{A3} is attached is as follows: 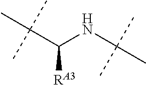
wherein the dashed lines indicate attachment to the remainder of the compound of formula (I);
and wherein when R^{A4} is Cl, the CH₂ group is unsubstituted or is substituted by one R^{A3}; or
a salt and/or solvate of any of the above.
